SEQUENCE LISTING (116 sequences)

SEQ ID NO: 1 - DNA, 21 bp, Artificial Sequence
  Description of Artificial Sequence; note = synthetic construct
ctggcagaccagcctgtgttt

SEQ ID NO: 2 - DNA, 21 bp, Artificial Sequence
  Description of Artificial Sequence; note = synthetic construct
gtggcagctgacatgaatgtt

SEQ ID NO: 3 - DNA, 21 bp, Artificial Sequence
  Description of Artificial Sequence; note = synthetic construct
gctcaactcagcacacacttt

SEQ ID NO: 4 - DNA, 19 bp, Artificial Sequence
  Description of Artificial Sequence; note = synthetic construct
ggcgacacgcatgccaaag

SEQ ID NO: 5 - DNA, 19 bp, Artificial Sequence
  Description of Artificial Sequence; note = synthetic construct
ggcctctaggcgtcctaga

SEQ ID NO: 6 - DNA, 19 bp, Artificial Sequence
  Description of Artificial Sequence; note = synthetic construct
ggcgctctgggaccactct

SEQ ID NO: 7 - DNA, 19 bp, Artificial Sequence
  Description of Artificial Sequence; note = synthetic construct
tccacgtagtttagtaagt

SEQ ID NO: 8 - DNA, 19 bp, Artificial Sequence
  Description of Artificial Sequence; note = synthetic construct
gtggttctgcttgtctttc

SEQ ID NO: 9 - DNA, 19 bp, Artificial Sequence
  Description of Artificial Sequence; note = synthetic construct
tgcctgtactgactaatat

SEQ ID NO: 10 - DNA, 19 bp, Artificial Sequence
  Description of Artificial Sequence; note = synthetic construct
catgcctgtactgactaat

SEQ ID NO: 11 - DNA, 19 bp, Artificial Sequence
  Description of Artificial Sequence; note = synthetic construct
gcgcagctctgggcagaag

SEQ ID NO: 12 - DNA, 19 bp, Artificial Sequence
  Description of Artificial Sequence; note = synthetic construct
gtccgagttctgtgaagaa

SEQ ID NO: 13 - DNA, 19 bp, Artificial Sequence
  Description of Artificial Sequence; note = synthetic construct
ggctgtgacctgccagaaa

SEQ ID NO: 14 - DNA, 19 bp, Artificial Sequence
  Description of Artificial Sequence; note = synthetic construct
ggccgtcagtaatgtttca

SEQ ID NO: 15 - DNA, 19 bp, Artificial Sequence
  Description of Artificial Sequence; note = synthetic construct
gtgcgattcttgacattga

SEQ ID NO: 16 - DNA, 23 bp, Artificial Sequence
  Description of Artificial Sequence; note = synthetic construct
aaggtttgtacctcaaatgaatt

SEQ ID NO: 17 - DNA, 21 bp, Artificial Sequence
  Description of Artificial Sequence; note = synthetic construct
ggagaagtggcaaccatcatt

SEQ ID NO: 18 - DNA, 19 bp, Artificial Sequence
  Description of Artificial Sequence; note = synthetic construct
gacgcagacacagctataa

SEQ ID NO: 19 - DNA, 23 bp, Artificial Sequence
  Description of Artificial Sequence; note = synthetic construct
caaggctaacttcccatttagcc

SEQ ID NO: 20 - DNA, 23 bp, Artificial Sequence
  Description of Artificial Sequence; note = synthetic construct
taccgcccatctccagagtaagg

SEQ ID NO: 21 - DNA, 19 bp, Artificial Sequence
  Description of Artificial Sequence; note = synthetic construct
tcctgattctggtgggact

SEQ ID NO: 22 - DNA, 19 bp, Artificial Sequence
  Description of Artificial Sequence; note = synthetic construct
actcttcacacctgtcagc

SEQ ID NO: 23 - DNA, 19 bp, Artificial Sequence
  Description of Artificial Sequence; note = synthetic construct
atccatgacagttgcaaat

SEQ ID NO: 24 - DNA, 23 bp, Artificial Sequence
  Description of Artificial Sequence; note = synthetic construct
caggtgagaagccttatcattgc

SEQ ID NO: 25 - DNA, 23 bp, Artificial Sequence
  Description of Artificial Sequence; note = synthetic construct
aagtgcctagctgccactccatt

SEQ ID NO: 26 - DNA, 23 bp, Artificial Sequence
  Description of Artificial Sequence; note = synthetic construct
aagatgcagagaatcaccgaatt

SEQ ID NO: 27 - DNA, 19 bp, Artificial Sequence
  Description of Artificial Sequence; note = synthetic construct
ggtgcctgcagaagcatat

SEQ ID NO: 28 - DNA, 19 bp, Artificial Sequence
  Description of Artificial Sequence; note = synthetic construct
cccactccaacttctaagt

SEQ ID NO: 29 - DNA, 19 bp, Artificial Sequence
  Description of Artificial Sequence; note = synthetic construct
cacgggagacagaggtttc

SEQ ID NO: 30 - DNA, 23 bp, Artificial Sequence
  Description of Artificial Sequence; note = synthetic construct
aagcctttcatcccagtatcatt

SEQ ID NO: 31 - DNA, 23 bp, Artificial Sequence
  Description of Artificial Sequence; note = synthetic construct
aactgtccacttggagccctgtt

SEQ ID NO: 32 - DNA, 21 bp, Artificial Sequence
  Description of Artificial Sequence; note = synthetic construct
gagagagacaaggatgacctt

SEQ ID NO: 33 - DNA, 19 bp, Artificial Sequence
  Description of Artificial Sequence; note = synthetic construct
tgcggattgtgcagaaaca

SEQ ID NO: 34 - DNA, 21 bp, Artificial Sequence
  Description of Artificial Sequence; note = synthetic construct
gactgtgaaacacaaattttt

SEQ ID NO: 35 - DNA, 21 bp, Artificial Sequence
  Description of Artificial Sequence; note = synthetic construct
tggcaaggactgggaatattt

SEQ ID NO: 36 - DNA, 21 bp, Artificial Sequence
  Description of Artificial Sequence; note = synthetic construct
gctggaagtcagcacaaattt

SEQ ID NO: 37 - DNA, 21 bp, Artificial Sequence
  Description of Artificial Sequence; note = synthetic construct
ggttacccacgaaccccactt

SEQ ID NO: 38 - DNA, 19 bp, Artificial Sequence
  Description of Artificial Sequence; note = synthetic construct
tgcggtgttcctgaattag

SEQ ID NO: 39 - DNA, 19 bp, Artificial Sequence
  Description of Artificial Sequence; note = synthetic construct
gttgcagctgatatgaatg

SEQ ID NO: 40 - DNA, 19 bp, Artificial Sequence
  Description of Artificial Sequence; note = synthetic construct
gctcttaccgaagcaacaa

SEQ ID NO: 41 - DNA, 21 bp, Artificial Sequence
  Description of Artificial Sequence; note = synthetic construct
aagtggcagctgacatgaatg

SEQ ID NO: 42 - DNA, 21 bp, Artificial Sequence
  Description of Artificial Sequence; note = synthetic construct
aggtcgccgcggacatgaacg

SEQ ID NO: 43 - DNA, 64 bp, Artificial Sequence
  Description of Artificial Sequence; note = synthetic construct
gatccccgaggaggcaacggaattccttcaagagaggaattccgttgcctcctctttttggaaa

SEQ ID NO: 44 - DNA, 64 bp, Artificial Sequence
  Description of Artificial Sequence; note = synthetic construct
agcttttccaaaaagaggaggcaacggaattcctctcttgaaggaattccgttgcctcctcggg

SEQ ID NO: 45 - DNA, 64 bp, Artificial Sequence
  Description of Artificial Sequence; note = synthetic construct
gatccccggacacacaccgaggactcttcaagagagagtcctcggtgtgtgtcctttttggaaa

SEQ ID NO: 46 - DNA, 64 bp, Artificial Sequence
  Description of Artificial Sequence; note = synthetic construct
agcttttccaaaaaggacacacaccgaggactctctcttgaagagtcctcggtgtgtgtccggg

SEQ ID NO: 47 - DNA, 64 bp, Artificial Sequence
  Description of Artificial Sequence; note = synthetic construct
gatccccgtgcaagtgcaaaccagacttcaagagagtctggtttgcacttgcactttttggaaa

SEQ ID NO: 48 - DNA, 64 bp, Artificial Sequence
  Description of Artificial Sequence; note = synthetic construct
agcttttccaaaaagtgcaagtgcaaaccagactctcttgaagtctggtttgcacttgcacggg

SEQ ID NO: 49 - DNA, 63 bp, Artificial Sequence
  Description of Artificial Sequence; note = synthetic construct
gatcccagccgaatgtcgcagaaccttcaagagaggttctgcgacattcggcttttttggaaa

SEQ ID NO: 50 - DNA, 64 bp, Artificial Sequence
  Description of Artificial Sequence; note = synthetic construct
agcttttccaaaaaagccgaatgtcgcagaacctctcttgaaggttctgcgacattcggctggg

SEQ ID NO: 51 - DNA, 64 bp, Artificial Sequence
  Description of Artificial Sequence; note = synthetic construct
gatccccgtgaatttacggcagaaacttcaagagagtttctgccgtaaattcactttttggaaa

SEQ ID NO: 52 - DNA, 63 bp, Artificial Sequence
  Description of Artificial Sequence; note = synthetic construct
agcttttccaaaaagtgaatttacggcagaaacctcttgaagtttctgccgtaaattcacggg

SEQ ID NO: 53 - DNA, 64 bp, Artificial Sequence
  Description of Artificial Sequence; note = synthetic construct
gatccccggagataggaatgagtttcttcaagagagaaactcattcctatctcctttttggaaa

SEQ ID NO: 54 - DNA, 64 bp, Artificial Sequence
  Description of Artificial Sequence; note = synthetic construct
agcttttccaaaaaggagataggaatgagtttctctcttgaagaaactcattcctatctccggg

SEQ ID NO: 55 - DNA, 64 bp, Artificial Sequence
  Description of Artificial Sequence; note = synthetic construct
gatcccccacgcagagtaaggactttttcaagagaaaagtccttactctgcgtgtttttggaaa

SEQ ID NO: 56 - DNA, 64 bp, Artificial Sequence
  Description of Artificial Sequence; note = synthetic construct
agcttttccaaaaacacgcagagtaaggacttttctcttgaaaaagtccttactctgcgtgggg

SEQ ID NO: 57 - DNA, 62 bp, Artificial Sequence
  Description of Artificial Sequence; note = synthetic construct
gatccccgcagcctctcatttaataattcaagattattaaatgagaggctgctttttggaaa

SEQ ID NO: 58 - DNA, 64 bp, Artificial Sequence
  Description of Artificial Sequence; note = synthetic construct
agcttttccaaaaagcagcctctcatttaataatctcttgaattattaaatgagaggctgcggg

SEQ ID NO: 59 - DNA, 64 bp, Artificial Sequence
  Description of Artificial Sequence; note = synthetic construct
gatccccgccgctgtcactactgaaattcaagagatttcagtagtgacagcggctttttggaaa

SEQ ID NO: 60 - DNA, 64 bp, Artificial Sequence
  Description of Artificial Sequence; note = synthetic construct
agcttttccaaaaagccgctgtcactactgaaatctcttgaatttcagtagtgacagcggcggg

SEQ ID NO: 61 - DNA, 68 bp, Artificial Sequence
  Description of Artificial Sequence; note = synthetic construct
gatcccccctaacatgtcctgagttatattcaagagatataactcaggacatgttaggtttttggaaa

SEQ ID NO: 62 - DNA, 68 bp, Artificial Sequence
  Description of Artificial Sequence; note = synthetic construct
agcttttccaaaaacctaacatgtcctgagttatatctcttgaatataactcaggacatgttaggggg

SEQ ID NO: 63 - DNA, 68 bp, Artificial Sequence
  Description of Artificial Sequence; note = synthetic construct
gatcccctggtcagtctgttggcttatattcaagagatataagccaacagactgaccatttttggaaa

SEQ ID NO: 64 - DNA, 68 bp, Artificial Sequence
  Description of Artificial Sequence; note = synthetic construct
agcttttccaaaaatggtcagtctgttggcttatatctcttgaatataagccaacagactgaccaggg

SEQ ID NO: 65 - DNA, 64 bp, Artificial Sequence
  Description of Artificial Sequence; note = synthetic construct
gatcccctatctgcctcacagctggcttcaagagagccagctgtgaggcagatatttttggaaa

SEQ ID NO: 66 - DNA, 64 bp, Artificial Sequence
  Description of Artificial Sequence; note = synthetic construct
agattttccaaaaatatctgcctcacagctggctctcttgaagccagctgtgaggcagataggg

SEQ ID NO: 67 - DNA, 64 bp, Artificial Sequence
  Description of Artificial Sequence; note = synthetic construct
gatccccgaagaatcaatcaaagtgtttcaagagaacactttgattgattcttctttttggaaa

SEQ ID NO: 68 - DNA, 64 bp, Artificial Sequence
  Description of Artificial Sequence; note = synthetic construct
agcttttccaaaaagaagaatcaatcaaagtgttctcttgaaacactttgattgattcttcggg

SEQ ID NO: 69 - DNA, 64 bp, Artificial Sequence
  Description of Artificial Sequence; note = synthetic construct
gatcccccagcatatatctcctaatcttcaagagagattaggagatatatgctgtttttggaaa

SEQ ID NO: 70 - DNA, 64 bp, Artificial Sequence
  Description of Artificial Sequence; note = synthetic construct
agcttttccaaaaacagcatatatctcctaatctctcttgaagattaggagatatatgctgggg

SEQ ID NO: 71 - DNA, 19 bp, Artificial Sequence
  Description of Artificial Sequence; note = synthetic construct
ggagacaccaagcaagaaa

SEQ ID NO: 72 - DNA, 18 bp, Artificial Sequence
  Description of Artificial Sequence; note = synthetic construct
acaaggagcccaggagat

SEQ ID NO: 73 - DNA, 18 bp, Artificial Sequence
  Description of Artificial Sequence; note = synthetic construct
acccaaatgcgtcaagtt

SEQ ID NO: 74 - DNA, 18 bp, Artificial Sequence
  Description of Artificial Sequence; note = synthetic construct
gctgcttcatccaccata

SEQ ID NO: 75 - DNA, 20 bp, Artificial Sequence
  Description of Artificial Sequence; note = synthetic construct
ccgagagtttaaagctgagg

SEQ ID NO: 76 - DNA, 26 bp, Artificial Sequence
  Description of Artificial Sequence; note = synthetic construct
ccaggagaatcgcagtagaagtctgg

SEQ ID NO: 77 - DNA, 20 bp, Artificial Sequence
  Description of Artificial Sequence; note = synthetic construct
accacagtccatgccatcac

SEQ ID NO: 78 - DNA, 20 bp, Artificial Sequence
  Description of Artificial Sequence; note = synthetic construct
tccaccaccctgttgctgta

SEQ ID NO: 79 - DNA, 19 bp, Artificial Sequence
  Description of Artificial Sequence; note = synthetic construct
tgagttcgcagctcaactc

SEQ ID NO: 80 - DNA, 31 bp, Artificial Sequence
  Description of Artificial Sequence; note = synthetic construct
tcaggttactaaattgaagagcttggaaatc

SEQ ID NO: 81 - DNA, 18 bp, Artificial Sequence
  Description of Artificial Sequence; note = synthetic construct
ccacatccagacatcgtc

SEQ ID NO: 82 - DNA, 19 bp, Artificial Sequence
  Description of Artificial Sequence; note = synthetic construct
taccagggagatgatctgg

SEQ ID NO: 83 - DNA, 17 bp, Artificial Sequence
  Description of Artificial Sequence; note = synthetic construct
tgaattcagtgctgggc

SEQ ID NO: 84 - DNA, 20 bp, Artificial Sequence
  Description of Artificial Sequence; note = synthetic construct
cactgcctccacctctttag

SEQ ID NO: 85 - DNA, 23 bp, Artificial Sequence
  Description of Artificial Sequence; note = synthetic construct
atgatgatgacaacgacataatg

SEQ ID NO: 86 - DNA, 24 bp, Artificial Sequence
  Description of Artificial Sequence; note = synthetic construct
gatgacaacgacataatggaaacg

SEQ ID NO: 87 - DNA, 22 bp, Artificial Sequence
  Description of Artificial Sequence; note = synthetic construct
atcctgttcctacctcatatgc

SEQ ID NO: 88 - DNA, 20 bp, Artificial Sequence
  Description of Artificial Sequence; note = synthetic construct
ctggatctgcaactgaaact

SEQ ID NO: 89 - DNA, 21 bp, Artificial Sequence
  Description of Artificial Sequence; note = synthetic construct
tcctcacgcggtagagatcag

SEQ ID NO: 90 - DNA, 19 bp, Artificial Sequence
  Description of Artificial Sequence; note = synthetic construct
gtggaggtactcgttgcgg

SEQ ID NO: 91 - DNA, 20 bp, Artificial Sequence
  Description of Artificial Sequence; note = synthetic construct
gcaagtgccttacgtggtca

SEQ ID NO: 92 - DNA, 22 bp, Artificial Sequence
  Description of Artificial Sequence; note = synthetic construct
gcttcagcaagccatgtttctt

SEQ ID NO: 93 - DNA, 19 bp, Artificial Sequence
  Description of Artificial Sequence; note = synthetic construct
atggagtacgcatggggac

SEQ ID NO: 94 - DNA, 23 bp, Artificial Sequence
  Description of Artificial Sequence; note = synthetic construct
gattaccagggagatgatctgga

SEQ ID NO: 95 - DNA, 19 bp, Artificial Sequence
  Description of Artificial Sequence; note = synthetic construct
gtgtggtgcagatcgcagt

SEQ ID NO: 96 - DNA, 21 bp, Artificial Sequence
  Description of Artificial Sequence; note = synthetic construct
atcatgccttcggacttgatg

SEQ ID NO: 97 - DNA, 22 bp, Artificial Sequence
  Description of Artificial Sequence; note = synthetic construct
agcttgtggtaagacatgcttg

SEQ ID NO: 98 - DNA, 23 bp, Artificial Sequence
  Description of Artificial Sequence; note = synthetic construct
gtgtcccataaagccaactctac

SEQ ID NO: 99 - DNA, 21 bp, Artificial Sequence
  Description of Artificial Sequence; note = synthetic construct
catcgagtaccagaacatgcg

SEQ ID NO: 100 - DNA, 20 bp, Artificial Sequence
  Description of Artificial Sequence; note = synthetic construct
gaagagcgagcacaggaact

SEQ ID NO: 101 - DNA, 24 bp, Artificial Sequence
  Description of Artificial Sequence; note = synthetic construct
acctcaagtctcacgcggaagaaa

SEQ ID NO: 102 - DNA, 24 bp, Artificial Sequence
  Description of Artificial Sequence; note = synthetic construct
tgacacaggaagtccttgcatcct

SEQ ID NO: 103 - DNA, 64 bp, Artificial Sequence
  Description of Artificial Sequence; note = synthetic construct
gatcccctctagatgtatgttagcatttcaagagaatgctaacatacatctagatttttggaaa

SEQ ID NO: 104 - DNA, 63 bp, Artificial Sequence
  Description of Artificial Sequence; note = synthetic construct
agcttttccaaaaatctagatgtatgttagcattctcttgaaatgctaactacatctagaggg

SEQ ID NO: 105 - DNA, 64 bp, Artificial Sequence
  Description of Artificial Sequence; note = synthetic construct
gatccccgtgcctagctgccactccattcaagagatggagtggcagctaggcactttttggaaa

SEQ ID NO: 106 - DNA, 64 bp, Artificial Sequence
  Description of Artificial Sequence; note = synthetic construct
agcttttccaaaaagtgcctagctgccactccatctcttgaatggagtggcagctaggcacggg

SEQ ID NO: 107 - DNA, 23 bp, Artificial Sequence
  Description of Artificial Sequence; note = synthetic construct
gggtgtttcgtcgattatcaaga

SEQ ID NO: 108 - DNA, 21 bp, Artificial Sequence
  Description of Artificial Sequence; note = synthetic construct
tcgcccatacttgttggagat

SEQ ID NO: 109 - DNA, 23 bp, Artificial Sequence
  Description of Artificial Sequence; note = synthetic construct
tatcaccactattgctggagtca

SEQ ID NO: 110 - DNA, 23 bp, Artificial Sequence
  Description of Artificial Sequence; note = synthetic construct
acgaagcagttgaactttctgtt

SEQ ID NO: 111 - DNA, 24 bp, Artificial Sequence
  Description of Artificial Sequence; note = synthetic construct
tctcaggaggtgcacgtttcatca

SEQ ID NO: 112 - DNA, 24 bp, Artificial Sequence
  Description of Artificial Sequence; note = synthetic construct
attccatcttccgtttccaagggc

SEQ ID NO: 113 - DNA, 19 bp, Artificial Sequence
  Description of Artificial Sequence; note = synthetic construct
tggttgcagtctacggacc

SEQ ID NO: 114 - DNA, 21 bp, Artificial Sequence
  Description of Artificial Sequence; note = synthetic construct
tcaggaagacaagcatctggg

SEQ ID NO: 115 - DNA, 21 bp, Artificial Sequence
  Description of Artificial Sequence; note = synthetic construct
atgacctagacgagaccatcc

SEQ ID NO: 116 - DNA, 19 bp, Artificial Sequence
  Description of Artificial Sequence; note = synthetic construct
gtcgcactcaagcatgtcg

(19) United States
(12) Patent Application Publication
(10) Pub. No.: US 2012/0114670 A1
(43) Pub. Date: May 10, 2012

(54) METHODS AND COMPOSITIONS RELATED TO SYNERGISTIC RESPONSES TO ONCOGENIC MUTATIONS

(75) Inventors: Hartmut Land, Rochester, NY (US); Helene R. McMurray, Rochester, NY (US); Erik R. Sampson, Holden, MA (US)

(73) Assignee: UNIVERSITY OF ROCHESTER, Rochester, NY (US)

(21) Appl. No.: 13/011,901

(22) Filed: Jan. 23, 2011

Related U.S. Application Data
(63) Continuation-in-part of application No. 12/678,351, filed on Jun. 16, 2010.
(60) Provisional application No. 60/977,052, filed on Oct. 2, 2007.
(60) Provisional application No. 61/044,372, filed on Apr. 11, 2008.

(51) Int. Cl. A61K 39/395; G01N 33/566; C40B 30/04; A61K 38/17; A61K 31/704; A61K 31/713; A61K 31/65; A61K 31/616; A61K 31/565; A61K 31/43; A61K 31/5415; A61K 31/506; A61K 31/52; A61K 31/485; A61K 31/365; A61P 35/00; A61P 35/02; A61P 35/04; C12Q 1/68

(52) U.S. Cl. 424/174.1; 435/6.12; 435/6.14; 435/7.1; 506/9; 514/19.3; 514/19.4; 514/19.5; 514/19.6; 514/34; 514/44 A; 514/152; 514/159; 514/182; 514/197; 514/226.2; 514/252.15; 514/263.2; 514/289; 514/450

(57) ABSTRACT

Disclosed are compositions and methods related to new targets for cancer treatment.

This work was supported in part by NIH grants CA90663, CA120317, GM075299; T32 CA09363; NCI R01-CA138249-02, NCI P30-CA147880-01, and NLM R00-LM009477-02. The government has certain rights in the invention.

This application claims priority to U.S. application Ser. No. 12/678,351 which is a 371 National Stage Application of PCT Application No. PCT/US08/11375, filed on Oct. 2, 2008, which claims the benefit of U.S. Provisional Application No. 60/977,052, filed on Oct. 2, 2007 and U.S. Provisional Application No. 61/044,372, filed on Apr. 11, 2008, which are incorporated by reference herein in their entirety.

### I. BACKGROUND

Understanding the molecular underpinnings of cancer is of critical importance to developing targeted intervention strategies. Identification of such targets, however, is notoriously difficult and unpredictable. Malignant cell transformation requires the cooperation of a few oncogenic mutations that cause substantial reorganization of many cell features (Hanahan, D. & Weinberg, R. A. (2000) Cell 100, 57-70) and induce complex changes in gene expression patterns (Yu, J. et al. (1999) Proc Natl Acad Sci USA 96, 14517-22 (1999); Zhao, R. et al. (2000) Genes Dev 14, 981-93; Schulze, A., et al. (2000) Genes Dev 15, 981-94; Huang, E. et al. (2003) Nat Genet. 34, 226-30; Boiko, A. D. et al. A (2006) Genes Dev 20, 236-52). Genes critical to this multi-faceted cellular phenotype thus only have been identified following signaling pathway analysis (Vogelstein, B., et al. (2000) Nature 408, 307-10; Vousden, K. H. & Lu, X. (2002) Nat Rev Cancer 2, 594-604; Downward, J. (2003) Nat Rev Cancer 3, 11-22; Rodriguez-Viciana, P. et al. (2005) Cold Spring Harb Symp Quant Biol 70, 461-7) or on an ad hoc basis (Schulze, A., et al. (2000) Genes Dev 15, 981-94; Okada, F. et al. (1998) Proc Natl Acad Sci USA 95, 3609-14; Clark, E. A., et al. (2000) Nature 406, 532-5; Yang, J. et al. (2004) Cell 117, 927-39; Minn, A. J. et al. (2005) Nature 436, 518-24). Thus, there is a need for new methods of identifying genes critical to the formation, proliferation and maintenance of cancer.

### II. SUMMARY

Disclosed are methods and compositions related to in one aspect methods for identifying targets for the treatment of a cancer. In other aspect, disclosed herein are methods for screening for an agent that treats a cancer. Also disclosed herein are methods of treating cancer. Further disclosed are methods related to determining whether a cancer is susceptible to treatment.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows the differential expression and synergy scores of CRGs in mp53/Ras cells and CRG co-regulation in human colon cancer. Bar graphs ranking CRG expression measured by microarray in mp53/Ras vs. YAMC cells (A) and CRG synergy scores (B). Bars are coded for gene-associated biological processes according to Gene Ontology (GO) database. C) Table summarizing co-regulation of CRGs in mp53/Ras cells and human cancer based on literature survey for a variety of human cancers and two independent expression analyses of primary human colon cancers. Up- or down-regulation of CRG expression vs. controls is indicated, lack of CRG representation on arrays by (/). Arrows indicate genes perturbed in this study.

FIG. 2 shows the assessment of co-regulation for CRG expression in human colon cancer and murine colon cancer cell model. T-statistics of CRG expression for a total of 75 out of 95 genes are shown for human colon cancer, as compared to normal tissue samples plotted against t-statistics of expression values for the same genes in mp53/Ras cells, as compared to YAMC. Data points in lower left and upper right hand quadrants show co-regulation of the indicated genes in the murine model and human colon cancer. FIG. 2A shows plot based on cDNA microarray data as described in Supplemental Methods. Of the 95 CRG identified in mp53/Ras cells, 69 genes are represented on these cDNA arrays. Names are indicated for the 33 genes that appear co-regulated. Of these, 17 are significantly differentially expressed (t-test, unadjusted, p<0.05) in this human dataset, indicated. FIG. 2B shows plot based on oligonucleotide microarray data, as described in Supplemental Methods. Of the 95 CRG identified in mp53/Ras cells, 38 genes are represented on these microarrays. Names are indicated for the 20 genes that appear co-regulated. Of these, 6 are significantly differentially expressed (t-test, unadjusted, p<0.05) in this human dataset, indicated. All CRGs are significantly differentially expressed in our murine data set.

FIG. 3 shows the differential expression and synergy score ranking of genetically perturbed non-CRGs in mp53/Ras cells. Bar graphs indicate fold-change expression (log₂) in mp53/Ras vs. YAMC cells (A) and synergy scores (B) derived from Affymetrix microarray data for non-CRGs selected for gene perturbation experiments. Color code illustrates gene-associated biological process according to GO.

FIG. 4 shows the synergistic response of downstream genes to oncogenic mutations is a strong predictor for critical role in malignant transformation. FIG. 4A shows bar graphs indicating percent change in endpoint tumor volume following CRG and non-CRG perturbations in mp53/Ras cells (left and right panel, respectively). Perturbations significantly decreasing tumor size, as compared to matched controls are shown (***, p<0.001; **, p<0.01; *, p<0.05; Wilcoxn signed-rank and t-test). FIG. 4B shows the distribution of gene perturbations over the set of genes differentially expressed in mp53/Ras cells, rank-ordered by synergy score. Bars, color-coded as above, indicate perturbed genes. CRG cut-off synergy score (0.9) is indicated by horizontal line.

FIG. 5 shows the Synergy score ranking of CRGs in mp53/Ras cells. Graph showing synergy scores for the entire list of 95 CRGs identified in this study derived from Affymetrix microarray data, as described in Methods. Individual synergy scores and associated estimated p values are indicated in Table 1. Bars indicate CRGs chosen for gene perturbation experiments. Perturbations causing significant tumor reduction are indicated in by a darker line; those not causing reduction are lightly marked.

FIG. 6 shows the resetting mRNA expression levels in mp53/Ras cells to approximate mRNA levels in normal YAMC cells via gene perturbations. Each panel shows the relative expression levels of an individual gene following its perturbation in mp53/Ras cells together with its expression levels in the matching vector control mp53/Ras cells and the parental YAMC cells, as measured by SYBR Green QPCR. Error bars indicate standard deviation of triplicate samples. Independent derivations of the perturbed cells and controls are shown individually. Injection numbers relating to xenograft assays are shown for each cell derivation, vector followed by perturbed cells. FIG. 6A shows the Re-expression of down-regulated CRGs in mp53/Ras cells. For CRGs identified as critical for tumor formation, levels of cDNA re-expression in the respective cell populations were below, at or marginally above mRNA expression levels of the corresponding endogenous gene in YAMC cells, although the possibility of over-expression at the protein level cannot be excluded. For CRGs determined to be non-critical, tumor-inhibitory effects were not observed over a wide range of re-expression levels, including strong over-expression. FIG. 6B shows the shRNA-mediated knock-down of up-regulated CRGs in mp53/Ras cells. FIG. 6C shows the re-expression of down-regulated non-CRGs in mp53/Ras cells. For non-CRGs determined to be non-critical, tumor-inhibitory effects were not observed over a wide range of re-expression levels, including strong over-expression. The tumor-inhibitory effect of Tbx18 may be due to over-expression, as only cell populations expressing levels of Tbx18 RNA 10-30× above YAMC levels were obtained. Similarly, the tumor-promoting effect of the Cox6b2 perturbation may be due to over-expression. FIG. 6D shows shRNA-mediated knock-down of up-regulated non-CRGs in mp53/Ras cells. FIG. 6E shows the combined re-expression of Fas and Rprm in mp53/Ras cells.

FIG. 7 shows the altered CRG expression in human colon cancer cells following gene perturbations. Each panel shows the relative mRNA expression levels of the indicated gene following its perturbation in DLD-1 or HT-29 cells together with its mRNA expression level in the matching vector control cells, as measured by SYBR Green QPCR. Error bars indicate standard deviation of triplicate samples. Independent derivations of the perturbed cells and controls are shown individually. Injection numbers relating to xenograft assays are shown for each cell derivation, vector followed by perturbed cells. FIG. 7A shows the expression of human cDNA for HoxC13 and murine cDNAs for Jag2, Dffb, Perp and Zfp385 in DLD-1 and HT-29 cells. As qPCR primers for murine genes do not cross-react with endogenous human RNA, differential gene expression values become artificially large. FIG. 7B shows the shRNA-mediated knock-down of Plac8 in HT-29 cells. FIG. 7C shows the expression of murine Fas and murine Rprm in human DLD-1 cells. Primers for mFas do not cross-react with endogenous human RNA resulting in artificially large values for differential expression. For Rprm, cross-reactive primers were used, giving lower expression values due to detection of endogenous RNA.

FIG. 8 shows that synergistically regulated genes downstream genes of oncogenic mutations play a critical role in malignant transformation. FIG. 8A shows Bar graphs indicating percent change in endpoint tumor volume following CRG and non-CRG perturbations in mp53/Ras cells (left and right panel, respectively). Perturbations significantly decreasing tumor size, as compared to matched controls are shown (***, p<0.001; **, p<0.01; *, p<0.05; Wilcoxn signed-rank and t-test). FIG. 8B shows the impact of CRG perturbations on tumor formation of mp53/Ras cells. Individual CRG perturbations are shown. Box plots indicate volume (cm3) of tumors formed four weeks after injection of cell populations with indicated CRG perturbations, as compared with matched vector controls, colored as above. The box indicates the range from the first quartile to the third quartile of the data. The line in the box indicates the median value. The whiskers or error bars indicate the highest and lowest values in the data. Plots are ranked by % change in tumor volume.

FIG. 9 shows that resetting mRNA expression levels in mp53/Ras cells to approximate mRNA levels in normal YAMC cells via gene perturbations. Each panel shows the relative expression levels of an individual gene following its perturbation in mp53/Ras cells together with its expression levels in the matching vector control mp53/Ras cells and the parental YAMC cells, as measured by SYBR Green QPCR. Error bars indicate standard deviation of triplicate samples. Independent derivations of the perturbed cells and controls are shown individually. For CRGs identified as critical for tumor formation, levels of cDNA re-expression in the respective cell populations were below, at or marginally above mRNA expression levels of the corresponding endogenous gene in YAMC cells, although the possibility of over-expression at the protein level cannot be excluded. For CRGs determined to be non-critical, tumor-inhibitory effects were not observed over a wide range of re-expression levels, including strong over-expression.

FIG. 10 shows that cooperation response genes are highly co-regulated in human colon cancer, pancreatic cancer, prostate cancer, lung cancer, and melanoma. Table summarizing co-regulation of CRGs in mp53/Ras cells and human cancer based on independent expression analyses of primary human colon cancer, pancreatic cancer, prostate cancer, lung cancer, melanoma. Up- or down-regulation of CRG expression vs. controls is indicated, by dark or light shading, respectively. Lack of CRG representation on arrays is indicated by (/). Effects of gene perturbations in mp53/Ras cells are indicated by presence of shading around text (shaded text box, tumor inhibitory; no shade, not inhibitory/not tested).

FIG. 11 shows the assessment of co-regulation for CRG expression in human pancreatic and prostate cancer and murine colon cancer cell model. Data points in lower left and upper right hand quadrants show co-regulation of the indicated genes in the murine model and human colon cancer. FIG. 11A shows T-statistics of CRG expression for a total of 69 out of 95 genes are shown for human pancreatic cancer, as compared to normal tissue samples, plotted against t-statistics of expression values for the same genes in mp53/Ras cells, as compared to YAMC. Names are indicated for the 33 genes that appear co-regulated. Of these, 25 are significantly differentially expressed (t-test, unadjusted, p<0.05) in this human dataset, indicated in blue. FIG. 11B shows the T-statistics of CRG expression for a total of 47 out of 95 genes are shown for human prostate cancer, as compared to normal tissue samples, plotted against t-statistics of expression values for the same genes in mp53/Ras cells, as compared to YAMC. Names are indicated for the 31 genes that appear co-regulated. Of these, 23 are significantly differentially expressed (t-test, unadjusted, p<0.05) in this human dataset, indicated in blue. All CRGs are significantly differentially expressed in the murine data set.

FIG. 12 shows that HDAC inhibitors reverse the CRG signature in human cancer cells. Histograms depicting expression pattern of CRGs (log₂). FIG. 12A shows the TLDA derived values for CRG expression in mp53/Ras cells as compared to YAMC cells. FIG. 12B shows Affymetrix microarray data obtained from the CMap database, comparing VA-treated human breast cancer cells (MCF7) with untreated control cells.

FIG. 13 shows the effects of HDACi on mp53/Ras and YAMC cell cycle progression and apoptosis. mp53/Ras and YAMC were plated at microarray density onto 15 cm collagen IV-coated dishes in 10% FBS medium at 39° C. for two days. The cells were re-plated at 458,000 cells per 15 cm dish in 10% FBS medium and treated for three days with 2.5 mM NB or VA at 39° C. Cells were then trypsinized and (A), (B) suspended in methylcellulose supplemented with fresh NB or VA, 10% FBS, and ITS-A at 37,000 cells per mL, or (C) suspended in methylcellulose w/o FBS, or ITS-A at 150,000 cells per mL and incubated at 39° C. for three days. Cells were extracted from the methylcellulose by repeated re-suspension in PBS w/1% BSA and centrifugation, and briefly trypsinized to break up cell aggregates. The extracted cells were labeled with 10 μM BrdU for ninety minutes prior to harvesting, fixed in cold 80% ethanol, and stained with an anti-BrdU antibody and propidium iodide to measure cell cycle progression (A), or fixed in 4% paraformaldehyde, and TUNEL-stained to measure cell death (B), (C). Error bars represent standard deviation values derived from multiple independent measurements for each sample. The asterisk denotes a statistically significant difference (p-value <0.05) versus untreated cells.

FIG. 14 shows that HDAC inhibitors antagonize the CRG signature and behavior of mp53/Ras cells. FIG. 14A shows RNA from mp53/Ras cells treated with 2.5 mM VA or NB for 3 days was analyzed for changes in CRG expression via TaqMan Low Density arrays. Four replicates were performed for each condition. Histograms indicate differential CRG expression, assessed by the t statistic, in mp53/Ras cells as compared to normal YAMC cells (upper panel), VA-treated mp53/Ras cells as compared to untreated controls (middle panel) and NB-treated mp53/Ras cells as compared to untreated controls (lower panel). FIG. 14B shows Histogram showing cell death, measured by TUNEL staining, in cell populations treated with 2.5 mM VA or NB for 3 days in adherent culture, or untreated controls. Bars represent the mean of triplicate experiments, ±SEM. (C) Histogram showing cell death in cell populations pre-treated with 2.5 mM VA or NB, or untreated controls, suspended in methylcellulose for an additional 3 days. Bars represent the mean of triplicate experiments, ±SEM. (D) Histogram showing volume of tumors formed by untreated mp53/Ras cells (n=6), or by mp53/Ras cells pre-treated with either 2.5 mM NB (n=8), or 2.5 mM VA (n=4) at four weeks post-injection, represented as mean±SEM. **, p<0.01, Wilcoxon signed-rank test.

FIG. 15 shows increased histone acetylation at CRG promoters in HDACi-treated cells. YAMC and Mp53/Ras cells were treated with 2.5 mM NB for three days, cross-linked, and harvested for immunoprecipitation using an acetyl-histone H3 immunoprecipitation (ChIP) assay kit (Millipore). QPCR was run to detect presence and abundance of the promoters of five HDACi-sensitive (A) and four HDACi-insensitive (B) CRGs.

FIG. 16 shows that RNA interference reduces CRG induction by HDACi in mp53/Ras cells. mp53/Ras cells stably expressing shRNA molecules targeting Dapk, Fas, Noxa, Perp or Sfrp2 (A), shRNA molecules and shRNA-resistant cDNAs for Noxa or Perp (B), or shRNA molecules targeting Elk3 or Etv1 (C) were treated with 2.5 mM VA or NB as indicated for 3 days. RNA was isolated and RT-QPCR was performed to assess expression of indicated CRGs, relative to untreated cells. Histograms show mean expression in perturbed cells by shRNA construct, as compared to matched vector control cells, ±SEM.

FIG. 17 shows that Anoikis induction by HDACi depends on multiple CRGs. Mp53/Ras cells stably expressing the indicated shRNA molecules were pre-treated with 2.5 mM NB or VA for 3 days and then suspended in methylcellulose for an additional 3 days in the presence of NB or VA. Anoikis was measured by TUNEL staining and flow cytometry, expressed as % TUNEL positive cells. Data show mean of duplicate or triplicate samples±SEM. *, p<0.001 versus untreated empty vector cells; #, p<0.05 versus NB-treated empty vector cells; †, p<0.05 versus VA-treated empty vector cells; Wilcoxon signed-rank and t-test. FIG. 17A shows Apoptosis in mp53/Ras cells expressing shRNA molecules targeting Dapk, Fas, Noxa, Perp or Sfrp2, compared to cells expressing the empty vector. FIG. 17B shows Apoptosis in mp53/Ras cells expressing the empty vector, Noxa shRNA, or Noxa shRNA plus a shRNA-resistant Noxa cDNA. FIG. 17C shows Apoptosis of mp53/Ras cells expressing shRNA molecules targeting Etv1 or Elk3 or empty vector.

FIG. 18 shows Anoikis induction by HDACi depends on multiple CRGs. mp53/Ras cells stably expressing the indicated shRNA molecules were pre-treated with 2.5 mM NB or VA for 3 days and then suspended in methylcellulose for an additional 3 days in the presence of NB or VA. Anoikis was measured by TUNEL staining and flow cytometry, expressed as % TUNEL positive cells. Data show mean of duplicate or triplicate samples by shRNA construct±SEM. *, p<0.001 versus untreated empty vector cells; #, p<0.05 versus NB-treated empty vector cells; †, p<0.05 versus VA-treated empty vector cells; Wilcoxon signed-rank and t-test.

FIG. 19 shows that pharmacologic agents target different subsets of CRGs. Histograms depicting expression pattern of CRGs (log₂). Affymetrix microarray data obtained from the CMap database, comparing HDACi valproic acid-treated MCF7 with untreated control cells (top panel) or PI3-kinase inhibitor LY294002-treated MCF7 with untreated controls (bottom panel).

FIG. 20 shows that synergistically regulated genes downstream genes of oncogenic mutations play a critical role in malignant transformation. FIG. 20A shows bar graphs indicating percent change in endpoint tumor volume following CRG perturbations in mp53/Ras cells. Perturbations significantly decreasing tumor size, as compared to matched controls are shown indicated by darker bars (p<0.05, Wilcoxn signed-rank and t-test). FIG. 20B shows the impact of combination CRG perturbations on tumor formation of mp53/Ras cells. Box plots indicate volume (cm³) of tumors formed four weeks after injection of cell populations with indicated CRG perturbations, as compared with matched vector controls, shaded as above. FIG. 20C shows the biological process of CRGs, tumor inhibitory CRGs and known oncogenes and tumor suppressors. Pie charts indicate the percentage of each gene class with indicated ascribed biological functions according to the Gene Ontology database.

FIG. 21 shows the impact of tumor inhibitory CRG perturbations on tumor formation of mp53/Ras cells. Box plots indicate volume (cm³) of tumors formed four weeks after injection of cell populations with indicated CRG perturbations (dark boxes), as compared with matched vector controls (white boxes). The box indicates the range from the first quartile to the third quartile of the data. The line in the box indicates the median value. Plots are ranked by % change in tumor volume.

FIG. 22 shows oncogene cooperation regulates gene expression at transcriptional and translation levels. Histograms show synergy scores for each CRG in total RNA, measured by TLDA, and in polysomal RNA (bottom panel), measured by Affymetrix microarray. Synergistically regulated genes are considered to have a synergy score below 0.9, indicated by the horizontal line. Bars are shaded to indicate the effect of perturbation of each CRG on tumor formation capacity of mp53/Ras cells (dark, significant reduction in tumor volume; gray, no significant change in tumor volume; white, not able to be perturbed).

FIG. 23 shows the insensitivity of gene expression patterns to extracellular signals specifically in mp53/Ras cells. Histograms show relative gene expression in indicated cell populations, as compared to normal YAMC cells, measured by TLDA using total RNA from cells grown in the presence or absence of FBS for 24 hours prior to cell harvesting.

FIG. 24 shows that CRGs regulate tumor formation capacity of human pancreatic and prostate cancer cells.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

A “decrease” can refer to any change that results in a smaller amount of a symptom, composition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed.

An “increase” can refer to any change that results in a larger amount of a symptom, composition, or activity. Thus, for example, an increase in the amount of Jag2 can include but is not limited to a 10%, 20%, 30%, 40%, 50%, 60%, 70%, 80%, 90%, or 100% increase.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Enhance,” “enhancing,” and “enhancement” mean to increase an activity, response, condition, disease, or other biological parameter. This can include but is not limited to the doubling, tripling, quadrupling, or any other factor of increase in activity, response, condition, or disease. This may also include, for example, a 10% increase in the activity, response, condition, or disease as compared to the native or control level. Thus, the increase can be a 10, 20, 30, 40, 50, 60, 70, 80, 90, 100, 150, 200, 300, 400, 500% or any amount of increase in between as compared to native or control levels.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. METHODS OF USING THE COMPOSITIONS

1. Methods of Identifying Targets for the Treatment of Cancer

Despite recognition of the multifaceted cellular phenotype of cancers and the need for targeted intervention strategies, identification of such targets, however, is notoriously difficult and unpredictable using techniques known in the art. Therefore, disclosed herein are methods for identifying targets for the treatment of a cancer comprising performing an assay that measures differential expression of a gene or protein and identifying those genes, proteins, or micro RNAs that respond synergistically to the combination of two or more cancer genes.

As used herein, “cancer gene” can refer to any gene that has an effect on the formation, maintenance, proliferation, death, or survival of a cancer. It is understood and herein contemplated that “cancer gene” can comprise oncogenes, tumor suppressor genes, as well as gain or loss of function mutants there of It is further understood and herein contemplated that where a particular combination of two or more cancer genes is discussed, disclosed herein are each and every permutation of the combination including the use of the gain or loss of functions mutants of the particular genes in the combination. It is further understood and herein contemplated that the disclosed combinations can include an oncogene and a tumor suppressor gene, two oncogenes, two tumor suppressor genes, or any variation thereof where gain or loss of function mutants are used. Thus, for example, disclosed herein are any combination of two or more of the cancer genes selected from the group consisting of ABL1, ABL2, AF15Q14, AF1Q, AF3p21, AF5q31, AKT, AKT2, ALK, ALO17, AML1, AP1, APC, ARHGEF, ARHH, ARNT, ASPSCR1, ATIC, ATM, AXL, BCL10, BCL11A, BCL11B, BCL2, BCL3, BCL5, BCL6, BCL7A, BCL9, BCR, BHD, BIRC3, BLM, BMPR1A, BRCA1, BRCA2, BRD4, BTG1, CBFA2T1, CBFA2T3, CBFB, CBL, CCND1, c-fos, CDH1, c-jun, CDK4, c-kit, CDKN2A-p14ARF, CDKN2A-p16INK4A, CDX2, CEBPA, CEP1, CHEK2, CHIC2, CHN1, CLTC, c-met, c-myc, COL1A1, COPEB, COX6C, CREBBP, c-ret, CTNNB1, CYLD, D10S170, DDB2, DDIT3, DDX10, DEK, EGFR, EIF4A2, ELKS, ELL, EP300, EPS15, erbB, ERBB2, ERCC2, ERCC3, ERCC4, ERCC5, ERG, ETV1, ETV4, ETV6, EVI1, EWSR1, EXT1, EXT2, FACL6, FANCA, FANCC, FANCD2, FANCE, FANCF, FANCG, FEV, FGFR1, FGFR1OP, FGFR2, FGFR3, FH, FIP1L1, FLI1, FLT3, FLT4, FMS, FNBP1, FOXO1A, FOXO3A, FPS, FSTL3, FUS, GAS7, GATA1, GIP, GMPS, GNAS, GOLGA5, GPC3, GPHN, GRAF, HEI10, HER3, HIP1, HIST1H4I, HLF, HMGA2, HOXA11, HOXA13, HOXA9, HOXC13, HOXD11, HOXD13, HRAS, HRPT2, HSPCA, HSPCB, hTERT, IGH, IGK, IGL, IL21R, IRF4, IRTA1, JAK2, KIT, KRAS2, LAF4, LASP1, LCK, LCP1, LCX, LHFP, LMO1, LMO2, LPP, LYL1, MADH4, MALT1, MAML2, MAP2K4, MDM2, MECT1, MEN1, MET, MHC2TA, MLF1, MLH1, MLL, MLLT1, MLLT10, MLLT2, MLLT3, MLLT4, MLLT6, MLLT7, MLM, MN1, MSF, MSH2, MSH6, MSN, MTS1, MUTYH, MYC, MYCL1, MYCN, MYH11, MYH9, MYST4, NACA, NBS1, NCOA2, NCOA4, NF1, NF2, NOTCH1, NPM1, NR4A3, NRAS, NSD1, NTRK1, NTRK3, NUMA1, NUP214, NUP98, NUT, OLIG2, p53, p27, p57, p16, p21, p73, PAX3, PAX5, PAX7, PAX8, PBX1, PCM1, PDGFB, PDGFRA, PDGFRB, PICALM, PIM1, PML, PMS1, PMS2, PMX1, PNUTL1, POU2AF1, PPARG, PRAD-1, PRCC, PRKAR1A, PRO1073, PSIP2, PTCH, PTEN, PTPN11, RAB5EP, RAD51L1, RAF, RAP1GDS1, RARA, RAS, Rb, RB1, RECQL4, REL, RET, RPL22, RUNX1, RUNXBP2, SBDS, SDHB, SDHC, SDHD, SEPT6, SET, SFPQ, SH3GL1, SIS, SMAD2, SMAD3, SMAD4, SMARCB1, SMO, SRC, SS18, SS18L1, SSH3BP1, SSX1, SSX2, SSX4, Stathmin, STK11, STL, SUFU, TAF15, TAL1, TAL2, TCF1, TCF12, TCF3, TCL1A, TEC, TCF12, TFE3, TFEB, TFG, TFPT, TFRC, TIF1, TLX1, TLX3, TNFRSF6, TOP1, TP53, TPM3, TPM4, TPR, TRA, TRB, TRD, TRIM33, TRIP11, TRK, TSC1, TSC2, TSHR, VHL, WAS, WHSC1L18, WRN, WT1, XPA, XPC, ZNF145, ZNF198, ZNF278, ZNF384, and ZNFN1A1. It is further understood that the disclosed combinations of two or more cancer genes can comprise 2, 3, 4, 5, 6, 7, 8, 9, or 10 cancer genes.

As discussed above, disclosed herein are combinations of cancer genes, wherein the cancer genes comprise an oncogene and loss of function of a tumor suppressor gene. It is understood and herein contemplated that there are many oncogenes known in the art. Thus, for example, disclosed herein are cancer gene combinations comprising an oncogene and a tumor suppressor gene wherein the oncogene is selected from the list of oncogenes consisting of ras, raf, Bcl-2, Akt, Sis, src, Notch, Stathmin, mdm2, abl, hTERT, c-fos, c-jun, c-myc, erbB, HER2/Neu, HER3, c-kit, c-met, c-ret, flt3, AP1, AML1, axl, alk, fms, fps, gip, lck, MLM, PRAD-1, and trk. Therefore, disclosed herein are methods for identifying targets for the treatment of a cancer comprising performing an assay that measures differential expression of a gene, protein or micro RNAs and identifying those genes, proteins or micro RNAs that respond synergistically to the combination of two or more cancer genes, wherein the combination of two or more cancer genes comprises an oncogene and a tumor suppressor gene wherein the oncogene is selected from the list of oncogenes consisting of ras, raf, Bcl-2, Akt, Sis, src, Notch, Stathmin, mdm2, abl, hTERT, c-fos, c-jun, c-myc, erbB, HER2/Neu, HER3, c-kit, c-met, c-ret, flt3, AP1, AML1, axl, alk, fms, fps, gip, lck, MLM, PRAD-1, and trk. It is understood that there are other means known in the art to accomplish this task orther than evaluating synergistic response of gene expression to a combination of cancer genes. One such method, for example, involves developing rank-order by synergy score, multiplicativity score, or maximum p-value by N-test. While the multiplicativity score and differential expression via the N-test identify somewhat different sets of genes than the additive synergy score, all three methods perform similarly at isolating genes critical to tumor formation from non-essential genes. Thus, disclosed herein are methods for identifying targets for the treatment of a cancer comprising performing an assay that measures differential expression of a gene, protein or micro RNAs, evaluating the expression via additive synergy score, multiplicative synergy score, or N-test, and identifying those genes, proteins or micro RNAs that have differential expression in response to the combination of two or more cancer genes relative to the absence of said cancer genes or the presence of one cancer gene, wherein the combination of two or more cancer genes comprises an oncogene and a tumor suppressor gene wherein the oncogene is selected from the list of oncogenes consisting of ras, raf, Bcl-2, Akt, Sis, src, Notch, Stathmin, mdm2, abl, hTERT, c-fos, c-jun, c-myc, erbB, HER2/Neu, HER3, c-kit, c-met, c-ret, flt3, AP1, AML1, axl, alk, fms, fps, gip, lck, MLM, PRAD-1, and trk.

Further disclosed are cancer gene combinations comprising an oncogene and a tumor suppressor gene and/or their gain or loss of function mutants wherein the tumor suppressor gene is selected from the list of tumor suppressor genes consisting of p53, Rb, PTEN, BRCA-1, BRCA-2, APC, p57, p27, p16, p21, p73, p14ARF, Chek2, NF1, NF2, VHL, WRN, WT1, MEN1, MTS1, SMAD2, SMAD3, and SMAD4. Therefore, disclosed herein are methods for identifying targets for the treatment of a cancer comprising performing an assay that measures differential expression of a gene or protein and identifying those genes, proteins, or micro RNAs that respond synergistically to the combination of two or more cancer genes, wherein the combination of two or more cancer genes comprises an oncogene and a tumor suppressor gene and/or their gain or loss of function mutants wherein the tumor suppressor gene is selected from the list of tumor suppressor genes consisting of p53, Rb, PTEN, BRCA-1, BRCA-2, APC, p57, p27, p16, p21, p73, p14ARF, Chek2, NF1, NF2, VHL, WRN, WT1, MEN1, MTS1, SMAD2, SMAD3, and SMAD4. Therefore disclosed herein are methods for identifying targets for the treatment of a cancer comprising performing an assay that measures differential expression of a gene or protein and identifying those genes, proteins, or micro RNAs that respond synergistically to the combination of two or more cancer genes, wherein the combination of two or more cancer genes comprises an oncogene and a tumor suppressor gene wherein the oncogene is selected from the list of oncogenes consisting of ras, raf, Bcl-2, Akt, Sis, src, Notch, Stathmin, mdm2, abl, hTERT, c-fos, c-jun, c-myc, erbB, HER2/Neu, HER3, c-kit, c-met, c-ret, flt3, AP1, AML1, axl, alk, fms, fps, gip, lck, MLM, PRAD-1, and trk and wherein the tumor suppressor gene is selected from the list of tumor suppressor genes consisting of p53, Rb, PTEN, BRCA-1, BRCA-2, APC, p57, p27, p16, p21, p73, p14ARF, Chek2, NF1, NF2, VHL, WRN, WT1, MEN¹, MTS1, SMAD2, SMAD3, and SMAD4. Thus, for example, specifically disclosed are cancer gene combinations comprising p53 and Ras.

It is understood that the cancer gene combinations can include combinations of only oncogenes and/or their gain or loss of function mutants. Therefore, disclosed herein are methods for identifying targets for the treatment of a cancer comprising performing an assay that measures differential expression of a gene or protein and identifying those genes, proteins, or micro RNAs that respond synergistically to the combination of two or more cancer genes, wherein the combination of two or more cancer genes comprises two or more oncogenes lwherein the oncogenes are selected from the list of oncogenes consisting of ras, raf, Bcl-2, Akt, Sis, src, Notch, Stathmin, mdm2, abl, hTERT, c-fos, c-jun, c-myc, erbB, HER2/Neu, HER3, c-kit, c-met, c-ret, flt3, AP1, AML1, axl, alk, fms, fps, gip, lck, MLM, PRAD-1, and trk. Likewise, it is understood that the cancer gene combinations can include combinations of only tumor suppressor genes and/or their gain or loss of function mutants. Therefore, disclosed herein are methods for identifying targets for the treatment of a cancer comprising performing an assay that measures differential expression of a gene or protein and identifying those genes, proteins, or micro RNAs that respond synergistically to the combination of two or more cancer genes, wherein the combination of two or more cancer genes comprises two or more tumor suppressor genes wherein the tumor suppressor gene is selected from the list of tumor suppressor genes consisting of p53, Rb, PTEN, BRCA-1, BRCA-2, APC, p57, p27, p16, p21, p73, p14ARF, Chek2, NF1, NF2, VHL, WRN, WT1, MEN1, MTS1, SMAD2, SMAD3, and SMAD4.

The methods disclosed herein can be assayed by any means to measure differential expression of a gene or protein known in the art. Specifically contemplated herein are methods of identifying targets for the treatment of a cancer comprising performing an assay that measures differential expression of a gene. Specifically contemplated are methods of identifying targets for the treatment of a cancer comprising performing an assay that measures differential gene expression, wherein the assay is selected from the group of assays consisting of, Northern analysis, RNAse protection assay, PCR, QPCR, genome microarray, low density PCR array, oligo array, SAGE and high throughput sequencing. Also disclosed herein are methods of identifying targets for the treatment of a cancer comprising performing an assay that measures differential expression of a protein. Specifically contemplated are methods of identifying targets for the treatment of a cancer comprising performing an assay that measures differential protein expression wherein the assay is selected from the group of assays consisting of protein microarray, antibody-based or protein activity-based detection assays and mass spectrometry.

It is understood and herein contemplated that the methods disclosed herein can be combined with additional methods known in the art to further identify the targets, assess the effect of the targets on a cancer or screen for agents that interact with the targets and through the interaction modulate cancer. Therefore, disclosed herein are methods of identifying targets for the treatment of a cancer comprising performing an assay that measures differential expression of a gene or protein and identifying those genes, proteins, or micro RNAs that respond synergistically to the combination of two or more cancer genes and further comprising measuring the effect of the targets on neoplastic cell transformation in vitro, in vitro cell death, in vitro survival, in vivo cell death, in vivo survival, in vitro angiogenesis, in vivo tumor angiogenesis, tumor formation, tumor maintenance, or tumor proliferation. It is also understood that there are many means known in the art for measuring the effect of the targets. One such method is through the perturbation of one or more targets and assaying for a change in the tumor or cancer cells relative to a control. Thus, for example, disclosed herein are methods, wherein the effect of the targets is measured through the perturbation of one or more targets and assaying for a change in the tumor or cancer cells relative to a control wherein a difference in the tumor or cancer cells relative to a control indicates a target that affects the tumor.

It is understood that the disclosed compositions and methods can be used to treat, identify targets for treatment of, or screen for agents that treat any disease where uncontrolled cellular proliferation occurs such as cancers. A non-limiting list of different types of cancers is as follows: lymphomas (Hodgkins and non-Hodgkins), leukemias, carcinomas, carcinomas of solid tissues, squamous cell carcinomas, adenocarcinomas, sarcomas, gliomas, high grade gliomas, blastomas, neuroblastomas, plasmacytomas, histiocytomas, melanomas, adenomas, hypoxic tumours, myelomas, AIDS-related lymphomas or sarcomas, metastatic cancers, or cancers in general.

A representative but non-limiting list of cancers that the disclosed compositions can be used to treat is the following: lymphoma, B cell lymphoma, T cell lymphoma, mycosis fungoides, Hodgkin's Disease, leukemias, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, gastric cancer, colon cancer, cervical cancer, cervical carcinoma, breast cancer, and epithelial cancer, bone cancers, renal cancer, bladder cancer, genitourinary cancer, esophageal carcinoma, large bowel cancer, metastatic cancers hematopoietic cancers, sarcomas, Ewing's sarcoma, synovial cancer, soft tissue cancers; and testicular cancer. Thus disclosed herein are methods of treating wherein the cancer is selected form the group of cancers consisting of lymphoma, B cell lymphoma, T cell lymphoma, mycosis fungoides, Hodgkin's Disease, leukemias, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, gastric cancer, colon cancer, cervical cancer, cervical carcinoma, breast cancer, and epithelial cancer, bone cancers, renal cancer, bladder cancer, genitourinary cancer, esophageal carcinoma, large bowel cancer, metastatic cancers hematopoietic cancers, sarcomas, Ewing's sarcoma, synovial cancer, soft tissue cancers; and testicular cancer.

Compounds and methods disclosed herein may also be used for the treatment of precancer conditions such as cervical and anal dysplasias, other dysplasias, severe dysplasias, hyperplasias, atypical hyperplasias, and neoplasias.

It is further understood that the targets in the disclosed methods can be cooperation response genes selected from the list of cooperation response genes consisting of Abat, Abcal, Ank, Ankrd1, Arhgap24, Atp8a1, Bbs7, Bex1, Ccl9, Centd3, Chst1, Ckmt1, Col9a3, Cpz, Cxcl1, Cxcl15, Daf1, Dapk1, Dffb, Dgka, Dixdc, Dusp15, Elay12, Eno3, Ephb2, Espn, Eva1, Fas, F2r11, Fgf18, Fgf7, Fhod3, FHOS2, Garn13, Gca, Gpr149, Hbegf, Hey2, Hmga1, Hmga2, Hoxc13, Id2, Id4, Igfbp2, Igsf4a, Jag2, Kctd15, Lass4, Ldhb, Man2b1, Mcam, Mmp15, Mpp7, Mrpl15, Mrpplf4, Ms4a10, Mtus1, Nbea, Notch3, Noxa, Oaf, Parvb, Pard6g, Perp, Plac8, Pla2g7, Pitx2, Pltp, Plxdc2, Prkcm, Prkg, Prss22, Pyr14, Rab40b, Rai2, Rasl11a, Rb1, Rgs2, Rprm, Rspo3, Satb1, Sbk1, Sbsn, Scn3b, Sema3d, Sema7a, Serpinb2, Sfrp2, Slc14a1, Slc27a3, Sms, Sod3, Stmn4, Tex15, Tnfrsf18, Tnnt2, Unc45b, Wnt9a, Zac1, Zfp385, and the cooperation response genes identified by the Genbank accession numbers AV133559, BM118398, BB353853, BB381558, AV231983, AI848263, AV244175, BF159528, AV231424, AV234963, BC013499, AV254040, BG071013, AK003981, BG066186, AK005731, BC027185, AK009671, AV323203, AI509011, BM220576, BQ173895, AV024662, BB207363, BC026627, AK017369, BQ031255, BC007193, BE949277, AK018275, BB704967, BB312717, AK018112, BI905111, BE957307, BG066982, BB358264, BB478071, AV298358, BB767109, AA266723, AV241486, BB133117, AI450842, and AW543723. It is a further embodiment that the target is a cooperation response gene selected from the group of cooperation response genes consisting of Abca1, Ank, Arhgap24, Atp8a1, Bbs7, Bnip3, Cox6b2, Cxcl1, Daf1, Dap, Dapk1, Dffb, Dgka, Dixdc, Eno3, Ephb2, Eva1, Fas, Fgf7, Gpr149, Hbegf, Hey2, Hmga1, Hoxc13, Id2, Id4, Igsf4a, Jag2, Mcam, Notch3, Noxa, Nrp2, Oaf, Pard6g, Perp, Pitx2, Plac8, Pla2g7, Pltp, Plxdc2, Prkg, Pyr14, Rab40b, Rb1, Rgs2, Rprm, Satb1, Sbk1, Sema3d, Sfrp2, Slc14a1, Sod3, Stmn4, Unc45b, Wnt9a, Zac1, and Zfp385.

It is also understood and herein contemplated that there can be instances where despite up or down-regulation of a CRG, pertubrbation of a single CRG does not result in an inhibition of the disease or condition, but perturbation of more than one CRG does result in inhibition. Thus, disclosed herein are combinations one or more targets are selected from the group of targets consisting of Abca1, Ank, Arhgap24, Atp8a1, Bbs7, Bnip3, Cox6b2, Cxcl1, Daf1, Dap, Dapk1, Dffb, Dgka, Dixdc, Eno3, Ephb2, Eva1, Fas, Fgf7, Gpr149, Hbegf, Hey2, Hmga1, Hoxc13, Id2, Id4, Igsf4a, Jag2, Mcam, Notch3, Noxa, Nrp2, Oaf, Pard6g, Perp, Pitx2, Plac8, Pla2g7, Pltp, Plxdc2, Prkg, Pyr14, Rab40b, Rb1, Rgs2, Rprm, Satb1, Sbk1, Sema3d, Sfrp2, Slc14a1, Sod3, Stmn4, Unc45b, Wnt9a, Zac1, and Zfp385. For example, disclosed herein are methods of identifying targets wherein the one or more targets are combinations of CRGs such as Dapk and Noxa; Dapk and Perp; Dapk and Sfrp2; Dffb and Sfrp2; Fas and Rprm; Noxa and Rprm; Noxa and Sfrp2; and Rprm and Sfrp2.

2. Methods for Screening for Agents that Treat Cancer

It is understood and herein contemplated that the targets identified through the methods disclosed herein have many uses, for example, as targets for drug treatment or screening for agents that modulate the targets identified by the methods disclosed herein. Agents identified though screening for affects on the targets can inhibit cancer. Thus disclosed herein are methods for screening for an agent that treats a cancer comprising contacting the agent with a target identified by the methods disclosed herein, wherein an agent that modulates the target such that tumor activity is inhibited is an agent that treats cancer. Specifically, disclosed herein are methods for screening for an agent that treats a cancer comprising contacting the agent with a target identified by performing an assay that measures differential expression of a gene or protein and identifying those genes, proteins, or micro RNAs that respond synergistically to the combination of two or more cancer genes, wherein an agent that modulates the target such that tumor activity is inhibited is an agent that treats cancer. Also disclosed are methods wherein the differential expression of a gene or protein is identified by N-test, T-test, or multiplicative synergy score, or additive synergy score.

Numerous studies indicate the utility of gene expression-based strategies for identifying drugs that mimic or reverse biological states across different cell types and species (Hassane et al., 2008; Hieronymus et al., 2006; Hughes et al., 2000; Lamb et al., 2006; Stegmaier et al., 2004; Stegmaier et al., 2007; Wei et al., 2006). To facilitate such comparisons, the Connectivity Map (CMap) was created (Lamb et al., 2006).

a) Connectivity Map

The Connectivity Map is a gene expression repository comprising a compendium of microarray gene expression data obtained from cells in a particular biological state. Generally, such states can arise from exposure to small molecules/drugs, RNAi, gene transduction, gene knockout, mutation, or disease. Connectivity Map is able to independently obtain a gene expression signature arising from a treatment of interest (query signature) and identify instances of biological states within the Connectivity Map most similar to this query signature. Thus, any known or unknown biological state can be connected to a known biological state based on microarray gene expression data. Therefore, disclosed herein are methods of identifying compositions having anti-cancer activity, wherein the process of identifying of molecules which modulate the related gene set is performed by using the connectivity map. Positive connectivity can identify common biological effects of compounds (Lamb et al., 2006). The CMap can also identify antagonists of disease states, via negative connectivity, including novel putative inhibitors of Alzheimer's disease, dexamethasone-resistant acute lymphoblastic leukemia and acute myeloid leukemia stem cells (Hassane et al., 2008; Lamb et al., 2006; Wei et al., 2006). Herein, the CMap was utilized to identify instances of negative connectivity to the CRG signature, in order to find pharmacologic agents that reverse the CRG signature and function to inhibit malignant transformation.

b) Random Forest

RANDOM FOREST® is an algorithm based classifier decision tree which provides data on the correlation and strength of individual datapoints called trees.

c) Gene Expression Omnibus

The Gene Expression Omnibus (GEO) is a public gene expression repository which is updated through submission of experimental date of microarray analysis measiuring mRNA, miRNA, genomic DNA (arrayCGH, ChIP-chip, and SNP), and protein abundance as well as serial analysis of gene expression (SAGE). The database holds over 500 million gene expression measurements.

It is understood and herein contemplated that a single agent may not be effective in the treatment of a cancer or the modulation of one or more of the targets identified by the methods disclosed herein. Thus, disclosed herein are methods for screening for a combination of two or more agents that treats a cancer comprising contacting the agent with a target identified by the methods disclosed herein, wherein an agent that modulates the target such that tumor activity is inhibited is an agent that treats cancer.

It is further understood that, as noted above, the targets in the disclosed methods can be cooperation response genes selected from the list of cooperation response genes consisting of Abat, Abca1, Ank, Ankrd1, Arhgap24, Atp8a1, Bbs7, Bex1, Ccl9, Centd3, Chst1, Ckmt1, Col9a3, Cpz, Cxcl1, Cxcl15, Daf1, Dapk1, Dffb, Dgka, Dixdc, Dusp15, Elav12, Eno3, Ephb2, Espn, Eva1, Fas, F2r11, Fgf18, Fgf7, Fhod3, FHOS2, Garn13, Gca, Gpr149, Hbegf, Hey2, Hmga1, Hmga2, Hoxc13, Id2, Id4, Igfbp2, Igsf4a, Jag2, Kctd15, Lass4, Ldhb, Man2b1, Mcam, Mmp15, Mpp7, Mrpl15, Mrpplf4, Ms4a10, Mtus1, Nbea, Notch3, Noxa, Oaf, Parvb, Pard6g, Perp, Plac8, P1a2g7, Pitx2, Pltp, Plxdc2, Prkcm, Prkg, Prss22, Pyr14, Rab40b, Rai2, Rasl11a, Rb1, Rgs2, Rprm, Rspo3, Satb1, Sbk1, Sbsn, Scn3b, Sema3d, Sema7a, Serpinb2, Sfrp2, Slc14a1, Slc27a3, Sms, Sod3, Stmn4, Tex15, Tnfrsf18, Tnnt2, Unc45b, Wnt9a, Zac1, Zfp385, and the cooperation response genes identified by the Genbank accession numbers AV133559, BM118398, BB353853, BB381558, AV231983, AI848263, AV244175, BF159528, AV231424, AV234963, BC013499, AV254040, BG071013, AK003981, BG066186, AK005731, BC027185, AK009671, AV323203, AI509011, BM220576, BQ173895, AV024662, BB207363, BC026627, AK017369, BQ031255, BC007193, BE949277, AK018275, BB704967, BB312717, AK018112, BI905111, BE957307, BG066982, BB358264, BB478071, AV298358, BB767109, AA266723, AV241486, BB133117, AI450842, and AW543723. It is a further embodiment that the target is a cooperation response gene selected from the group of cooperation response genes consisting of Abca1, Ank, Arhgap24, Atp8a1, Bbs7, Bnip3, Cox6b2, Cxcl1, Daf1, Dap, Dapk1, Dffb, Dgka, Dixdc, Eno3, Ephb2, Eva1, Fas, Fgf7, Gpr149, Hbegf, Hey2, Hmga1, Hoxc13, Id2, Id4, Igsf4a, Jag2, Mcam, Notch3, Noxa, Nrp2, Oaf, Pard6g, Perp, Pitx2, Plac8, P1a2g7, Pltp, Plxdc2, Prkg, Pyr14, Rab40b, Rb1, Rgs2, Rprm, Satb1, Sbk1, Sema3d, Sfrp2, Slc14a1, Sod3, Stmn4, Unc45b, Wnt9a, Zac1, and Zfp385. Thus, specifically disclosed herein are methods for screening for one or more agents (such as a combination of two or more agents) that treats cancer comprising contacting the agent with the one or more targets, wherein the agent modulates the activity of the target in a manner such that tumor survival or growth (including but not limited to neoplastic cell transformation in vitro, in vitro cell death, in vivo cell death, in vitro angiogenesis, in vivo tumor angiogenesis, tumor formation, tumor maintenance, or tumor proliferation or further decrease in in vitro or in vivo survival) is inhibited, and wherein the targets are selected from the group of targets consisting of Abat, Abca1, Ank, Ankrd1, Arhgap24, Atp8a1, Bbs7, Bex1, Ccl9, Centd3, Chst1, Ckmt1, Col9a3, Cpz, Cxcl1, Cxcl15, Daf1, Dapk1, Dffb, Dgka, Dixdc, Dusp15, Elav12, Eno3, Ephb2, Espn, Eva1, Fas, F2r11, Fgf18, Fgf7, Fhod3, FHOS2, Garn13, Gca, Gpr149, Hbegf, Hey2, Hmga1, Hmga2, Hoxc13, Id2, Id4, Igfbp2, Igsf4a, Jag2, Kctd15, Lass4, Ldhb, Man2b1, Mcam, Mmp15, Mpp7, Mrpl15, Mrpplf4, Ms4a10, Mtus1, Nbea, Notch3, Noxa, Oaf, Parvb, Pard6g, Perp, Plac8, P1a2g7, Pitx2, Pltp, Plxdc2, Prkcm, Prkg, Prss22, Pyr14, Rab40b, Rai2, Rasl11a, Rb1, Rgs2, Rprm, Rspo3, Satb1, Sbk1, Sbsn, Scn3b, Sema3d, Sema7a, Serpinb2, Sfrp2, Slc14a1, Slc27a3, Sms, Sod3, Stmn4, Tex15, Tnfrsf18, Tnnt2, Unc45b, Wnt9a, Zac1, Zfp385, and the cooperation response genes identified by the Genbank accession numbers AV133559, BM118398, BB353853, BB381558, AV231983, AI848263, AV244175, BF159528, AV231424, AV234963, BC013499, AV254040, BG071013, AK003981, BG066186, AK005731, BC027185, AK009671, AV323203, AI509011, BM220576, BQ173895, AV024662, BB207363, BC026627, AK017369, BQ031255, BC007193, BE949277, AK018275, BB704967, BB312717, AK018112, BI905111, BE957307, BG066982, BB358264, BB478071, AV298358, BB767109, AA266723, AV241486, BB133117, AI450842, and AW543723. It is understood that the one or more agents can comprise 1, 2, 3, 4, 5, 6, 7, 8, 9, or 10 agents. Thus, disclosed herein are methods for screening comprising one agent. Also disclosed are methods for screening for a combination of two or more agents that treats cancer comprising contacting the agent with the one or more targets, wherein the agent modulates the activity of the target in a manner such that tumor proliferation is inhibited, and wherein the targets are selected from the group of targets consisting of Abat, Abca1, Ank, Ankrd1, Arhgap24, Atp8a1, Bbs7, Bex1, Ccl9, Centd3, Chst1, Ckmt1, Col9a3, Cpz, Cxcl1, Cxcl15, Daf1, Dapk1, Dffb, Dgka, Dixdc, Dusp15, Elav12, Eno3, Ephb2, Espn, Eva1, Fas, F2r11, Fgf18, Fgf7, Fhod3, FHOS2, Garn13, Gca, Gpr149, Hbegf, Hey2, Hmga1, Hmga2, Hoxc13, Id2, Id4, Igfbp2, Igsf4a, Jag2, Kctd15, Lass4, Ldhb, Man2b1, Mcam, Mmp15, Mpp7, Mrpl15, Mrpplf4, Ms4a10, Mtus1, Nbea, Notch3, Noxa, Oaf, Parvb, Pard6g, Perp, Plac8, P1a2g7, Pitx2, Pltp, Plxdc2, Prkcm, Prkg, Prss22, Pyr14, Rab40b, Rai2, Rasl11a, Rb1, Rgs2, Rprm, Rspo3, Satb1, Sbk1, Sbsn, Scn3b, Sema3d, Sema7a, Serpinb2, Sfrp2, Slc14a1, Slc27a3, Sms, Sod3, Stmn4, Tex15, Tnfrsf18, Tnnt2, Unc45b, Wnt9a, Zac1, Zfp385, and the cooperation response genes identified by the Genbank accession numbers AV133559, BM118398, BB353853, BB381558, AV231983, AI848263, AV244175, BF159528, AV231424, AV234963, BC013499, AV254040, BG071013, AK003981, BG066186, AK005731, BC027185, AK009671, AV323203, AI509011, BM220576, BQ173895, AV024662, BB207363, BC026627, AK017369, BQ031255, BC007193, BE949277, AK018275, BB704967, BB312717, AK018112, BI905111, BE957307, BG066982, BB358264, BB478071, AV298358, BB767109, AA266723, AV241486, BB133117, AI450842, and AW543723.

It is also understood and herein contemplated that there can be instances where despite up or down-regulation of a CRG, pertubrbation of a single CRG does not result in an inhibition of the disease or condition, but perturbation of more than one CRG does result in inhibition. Thus, disclosed herein are methods of screening where the screen is conducted on more than one target and wherein the one or more targets are selected from the group of targets consisting of Abca1, Ank, Arhgap24, Atp8a1, Bbs7, Bnip3, Cox6b2, Cxcl1, Daf1, Dap, Dapk1, Dffb, Dgka, Dixdc, Eno3, Ephb2, Eva1, Fas, Fgf7, Gpr149, Hbegf, Hey2, Hmga1, Hoxc13, Id2, Id4, Igsf4a, Jag2, Mcam, Notch3, Noxa, Nrp2, Oaf, Pard6g, Perp, Pitx2, Plac8, P1a2g7, Pltp, Plxdc2, Prkg, Pyr14, Rab40b, Rb1, Rgs2, Rprm, Satb1, Sbk1, Sema3d, Sfrp2, Slc14a1, Sod3, Stmn4, Unc45b, Wnt9a, Zac1, and Zfp385. For example, disclosed herein are methods of screening wherein the one or more targets are combinations of CRGs such as Dapk and Noxa; Dapk and Perp; Dapk and Sfrp2; Dffb and Sfrp2; Fas and Rprm; Noxa and Rprm; Noxa and Sfrp2; and Rprm and Sfrp2.

It is understood and herein contemplated that the desired effect of the agent on the cooperation response gene depends on the activity of the cooperation response gene and its effect on the cancer. In some cases for inhibition of the cancer to occur, the cooperation response gene must be inhibited and in other cases enhanced. Thus, it is understood and herein contemplated that disclosed agents can modulate the activity of the target through inhibition or enhancement. Therefore, disclosed herein are methods for screening for an agent that treats cancer comprising contacting the agent with the one or more targets, wherein the agent modulates the activity of the target in a manner such that tumor proliferation is inhibited, wherein the agent modulation of the activity of the target is inhibition. In particular, disclosed herein are methods for screening for an agent that treats cancer comprising contacting the agent with the one or more targets, wherein the agent inhibits the activity of the target in a manner such that tumor proliferation is inhibited, wherein the target is a cooperation response gene. Further disclosed are methods wherein the cooperation response gene selected from the group consisting of Ank, Cxcl1, Eno3, Fgf7, Gpr149, Hmga1, Id4, Igsf4a, Oaf, P1a2g7, Plac8, Pltp, Plxdc2, Rgs2, and Sod3.

Also disclosed herein are methods for screening for an agent that treats cancer comprising contacting the agent with the one or more targets, wherein the agent modulates the activity of the target in a manner such that tumor proliferation is inhibited, wherein the agent modulation of the activity of the target is enhanced. In particular, disclosed herein are methods for screening for an agent that treats cancer comprising contacting the agent with the one or more targets, wherein the agent enhances the activity of the target in a manner such that tumor proliferation is inhibited, wherein the target is a cooperation response gene. Further disclosed are methods wherein the cooperation response gene selected from the group consisting of Abca1, Arhgap24, Atp8a1, Bbs7, Daf1, Dapk1, Dffb, Dgka, Dixdc, Ephb2, Eva1, Fas, Hey2, Hmga1, Hoxc13, Id2, Jag2, Mcam, Notch3, Noxa, Pard6g, Perp, Pitx2, Prkg, Pvr14, Rab40b, Rb1, Rprm, Satb1, Sbk1, Sema3d, Sfrp2, Slc14a1, Stmn4, Unc45b, Wnt9a, Zac1, and Zfp385.

3. Method of Treating Cancer

The agents identified by the screening methods disclosed herein have many uses, for example, the treatment of a cancer. Disclosed herein are methods of treating a cancer in a subject comprising administering to the subject one or more agents that modulate the activity of one or more cooperation response genes.

“Treatment,” “treat,” or “treating” mean a method of reducing the effects of a disease or condition. Treatment can also refer to a method of reducing the disease or condition itself rather than just the symptoms. The treatment can be any reduction from native levels and can be but is not limited to the complete ablation of the disease, condition, or the symptoms of the disease or condition. Therefore, in the disclosed methods, “treatment” can refer to a 10%, 20%, 30%, 40%, 50%, 60%, 70%, 80%, 90%, or 100% reduction in the severity of an established disease or the disease progression. For example, a disclosed method for reducing the effects of prostate cancer is considered to be a treatment if there is a 10% reduction in one or more symptoms of the disease in a subject with the disease when compared to native levels in the same subject or control subjects. Thus, the reduction can be a 10, 20, 30, 40, 50, 60, 70, 80, 90, 100%, or any amount of reduction in between as compared to native or control levels. It is understood and herein contemplated that “treatment” does not necessarily refer to a cure of the disease or condition, but an improvement in the outlook of a disease or condition.

It is understood and herein contemplated that the one or more agents can modulate that activity of any of the targets disclosed herein. Thus, disclosed herein in one embodiment are methods wherein the one of more agents modulate the activity of one or more targets. Further disclosed are methods wherein the one or more targets are one or more cooperation response genes. Thus disclosed herein in one embodiment are methods wherein the one of more agents modulate the activity of one or more cooperation response genes selected for the group consisting of Abat, Abca1, Ank, Ankrd1, Arhgap24, Atp8a1, Bbs7, Bex1, Ccl9, Centd3, Chst1, Ckmt1, Col9a3, Cpz, Cxcl1, Cxcl15, Daf1, Dapk1, Dffb, Dgka, Dixdc, Dusp15, Elav12, Eno3, Ephb2, Espn, Eva1, Fas, F2r11, Fgf18, Fgf7, Fhod3, FHOS2, Garn13, Gca, Gpr149, Hbegf, Hey2, Hmga1, Hmga2, Hoxc13, Id2, Id4, Igfbp2, Igsf4a, Jag2, Kctd15, Lass4, Ldhb, Man2b1, Mcam, Mmp15, Mpp7, Mrpl15, Mrpplf4, Ms4a10, Mtus1, Nbea, Notch3, Noxa, Oaf, Parvb, Pard6g, Perp, Plac8, P1a2g7, Pitx2, Pltp, Plxdc2, Prkcm, Prkg, Prss22, Pyr14, Rab40b, Rai2, Rasl11a, Rb1, Rgs2, Rprm, Rspo3, Satb1, Sbk1, Sbsn, Scn3b, Sema3d, Sema7a, Serpinb2, Sfrp2, Slc14a1, Slc27a3, Sms, Sod3, Stmn4, Tex15, Tnfrsf18, Tnnt2, Unc45b, Wnt9a, Zac1, Zfp385, as well as the cooperation response genes identified by the Genbank accession number AV133559, BM118398, BB353853, BB381558, AV231983, AI848263, AV244175, BF159528, AV231424, AV234963, BC013499, AV254040, BG071013, AK003981, BG066186, AK005731, BC027185, AK009671, AV323203, AI509011, BM220576, BQ173895, AV024662, BB207363, BC026627, AK017369, BQ031255, BC007193, BE949277, AK018275, BB704967, BB312717, AK018112, BI905111, BE957307, BG066982, BB358264, BB478071, AV298358, BB767109, AA266723, AV241486, BB133117, AI450842, and AW543723. In a further aspect, disclosed herein are methods of treating cancer wherein the one or more cooperation response genes are selected from the group consisting of Abca1, Ank, Arhgap24, Atp8a1, Bbs7, Bnip3, Cox6b2, Cxcl1, Daf1, Dap, Dapk1, Dffb, Dgka, Dixdc, Eno3, Ephb2, Eva1, Fas, Fgf7, Gpr149, Hbegf, Hey2, Hmga1, Hoxc13, Id2, Id4, Igsf4a, Jag2, Mcam, Notch3, Noxa, Nrp2, Oaf, Pard6g, Perp, Pitx2, Plac8, P1a2g7, Pltp, Plxdc2, Prkg, Pyr14, Rab40b, Rb1, Rgs2, Rprm, Satb1, Sbk1, Sema3d, Sfrp2, Slc14a1, Sod3, Stmn4, Unc45b, Wnt9a, Zac1, and Zfp385

It is understood and herein contemplated that the activity of the cooperation response gene can be modulated by modulating the expression of one or more, two or more, three or more, four or more, or five or more of the CRG. It is further understood and herein contemplated that the expression can be inhibited or enhanced. It is understood and herein contemplated that those of skill in the art will understand whether to inhibit or enhance the activity of one or more cooperation response genes. For example, one of skill in the art will understand that where the expression of a particular CRG is up-regulated in a cancer, one of skill in the art will want to administer an agent that decreases or inhibits the up-regulation of the CRG. Similarly, where the expression of a particular CRG is down-regulated in a cancer, one of skill in the art will want to administer an agent that increases or enhances the expression of the down-regulated CRG. However, it is also understood that in some cases such as, for example, Pltp, when a down-regulated CRG is enhanced tumor size increases. It is understood that those of skill in the art will recognize that for those down-regulated CRG's that result in increased tumor size when the CRG expression or activity is increased, are treated with an agent that decreases expression or the activity of the CRG. Similarly, where an up-regulated CRG when inhibited leads to increased tumor volume (as happens with Slc14a1), treatment involves enhancing or increasing expression or activity of the CRG. Moreover, it is contemplated herein that one method of treating cancer is to administer an agent that targets down-regulated CRG's in combination with an agent that targets up-regulated CRG's. Therefore, for example, disclosed herein are methods of treating cancer comprising administering to the subject one or more agents that inhibits the activity of one or more cooperation response genes. Also disclosed are methods wherein the cooperation response gene is selected from the group consisting of Ank, Cxcl1, Eno3, Fgf7, Gpr149, Hmga1, Id4, Igsf4a, Oaf, P1a2g7, Plac8, Pltp, Plxdc2, Rgs2, and Sod3. Also disclosed are methods of treating cancer comprising administering to the subject one or more agents that enhances the activity of one or more cooperation response genes. Also disclosed are methods wherein the cooperation response gene is selected from the group consisting of Abca1, Arhgap24, Atp8a1, Bbs7, Daf1, Dapk1, Dffb, Dgka, Dixdc, Ephb2, Eva1, Fas, Hey2, Hmga1, Hoxc13, Id2, Jag2, Mcam, Notch3, Noxa, Pard6g, Perp, Pitx2, Prkg, Pyr14, Rab40b, Rb1, Rprm, Satb1, Sbk1, Sema3d, Sfrp2, Slc14a1, Stmn4, Unc45b, Wnt9a, Zac1, and Zfp385. Thus, for example, disclosed herein are method of treating a cancer comprising administering to a subject one or more agents such as (+)-chelidonine, 0179445-0000, 0198306-0000, 1,4-chrysenequinone, 15-delta prostaglandin J2, 2,6-dimethylpiperidine, 4-hydroxyphenazone, 5186223, 6-azathymine, acenocoumarol, alpha-estradiol, altizide, alverine, alvespimycin, amikacin, aminohippuric acid, amoxicillin, amprolium, ampyrone, antimycin A, arachidonyltrifluoromethane, atractyloside, azathioprine, azlocillin, bacampicillin, baclofen, bambuterol, beclometasone, benzylpenicillin, betaxolol, betulinic acid, biperiden, boldine, bromocriptine, bufexamac, buspirone, butacaine, butirosin, calycanthine, canadine, canavanine, carbarsone, carbenoxolone, carbimazole, carcinine, carmustine, cefalotin, cefepime, ceftazidime, cephaeline, chenodeoxycholic acid, chlorhexidine, chlorogenic acid, chlorpromazine, chlortalidone, cinchonidine, cinchonine, clemizole, co-dergocrine mesilate, CP-320650-01, CP-690334-01, dacarbazine, demeclocycline, dexibuprofen, dextromethorphan, dicycloverine, diethylstilbestrol, diflorasone, diflunisal, dihydroergotamine, diloxanide, dinoprostone, diphemanil metilsulfate, diphenylpyraline, doxylamine, droperidol, epirizole, epitiostanol, esculetin, estradiol, estropipate, ethionamide, etofenamate, etomidate, eucatropine, famotidine, famprofazone, fendiline, fisetin, fludrocortisone, flufenamic acid, flupentixol, fluphenazine, fluticasone, fluvastatin, fosfosal, fulvestrant, gabexate, galantamine, gemfibrozil, genistein, glibenclamide, gliquidone, glycocholic acid, gossypol, gramine, guanadrel, halcinonide, haloperidol, harpagoside, hexamethonium bromide, homochlorcyclizine, hydroxyzine, idoxuridine, ifosfamide, indapamide, iobenguane, iopanoic acid, iopromide, isoetarine, isoxsuprine, isradipine, ketorolac, ketotifen, lanatoside C, lansoprazole, laudanosine, letrozole, levodopa, levomepromazine, lidocaine, liothyronine, lisinopril, lisuride, LY-294002, lynestrenol, meclofenamic acid, meclofenoxate, medrysone, mefloquine, mepacrine, methapyrilene, methazolamide, methyldopa, methylergometrine, metoclopramide, mevalolactone, mometasone, monensin, monorden, naftopidil, nalbuphine, naltrexone, napelline, naphazoline, naringin, niclosamide, niflumic acid, nimesulide, nomifensine, noretynodrel, norfloxacin, orphenadrine, oxolinic acid, oxprenolol, papaverine, pentolonium, pepstatin, perphenazine, PF-00562151-00, phenelzine, phenindione, pheniramine, phthalylsulfathiazole, pinacidil, pioglitazone, piperine, piretanide, piribedil, pirlindole, PNU-0230031, pralidoxime, pramocaine, praziquantel, prednisone, Prestwick-1100, Prestwick-981, probenecid, prochlorperazine, proglumide, propofol, protriptyline, racecadotril, riboflavin, rifabutin, rimexolone, roxithromycin, santonin, SB-203580, SC-560, scopoletin, scriptaid, seneciphylline, sirolimus, sitosterol, sodium phenylbutyrate, solanine, spectinomycin, spiradoline, SR-95531, SR-95639A, sulfadimidine, sulfaguanidine, sulfanilamide, sulfathiazole, tanespimycin, terbutaline, terguride, thalidomide, thiamazole, thiamphenicol, thioridazine, ticarcillin, ticlopidine, timidazole, tiratricol, tolfenamic acid, tremorine, trichostatin A, trifluoperazine, troglitazone, tyloxapol, ursodeoxycholic acid, valproic acid, vanoxerine, vidarabine, vincamine, vorinostat, wortmannin, yohimbic acid, yohimbine, or zidovudine.

Also disclosed are methods of treating a cancer comprising administering to the subject one or more, two or more, three or more, four or more, or five or more agents that enhance the activity of one or more CRG's in combination with one or more, two or more, three or more, four or more, or five or more agents that enhance the activity of one or more CRG's. Also disclosed are methods wherein the CRG's that are enhanced are selected from the group consisting of Abca1, Arhgap24, Atp8a1, Bbs7, Daf1, Dapk 1, Dffb, Dgka, Dixdc, Ephb2, Eva1, Fas, Hey2, Hmga1, Hoxc13, Id2, Jag2, Mcam, Notch3, Noxa, Pard6g, Perp, Pitx2, Prkg, Pyr14, Rab40b, Rb1, Rprm, Satb1, Sbk1, Sema3d, Sfrp2, Slc14a1, Stmn4, Unc45b, Wnt9a, Zac1, and Zfp385. Examples of agent that enhance CRG expression or activity include, but are not limited to 6-benzylaminopurine, 8-azaguanine, acetylsalicylic acid, allantoin, alpha-yohimbine, azlocillin, bemegride, benfluorex, benfotiamine, berberine, bromopride, cantharidin, carbachol, chloramphenicol, cinoxacin, citiolone, daunorubicin, desoxycortone, dicloxacillin, dosulepin, epitiostanol, ethaverine, ethotoin, etofylline, etynodiol, fenoprofen, fluorometholone, geldanamycin, ginkgolide A, hesperetin, iohexyl, ioversol, ioxaglic acid, ipratropium bromide, isoxsuprine, lisinopril, mebendazole, meclofenoxate, mephenesin, mestranol, meticrane, metoclopramide, metolazone, metoprolol, morantel, MS-275, napelline, neostigmine bromide, phenelzine, picrotoxinin, pimethixene, pipenzolate bromide, procainamide, pronetalol, propafenone, propantheline bromide, pyrimethamine, pyrvinium, quinidine, rifabutin, rolitetracycline, sanguinarine, skimmianine, S-propranolol, sulconazole, sulfametoxydiazine, sulfaphenazole, suloctidil, syrosingopine, tacrine, tanespimycin, thioguanosine, tolazamide, tracazolate, trichostatin A, trifluridine, triflusal, trimetazidine, trioxysalen, valproic acid, vidarabine, or vorinostat. Further disclosed are methods wherein the CRG's that are inhibited are selected from the group consisting of Ank, Cxcl1, Eno3, Fgf7, Gpr149, Hmga1, Id4, Igsf4a, Oaf, P1a2g7, Plac8, Pltp, Plxdc2, Rgs2, and Sod3. Examples of agent that inhibit CRG expression or activity include, but are not limited to (−)-MK-801, (+/−)-catechin, 0317956-0000, 15-delta prostaglandin J2, 2-aminobenzenesulfonamide, 3-acetamidocoumarin, 5155877, 5186324, 5194442, 7-aminocephalosporanic acid, abamectin, acebutolol, aceclofenac, acepromazine, adiphenine, AH-6809, alclometasone, alfuzosin, allantoin, alpha-ergocryptine, alprenolol, alprostadil, amantadine, ambroxol, amiloride, aminophylline, ampicillin, anabasine, arcaine, ascorbic acid, atovaquone, atracurium besilate, atropine, aztreonam, bambuterol, BCB000040, bemegride, benserazide, benzamil, benzbromarone, benzethonium chloride, benzocaine, benzonatate, benzydamine, bergenin, betamethasone, bethanechol, betonicine, brinzolamide, bucladesine, bumetanide, buspirone, butirosin, capsaicin, carbachol, carbarsone, carteolol, cefaclor, cefalonium, cefamandole, cefixime, ceforanide, cefotaxime, cefoxitin, cefuroxime, chlorcyclizine, chlorphenesin, chlortalidone, chlorzoxazone, ciclacillin, cimetidine, cinchonidine, cinchonine, clebopride, clemastine, clobetasol, clorsulon, clotrimazole, clozapine, clozapine, colchicines, colforsin, colistin, convolamine, coralyne, CP-690334-01, CP-863187, cyclopentolate, cytochalasin B, daunorubicin, decamethonium bromide, decitabine, demecarium bromide, dexamethasone, diazoxide, diclofenac, dicloxacillin, dicoumarol, dicycloverine, diethylcarbamazine, diflunisal, dihydroergocristine, dilazep, diloxanide, dinoprost, dinoprostone, diperodon, diphenhydramine, diphenylpyraline, disulfuram, dl-alpha tocopherol, dobutamine, dosulepin, doxepin, doxycycline, dropropizine, dyclonine, edrophonium chloride, enalapril, epivincamine, erythromycin, esculin, estradiol, estriol, estrone, ethotoin, etilefrine, F0447-0125, famprofazone, fasudil, felbinac, fenbendazole, fenofibrate, finasteride, florfenicol, flufenamic acid, fluocinonide, fluorocurarine, fluoxetine, fluphenazine, flurbiprofen, fluspirilene, flutamide, fluticasone, fluvastatin, fluvoxamine, foliosidine, fosfosal, fulvestrant, furosemide, fursultiamine, gabexate, geldanamycin, genistein, gentamicin, gibberellic acid, Gly-His-Lys, guanabenz, H-89, halcinonide, halofantrine, haloperidol, harmaline, harmalol, harmine, harpagoside, hecogenin, heliotrine, helveticoside, heptaminol, hydrocotamine, hydroquinine, ikarugamycin, iodixanol, iohexyl, iopamidol, ioversol, isoniazid, isopropamide iodide, isotretinoin, josamycin, kaempferol, kawain, ketanserin, ketoprofen, khellin, lactobionic acid, levobunolol, levodopa, lincomycin, lisuride, lisuride, lobelanidine, lomefloxacin, loperamide, loxapine, lumicolchicine, LY-294002, meclocycline, meclofenamic acid, mefloquine, mepyramine, merbromin, mesalazine, metamizole sodium, metampicillin, metanephrine, meteneprost, metergoline, methazolamide, methocarbamol, methoxamine, methoxsalen, methylbenzethonium chloride, methyldopate, methylergometrine, methylprednisolone, metitepine, metixene, metoclopramide, metolazone, metrizamide, metronidazole, mexiletine, mifepristone, mimosine, minaprine, minocycline, minoxidil, molindone, monastrol, monensin, moxonidine, myricetin, nabumetone, nadolol, nafcillin, naftidrofuryl, naftifine, naphazoline, naproxen, neomycin, neostigmine bromide, nimodipine, nitrofural, nizatidine, nomegestrol, norcyclobenzaprine, nordihydroguaiaretic acid, orlistat, orphenadrine, oxamniquine, oxaprozin, oxetacaine, oxolamine, oxprenolol, oxybutynin, oxymetazoline, palmatine, parbendazole, parthenolide, penbutolol, pentetrazol, pergolide, PF-00539745-00, PHA-00745360, PHA-00767505E, PHA-00851261E, phenazone, phenelzine, pheneticillin, phenoxybenzamine, phentolamine, pinacidil, pioglitazone, pirenperone, pivmecillinam, pizotifen, PNU-0230031, PNU-0251126, PNU-0293363, podophyllotoxin, practolol, prednicarbate, prenylamine, Prestwick-642, Prestwick-674, Prestwick-675, Prestwick-682, Prestwick-685, Prestwick-857, Prestwick-967, Prestwick-983, primidone, probenecid, probucol, prochlorperazine, propafenone, propranolol, pyrithyldione, quipazine, raloxifene, ramipril, R-atenolol, ribavirin, ribostamycin, rifampicin, riluzole, risperidone, rofecoxib, rolitetracycline, rosiglitazone, rotenone, rottlerin, santonin, SB-203580, scopolamine N-oxide, securinine, sertaconazole, simvastatin, sirolimus, sodium phenylbutyrate, sotalol, spiradoline, splitomicin, S-propranolol, SR-95639A, stachydrine, sulfachlorpyridazine, sulfadoxine, sulfamerazine, sulfamethoxypyridazine, sulfamonomethoxine, sulfathiazole, sulindac, syrosingopine, tacrine, tamoxifen, tanespimycin, terazosin, terguride, tetracycline, tetrandrine, tetryzoline, thapsigargin, thiamazole, thiamphenicol, thiostrepton, tiaprofenic acid, tiletamine, timidazole, tocamide, tolnaftate, topiramate, tracazolate, tranexamic acid, trapidil, tretinoin, tribenoside, trichostatin A, tridihexethyl, trifluoperazine, triflupromazine, trimethadione, trimethobenzamide, troglitazone, tubocurarine chloride, tyrphostin AG-1478, ursolic acid, valproic acid, vinblastine, vincamine, vinpocetine, vitexin, withaferin A, wortmannin, yohimbic acid, yohimbine, zalcitabine, zaprinast, zardaverine, zoxazolamine, and zuclopenthixol. It is understood and herein contemplated that any of the disclosed agents can be administered in combination. For example, disclosed herein are methods of treating a cancer comprising administering a first agent that enhances the expression or acitivity of one or more CRG's and a second agent the inhibits the expression or activity of one or more CRG's.

It is understood and contemplated herein that one means of treating cancer is through the administration of a single agent that modulates the expression or activity of one or more, two or more, three or more, four or more, or five or more cooperative response genes. It is understood and herein contemplated that modulation of expression is not the only means for modulating the activity of one or more cooperation response genes and such means can be accomplished by any manner known to those of skill in the art. Therefore, for example, disclosed herein are methods of treating cancer wherein the activity of the cooperation response gene is inhibited by the administration of an antibody, siRNA, small molecule inhibitory drug, shRNA, or peptide mimetic that is specific for the protein encoded by the cooperation response gene. Also disclosed are methods wherein the antibody, siRNA, small molecule inhibitory drug, or peptide mimetic is specific for the protein encoded by Ank, Cxcl1, Eno3, Fgf7, Gpr149, Hmga1, Id4, Igsf4a, Oaf, P1a2g7, Plac8, Pltp, Plxdc2, Rgs2, and Sod3.

In another aspect, the disclosed methods of treating cancer can be combined with anti-cancer agents such as, for example, chemotherapeutics or anti-oxidants known in the art. Therefore, disclosed herein are methods of treating a cancer in a subject comprising administering to the subject one or more anti-cancer agents and one or more agents that modulate the activity of one or more cooperation response genes. Further disclosed are methods wherein the anti-cancer agent is a chemotherapeutic or antioxidant compound. Also disclosed are methods wherein the anti-cancer agent is a histone deacetylase inhibitor.

Gene expression is highly dependent upon chromatin structure that is in turn regulated by the opposing activities of histone acetyltransferases (Baeg et al., 1995) and histone deacetylases (HDACs) (Marks et al., 2000). HDACs remove acetyl groups from lysine residues on histone tails, condensing chromatin structure and preventing transcription factor binding (Marks et al., 2000). Histone deacetylation is thus associated with heterochromatin and transcriptional silencing (Iizuka and Smith, 2003; Jenuwein and Allis, 2001), and this level of gene expression regulation is necessary for normal development as HDAC1 loss-of-function results in embryonic lethality (Lagger et al., 2002), knock out of HDAC4 results in defective skeletonogenesis (Vega et al., 2004), and knock out of HDACS or HDAC9 results in cardiac hypertrophy (Zhang et al., 2002).

There are four distinct classes of HDACs, the first two of which have been extensively characterized and are evolutionarily conserved among eukaryotic organisms (Minucci and Pelicci, 2006). HDAC1-3 and HDAC8 comprise class 1 and are related to the yeast RPD3 HDAC, and HDAC4-7, HDAC9, and HDAC10 comprise class 2 and are related to the yeast HDA1 HDAC (Minucci and Pelicci, 2006). While the members of both classes have a zinc-dependent catalytic domain, class 1 HDACs are constitutively nuclear proteins and class 2 HDACs shuttle between the cytoplasm and the nucleus (Minucci and Pelicci, 2006; Verdin et al., 2003). Class 1 HDACs are ubiquitously expressed, while class 2 HDACs exhibit varying degrees of tissue specificity (Minucci and Pelicci, 2006), which likely accounts for the embryonic lethality of knocking out HDAC1 versus the tissue-specific phenotypes of HDAC4, 5, and 9 knock-out mice (Lagger et al., 2002; Vega et al., 2004; Zhang et al., 2002).

The role of HDACs in cancer was first demonstrated in acute promyelocytic leukemia (Aplin et al.) where oncoproteins generated by the fusion of the retinoic acid receptor-α gene and either the promyelocytoic leukemia or promyeloctyic leukemia zinc finger genes arrest the differentiation of leukemic cells (Minucci et al., 2001). These fusion proteins repress the transcription of genes involved in myeloid differentiation by recruiting HDAC-containing complexes (Minucci and Pelicci, 2006). In addition, the BCL6 transcriptional repressor and AML1-ETO fusion protein induce non-Hodgkin's lymphoma and acute myelogenous leukemia (AML), respectively, by recruiting transcriptional repression complexes that contain HDACs (Marks et al., 2000). The importance of HDACs in solid tumorigenesis is supported by the correlation of the risk for tumor recurrence in low-grade prostate cancer with distinct patterns of histone modifications (Seligson et al., 2005), the global loss of histone 4 monoacetylation in cancer cell lines and primary tumor samples (Fraga et al., 2005), and the functional interaction of HDAC2 over-expression with loss of the APC tumor suppressor gene in colon cancer cells (Zhu et al., 2004).

A variety of natural and synthetic compounds function as HDAC inhibitors (HDACi) by binding to the active site and chelating the zinc atom required for HDAC enzymatic activity (Minucci and Pelicci, 2006). These compounds vary greatly in terms of stability, potency, efficacy and toxicity and inhibit both class 1 and class 2 HDACs (Minucci and Pelicci, 2006). HDACi induce cell cycle arrest, differentiation, and apoptosis in human cancer cell lines in vitro (Butler et al., 2000; Gottlicher et al., 2001; Hague et al., 1993; Heerdt et al., 1994). In contrast, normal cells are relatively resistant to these compounds (Marks et al., 2000), although HDACi have widespread effects on transcription, as about 20 percent of genes are influenced by HDACi with an equal number of up- or down-regulated genes (Glaser et al., 2003; Mitsiades et al., 2004; Peart et al., 2005; Van Lint et al., 1996).

The tumor-selective biological effects of HDACi are attributed to the induction of anti-growth and apoptotic genes in cancer cells (Insing a et al., 2005; Nebbioso et al., 2005; Villar-Garea and Esteller, 2004), notably the p53-independent up-regulation of p21 and associated cell cycle arrest (Archer et al., 1998; Gui et al., 2004; Richon et al., 2000). HDACi selectively induce apoptosis in APL cells versus normal lymphocytes and these effects are dependent on the increased expression of tumor-necrosis factor-related apoptosis-inducing ligand (TRAIL), death receptor 5 (DR5), Fas, and Fas ligand (FasL) (Insing a et al., 2005). HDACi are currently under clinical evaluation as single agents (Carducci et al., 2001; Gilbert et al., 2001; Gore et al., 2002; Kelly et al., 2005; Kelly et al., 2003; Patnaik et al., 2002) or in combination with existing chemotherapeutics (Kuendgen et al., 2006). These trials have determined that HDACi are generally associated with low toxicity and in some cases a maximal tolerated dose was not reached (Minucci and Pelicci, 2006). Although all HDACi tested had some clinical effects, many have low potency and patients succumbed to disease after treatment ceased (Minucci and Pelicci, 2006). There are currently no criteria to determine which patients are most likely to benefit from HDACi treatment, although elucidating the molecular basis for the tumor-selective effects of these compounds can promote the development of improved HDACi.

The selective induction of Fas in HDACi-treated APL cells versus normal lymphocytes (Insing a et al., 2005) raised the possibility that HDACi could restore the expression of Fas and other down-regulated pro-apoptotic or growth-inhibitory genes in malignant cells transformed by multiple oncogenic mutations. Indeed, young adult mouse colon cells transformed by cooperating oncogenic mutations such as Ras activation and p53 loss-of-function (Xia and Land, 2007) responded with altered morphology and proliferation to HDACi treatment and completely inhibited the ability of these cells to form colonies in soft agar in vitro and tumors in nude mice in vivo, presumably via sensitization to anoikis. Additionally, these biological effects are causally linked to the restored expression of a series of cooperation response genes that are synergistically down-regulated following expression of mutant p53 and activated Ras. Notably, interfering with the re-expression of six of these genes abrogated the effects of the HDACi and rescued tumor formation in vivo indicating that the restored expression of all six genes is required for HDACi to antagonize the transformed phenotype.

Thus, for example, disclosed herein are methods of treating a cancer in a subject comprising administering to the subject one or more anti-cancer agents and an agent that modulates the activity of one or more cooperation response genes, wherein the anti-cancer agent is a histone deacetylase inhibitor, and wherein the cooperation response genes are selected from the group consisting of Abat, Abca1, Ank, Ankrd1, Arhgap24, Atp8a1, Bbs7, Bex1, Ccl9, Centd3, Chst1, Ckmt1, Col9a3, Cpz, Cxcl1, Cxcl15, Daf1, Dapk1, Dffb, Dgka, Dixdc, Dusp15, Elav12, Eno3, Ephb2, Espn, Eva1, Fas, F2r11, Fgf18, Fgf7, Fhod3, FHOS2, Garn13, Gca, Gpr149, Hbegf, Hey2, Hmga1, Hmga2, Hoxc13, Id2, Id4, Igfbp2, Igsf4a, Jag2, Kctd15, Lass4, Ldhb, Man2b1, Mcam, Mmp15, Mpp7, Mrpl15, Mrpplf4, Ms4a10, Mtus1, Nbea, Notch3, Noxa, Oaf, Parvb, Pard6g, Perp, Plac8, P1a2g7, Pitx2, Pltp, Plxdc2, Prkcm, Prkg, Prss22, Pvr14, Rab40b, Rai2, Rasl11a, Rb1, Rgs2, Rprm, Rspo3, Satb1, Sbk1, Sbsn, Scn3b, Sema3d, Sema7a, Serpinb2, Sfrp2, Slc14a1, Slc27a3, Sms, Sod3, Stmn4, Tex15, Tnfrsf18, Tnnt2, Unc45b, Wnt9a, Zac1, and Zfp385. Also disclosed are methods wherein the cooperation response genes are selected from the group consisting of Dapk1, Dffb, Fas, Noxa, Perp, Rprm, Sfrp2, and Zac1. It is understood that any agent known in the art that enhances or inhibits one or more CRG's may by used in the treatment methods disclosed herein. Thus, for example, also disclosed are methods of treating a cancer comprising administering an agent wherein the agent is selected from the any one or more of the agents listed on Tables, 12, 15, 16, or 17). Thus, for example, an agent for treating cancer by modulating the expression or activity of one or more CRGs includes but is not limited to (+)-chelidonine, 0179445-0000, 0198306-0000, 1,4-chrysenequinone, 15-delta prostaglandin J2, 2,6-dimethylpiperidine, 4-hydroxyphenazone, 5186223, 6-azathymine, acenocoumarol, alpha-estradiol, altizide, alverine, alvespimycin, amikacin, aminohippuric acid, amoxicillin, amprolium, ampyrone, antimycin A, arachidonyltrifluoromethane, atractyloside, azathioprine, azlocillin, bacampicillin, baclofen, bambuterol, beclometasone, benzylpenicillin, betaxolol, betulinic acid, biperiden, boldine, bromocriptine, bufexamac, buspirone, butacaine, butirosin, calycanthine, canadine, canavanine, carbarsone, carbenoxolone, carbimazole, carcinine, carmustine, cefalotin, cefepime, ceftazidime, cephaeline, chenodeoxycholic acid, chlorhexidine, chlorogenic acid, chlorpromazine, chlortalidone, cinchonidine, cinchonine, clemizole, co-dergocrine mesilate, CP-320650-01, CP-690334-01, dacarbazine, demeclocycline, dexibuprofen, dextromethorphan, dicycloverine, diethylstilbestrol, diflorasone, diflunisal, dihydroergotamine, diloxanide, dinoprostone, diphemanil metilsulfate, diphenylpyraline, doxylamine, droperidol, epirizole, epitiostanol, esculetin, estradiol, estropipate, ethionamide, etofenamate, etomidate, eucatropine, famotidine, famprofazone, fendiline, fisetin, fludrocortisone, flufenamic acid, flupentixol, fluphenazine, fluticasone, fluvastatin, fosfosal, fulvestrant, gabexate, galantamine, gemfibrozil, genistein, glibenclamide, gliquidone, glycocholic acid, gossypol, gramine, guanadrel, halcinonide, haloperidol, harpagoside, hexamethonium bromide, homochlorcyclizine, hydroxyzine, idoxuridine, ifosfamide, indapamide, iobenguane, iopanoic acid, iopromide, isoetarine, isoxsuprine, isradipine, ketorolac, ketotifen, lanatoside C, lansoprazole, laudanosine, letrozole, levodopa, levomepromazine, lidocaine, liothyronine, lisinopril, lisuride, LY-294002, lynestrenol, meclofenamic acid, meclofenoxate, medrysone, mefloquine, mepacrine, methapyrilene, methazolamide, methyldopa, methylergometrine, metoclopramide, mevalolactone, mometasone, monensin, monorden, naftopidil, nalbuphine, naltrexone, napelline, naphazoline, naringin, niclosamide, niflumic acid, nimesulide, nomifensine, noretynodrel, norfloxacin, orphenadrine, oxolinic acid, oxprenolol, papaverine, pentolonium, pepstatin, perphenazine, PF-00562151-00, phenelzine, phenindione, pheniramine, phthalylsulfathiazole, pinacidil, pioglitazone, piperine, piretanide, piribedil, pirlindole, PNU-0230031, pralidoxime, pramocaine, praziquantel, prednisone, Prestwick-1100, Prestwick-981, probenecid, prochlorperazine, proglumide, propofol, protriptyline, racecadotril, riboflavin, rifabutin, rimexolone, roxithromycin, santonin, SB-203580, SC-560, scopoletin, scriptaid, seneciphylline, sirolimus, sitosterol, sodium phenylbutyrate, solanine, spectinomycin, spiradoline, SR-95531, SR-95639A, sulfadimidine, sulfaguanidine, sulfanilamide, sulfathiazole, tanespimycin, terbutaline, terguride, thalidomide, thiamazole, thiamphenicol, thioridazine, ticarcillin, ticlopidine, timidazole, tiratricol, tolfenamic acid, tremorine, trichostatin A, trifluoperazine, troglitazone, tyloxapol, ursodeoxycholic acid, valproic acid, vanoxerine, vidarabine, vincamine, vorinostat, wortmannin, yohimbic acid, yohimbine, and zidovudine.

As disclosed above the compositions and methods disclosed herein can be used to treat any disease where uncontrolled cellular proliferation occurs such as cancers. A non-limiting list of different types of cancers is as follows: lymphomas (Hodgkins and non-Hodgkins), leukemias, carcinomas, carcinomas of solid tissues, squamous cell carcinomas, adenocarcinomas, sarcomas, gliomas, high grade gliomas, blastomas, neuroblastomas, plasmacytomas, histiocytomas, melanomas, adenomas, hypoxic tumours, myelomas, AIDS-related lymphomas or sarcomas, metastatic cancers, or cancers in general.

A representative but non-limiting list of cancers that the disclosed compositions can be used to treat is the following: lymphoma, B cell lymphoma, T cell lymphoma, mycosis fungoides, Hodgkin's Disease, leukemias, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, gastric cancer, colon cancer, cervical cancer, cervical carcinoma, breast cancer, and epithelial cancer, bone cancers, renal cancer, bladder cancer, genitourinary cancer, esophageal carcinoma, large bowel cancer, metastatic cancers hematopoietic cancers, sarcomas, Ewing's sarcoma, synovial cancer, soft tissue cancers; and testicular cancer. Thus disclosed herein are methods of treating wherein the cancer is selected form the group of cancers consisting of lymphoma, B cell lymphoma, T cell lymphoma, mycosis fungoides, Hodgkin's Disease, leukemias, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, gastric cancer, colon cancer, cervical cancer, cervical carcinoma, breast cancer, and epithelial cancer, bone cancers, renal cancer, bladder cancer, genitourinary cancer, esophageal carcinoma, large bowel cancer, metastatic cancers hematopoietic cancers, sarcomas, Ewing's sarcoma, synovial cancer, soft tissue cancers; and testicular cancer.

However, it is recognized herein that perturbation of some CRGs may have an effect for one type of cancer and not have an effect in another type of cancer. For example, disclosed herein are methods of treating colon cancer comprising administering to a subject with colon cancer an agent that modulates the expression or activity of one or more CRGs such as Dffb, Dgka, Dixdc1, Eno3, Fas, Fgf7, Gpr149, Hmga1, Hmga2, HoxC13, Id2, Id4, Igsf4a, Jag2, Noxa, Oaf, Perp, P1a2g7, Plac8, Plxdc2, Rgs2, Rprm, Satb1, Sema3d, Sfrp2, Slc14a1, Sod3, Stmn4, Unc45b, and Spf385. Similarly, disclosed herein are methods of treating pancreatic cancer comprising administering to a subject with pancreatic cancer an agent that modulates the expression or activity of one or more CRGs such as Arhgap24, Dapk1, Dixdc1, Eno3, Fgf7, Hey2, HoxC13, Jag2, P1a2g7, Plac8, Rab40b, Rprm, Satb1, and Unc45b. Also disclosed are methods of treating prostate cancer comprising administering to a subject with prostate cancer an agent that modulates the expression or activity of one or more CRGs such as Arhgap24, Daf1, Eva1, HoxC13, Mcam, Notch3, Noxa, Oaf, Pard6g, Perp, P1a2g7, Sfrp2, and Zfp385. Also disclosed are methods of treating a melanoma comprising administering to a subject with a melanoma an agent that modulates the expression or activity of one or more CRGs such as Arhgap24, Atp8a1, Bbs7, Cxcl1, Dixdc1, Fas, Hey2, Jag2, Notch3, Noxa, Pitx2, P1a2g7, Plac8, Prkg1, Rab40b, Satb1, and Stmn4. Also disclosed are methods of treating lung cancer comprising administering to a subject with lung cancer an agent that modulates the expression or activity of one or more CRGs such as Abca1, Arhgap24, Bbs7, Daf1, Dixdc1, Eno3, Fas, Hey2, Mcam, P1a2g7, Prkg1, Satb1, Sfrp2, and Unc45b.

4. Methods of Diagnosing or Assessing the Efficacy of a Treatment.

The activity of the cooperation response genes identified herein can have tremendous affect on the effectiveness of a treatment. By determining whether one or more cooperation response genes are suppressed, expressed, or over-expressed in a cancer relative to a control, a determination can be made as to the susceptibility or resistance of an individual to a treatment can be made as well as the determination of the efficacy of a treatment for a cancer given the cancers expression profile of cooperation response genes. In this way, known compounds can be tested for effectiveness in modulating the activity of one or more cooperation response genes in a manner that inhibits a cancer. Thus, disclosed herein are methods for determining whether a cancer is susceptible to treatment with an agent comprising measuring the expression of the cooperation response gene panel in the cancer relative to a control, wherein the responsiveness of one or more cooperation response genes indicates sensitivity to treatment. It is understood the anti-cancer agent can be any new or old composition known in the art regardless of the known effectiveness in treating cancer. Thus, disclosed in one aspect are methods wherein the anti-cancer agent is a chemotherapeutic or anti-oxidant. Also disclosed are methods wherein the anti-cancer agent is a histone deacetylase inhibitor (HDACi). Thus, for example, disclosed herein are methods wherein expression of Dapk1, Dffb, Fas, Noxa, Perp, Rprm, Sfrp2, and Zac1 indicates susceptibility to histone deacetylase inhibitors. Also disclosed are methods wherein more than one anti-cancer agent. Thus, disclosed herein are methods for determining whether a cancer is susceptible to treatment with one or more anti-cancer agents comprising measuring the expression of the cooperation response gene panel in the cancer relative to a control, wherein the responsiveness of one or more cooperation response genes indicates sensitivity to treatment.

It is understood that the cooperation response gene panel will vary depending on the particular cell type or cancer. Thus, disclosed herein are methods, wherein the cooperation response gene is selected from the group consisting of Abat, Abca1, Ank, Ankrd1, Arhgap24, Atp8a1, Bbs7, Bex1, Ccl9, Centd3, Chst1, Ckmt1, Col9a3, Cpz, Cxcl1, Cxcl15, Daf1, Dapk1, Dffb, Dgka, Dixdc, Dusp15, Elav12, Eno3, Ephb2, Espn, Eva1, Fas, F2r11, Fgf18, Fgf7, Fhod3, FHOS2, Garn13, Gca, Gpr149, Hbegf, Hey2, Hmga1, Hmga2, Hoxc13, Id2, Id4, Igfbp2, Igsf4a, Jag2, Kctd15, Lass4, Ldhb, Man2b1, Mcam, Mmp15, Mpp7, Mrpl15, Mrpplf4, Ms4a10, Mtus1, Nbea, Notch3, Noxa, Oaf, Parvb, Pard6g, Perp, Plac8, P1a2g7, Pitx2, Pltp, Plxdc2, Prkcm, Prkg, Prss22, Pyr14, Rab40b, Rai2, Rasl11a, Rb1, Rgs2, Rprm, Rspo3, Satb1, Sbk1, Sbsn, Scn3b, Sema3d, Sema7a, Serpinb2, Sfrp2, Slc14a1, Slc27a3, Sms, Sod3, Stmn4, Tex15, Tnfrsf18, Tnnt2, Unc45b, Wnt9a, Zac1, Zfp385, as well as the cooperation response genes identified by the Genbank accession number AV133559, BM118398, BB353853, BB381558, AV231983, AI848263, AV244175, BF159528, AV231424, AV234963, BC013499, AV254040, BG071013, AK003981, BG066186, AK005731, BC027185, AK009671, AV323203, AI509011, BM220576, BQ173895, AV024662, BB207363, BC026627, AK017369, BQ031255, BC007193, BE949277, AK018275, BB704967, BB312717, AK018112, BI905111, BE957307, BG066982, BB358264, BB478071, AV298358, BB767109, AA266723, AV241486, BB133117, AI450842, and AW543723. It is understood and herein contemplated that the disclosed cooperation response genes can have pro-apoptotic or anti-proliferative activity. Therefore, disclosed herein are methods, wherein the activated cooperation response gene has pro-apoptotic or anti-proliferation activity. Thus, for example, in one embodiment, disclosed herein are methods wherein the cooperation response gene is selected from the group consisting of Dapk1, Dffb, Fas, Noxa, Perp, Rprm, Sfrp2, and Zac1.

The disclosed methods can be used to determine the susceptibility or resistance of any subject or cell as well as the efficacy in any type of cancer. Thus, disclosed herein are methods for determining whether a cancer is susceptible or resistant to treatment with an anti-cancer agent wherein the cancer comprises but is not limited to lymphoma, B cell lymphoma, T cell lymphoma, mycosis fungoides, Hodgkin's Disease, leukemias, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, gastric cancer, colon cancer, cervical cancer, cervical carcinoma, breast cancer, and epithelial cancer, bone cancers, renal cancer, bladder cancer, genitourinary cancer, esophageal carcinoma, large bowel cancer, metastatic cancers hematopoietic cancers, sarcomas, Ewing's sarcoma, synovial cancer, soft tissue cancers; and testicular cancer.

5. Methods of Using the Compositions as Research Tools

The compositions can be used for example as targets in combinatorial chemistry protocols or other screening protocols to isolate molecules that possess desired functional properties related to inhibiting a cancer.

The disclosed compositions can also be used diagnostic tools related to diseases, such as cancer.

The disclosed compositions can be used as discussed herein as either reagents in micro arrays or as reagents to probe or analyze existing microarrays. The disclosed compositions can be used in any known method for isolating or identifying single nucleotide polymorphisms. The compositions can also be used in any known method of screening assays, related to chip/micro arrays. The compositions can also be used in any known way of using the computer readable embodiments of the disclosed compositions, for example, to study relatedness or to perform molecular modeling analysis related to the disclosed compositions.

### C. COMPOSITIONS

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular cancer gene or cooperation response gene is disclosed and discussed and a number of modifications that can be made to a number of molecules including the cancer gene or cooperation response gene are discussed, specifically contemplated is each and every combination and permutation of cancer gene or cooperation response gene and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

1. Nucleic Acids

There are a variety of molecules disclosed herein that are nucleic acid based, including for example the nucleic acids that encode, for example, Abat, Abca1, Ank, Ankrd1, Arhgap24, Atp8a1, Bbs7, Bex1, Ccl9, Centd3, Chst1, Ckmt1, Col9a3, Cpz, Cxcl1, Cxcl15, Daf1, Dapk1, Dffb, Dgka, Dixdc, Dusp15, Elav12, Eno3, Ephb2, Espn, Eva1, Fas, F2r11, Fgf18, Fgf7, Fhod3, FHOS2, Garn13, Gca, Gpr149, Hbegf, Hey2, Hmga1, Hmga2, Hoxc13, Id2, Id4, Igfbp2, Igsf4a, Jag2, Kctd15, Lass4, Ldhb, Man2b1, Mcam, Mmp15, Mpp7, Mrpl15, Mrpplf4, Ms4a10, Mtus1, Nbea, Notch3, Noxa, Oaf, Parvb, Pard6g, Perp, Plac8, Pla2g7, Pitx2, Pltp, Plxdc2, Prkcm, Prkg, Prss22, Pyr14, Rab40b, Rai2, Rasl11a, Rb1, Rgs2, Rprm, Rspo3, Satb1, Sbk1, Sbsn, Scn3b, Sema3d, Sema7a, Serpinb2, Sfrp2, Slc14a1, Slc27a3, Sms, Sod3, Stmn4, Tex15, Tnfrsf18, Tnnt2, Unc45b, Wnt9a, Zac1, and Zfp385 as well as any other proteins disclosed herein, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantagous that the antisense molecule be made up of nucleotide analogs that reduce the degradation of the antisense molecule in the cellular environment.

a) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenine-9-yl (A), cytosine-1-yl (C), guanine-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556),

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

b) Sequences

There are a variety of sequences related to, for example, Abat, Abca1, Ank, Ankrd1, Arhgap24, Atp8a1, Bbs7, Bex1, Ccl9, Centd3, Chst1, Ckmt1, Col9a3, Cpz, Cxcl1, Cxcl15, Daf1, Dapk1, Dffb, Dgka, Dixdc, Dusp15, Elav12, Eno3, Ephb2, Espn, Eva1, Fas, F2r11, Fgf18, Fgf7, Fhod3, FHOS2, Garn13, Gca, Gpr149, Hbegf, Hey2, Hmga1, Hmga2, Hoxc13, Id2, Id4, Igfbp2, Igsf4a, Jag2, Kctd15, Lass4, Ldhb, Man2b1, Mcam, Mmp15, Mpp7, Mrpl15, Mrpplf4, Ms4a10, Mtus1, Nbea, Notch3, Noxa, Oaf, Parvb, Pard6g, Perp, Plac8, P1a2g7, Pitx2, Pltp, Plxdc2, Prkcm, Prkg, Prss22, Pyr14, Rab40b, Rai2, Rasl11a, Rb1, Rgs2, Rprm, Rspo3, Satb1, Sbk1, Sbsn, Scn3b, Sema3d, Sema7a, Serpinb2, Sfrp2, Slc14a1, Slc27a3, Sms, Sod3, Stmn4, Tex15, Tnfrsf18, Tnnt2, Unc45b, Wnt9a, Zac1, and Zfp385 as well as any other protein disclosed herein that are disclosed on Genbank, and these sequences and others are herein incorporated by reference in their entireties as well as for individual subsequences contained therein.

A variety of sequences are provided herein and these and others can be found in Genbank. Those of skill in the art understand how to resolve sequence discrepancies and differences and to adjust the compositions and methods relating to a particular sequence to other related sequences. Primers and/or probes can be designed for any sequence given the information disclosed herein and known in the art.

c) Primers and Probes

Disclosed are compositions including primers and probes, which are capable of interacting with the genes disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the nucleic acid or region of the nucleic acid or they hybridize with the complement of the nucleic acid or complement of a region of the nucleic acid.

d) Functional Nucleic Acids

Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, shRNAs, siRNAs, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of Abat, Abca1, Ank, Ankrd1, Arhgap24, Atp8a1, Bbs7, Bex1, Ccl9, Centd3, Chst1, Ckmt1, Col9a3, Cpz, Cxcl1, Cxcl15, Daf1, Dapk1, Dffb, Dgka, Dixdc, Dusp15, Elav12, Eno3, Ephb2, Espn, Eva1, Fas, F2r11, Fgf18, Fgf7, Fhod3, FHOS2, Garn13, Gca, Gpr149, Hbegf, Hey2, Hmga1, Hmga2, Hoxc13, Id2, Id4, Igfbp2, Igsf4a, Jag2, Kctd15, Lass4, Ldhb, Man2b1, Mcam, Mmp15, Mpp7, Mrpl15, Mrpplf4, Ms4a10, Mtus1, Nbea, Notch3, Noxa, Oaf, Parvb, Pard6g, Perp, Plac8, P1a2g7, Pitx2, Pltp, Plxdc2, Prkcm, Prkg, Prss22, Pvr14, Rab40b, Rai2, Rasl11a, Rb1, Rgs2, Rprm, Rspo3, Satb1, Sbk1, Sbsn, Scn3b, Sema3d, Sema7a, Serpinb2, Sfrp2, Slc14a1, Slc27a3, Sms, Sod3, Stmn4, Tex15, Tnfrsf18, Tnnt2, Unc45b, Wnt9a, Zac1, and Zfp385 or the genomic DNA of Abat, Abca1, Ank, Ankrd1, Arhgap24, Atp8a1, Bbs7, Bex1, Ccl9, Centd3, Chst1, Ckmt1, Col9a3, Cpz, Cxcl1, Cxcl15, Daf1, Dapk1, Dffb, Dgka, Dixdc, Dusp15, Elav12, Eno3, Ephb2, Espn, Eva1, Fas, F2r11, Fgf18, Fgf7, Fhod3, FHOS2, Garn13, Gca, Gpr149, Hbegf, Hey2, Hmga1, Hmga2, Hoxc13, Id2, Id4, Igfbp2, Igsf4a, Jag2, Kctd15, Lass4, Ldhb, Man2b1, Mcam, Mmp15, Mpp7, Mrpl15, Mrpplf4, Ms4a10, Mtus1, Nbea, Notch3, Noxa, Oaf, Parvb, Pard6g, Perp, Plac8, P1a2g7, Pitx2, Pltp, Plxdc2, Prkcm, Prkg, Prss22, Pvr14, Rab40b, Rai2, Rasl11a, Rb1, Rgs2, Rprm, Rspo3, Satb1, Sbk1, Sbsn, Scn3b, Sema3d, Sema7a, Serpinb2, Sfrp2, Slc14a1, Slc27a3, Sms, Sod3, Stmn4, Tex15, Tnfrsf18, Tnnt2, Unc45b, Wnt9a, Zac1, and Zfp385 or they can interact with the polypeptide. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (kd) less than or equal to 10-6, 10-8, 10-10, or 10-12. A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with kds from the target molecule of less than 10-12 M. It is preferred that the aptamers bind the target molecule with a kd less than 10-6, 10-8, 10-10, or 10-12. Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a kd with the target molecule at least 10, 100, 1000, 10,000, or 100,000 fold lower than the kd with a background binding molecule. It is preferred when doing the comparison for a polypeptide for example, that the background molecule be a different polypeptide. For example, when determining the specificity of Abat, Abca1, Ank, Ankrd1, Arhgap24, Atp8a1, Bbs7, Bex1, Ccl9, Centd3, Chst1, Ckmt1, Col9a3, Cpz, Cxcl1, Cxcl15, Daf1, Dapk1, Dffb, Dgka, Dixdc, Dusp15, Elav12, Eno3, Ephb2, Espn, Eva1, Fas, F2r11, Fgf18, Fgf7, Fhod3, FHOS2, Garn13, Gca, Gpr149, Hbegf, Hey2, Hmga1, Hmga2, Hoxc13, Id2, Id4, Igfbp2, Igsf4a, Jag2, Kctd15, Lass4, Ldhb, Man2b1, Mcam, Mmp15, Mpp7, Mrpl15, Mrpplf4, Ms4a10, Mtus1, Nbea, Notch3, Noxa, Oaf, Parvb, Pard6g, Perp, Plac8, P1a2g7, Pitx2, Pltp, Plxdc2, Prkcm, Prkg, Prss22, Pvr14, Rab40b, Rai2, Rasl11a, Rb1, Rgs2, Rprm, Rspo3, Satb1, Sbk1, Sbsn, Scn3b, Sema3d, Sema7a, Serpinb2, Sfrp2, Slc14a1, Slc27a3, Sms, Sod3, Stmn4, Tex15, Tnfrsf18, Tnnt2, Unc45b, Wnt9a, Zac1, and Zfp385 aptamers, the background protein could be Abat, Abca1, Ank, Ankrd1, Arhgap24, Atp8a1, Bbs7, Bex1, Ccl9, Centd3, Chst1, Ckmt1, Col9a3, Cpz, Cxcl1, Cxcl15, Daf1, Dapk1, Dffb, Dgka, Dixdc, Dusp15, Elav12, Eno3, Ephb2, Espn, Eva1, Fas, F2r11, Fgf18, Fgf7, Fhod3, FHOS2, Garn13, Gca, Gpr149, Hbegf, Hey2, Hmga1, Hmga2, Hoxc13, Id2, Id4, Igfbp2, Igsf4a, Jag2, Kctd15, Lass4, Ldhb, Man2b1, Mcam, Mmp15, Mpp7, Mrpl15, Mrpplf4, Ms4a10, Mtus1, Nbea, Notch3, Noxa, Oaf, Parvb, Pard6g, Perp, Plac8, Pla2g7, Pitx2, Pltp, Plxdc2, Prkcm, Prkg, Prss22, Pvr14, Rab40b, Rai2, Rasl11a, Rb1, Rgs2, Rprm, Rspo3, Satb1, Sbk1, Sbsn, Scn3b, Sema3d, Sema7a, Serpinb2, Sfrp2, Slc14a1, Slc27a3, Sms, Sod3, Stmn4, Tex15, Tnfrsf18, Tnnt2, Unc45b, Wnt9a, Zac1, and Zfp385. Representative examples of how to make and use aptamers to bind a variety of different target molecules can be found in the following non-limiting list of U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos. 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos. 5,595,873 and 5,652,107). There are also a number of ribozymes that are not found in natural systems, but which have been engineered to catalyze specific reactions de novo (for example, but not limited to the following U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a kd less than 10-6, 10-8, 10-10, or 10-12. Representative examples of how to make and use triplex forming molecules to bind a variety of different target molecules can be found in the following non-limiting list of U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukarotic cells. (Yuan et al., Proc. Natl. Acad. Sci. USA 89:8006-8010 (1992); WO 93/22434 by Yale; WO 95/24489 by Yale; Yuan and Altman, EMBO J. 14:159-168 (1995), and Carrara et al., Proc. Natl. Acad. Sci. (USA) 92:2627-2631 (1995)). Representative examples of how to make and use EGS molecules to facilitate cleavage of a variety of different target molecules be found in the following non-limiting list of U.S. Pat. Nos. 5,168,053, 5,624,824, 5,683,873, 5,728,521, 5,869,248, and 5,877,162.

2. Nucleic Acid Delivery

In the methods described above which include the administration and uptake of exogenous DNA into the cells of a subject (i.e., gene transduction or transfection), the disclosed nucleic acids can be in the form of naked DNA or RNA, or the nucleic acids can be in a vector for delivering the nucleic acids to the cells, whereby the antibody-encoding DNA fragment is under the transcriptional regulation of a promoter, as would be well understood by one of ordinary skill in the art. The vector can be a commercially available preparation, such as an adenovirus vector (Quantum Biotechnologies, Inc. (Laval, Quebec, Canada). Delivery of the nucleic acid or vector to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaR^{x}Pharmaceutical Corp., Tucson, Ariz.).

As one example, vector delivery can be via a viral system, such as a retroviral vector system which can package a recombinant retroviral genome (see e.g., Pastan et al., Proc. Natl. Acad. Sci. U.S.A. 85:4486, 1988; Miller et al., Mol. Cell. Biol. 6:2895, 1986). The recombinant retrovirus can then be used to infect and thereby deliver to the infected cells nucleic acid encoding a broadly neutralizing antibody (or active fragment thereof). The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naidini et al., Science 272:263-267, 1996), pseudotyped retroviral vectors (Agrawal et al., Exper. Hematol. 24:738-747, 1996). Physical transduction techniques can also be used, such as liposome delivery and receptor-mediated and other endocytosis mechanisms (see, for example, Schwartzenberger et al., Blood 87:472-478, 1996). This disclosed compositions and methods can be used in conjunction with any of these or other commonly used gene transfer methods.

As one example, if the antibody-encoding nucleic acid is delivered to the cells of a subject in an adenovirus vector, the dosage for administration of adenovirus to humans can range from about 107 to 109 plaque forming units (pfu) per injection but can be as high as 1012 pfu per injection (Crystal, Hum. Gene Ther. 8:985-1001, 1997; Alvarez and Curiel, Hum. Gene Ther. 8:597-613, 1997). A subject can receive a single injection, or, if additional injections are necessary, they can be repeated at six month intervals (or other appropriate time intervals, as determined by the skilled practitioner) for an indefinite period and/or until the efficacy of the treatment has been established.

Parenteral administration of the nucleic acid or vector, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. For additional discussion of suitable formulations and various routes of administration of therapeutic compounds, see, e.g., Remington: The Science and Practice of Pharmacy (19th ed.) ed. A. R. Gennaro, Mack Publishing Company, Easton, Pa. 1995.

3. Delivery of the Compositions to Cells

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

a) Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids, such as Abat, Abca1, Ank, Ankrd1, Arhgap24, Atp8a1, Bbs7, Bex1, Ccl9, Centd3, Chst1, Ckmt1, Col9a3, Cpz, Cxcl1, Cxcl15, Daf1, Dapk1, Dffb, Dgka, Dixdc, Dusp15, Elav12, Eno3, Ephb2, Espn, Eva1, Fas, F2r11, Fgf18, Fgf7, Fhod3, FHOS2, Garn13, Gca, Gpr149, Hbegf, Hey2, Hmga1, Hmga2, Hoxc13, Id2, Id4, Igfbp2, Igsf4a, Jag2, Kctd15, Lass4, Ldhb, Man2b1, Mcam, Mmp15, Mpp7, Mrpl15, Mrpplf4, Ms4a10, Mtus1, Nbea, Notch3, Noxa, Oaf, Parvb, Pard6g, Perp, Plac8, Pla2g7, Pitx2, Pltp, Plxdc2, Prkcm, Prkg, Prss22, Pvr14, Rab40b, Rai2, Rasl11a, Rb1, Rgs2, Rprm, Rspo3, Satb1, Sbk1, Sbsn, Scn3b, Sema3d, Sema7a, Serpinb2, Sfrp2, Slc14a1, Slc27a3, Sms, Sod3, Stmn4, Tex15, Tnfrsf18, Tnnt2, Unc45b, Wnt9a, Zac1, and Zfp385 into the cell without degradation and include a promoter yielding expression of the gene in the cells into which it is delivered. In some embodiments the vectors are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

(1) Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

(2) Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Ce1173:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

(3) Adeno-Asscociated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorproated by reference for material related to the AAV vector.

The disclosed vectors thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

(4) Large Payload Viral Vectors

Molecular genetic experiments with large human herpesviruses have provided a means whereby large heterologous DNA fragments can be cloned, propagated and established in cells permissive for infection with herpesviruses (Sun et al., Nature Genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA >150 kb to specific cells. EBV recombinants can maintain large pieces of DNA in the infected B-cells as episomal DNA. Individual clones carried human genomic inserts up to 330 kb appeared genetically stable the maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA >220 kb and to infect cells that can stably maintain DNA as episomes.

Other useful systems include, for example, replicating and host-restricted non-replicating vaccinia virus vectors.

b) Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci. USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

c) In Vivo/Ex Vivo

As described above, the compositions can be administered in a pharmaceutically acceptable carrier and can be delivered to the subject's cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

4. Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

a) Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell. Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell. Bio. 4: 1293 (1984)). They are usually between 10 and 300 by in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTF.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

b) Markers

The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

5. Antibodies

(1) Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with Abat, Abca1, Ank, Ankrd1, Arhgap24, Atp8a1, Bbs7, Bex1, Ccl9, Centd3, Chst1, Ckmt1, Col9a3, Cpz, Cxcl1, Cxcl15, Daf1, Dapk1, Dffb, Dgka, Dixdc, Dusp15, Elav12, Eno3, Ephb2, Espn, Eva1, Fas, F2r11, Fgf18, Fgf7, Fhod3, FHOS2, Garn13, Gca, Gpr149, Hbegf, Hey2, Hmga1, Hmga2, Hoxc13, Id2, Id4, Igfbp2, Igsf4a, Jag2, Kctd15, Lass4, Ldhb, Man2b1, Mcam, Mmp15, Mpp7, Mrpl15, Mrpplf4, Ms4a10, Mtus1, Nbea, Notch3, Noxa, Oaf, Parvb, Pard6g, Perp, Plac8, Pla2g7, Pitx2, Pltp, Plxdc2, Prkcm, Prkg, Prss22, Pvr14, Rab40b, Rai2, Rasl11a, Rb1, Rgs2, Rprm, Rspo3, Satb1, Sbk1, Sbsn, Scn3b, Sema3d, Sema7a, Serpinb2, Sfrp2, Slc14a1, Slc27a3, Sms, Sod3, Stmn4, Tex15, Tnfrsf18, Tnnt2, Unc45b, Wnt9a, Zac1, and Zfp385. The antibodies can be tested for their desired activity using the in vitro assays described herein, or by analogous methods, after which their in vivo therapeutic and/or prophylactic activities are tested according to known clinical testing methods.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc. Natl. Acad. Sci. USA, 81:6851-6855 (1984)).

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

(2) Human Antibodies

The disclosed human antibodies can be prepared using any technique. Examples of techniques for human monoclonal antibody production include those described by Cole et al. (Monoclonal Antibodies and Cancer Therapy, Alan R. Liss, p. 77, 1985) and by Boerner et al. (J. Immunol., 147(1):86-95, 1991). Human antibodies (and fragments thereof) can also be produced using phage display libraries (Hoogenboom et al., J. Mol. Biol., 227:381, 1991; Marks et al., J. Mol. Biol., 222:581, 1991).

The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

(3) Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an Fv, Fab, Fab′, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

(4) Administration of Antibodies

Administration of the antibodies can be done as disclosed herein. Nucleic acid approaches for antibody delivery also exist. The broadly neutralizing anti Abat, Abca1, Ank, Ankrd1, Arhgap24, Atp8a1, Bbs7, Bex1, Ccl9, Centd3, Chst1, Ckmt1, Col9a3, Cpz, Cxcl1, Cxcl15, Daf1, Dapk1, Dffb, Dgka, Dixdc, Dusp15, Elav12, Eno3, Ephb2, Espn, Eva1, Fas, F2r11, Fgf18, Fgf7, Fhod3, FHOS2, Garn13, Gca, Gpr149, Hbegf, Hey2, Hmga1, Hmga2, Hoxc13, Id2, Id4, Igfbp2, Igsf4a, Jag2, Kctd15, Lass4, Ldhb, Man2b1, Mcam, Mmp15, Mpp7, Mrpl15, Mrpplf4, Ms4a10, Mtus1, Nbea, Notch3, Noxa, Oaf, Parvb, Pard6g, Perp, Plac8, P1a2g7, Pitx2, Pltp, Plxdc2, Prkcm, Prkg, Prss22, Pvr14, Rab40b, Rai2, Rasl11a, Rb1, Rgs2, Rprm, Rspo3, Satb1, Sbk1, Sbsn, Scn3b, Sema3d, Sema7a, Serpinb2, Sfrp2, Slc14a1, Slc27a3, Sms, Sod3, Stmn4, Tex15, Tnfrsf18, Tnnt2, Unc45b, Wnt9a, Zac1, and Zfp385 antibodies and antibody fragments can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

6. Pharmaceutical Carriers/Delivery of Pharamceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines

b) Therapeutic Uses

Effective dosages and schedules for administering the compositions may be determined empirically, and making such determinations is within the skill in the art. The dosage ranges for the administration of the compositions are those large enough to produce the desired effect in which the symptoms/disorder are/is effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

Following administration of a disclosed composition, such as an antibody, for treating, inhibiting, or preventing a cancer, the efficacy of the therapeutic antibody can be assessed in various ways well known to the skilled practitioner. For instance, one of ordinary skill in the art will understand that a composition, such as an antibody, disclosed herein is efficacious in treating or inhibiting a cancer in a subject by observing that the composition reduces tumor size or prevents a further increase in other indicators of tumor survival or growth including but not limited to neoplastic cell transformation in vitro, in vitro cell death, in vivo cell death, in vitro angiogenesis, in vivo tumor angiogenesis, tumor formation, tumor maintenance, or tumor proliferation or further decrease in in vitro or in vivo survival.

The compositions that inhibit Abat, Abca1, Ank, Ankrd1, Arhgap24, Atp8a1, Bbs7, Bex1, Ccl9, Centd3, Chst1, Ckmt1, Col9a3, Cpz, Cxcl1, Cxcl15, Daf1, Dapk1, Dffb, Dgka, Dixdc, Dusp15, Elav12, Eno3, Ephb2, Espn, Eva1, Fas, F2r11, Fgf18, Fgf7, Fhod3, FHOS2, Garn13, Gca, Gpr149, Hbegf, Hey2, Hmga1, Hmga2, Hoxc13, Id2, Id4, Igfbp2, Igsf4a, Jag2, Kctd15, Lass4, Ldhb, Man2b1, Mcam, Mmp15, Mpp7, Mrpl15, Mrpplf4, Ms4a10, Mtus1, Nbea, Notch3, Noxa, Oaf, Parvb, Pard6g, Perp, Plac8, P1a2g7, Pitx2, Pltp, Plxdc2, Prkcm, Prkg, Prss22, Pyr14, Rab40b, Rai2, Rasl11a, Rb1, Rgs2, Rprm, Rspo3, Satb1, Sbk1, Sbsn, Scn3b, Sema3d, Sema7a, Serpinb2, Sfrp2, Slc14a1, Slc27a3, Sms, Sod3, Stmn4, Tex15, Tnfrsf18, Tnnt2, Unc45b, Wnt9a, Zac1, and Zfp385 interactions disclosed herein may be administered prophylactically to patients or subjects who are at risk for a cancer.

Other molecules that interact with Abat, Abca1, Ank, Ankrd1, Arhgap24, Atp8a1, Bbs7, Bex1, Ccl9, Centd3, Chst1, Ckmt1, Col9a3, Cpz, Cxcl1, Cxcl15, Daf1, Dapk1, Dffb, Dgka, Dixdc, Dusp15, Elav12, Eno3, Ephb2, Espn, Eva1, Fas, F2r11, Fgf18, Fgf7, Fhod3, FHOS2, Garn13, Gca, Gpr149, Hbegf, Hey2, Hmga1, Hmga2, Hoxc13, Id2, Id4, Igfbp2, Igsf4a, Jag2, Kctd15, Lass4, Ldhb, Man2b1, Mcam, Mmp15, Mpp7, Mrpl15, Mrpplf4, Ms4a10, Mtus1, Nbea, Notch3, Noxa, Oaf, Parvb, Pard6g, Perp, Plac8, Pla2g7, Pitx2, Pltp, Plxdc2, Prkcm, Prkg, Prss22, Pyr14, Rab40b, Rai2, Rasl11a, Rb1, Rgs2, Rprm, Rspo3, Satb1, Sbk1, Sbsn, Scn3b, Sema3d, Sema7a, Serpinb2, Sfrp2, Slc14a1, Slc27a3, Sms, Sod3, Stmn4, Tex15, Tnfrsf18, Tnnt2, Unc45b, Wnt9a, Zac1, and Zfp385 which do not have a specific pharmacuetical function, but which may be used for tracking changes within cellular chromosomes or for the delivery of diagnositc tools for example can be delivered in ways similar to those described for the pharmaceutical products.

The disclosed compositions and methods can also be used for example as tools to isolate and test new drug candidates for various cancers including but not limited to lymphoma, B cell lymphoma, T cell lymphoma, mycosis fungoides, Hodgkin's Disease, leukemias, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, gastric cancer, colon cancer, cervical cancer, cervical carcinoma, breast cancer, and epithelial cancer, bone cancers, renal cancer, bladder cancer, genitourinary cancer, esophageal carcinoma, large bowel cancer, metastatic cancers hematopoietic cancers, sarcomas, Ewing's sarcoma, synovial cancer, soft tissue cancers; and testicular cancer.

7. Chips and Micro Arrays

Disclosed are chips where at least one address is the sequences or part of the sequences set forth in any of the nucleic acid sequences disclosed herein. Also disclosed are chips where at least one address is the sequences or portion of sequences set forth in any of the peptide sequences disclosed herein.

Also disclosed are chips where at least one address is a variant of the sequences or part of the sequences set forth in any of the nucleic acid sequences disclosed herein. Also disclosed are chips where at least one address is a variant of the sequences or portion of sequences set forth in any of the peptide sequences disclosed herein.

8. Compositions Identified by Screening with Disclosed Compositions/Combinatorial Chemistry

a) Combinatorial Chemistry

The disclosed compositions can be used as targets for any combinatorial technique to identify molecules or macromolecular molecules that interact with the disclosed compositions in a desired way. Also disclosed are the compositions that are identified through combinatorial techniques or screening techniques in which the compositions disclosed in Table 1 or portions thereof, are used as the target in a combinatorial or screening protocol.

It is understood that when using the disclosed compositions in combinatorial techniques or screening methods, molecules, such as macromolecular molecules, will be identified that have particular desired properties such as inhibition or stimulation or the target molecule's function. The molecules identified and isolated when using the disclosed compositions, such as, Abat, Abca1, Ank, Ankrd1, Arhgap24, Atp8a1, Bbs7, Bex1, Ccl9, Centd3, Chst1, Ckmt1, Col9a3, Cpz, Cxcl1, Cxcl15, Daf1, Dapk1, Dffb, Dgka, Dixdc, Dusp15, Elav12, Eno3, Ephb2, Espn, Eva1, Fas, F2r11, Fgf18, Fgf7, Fhod3, FHOS2, Garn13, Gca, Gpr149, Hbegf, Hey2, Hmga1, Hmga2, Hoxc13, Id2, Id4, Igfbp2, Igsf4a, Jag2, Kctd15, Lass4, Ldhb, Man2b1, Mcam, Mmp15, Mpp7, Mrpl15, Mrpplf4, Ms4a10, Mtus1, Nbea, Notch3, Noxa, Oaf, Parvb, Pard6g, Perp, Plac8, Pla2g7, Pitx2, Pltp, Plxdc2, Prkcm, Prkg, Prss22, Pyr14, Rab40b, Rai2, Rasl11a, Rb1, Rgs2, Rprm, Rspo3, Satb1, Sbk1, Sbsn, Scn3b, Sema3d, Sema7a, Serpinb2, Sfrp2, Slc14a1, Slc27a3, Sms, Sod3, Stmn4, Tex15, Tnfrsf18, Tnnt2, Unc45b, Wnt9a, Zac1, and Zfp385, are also disclosed. Thus, the products produced using the combinatorial or screening approaches that involve the disclosed compositions, such as, Abat, Abca1, Ank, Ankrd1, Arhgap24, Atp8a1, Bbs7, Bex1, Ccl9, Centd3, Chst1, Ckmt1, Col9a3, Cpz, Cxcl1, Cxcl15, Daf1, Dapk1, Dffb, Dgka, Dixdc, Dusp15, Elav12, Eno3, Ephb2, Espn, Eva1, Fas, F2r11, Fgf18, Fgf7, Fhod3, FHOS2, Garn13, Gca, Gpr149, Hbegf, Hey2, Hmga1, Hmga2, Hoxc13, Id2, Id4, Igfbp2, Igsf4a, Jag2, Kctd15, Lass4, Ldhb, Man2b1, Mcam, Mmp15, Mpp7, Mrpl15, Mrpplf4, Ms4a10, Mtus1, Nbea, Notch3, Noxa, Oaf, Parvb, Pard6g, Perp, Plac8, P1a2g7, Pitx2, Pltp, Plxdc2, Prkcm, Prkg, Prss22, Pyr14, Rab40b, Rai2, Rasl11a, Rb1, Rgs2, Rprm, Rspo3, Satb1, Sbk1, Sbsn, Scn3b, Sema3d, Sema7a, Serpinb2, Sfrp2, Slc14a1, Slc27a3, Sms, Sod3, Stmn4, Tex15, Tnfrsf18, Tnnt2, Unc45b, Wnt9a, Zac1, and Zfp385, are also considered herein disclosed.

It is understood that the disclosed methods for identifying molecules that inhibit the interactions of, for example, Abat, Abca1, Ank, Ankrd1, Arhgap24, Atp8a1, Bbs7, Bex1, Ccl9, Centd3, Chst1, Ckmt1, Col9a3, Cpz, Cxcl1, Cxcl15, Daf1, Dapk1, Dffb, Dgka, Dixdc, Dusp15, Elav12, Eno3, Ephb2, Espn, Eva1, Fas, F2r11, Fgf18, Fgf7, Fhod3, FHOS2, Garn13, Gca, Gpr149, Hbegf, Hey2, Hmga1, Hmga2, Hoxc13, Id2, Id4, Igfbp2, Igsf4a, Jag2, Kctd15, Lass4, Ldhb, Man2b1, Mcam, Mmp15, Mpp7, Mrpl15, Mrpplf4, Ms4a10, Mtus1, Nbea, Notch3, Noxa, Oaf, Parvb, Pard6g, Perp, Plac8, Pla2g7, Pitx2, Pltp, Plxdc2, Prkcm, Prkg, Prss22, Pyr14, Rab40b, Rai2, Rasl11a, Rb1, Rgs2, Rprm, Rspo3, Satb1, Sbk1, Sbsn, Scn3b, Sema3d, Sema7a, Serpinb2, Sfrp2, Slc14a1, Slc27a3, Sms, Sod3, Stmn4, Tex15, Tnfrsf18, Tnnt2, Unc45b, Wnt9a, Zac1, and Zfp385 can be performed using high through put means. For example, putative inhibitors can be identified using Fluorescence Resonance Energy Transfer (FRET) to quickly identify interactions. The underlying theory of the techniques is that when two molecules are close in space, ie, interacting at a level beyond background, a signal is produced or a signal can be quenched. Then, a variety of experiments can be performed, including, for example, adding in a putative inhibitor. If the inhibitor competes with the interaction between the two signaling molecules, the signals will be removed from each other in space, and this will cause a decrease or an increase in the signal, depending on the type of signal used. This decrease or increasing signal can be correlated to the presence or absence of the putative inhibitor. Any signaling means can be used. For example, disclosed are methods of identifying an inhibitor of the interaction between any two of the disclosed molecules comprising, contacting a first molecule and a second molecule together in the presence of a putative inhibitor, wherein the first molecule or second molecule comprises a fluorescence donor, wherein the first or second molecule, typically the molecule not comprising the donor, comprises a fluorescence acceptor; and measuring Fluorescence Resonance Energy Transfer (FRET), in the presence of the putative inhibitor and the in absence of the putative inhibitor, wherein a decrease in FRET in the presence of the putative inhibitor as compared to FRET measurement in its absence indicates the putative inhibitor inhibits binding between the two molecules. This type of method can be performed with a cell system as well.

Combinatorial chemistry includes but is not limited to all methods for isolating small molecules or macromolecules that are capable of binding either a small molecule or another macromolecule, typically in an iterative process. Proteins, oligonucleotides, and sugars are examples of macromolecules. For example, oligonucleotide molecules with a given function, catalytic or ligand-binding, can be isolated from a complex mixture of random oligonucleotides in what has been referred to as “in vitro genetics” (Szostak, TIBS19:89, 1992). One synthesizes a large pool of molecules bearing random and defined sequences and subjects that complex mixture, for example, approximately 1015 individual sequences in 100 μg of a 100 nucleotide RNA, to some selection and enrichment process. Through repeated cycles of affinity chromatography and PCR amplification of the molecules bound to the ligand on the column, Ellington and Szostak (1990) estimated that 1 in 1010 RNA molecules folded in such a way as to bind a small molecule dyes. DNA molecules with such ligand-binding behavior have been isolated as well (Ellington and Szostak, 1992; Bock et al, 1992). Techniques aimed at similar goals exist for small organic molecules, proteins, antibodies and other macromolecules known to those of skill in the art. Screening sets of molecules for a desired activity whether based on small organic libraries, oligonucleotides, or antibodies is broadly referred to as combinatorial chemistry. Combinatorial techniques are particularly suited for defining binding interactions between molecules and for isolating molecules that have a specific binding activity, often called aptamers when the macromolecules are nucleic acids.

There are a number of methods for isolating proteins which either have de novo activity or a modified activity. For example, phage display libraries have been used to isolate numerous peptides that interact with a specific target. (See for example, U.S. Pat. Nos. 6,031,071; 5,824,520; 5,596,079; and 5,565,332 which are herein incorporated by reference at least for their material related to phage display and methods relate to combinatorial chemistry)

A preferred method for isolating proteins that have a given function is described by Roberts and Szostak (Roberts R. W. and Szostak J. W. Proc. Natl. Acad. Sci. USA, 94(23)12997-302 (1997). This combinatorial chemistry method couples the functional power of proteins and the genetic power of nucleic acids. An RNA molecule is generated in which a puromycin molecule is covalently attached to the 3′-end of the RNA molecule. An in vitro translation of this modified RNA molecule causes the correct protein, encoded by the RNA to be translated. In addition, because of the attachment of the puromycin, a peptdyl acceptor which cannot be extended, the growing peptide chain is attached to the puromycin which is attached to the RNA. Thus, the protein molecule is attached to the genetic material that encodes it. Normal in vitro selection procedures can now be done to isolate functional peptides. Once the selection procedure for peptide function is complete traditional nucleic acid manipulation procedures are performed to amplify the nucleic acid that codes for the selected functional peptides. After amplification of the genetic material, new RNA is transcribed with puromycin at the 3′-end, new peptide is translated and another functional round of selection is performed. Thus, protein selection can be performed in an iterative manner just like nucleic acid selection techniques. The peptide which is translated is controlled by the sequence of the RNA attached to the puromycin. This sequence can be anything from a random sequence engineered for optimum translation (i.e. no stop codons etc.) or it can be a degenerate sequence of a known RNA molecule to look for improved or altered function of a known peptide. The conditions for nucleic acid amplification and in vitro translation are well known to those of ordinary skill in the art and are preferably performed as in Roberts and Szostak (Roberts R. W. and Szostak J. W. Proc. Natl. Acad. Sci. USA, 94(23)12997-302 (1997)).

Another preferred method for combinatorial methods designed to isolate peptides is described in Cohen et al. (Cohen B. A., et al., Proc. Natl. Acad. Sci. USA 95(24):14272-7 (1998). This method utilizes and modifies two-hybrid technology. Yeast two-hybrid systems are useful for the detection and analysis of protein:protein interactions. The two-hybrid system, initially described in the yeast Saccharomyces cerevisiae, is a powerful molecular genetic technique for identifying new regulatory molecules, specific to the protein of interest (Fields and Song, Nature 340:245-6 (1989)). Cohen et al., modified this technology so that novel interactions between synthetic or engineered peptide sequences could be identified which bind a molecule of choice. The benefit of this type of technology is that the selection is done in an intracellular environment. The method utilizes a library of peptide molecules that attached to an acidic activation domain. A peptide of choice is attached to a DNA binding domain of a transcriptional activation protein, such as Gal 4. By performing the Two-hybrid technique on this type of system, molecules that bind the extracellular portion of the protein from which the peptide was derived can be identified.

Using methodology well known to those of skill in the art, in combination with various combinatorial libraries, one can isolate and characterize those small molecules or macromolecules, which bind to or interact with the desired target. The relative binding affinity of these compounds can be compared and optimum compounds identified using competitive binding studies, which are well known to those of skill in the art.

Techniques for making combinatorial libraries and screening combinatorial libraries to isolate molecules which bind a desired target are well known to those of skill in the art. Representative techniques and methods can be found in but are not limited to U.S. Pat. Nos. 5,084,824, 5,288,514, 5,449,754, 5,506,337, 5,539,083, 5,545,568, 5,556,762, 5,565,324, 5,565,332, 5,573,905, 5,618,825, 5,619,680, 5,627,210, 5,646,285, 5,663,046, 5,670,326, 5,677,195, 5,683,899, 5,688,696, 5,688,997, 5,698,685, 5,712,146, 5,721,099, 5,723,598, 5,741,713, 5,792,431, 5,807,683, 5,807,754, 5,821,130, 5,831,014, 5,834,195, 5,834,318, 5,834,588, 5,840,500, 5,847,150, 5,856,107, 5,856,496, 5,859,190, 5,864,010, 5,874,443, 5,877,214, 5,880,972, 5,886,126, 5,886,127, 5,891,737, 5,916,899, 5,919,955, 5,925,527, 5,939,268, 5,942,387, 5,945,070, 5,948,696, 5,958,702, 5,958,792, 5,962,337, 5,965,719, 5,972,719, 5,976,894, 5,980,704, 5,985,356, 5,999,086, 6,001,579, 6,004,617, 6,008,321, 6,017,768, 6,025,371, 6,030,917, 6,040,193, 6,045,671, 6,045,755, 6,060,596, and 6,061,636.

Combinatorial libraries can be made from a wide array of molecules using a number of different synthetic techniques. For example, libraries containing fused 2,4-pyrimidinediones (U.S. Pat. No. 6,025,371) dihydrobenzopyrans (U.S. Pat. Nos. 6,017,768 and 5,821,130), amide alcohols (U.S. Pat. No. 5,976,894), hydroxy-amino acid amides (U.S. Pat. No. 5,972,719) carbohydrates (U.S. Pat. No. 5,965,719), 1,4-benzodiazepin-2,5-diones (U.S. Pat. No. 5,962,337), cyclics (U.S. Pat. No. 5,958,792), biaryl amino acid amides (U.S. Pat. No. 5,948,696), thiophenes (U.S. Pat. No. 5,942,387), tricyclic Tetrahydroquinolines (U.S. Pat. No. 5,925,527), benzofurans (U.S. Pat. No. 5,919,955), isoquinolines (U.S. Pat. No. 5,916,899), hydantoin and thiohydantoin (U.S. Pat. No. 5,859,190), indoles (U.S. Pat. No. 5,856,496), imidazol-pyrido-indole and imidazol-pyrido-benzothiophenes (U.S. Pat. No. 5,856,107) substituted 2-methylene-2,3-dihydrothiazoles (U.S. Pat. No. 5,847,150), quinolines (U.S. Pat. No. 5,840,500), PNA (U.S. Pat. No. 5,831,014), containing tags (U.S. Pat. No. 5,721,099), polyketides (U.S. Pat. No. 5,712,146), morpholino-subunits (U.S. Pat. Nos. 5,698,685 and 5,506,337), sulfamides (U.S. Pat. No. 5,618,825), and benzodiazepines (U.S. Pat. No. 5,288,514).

As used herein combinatorial methods and libraries included traditional screening methods and libraries as well as methods and libraries used in interative processes.

b) Computer Assisted Drug Design

The disclosed compositions can be used as targets for any molecular modeling technique to identify either the structure of the disclosed compositions or to identify potential or actual molecules, such as small molecules, which interact in a desired way with the disclosed compositions. The nucleic acids, peptides, and related molecules disclosed herein can be used as targets in any molecular modeling program or approach.

It is understood that when using the disclosed compositions in modeling techniques, molecules, such as macromolecular molecules, will be identified that have particular desired properties such as inhibition or stimulation or the target molecule's function. The molecules identified and isolated when using the disclosed compositions, such as, Abat, Abca1, Ank, Ankrd1, Arhgap24, Atp8a1, Bbs7, Bex1, Ccl9, Centd3, Chst1, Ckmt1, Col9a3, Cpz, Cxcl1, Cxcl15, Daf1, Dapk1, Dffb, Dgka, Dixdc, Dusp15, Elav12, Eno3, Ephb2, Espn, Eva1, Fas, F2r11, Fgf18, Fgf7, Fhod3, FHOS2, Garn13, Gca, Gpr149, Hbegf, Hey2, Hmga1, Hmga2, Hoxc13, Id2, Id4, Igfbp2, Igsf4a, Jag2, Kctd15, Lass4, Ldhb, Man2b1, Mcam, Mmp15, Mpp7, Mrpl15, Mrpplf4, Ms4a10, Mtus1, Nbea, Notch3, Noxa, Oaf, Parvb, Pard6g, Perp, Plac8, P1a2g7, Pitx2, Pltp, Plxdc2, Prkcm, Prkg, Prss22, Pvr14, Rab40b, Rai2, Rasl11a, Rb1, Rgs2, Rprm, Rspo3, Satb1, Sbk1, Sbsn, Scn3b, Sema3d, Sema7a, Serpinb2, Sfrp2, Slc14a1, Slc27a3, Sms, Sod3, Stmn4, Tex15, Tnfrsf18, Tnnt2, Unc45b, Wnt9a, Zac1, and Zfp385, are also disclosed. Thus, the products produced using the molecular modeling approaches that involve the disclosed compositions, such as, Abat, Abca1, Ank, Ankrd1, Arhgap24, Atp8a1, Bbs7, Bex1, Ccl9, Centd3, Chst1, Ckmt1, Col9a3, Cpz, Cxcl1, Cxcl15, Daf1, Dapk1, Dffb, Dgka, Dixdc, Dusp15, Elav12, Eno3, Ephb2, Espn, Eva1, Fas, F2r11, Fgf18, Fgf7, Fhod3, FHOS2, Garn13, Gca, Gpr149, Hbegf, Hey2, Hmga1, Hmga2, Hoxc13, Id2, Id4, Igfbp2, Igsf4a, Jag2, Kctd15, Lass4, Ldhb, Man2b1, Mcam, Mmp15, Mpp7, Mrpl15, Mrpplf4, Ms4a10, Mtus1, Nbea, Notch3, Noxa, Oaf, Parvb, Pard6g, Perp, Plac8, Pla2g7, Pitx2, Pltp, Plxdc2, Prkcm, Prkg, Prss22, Pvr14, Rab40b, Rai2, Rasl11a, Rb1, Rgs2, Rprm, Rspo3, Satb1, Sbk1, Sbsn, Scn3b, Sema3d, Sema7a, Serpinb2, Sfrp2, Slc14a1, Slc27a3, Sms, Sod3, Stmn4, Tex15, Tnfrsf18, Tnnt2, Unc45b, Wnt9a, Zac1, and Zfp385, are also considered herein disclosed.

Thus, one way to isolate molecules that bind a molecule of choice is through rational design. This is achieved through structural information and computer modeling. Computer modeling technology allows visualization of the three-dimensional atomic structure of a selected molecule and the rational design of new compounds that will interact with the molecule. The three-dimensional construct typically depends on data from x-ray crystallographic analyses or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

Examples of molecular modeling systems are the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen, et al., 1988 Acta Pharmaceutica Fennica 97, 159-166; Ripka, New Scientist 54-57 (Jun. 16, 1988); McKinaly and Rossmann, 1989 Annu. Rev. Pharmacol. Toxiciol. 29, 111-122; Perry and Davies, QSAR: Quantitative Structure-Activity Relationships in Drug Design pp. 189-193 (Alan R. Liss, Inc. 1989); Lewis and Dean, 1989 Proc. R. Soc. Lond. 236, 125-140 and 141-162; and, with respect to a model enzyme for nucleic acid components, Askew, et al., 1989 J. Am. Chem. Soc. 111, 1082-1090. Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. Although these are primarily designed for application to drugs specific to particular proteins, they can be adapted to design of molecules specifically interacting with specific regions of DNA or RNA, once that region is identified.

Although described above with reference to design and generation of compounds which could alter binding, one could also screen libraries of known compounds, including natural products or synthetic chemicals, and biologically active materials, including proteins, for compounds which alter substrate binding or enzymatic activity.

9. Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended. For example, disclosed is a kit for assessing a subject's risk for acquiring colon cancer, comprising a panel of cooperation response genes on a microarray or protein array.

Throughout this application, various publications are referenced. The disclosures of these publications in their entireties are hereby incorporated by reference into this application in order to more fully describe the state of the art to which this invention pertains. The references disclosed are also individually and specifically incorporated by reference herein for the material contained in them that is discussed in the sentence in which the reference is relied upon.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### D. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### Analysis of Synergistic Response to Oncogenic Mutations Pinpoints Genes Essential for Cancer Phenotype

Recent observations that cell transformation by p53 loss-of-function and Ras activation depends on synergistic modulation of downstream signaling circuitry (Xia, M. & Land, H. (2007) Nat Struct Mol Biol 14, 215-23) suggested that malignant cell transformation is a highly cooperative process critically involving synergy at multiple molecular levels. Herein is demonstrated that the malignant state is critically dependent on a cohort of downstream genes controlled synergistically by cooperating oncogenic mutations such as loss-of-function p53 and Ras activation. Remarkably, 14 among 24 such ‘cooperation response genes’ (CRGs) were found to contribute strongly to tumor formation in gene perturbation experiments. In contrast, only one in 14 perturbations of genes responding in a non-synergistic manner had a similar effect. Synergistic control of gene expression by oncogenic mutations thus provides an attractive strategy for identifying intervention targets in gene networks downstream of oncogenic gain and loss-of-funtion mutations that underly malignant cell transformation.

Genes regulated synergistically by cooperating oncogenic mutations were identified by comparing mRNA expression profiles of young adult murine colon (YAMC) cells (Whitehead, R. H., et al. (1993) Proc Natl Acad Sci USA 90, 587-913) with those of YAMC cells expressing mutant p53175H (mp53), activated H-Ras 12V (Ras) or both mutant proteins together (mp53/Ras) (Xia, M. & Land, H. (2007) Nat Struct Mol Biol 14, 215-23) using Affymetrix mouse whole genome microarrays. Using a step-wise procedure, 538 genes (represented by 657 probe sets) were identified that were differentially expressed in mp53, Ras and mp53/Ras cells, as compared to YAMC control cells with a statistical cut off at p<0.01 (N-test, Westfall-Young adjusted). A further subset of 95 annotated genes that respond synergistically (24 up/67 down) to the combination of mutant p53 and Ras proteins, termed ‘cooperation response genes’ (CRG) was then determined using a synergy criterion, as described in methods (Table 1). A synergy score of 0.9 or less defines CRGs. Expression values for the CRGs derived from the microarrays also showed a strong positive correlation with expression values for the same genes obtained by TaqMan low-density QPCR arrays (TLDA) (Tables 1 and 2). Thus CRG identification was confirmed by independent methods, with final CRG selection based on microarray data, due to higher sample replication in this data set.

TABLE 1
Cooperation Response Genes

| | | | | Expression | Synergy | Expression | Synergy |
|---|---|---|---|---|---|---|---|
| | | | | mp53/Ras | Score, | mp53/Ras | Score, |
| | | | | vs. YAMC, | Raw | vs. YAMC, | Norm |
| GO Biological | | | | Raw Data | Data, | Norm Data | Data, |
| Process | Gene Symbol | GenBank ID | Affymetrix ID | (fold) | p < 0.01 | (fold) | p < 0.01 |
| Signal | Arhgap24 | BC025502 | 1424842_a_at | 0.08 | 0.29 | 0.07 | 0.31 |
| Transduction | Centd3 | AI851258 | 1419833_s_at | 3.64 | 0.87 | 3.39 | 0.83 |
| | Dgka | BC006713 | 1418578_at | 0.30 | 0.79 | 0.28 | 0.88 |
| | Dixdc1 | BB758432 | 1435207_at | 0.38 | 0.85 | 0.36 | 0.93 |
| | Dusp15 | AF357887 | 1426189_at | 0.57 | 0.84 | 0.51 | 0.89 |
| | Ephb2 | AV221401 | 1425016_at | 0.15 | 0.58 | 0.14 | 0.62 |
| | F2rl1 | NM_007974 | 1448931_at | 2.15 | 0.93** | 2.07 | 0.82 |
| | Fgf18 | NM_008005 | 1449545_at | 0.38 | 0.89 | 0.37 | 0.99# |
| | Fgf7 | NM_008008 | 1422243_at | 7.43 | 0.93** | 7.08 | 0.85 |
| | Garnl3 | BB131106 | 1433553_at | 0.28 | 0.88 | 0.27 | 0.93 |
| | Gpr149 | BB126999 | 1438210_at | 4.09 | 0.55 | 3.87 | 0.53 |
| | Hbegf | L07264 | 1418350_at | 4.57 | 0.99# | 4.44 | 0.90** |
| | Igfbp2 | AK011784 | 1454159_a_at | 0.15 | 0.37* | 0.15 | 0.43* |
| | Jag2 | AV264681 | 1426431_at | 0.24 | 0.86 | 0.23 | 0.91 |
| | Ms4a10 | AK008019 | 1432453_a_at | 0.24 | 0.73 | 0.24 | 0.82 |
| | Pard6g | NM_053117 | 1420851_at | 0.35 | 0.79 | 0.33 | 0.90 |
| | Plxdc2 | BB559706 | 1418912_at | 0.03 | 0.36 | 0.03 | 0.41 |
| | Prkcm | AV297026 | 1447623_s_at | 0.24 | 0.90* | 0.23 | 1.03# |
| | Prkg1 | BB516668 | 1444232_at | 0.23 | 0.86* | 0.23 | 0.95* |
| | Rab40b | AV364488 | 1436566_at | 0.32 | 0.85* | 0.31 | 0.93* |
| | Rasl11a | AK004371 | 1429444_at | 0.42 | 0.87 | 0.41 | 0.95 |
| | Rb1 | NM_009029 | 1417850_at | 0.28 | 0.74 | 0.27 | 0.83 |
| | Rgs2 | AF215668 | 1419248_at | 3.91 | 0.66 | 3.70 | 0.62 |
| | Rprm | NM_023396 | 1422552_at | 0.29 | 0.69 | 0.30 | 0.81 |
| | Sbk1 | BC025837 | 1451190_a_at | 0.40 | 0.81 | 0.41 | 0.91 |
| | Sema3d | BB499147 | 1429459_at | 0.17 | 0.72* | 0.16 | 0.80* |
| | Sema7a | AA144045 | 1459903_at | 4.77 | 0.68 | 4.41 | 0.61 |
| | Sfrp2 | NM_009144 | 1448201_at | 0.13 | 0.27 | 0.13 | 0.31 |
| | Stmn4 | NM_019675 | 1418105_at | 0.36 | 0.73 | 0.34 | 0.78 |
| | Wnt9a | AV273409 | 1436978_at | 0.37 | 0.89 | 0.35 | 1.00# |
| Metabolism/ | Abat | BF462185 | 1433855_at | 0.20 | 0.90* | 0.20 | 0.94# |
| Transport | Abca1 | BB144704 | 1421840_at | 0.14 | 0.59 | 0.13 | 0.65 |
| | Ank | NM_020332 | 1450627_at | 21.76 | 0.64 | 20.34 | 0.62 |
| | Atp8a1 | AW610650 | 1454728_s_at | 0.20 | 0.90* | 0.19 | 0.96# |
| | Chst1 | NM_023850 | 1449147_at | 7.98 | 0.74 | 7.61 | 0.70 |
| | Cpz | AF356844 | 1426251_at | 0.18 | 0.76 | 0.17 | 0.83 |
| | Eno3 | NM_007933 | 1417951_at | 5.46 | 0.77 | 4.69 | 0.75 |
| | Kctd15 | BB091366 | 1435339_at | 6.41 | 0.82 | 6.01 | 0.70 |
| | Ldhb | AV219418 | 1434499_a_at | 0.17 | 0.56 | 0.17 | 0.62 |
| | Man2b1 | BC005430 | 1416340_a_at | 0.31 | 0.83 | 0.29 | 0.91 |
| | Mtus1 | BB699957 | 1454824_s_at | 0.23 | 0.85** | 0.22 | 0.94* |
| | Nbea | AA986379 | 1452251_at | 0.24 | 0.81 | 0.23 | 0.90 |
| | Pla2g7 | AK005158 | 1430700_a_at | 11.07 | 0.55 | 10.67 | 0.50 |
| | Pltp | NM_011125 | 1417963_at | 0.33 | 0.88 | 0.30 | 0.98# |
| | Scn3b | BE951842 | 1435767_at | 0.08 | 0.59 | 0.07 | 0.57 |
| | Slc14a1 | AW556396 | 1428114_at | 9.25 | 0.42 | 9.20 | 0.39 |
| | Slc27a3 | BB147793 | 1427180_at | 0.32 | 0.81 | 0.31 | 0.89 |
| | Sms | NM_009214 | 1421052_a_at | 4.00 | 0.97# | 3.84 | 0.89 |
| | Sod3 | NM_011435 | 1417633_at | 3.98 | 0.96# | 4.03 | 0.90** |
| Cell | Ccl9 | AF128196 | 1417936_at | 8.07 | 0.92 | 7.90 | 0.82 |
| Adhesion | Col9a3 | BG074456 | 1460693_a_at | 0.25 | 0.39 | 0.25 | 0.43 |
| | Cxcl1 | NM_008176 | 1419209_at | 9.83 | 1.02# | 9.71 | 0.84 |
| | Cxcl15 | NM_011339 | 1421404_at | 16.13 | 0.83* | 15.43 | 0.70 |
| | Espn | NM_019585 | 1423005_a_at | 0.23 | 0.67 | 0.23 | 0.76 |
| | Eva1 | BC015076 | 1448265_x_at | 0.25 | 0.86* | 0.24 | 0.96# |
| | Fhod3 | BG066491 | 1435551_at | 0.19 | 0.61** | 0.17 | 0.67** |
| | Igsf4a | NM_018770 | 1417378_at | 18.17 | 0.71 | 16.89 | 0.70 |
| | Mcam | NM_023061 | 1416357_a_at | 0.15 | 0.63 | 0.15 | 0.70 |
| | Mmp15 | NM_008609 | 1422597_at | 0.31 | 0.83 | 0.30 | 0.90 |
| | Parvb | BI134721 | 1438672_at | 4.77 | 0.92** | 4.48 | 0.86 |
| | Pvrl4 | BC024948 | 1451690_a_at | 0.39 | 0.88 | 0.36 | 0.97# |
| Transcriptional | Ankrd1 | AK009959 | 1420992_at | 3.78 | 0.51 | 3.88 | 0.46 |
| Regulators | Hey2 | NM_013904 | 1418106_at | 0.20 | 0.73 | 0.20 | 0.79 |
| | Hmga1 | NM_016660 | 1416184_s_at | 12.21 | 0.83 | 11.38 | 0.82 |
| | Hmga2 | X58380 | 1450781_at | 14.96 | 0.90** | 14.88 | 0.87 |
| | Hoxc13 | AF193796 | 1425874_at | 0.42 | 0.83 | 0.43 | 0.97 |
| | Id2 | BF019883 | 1435176_a_at | 0.24 | 0.61 | 0.25 | 0.69 |
| | Id4 | BB121406 | 1423259_at | 0.10 | 0.39 | 0.09 | 0.41 |
| | Lass4 | BB006809 | 1417782_at | 0.27 | 0.69 | 0.25 | 0.72 |
| | Notch3 | NM_008716 | 1421965_s_at | 0.18 | 0.62 | 0.17 | 0.70 |
| | Pitx2 | U80011 | 1424797_a_at | 0.38 | 0.77 | 0.35 | 0.83 |
| | Satb1 | AV172776 | 1416007_at | 0.23 | 0.80* | 0.22 | 0.87* |
| Apoptosis | Dapk1 | BC021490 | 1427358_a_at | 0.17 | 0.58 | 0.16 | 0.62 |
| | Dffb | AV300013 | 1437051_at | 0.35 | 0.86 | 0.35 | 0.95 |
| | Fas | NM_007987 | 1460251_at | 0.35 | 0.83 | 0.35 | 0.96 |
| | Noxa | NM_021451 | 1418203_at | 0.05 | 0.26 | 0.05 | 0.27 |
| | Perp | NM_022032 | 1416271_at | 0.17 | 0.70 | 0.17 | 0.75 |
| Unknown | Bbs7 | BG074932 | 1454684_at | 0.50 | 0.89 | 0.50 | 1.01# |
| Function | Ckmt1 | NM_009897 | 1417089_a_at | 0.43 | 0.89 | 0.40 | 0.93* |
| | Elavl2 | BB105998 | 1421883_at | 0.40 | 0.72* | 0.39 | 0.83* |
| | Gca | BC021450 | 1451451_at | 0.34 | 0.85* | 0.33 | 0.95* |
| | Mpp7 | AK012883 | 1455179_at | 0.13 | 0.44 | 0.13 | 0.46 |
| | Mrpl15 | AV306676 | 1430798_x_at | 3.18 | 0.98# | 3.08 | 0.88 |
| | Oaf | BC025514 | 1424086_at | 5.01 | 0.99# | 5.08 | 0.90 |
| | Plac8 | AF263458 | 1451335_at | 3.40 | 0.89 | 3.21 | 0.88 |
| | Rai2 | BB770528 | 1452358_at | 0.26 | 0.80 | 0.25 | 0.85 |
| | Sbsn | AI507307 | 1459898_at | 0.41 | 0.72 | 0.38 | 0.78 |
| | Serpinb2 | NM_011111 | 1419082_at | 9.07 | 0.92# | 8.91 | 0.90* |
| | Tex15 | NM_031374 | 1420719_at | 0.16 | 0.59 | 0.15 | 0.59 |
| | Tnfrsf18 | AF229434 | 1422303_a_at | 0.20 | 0.56 | 0.20 | 0.65 |
| | Unc45b | AV220213 | 1436939_at | 0.22 | 0.83 | 0.21 | 0.82 |
| | Zfp385 | NM_013866 | 1418865_at | 0.36 | 0.85 | 0.37 | 0.98# |
| Other | Bex1 | NM_009052 | 1448595_a_at | 0.14 | 0.38* | 0.14 | 0.45* |
| | Daf1 | BE686894 | 1443906_at | 0.11 | 0.41 | 0.11 | 0.43 |
| | Tnnt2 | L47552 | 1424967_x_at | 9.42 | 0.87 | 10.11 | 0.80 |

Unnamed Cooperation Response Genes

| | | Affymetrix | Up/Down |
|---|---|---|---|
| Gene Symbol | GenBank ID | ID | Regulated |
| — | BB333822 | 1446179_at | Up |
| — | BB016042 | 1443437_at | Up |
| — | AV254043 | 1439944_at | Up |
| 2010204K13Rik | NM_023450 | 1421498_a_at | Up |
| 2310002L13Rik | AK009098 | 1453275_at | Up |
| 2610528A11Rik | BF580962 | 1435639_at | Up |
| A130040M12Rik | C85657 | 1428909_at | Up |
| AI467606 | BB234337 | 1433465_a_at | Up |
| AI467606 | BB234337 | 1433466_at | Up |
| B630019K06Rik | BB179847 | 1433452_at | Up |
| Prl2c2 /// Prl2c3 /// | X75557 | 1427760_s_at | Up |
| Prl2c4 | | | |
| — | AA266723 | 1448021_at | Down |
| — | AV133559 | 1459971_at | Down |
| — | BB767109 | 1439734_at | Down |
| — | BB133117 | 1441636_at | Down |
| — | AW543723 | 1441971_at | Down |
| — | BB353853 | 1438310_at | Down |
| — | BM118398 | 1435981_at | Down |
| — | BG076276 | 1445758_at | Down |
| — | BB306828 | 1455298_at | Down |
| — | BQ266693 | 1442073_at | Down |
| — | AV254764 | 1456951_at | Down |
| 1700007K13Rik | AK005731 | 1428705_at | Down |
| 2210023G05Rik | BC027185 | 1424968_at | Down |
| 2310038E17Rik | AK009671 | 1432976_at | Down |
| 2410066E13Rik | BB167663 | 1434581_at | Down |
| 6230424C14Rik | BE949277 | 1441972_at | Down |
| 8030476L19Rik | BB068813 | 1454354_at | Down |
| 9930013L23Rik | AK018112 | 1429987_at | Down |
| A930008G19Rik | BM248711 | 1455428_at | Down |
| A930037G23Rik | BE957307 | 1454628_at | Down |
| BC013672 | BC013672 | 1451777_at | Down |
| BC037703 | AV231983 | 1455241_at | Down |
| C030027H14Rik | BB358264 | 1442175_at | Down |
| C130026I21Rik /// | BC007193 | 1425078_x_at | Down |
| LOC100041885 | | | |
| C130092O11Rik | BG071013 | 1437306_at | Down |
| D330028D13Rik | BB478071 | 1434428_at | Down |
| Dzip1 /// | AI509011 | 1452792_at | Down |
| LOC100045776 | | | |
| Dzip1 /// | AI509011 | 1428469_a_at | Down |
| LOC100045776 | | | |
| LOC100044927 /// | NM_009398 | 1418424_at | Down |
| Tnfaip6 | | | |
| LOC100045546 | BB121406 | 1450928_at | Down |
| LOC100047292 | BI905111 | 1434889_at | Down |
| Acad11 | BQ031255 | 1433545_s_at | Down |
| Acad11 | BQ031255 | 1454647_at | Down |
| Adamts20 | AI450842 | 1456901_at | Down |
| AI956758 | AV234963 | 1460003_at | Down |
| Abi3bp | BC026627 | 1427054_s_at | Down |
| Adcy1 | AI848263 | 1456487_at | Down |
| Apol2 | BB312717 | 1441054_at | Down |
| Dmxl2 | AK018275 | 1428749_at | Down |
| Depdc7 | BC013499 | 1424303_at | Down |
| Ceecam1 | AV323203 | 1435345_at | Down |
| Brunol5 | BB381558 | 1434969_at | Down |
| Glis3 | BB207363 | 1430353_at | Down |
| Grhl3 | AV231424 | 1436932_at | Down |
| Gria3 | BM220576 | 1434728_at | Down |
| Limch1 | AV024662 | 1435106_at | Down |
| Limch1 | BM117827 | 1435321_at | Down |
| Mreg | AV298358 | 1437250_at | Down |
| Ms4a2 | AV241486 | 1443264_at | Down |
| Npr3 | BG066982 | 1435184_at | Down |
| Plekha7 | BF159528 | 1455343_at | Down |
| Ptpdc1 | AV254040 | 1433823_at | Down |
| Slain1 | BB704967 | 1424824_at | Down |
| Slc7a2 | AV244175 | 1436555_at | Down |
| Svop | AK003981 | 1452663_at | Down |

A synergy score smaller than 1 indicates a synergistic or non-additive change in gene expression in response to multiple as compared to single oncogenic mutations. The p-values estimate the level of confidence that the synergy score is less than one. Synergy scores and associated p-values were calculated as described in Methods. For all synergy scores, p-values are p < 0.01, except as indicated (**p < 0.05; *p < 0.1; #not significantly less than 1).

TABLE 2
TLDA assay ID numbers and corresponding synergy scores
for indicated CRGs.

| | | | Synergy | Synergy |
|---|---|---|---|---|
| Gene | | Public | Score | Score |
| Symbol | Assay ID | RefSeq | (TLDA) | (Arrays) |
| Abat | Mm00556951_m1 | NM_172961 | 0.73 | 0.9 |
| Abca1 | Mm00442646_m1 | NM_013454 | 0.75 | 0.59 |
| Ank | Mm00445047_m1 | NM_020332 | 0.57 | 0.62 |
| Ankrd1 | Mm00496512_m1 | NM_013468 | 0.31 | 0.46 |
| Arhgap24 | Mm00525303_m1 | NM_146161 | 0.30 | 0.29 |
| Atp8a1 | Mm00437712_m1 | NM_009727 | 0.91 | 0.9 |
| Bex1 | Mm00784371_s1 | NM_009052 | 0.44 | 0.38 |
| Ccl9 | Mm00441260_m1 | NM_011338 | 0.58 | 0.82 |
| Chst1 | Mm00517855_m1 | NM_023850 | 0.47 | 0.7 |
| Ckmt1 | Mm00438216_m1 | NM_009897 | 0.71 | 0.89 |
| Col9a3 | Mm00658509_m1 | NM_009936 | 1.00 | 0.39 |
| Cpz | Mm00462216_m1 | NM_153107 | 0.72 | 0.76 |
| Cxcl1 | Mm00433859_m1 | NM_008176 | 1.50 | 0.84 |
| Cxcl15 | Mm00441263_m1 | NM_011339 | 0.90 | 0.7 |
| Daf1 | Mm00438377_m1 | NM_010016 | 0.39 | 0.41 |
| Dapk1 | Mm00459400_m1 | NM_029653 | 0.39 | 0.58 |
| Dffb | Mm00432822_m1 | NM_007859 | 0.96 | 0.86 |
| Dgka | Mm00444048_m1 | NM_016811 | 0.79 | 0.79 |
| Eno3 | Mm00468264_g1 | NM_007933 | 0.56 | 0.75 |
| Eva1 | Mm00468397_m1 | NM_007962 | 1.34 | 0.86 |
| Fas | Mm00433237_m1 | NM_007987 | 0.84 | 0.83 |
| Fgf18 | Mm00433286_m1 | NM_008005 | 1.00 | 0.89 |
| Fgf7 | Mm00433291_m1 | NM_008008 | 0.66 | 0.85 |
| Fhod3 | Mm00614166_m1 | NM_175276 | 0.84 | 0.61 |
| Garnl3 | Mm00724806_m1 | NM_178888 | 0.72 | 0.88 |
| Gca | Mm00521120_m1 | NM_145523 | 1.03 | 0.85 |
| Gpr149 | Mm00805216_m1 | NM_177346 | 0.39 | 0.53 |
| Hbegf | Mm00439307_m1 | NM_010415 | 0.90 | 0.9 |
| Hey2 | Mm00469280_m1 | NM_013904 | 0.63 | 0.73 |
| Hmga1 | Mm00516662_m1 | NM_016660 | 0.67 | 0.82 |
| Hmga2 | Mm00780304_sH | X58380 | 0.90 | 0.87 |
| Hoxc13 | Mm00802798_m1 | NM_010464 | 0.96 | 0.83 |
| Idb2 | Mm00711781_m1 | NM_010496 | 0.58 | 0.61 |
| Idb4 | Mm00499701_m1 | NM_031166 | 0.23 | 0.39 |
| Igfbp2 | Mm00492632_m1 | NM_008342 | 0.66 | 0.37 |
| Igsf4a | Mm00457551_m1 | NM_018770 | 0.51 | 0.7 |
| Jag2 | Mm00439935_m1 | NM_010588 | 0.69 | 0.86 |
| Kctd15 | Mm00525397_m1 | NM_146188 | 0.64 | 0.7 |
| Lass4 | Mm00482658_m1 | NM_026058 | 0.87 | 0.69 |
| Ldh2 | Mm00493146_m1 | NM_008492 | 0.80 | 0.56 |
| Man2b1 | Mm00487585_m1 | NM_010764 | 0.95 | 0.83 |
| Mcam | Mm00522397_m1 | NM_023061 | 0.57 | 0.63 |
| Mmp15 | Mm00485062_m1 | NM_008609 | 0.60 | 0.83 |
| Mrpl15 | Mm00804108_m1 | NM_025300 | 1.81 | 0.88 |
| Ms4a10 | Mm00452322_m1 | NM_023529 | 0.37 | 0.73 |
| Mtus1 | Mm00628662_m1 | NM_001005864 | 1.08 | 0.85 |
| Notch3 | Mm00435270_m1 | NM_008716 | 0.63 | 0.62 |
| Noxa | Mm00451763_m1 | NM_021451 | 0.36 | 0.26 |
| Pard6g | Mm00474139_m1 | NM_053117 | 0.84 | 0.79 |
| Perp | Mm00480750_m1 | NM_022032 | 1.19 | 0.7 |
| Pla2g7 | Mm00479105_m1 | NM_013737 | 0.39 | 0.5 |
| Plac8 | Mm00507371_m1 | NM_139198 | 0.84 | 0.88 |
| Pltp | Mm00448202_m1 | NM_011125 | 1.03 | 0.88 |
| Plxdc2 | Mm00470649_m1 | NM_026162 | 0.82 | 0.36 |
| Prkcm | Mm00435790_m1 | NM_008858 | 1.38 | 0.9 |
| Prkg1 | Mm00440954_m1 | NM_001013833 | 0.76 | 0.86 |
| Rab40b | Mm00454800_m1 | NM_139147 | 1.04 | 0.85 |
| Rb1 | Mm00485586_m1 | NM_009029 | 0.83 | 0.74 |
| Rgs2 | Mm00501385_m1 | NM_009061 | 0.79 | 0.62 |
| Rprm | Mm00469773_s1 | NM_023396 | 0.77 | 0.69 |
| Sbk1 | Mm00455133_m1 | NM_145587 | 0.87 | 0.81 |
| Scn3b | Mm00463369_m1 | NM_153522 | 0.67 | 0.57 |
| Sema3d | Mm00712652_m1 | NM_028882 | 0.99 | 0.72 |
| Sema7a | Mm00441361_m1 | NM_011352 | 0.40 | 0.61 |
| Serpinb2 | Mm00440905_m1 | NM_011111 | 0.87 | 0.9 |
| Sfrp2 | Mm00485986_m1 | NM_009144 | 0.38 | 0.27 |
| Slc14a1 | Mm00472198_m1 | NM_028122 | 0.17 | 0.39 |
| Sms | Mm00786246_s1 | NM_009214 | 1.22 | 0.89 |
| Sod3 | Mm00448831_s1 | NM_011435 | 0.99 | 0.9 |
| Stmn4 | Mm00490524_m1 | NM_019675 | 0.33 | 0.73 |
| Tex15 | Mm00473190_m1 | NM_031374 | 0.33 | 0.59 |
| Tnfrsf18 | Mm00437136_m1 | NM_021985 | 0.61 | 0.56 |
| Tnnt2 | Mm00441922_m1 | NM_011619 | 0.76 | 0.8 |
| Unc45b | Mm00618472_m1 | NM_178680 | 0.32 | 0.82 |
| Wnt9a | Mm00460518_m1 | NM_139298 | 0.90 | 0.89 |
| Zfp385 | Mm00600201_m1 | NM_013866 | 1.15 | 0.85 |

The indicated assays were performed using TaqMan Low Density Arrays. Shown are 76 CRGs according to TLDA probe set availability. Synergy scores were calculated as described in Methods.

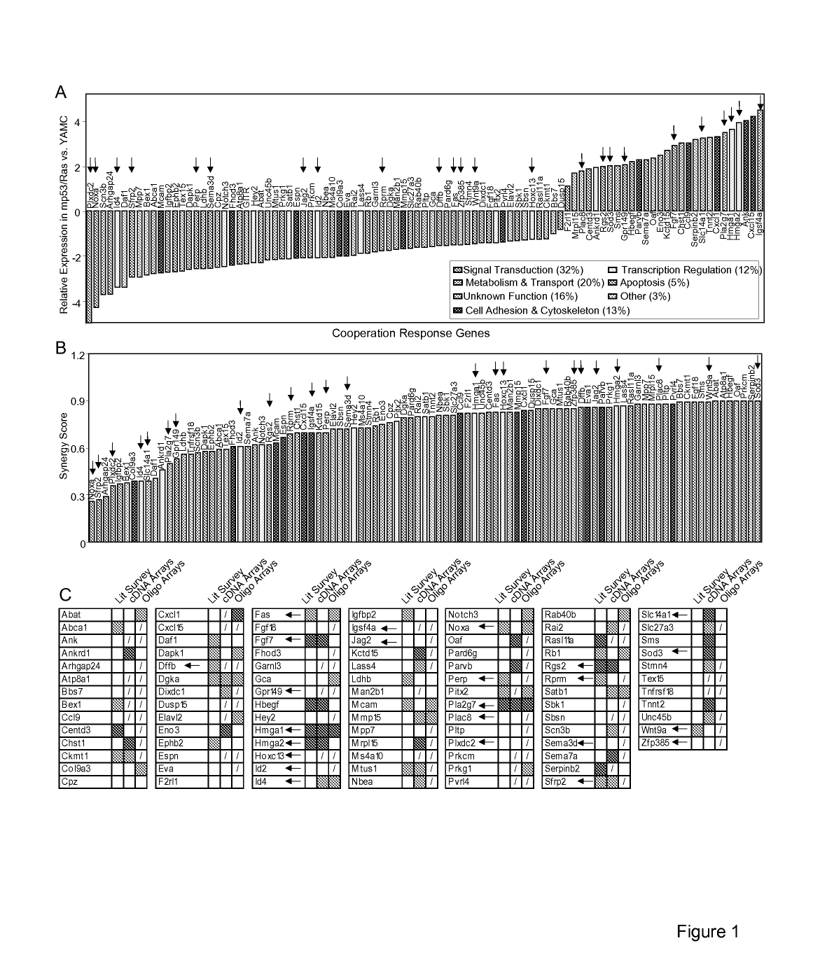

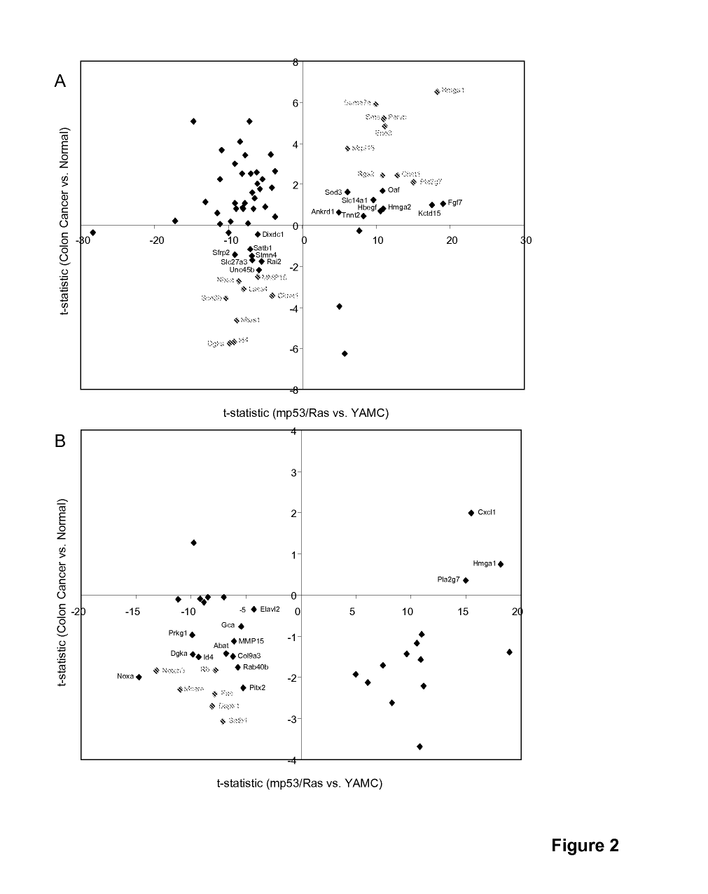

CRGs encode proteins involved in the regulation of cell signaling, transcription, apoptosis, metabolism, transport or adhesion (FIG. 1A, 1B, Table 1), and in large proportion appear misexpressed in human cancer. For 47 out of the 75 CRGs tested co-regulation was found in primary human colon cancer and our murine colon cancer cell model (FIG. 1C, FIG. 2). Moreover three of theses genes (EphB2, HB-EGF and Rb) also have been shown to play a causative role in tumor formation. In addition, altered expression of 29 CRGs has been found in a variety of human cancers (Table 1).

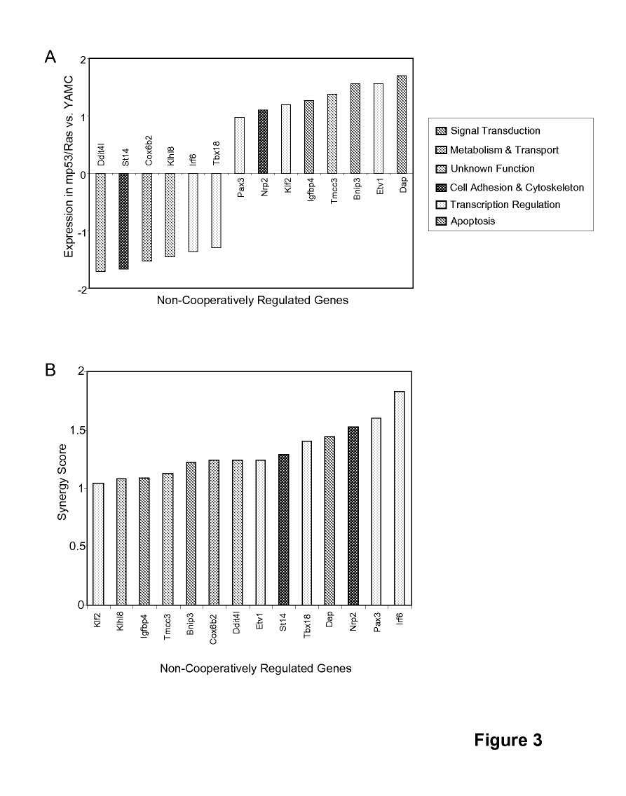

The relevance of differentially expressed genes for malignant cell transformation was assessed by genetic perturbation of a series of 24 CRGs (excluding those with an established role in tumor formation, EphB2, HB-EGF and Rb) and 14 genes responding to p53175H and/or activated H-Ras12V in a non-cooperative manner (non-CRGs). Perturbed genes were chosen across a broad range of biological functions, levels of differential expression and synergy scores (FIG. 1 and FIG. 3). These perturbations were carried out in mp53/Ras cells with the goal to reestablish expression of the manipulated genes at levels relatively close to those found in YAMC control cells, and to monitor subsequent tumor formation following sub-cutaneous injection of these cells into immuno-compromised mice. Of the perturbed genes 18 were up- and 20 down-regulated in mp53/Ras cells, relative to YAMC (Tables 3 and 4).

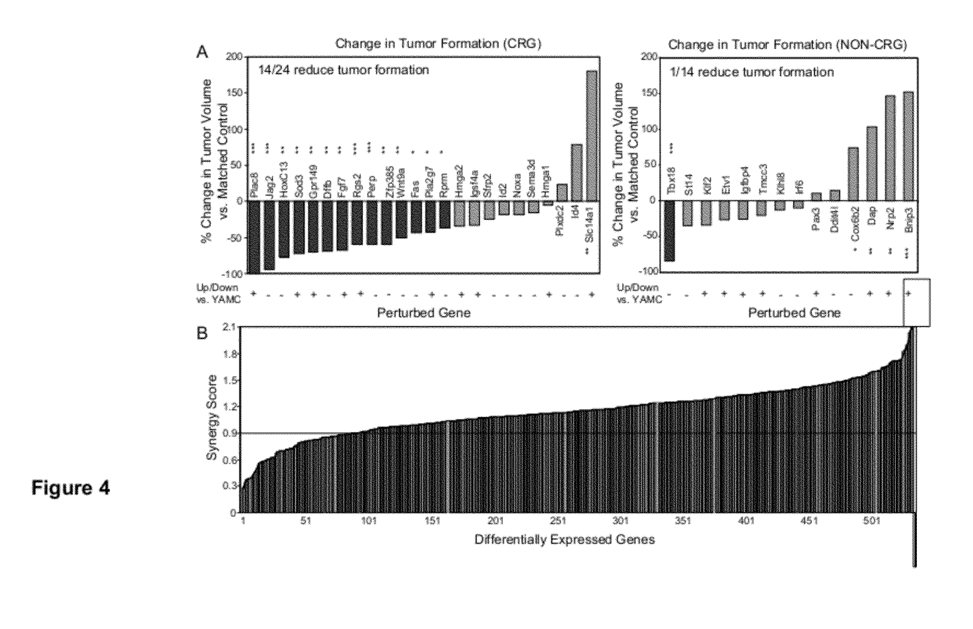

Tumor volume was measured weekly for 4 weeks following injection into nude mice of murine and human cancer cells. Reversal of the changes in CRG expression significantly reduced tumor formation by mp53/Ras cells in 14 out of 24 cases (Table 3, FIG. 4A), indicating a critical role in malignant transformation for a surprisingly large fraction of these genes. Perturbation of Plac8, Jag2 and HoxC13 gene expression had the strongest effects. In addition, perturbation of two CRGs, Fas and Rprm, that alone produced significant yet milder changes in tumor formation were combined. This yielded significantly increased efficacy in tumor inhibition as compared with the respective single perturbations (Wilcoxn test, Table 4). Thus, even genetic perturbations of CRGs that seem to have relatively smaller effects when examined on their own show evidence of being essential when analyzed in combination.

TABLE 3
Tumor formation by mp53/Ras cells following perturbation of individual
cooperation response genes (CRGs)

| | | | | | % Change in | | |
|---|---|---|---|---|---|---|---|
| | | | Expression | | Tumor Volume | | |
| Gene | Gene | Synergy | mp53/Ras vs. | Number of | (Perturbed vs. | p Value | p Value |
| Name | Function | Score | YAMC (fold) | Injections (n) | Control) | (Wilcoxn) | (t-test) |
| | | | | | Smaller | | |
| Plac8 | Unknown | 0.88 | 3.21 | 9 | −100 | 0.0006 | 0.0001 |
| Jag2 | Signaling | 0.86 | 0.24 | 8 | −94 | 0.0003 | 0.0007 |
| HoxC13 | Transcription | 0.83 | 0.42 | 8 | −76 | 0.005 | 0.002 |
| Sod3 | Metabolism | 0.90** | 4.03 | 16 | −72 | 0.004 | 0.001 |
| Gpr149 | Signaling | 0.53 | 3.87 | 12 | −70 | 0.006 | 0.05 |
| Dffb | Apoptosis | 0.86 | 0.35 | 8 | −69 | 0.005 | 0.01 |
| Fgf7 | Signaling | 0.85 | 7.08 | 6 | −68 | 0.004 | 0.01 |
| Rgs2 | Signaling | 0.62 | 3.70 | 18 | −60 | 0.0002 | 0.006 |
| Perp | Apoptosis | 0.70 | 0.17 | 16 | −59 | 0.0008 | 0.002 |
| Zfp385 | Unknown | 0.85 | 0.36 | 8 | −59 | 0.007 | 0.005 |
| Wnt9a | Signaling | 0.89 | 0.37 | 8 | −50 | 0.002 | 0.002 |
| Fas | Apoptosis | 0.83 | 0.35 | 10 | −43 | 0.02 | 0.02 |
| Pla2g7 | Metabolism | 0.50 | 10.67 | 14 | −42 | 0.02 | 0.04 |
| Rprm | Signaling | 0.69 | 0.29 | 12 | −36 | 0.01 | 0.04 |
| | | | | | No Significant | | |
| | | | | | Change | | |
| Hmga2 | Transcription | 0.87 | 14.88 | 10 | −34 | 0.96 | 0.43 |
| Igsf4a | Migration | 0.70 | 16.89 | 10 | −33 | 0.37 | 0.31 |
| Sfrp2 | Signaling | 0.27 | 0.13 | 10 | −25 | 0.23 | 0.24 |
| Id2 | Transcription | 0.61 | 0.24 | 6 | −18 | 0.70 | 0.41 |
| Noxa | Apoptosis | 0.26 | 0.05 | 8 | −18 | 0.30 | 0.33 |
| Sema3d | Signaling | 0.72* | 0.17 | 6 | −16 | 0.67 | 0.40 |
| Hmga1 | Transcription | 0.82 | 11.38 | 14 | −5 | 0.48 | 0.91 |
| Plxdc2 | Signaling | 0.36 | 0.03 | 6 | 24 | 0.13 | 0.08 |
| Id4 | Transcription | 0.39 | 0.10 | 6 | 79 | 0.20 | 0.14 |
| | | | | | Larger | | |
| Slc14a1 | Metabolism | 0.39 | 9.20 | 6 | 180 | 0.008 | 0.002 |

For each gene perturbation, tumor volumes were compared to matched vector controls in the same experiment. Corresponding to the number of injections performed with perturbed cells, matched vector tumors numbered between 6 and 18, with perturbation experiments performed for small groups of genes and matched vector control. A synergy score smaller than 1 indicates a synergistic or non-additive change in gene expression in response to multiple as compared to single oncogenic mutations. The lower synergy score derived from either raw or normalized microarray expression values are indicated. The p-values estimate the level of confidence that the synergy score is less than one. Synergy scores and associated p-values were calculated as described in Methods. For all synergy scores, p-values are p < 0.01, except as indicated (**p < 0.05; *p < 0.1).

TABLE 4
Tumor formation of mp53/Ras cells following dual CRG perturbations

| | | % Change in | | | | |
|---|---|---|---|---|---|---|
| | | Tumor Volume | p Value vs. Fas | p Value vs. | p Value vs. | p Value vs. |
| Gene | Number of | (Perturbed vs. | alone | Rprm alone | Fas alone | Rprm alone |
| Name | Injections (n) | Control) | (Wilcoxn) | (Wilcoxn) | (t-test) | (t-test) |
| Fas | 10 | −43 | | | | |
| Rprm | 12 | −36 | | | | |
| Fas + Rprm | 8 | −81 | 0.04 | 0.04 | 0.04 | 0.02 |

For each gene perturbation, tumor volumes were compared to matched vector controls in the same experiment. Corresponding to the number of injections performed with perturbed cells, matched vector tumors numbered between 6 and 18, with perturbation experiments performed for small groups of genes and matched vector control.

Given the increased efficacy of the Fas+Rprm combination in tumor inhibition as compared with their respective single perturbations, additional combinations of cooperation response genes were analyzed (Table 5). As noted below several combinations, such as, Dffb-Sfrp, Dapk-Perp, Dapk-Noxa, Noxa-Rprm, Rprm-Sfrp, Noxa-Sfrp, and Dapk-Sfrp resulted in significantly smaller tumor volume relative to the single perturbations. It is also important to note that not all combinations had this synergistic effect (e.g., Dffb-Rprm).

TABLE 5
Tumor formation of mp53/Ras cells following dual perturbation of
cooperation response genes

| | | | | | P Value |
|---|---|---|---|---|---|
| Gene | Number of | % | P Value | P Value | (vs. |
| Name | Injections (n) | Change | (vs. Vect) | (vs. Pert 1) | Pert 2) |
| Vector | 24 | | | | |
| Dffb | 8 | −67.84 | 0.000 | | |
| Perp | 16 | −55.87 | 0.000 | | |
| Rprm | 16 | −52.73 | 0.01 | | |
| Noxa | 12 | −43.19 | 0.088 | | |
| Fas | 10 | −32.93 | 0.012 | | |
| Dapk | 12 | −16.67 | 0.470 | | |
| Sfrp2 | 8 | −16.56 | 0.59 | | |

Tumor volume significantly smaller in dual than in single perturbations

| | | | | | |
|---|---|---|---|---|---|
| Dffb-Sfrp2 | 8 | −92.70 | 0.00 | 0.02 | 0.00 |
| Dapk-Perp | 8 | −84.46 | 0.00 | 0.00 | 0.00 |
| Dapk-Noxa | 8 | −83.64 | 0.00 | 0.00 | 0.00 |
| Noxa-Rprm | 8 | −71.73 | 0.00 | 0.00 | 0.03 |
| Fas-Rprm | 8 | −71.65 | 0.00 | 0.04 | 0.02 |
| Rprm-Sfrp2 | 7 | −70.66 | 0.00 | 0.01 | 0.01 |
| Noxa-Sfrp2 | 8 | −58.22 | 0.00 | 0.01 | 0.03 |
| Dapk-Sfrp2 | 8 | −48.91 | 0.00 | 0.05 | 0.04 |

Tumor volume not significantly smaller in dual than in single perturbations

| | | | | | |
|---|---|---|---|---|---|
| Dffb-Rprm | 8 | −74.22 | 0.00 | 0.15 | 0.00 |
| Dffb-Perp | 8 | −65.70 | 0.00 | 0.53 | 0.09 |
| Dapk-Fas | 8 | −64.49 | 0.00 | 0.02 | 0.10 |
| Fas-Perp | 8 | −62.64 | 0.00 | 0.16 | 0.15 |
| Fas-Sfrp2 | 8 | −59.97 | 0.00 | 0.20 | 0.03 |
| Dffb-Fas | 8 | −58.24 | 0.00 | 0.91 | 0.18 |
| Perp-Rprm | 8 | −57.50 | 0.00 | 0.96 | 0.50 |
| Perp-Sfrp2 | 8 | −51.53 | 0.00 | 0.80 | 0.06 |
| Noxa-Perp | 8 | −49.51 | 0.00 | 0.09 | 0.83 |
| Fas-Noxa | 8 | −43.13 | 0.00 | 0.85 | 0.12 |
| Dffb-Noxa | 8 | −33.16 | 0.01 | 0.27 | 0.18 |
| Dapk-Rprm | 8 | −16.80 | 0.01 | 0.31 | 0.84 |
| Dapk-Dffb | 8 | −13.80 | 0.01 | 0.03 | 0.41 |

For each gene perturbation, tumor volumes were compared to matched vector controls in the same experiment for calculation of change in tumor volume and statistical testing (T test, unequal variance).
For statistical tests on combined perturbation vs. single perturbation, each combo was tested against the first perturbation listed (Pert 1), and against the second perturbation listed (Pert 2).

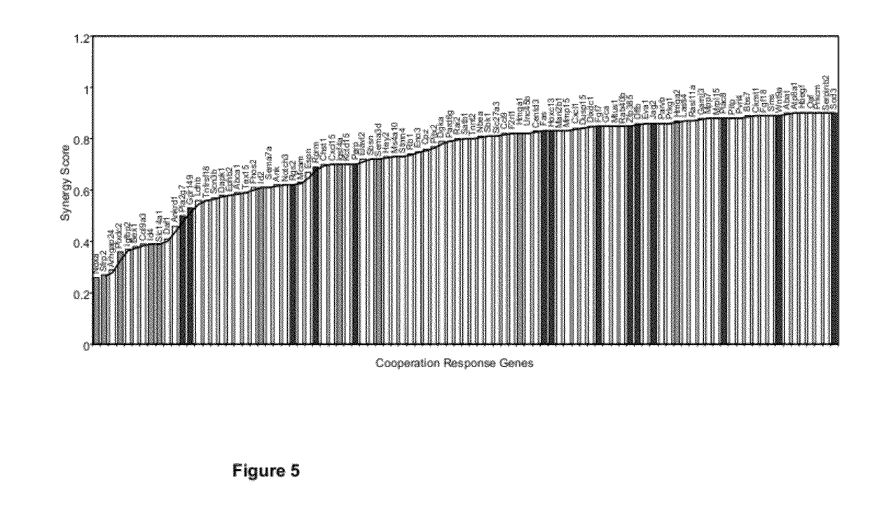

In contrast to the multitude of CRG-related effects on tumor inhibition, out of 14 perturbations of the non-cooperatively regulated genes, only one showed a significant reduction in tumor formation of mp53/Ras cells (FIG. 2A, right panel and Table 6). Taken together, the data indicate that among the genes differentially expressed in cancer cells, malignant transformation strongly relies on the class of genes synergistically regulated by cooperating oncogenic mutations (FIG. 2B and FIG. 5).

TABLE 6
Tumor formation by mp53/Ras cells following perturbation of non-cooperatively regulated
genes (non-CRGs)

| | | | | | | % Change in | | |
|---|---|---|---|---|---|---|---|---|
| | | | | | | Tumor | | |
| | | | Expression | Ras and/or | Number of | Volume | | p |
| Gene | Gene | Synergy | mp53/Ras vs. | mp53 | Injections | (Perturbed | p Value | Value |
| Name | Function | Scores | YAMC (fold) | Response | (n) | vs. Control) | (Wilcoxn) | (t-test) |
| | | | | | | Smaller | | |
| Tbx18 | Transcription | 1.40 | 0.41 | Ras | 8 | −84 | 0.0009 | 0.002 |
| | | | | | | No | | |
| | | | | | | Significant | | |
| | | | | | | Change | | |
| St14 | Migration | 1.29 | 0.32 | Ras & | 12 | −35 | 0.27 | 0.18 |
| | | | | mp53 | | | | |
| Klf2 | Transcription | 1.04 | 2.29 | Ras | 10 | −34 | 0.21 | 0.52 |
| Etv1 | Transcription | 1.24 | 2.94 | Ras | 13 | −27 | 1 | 0.54 |
| Igfbp4 | Signaling | 1.12 | 2.40 | Ras & | 6 | −26 | 0.48 | 0.24 |
| | | | | mp53 | | | | |
| Tmcc3 | Unknown | 1.13 | 2.59 | Ras | 8 | −20 | 0.62 | 0.44 |
| Klhl8 | Unknown | 1.08 | 0.37 | mp53 | 10 | −13 | 0.67 | 0.69 |
| Irf6 | Transcription | 1.83 | 0.39 | Ras & | 12 | −10 | 0.69 | 0.74 |
| | | | | mp53 | | | | |
| Pax3 | Transcription | 1.60 | 1.96 | Ras | 18 | 10 | 0.98 | 0.68 |
| Ddit41 | Unknown | 1.24 | 0.31 | mp53 | 11 | 15 | 0.55 | 0.56 |
| | | | | | | Larger | | |
| Cox6b2 | Metabolism | 1.24 | 0.35 | Ras & | 11 | 74 | 0.05 | 0.03 |
| | | | | mp53 | | | | |
| Dap | Apoptosis | 1.44 | 3.24 | Ras & | 14 | 104 | 0.004 | 0.001 |
| | | | | mp53 | | | | |
| Nrp2 | Migration | 1.53 | 2.15 | Ras | 6 | 147 | 0.003 | 0.02 |
| Bnip3 | Apoptosis | 1.22 | 2.94 | Ras | 14 | 153 | 0.0009 | 0.002 |

For each gene perturbation, tumor volumes were compared to matched vector controls in the same experiment. Corresponding to the number of injections performed with perturbed cells, matched vector tumors numbered between 6 and 18, with perturbation experiments performed for small groups of genes and matched vector control. A synergy score ≧1 indicates a non-synergistic change in gene expression in response to multiple as compared to single oncogenic mutations. The lower synergy score derived from either raw or normalized microarray expression values are indicated. Synergy scores were calculated as described in Methods.

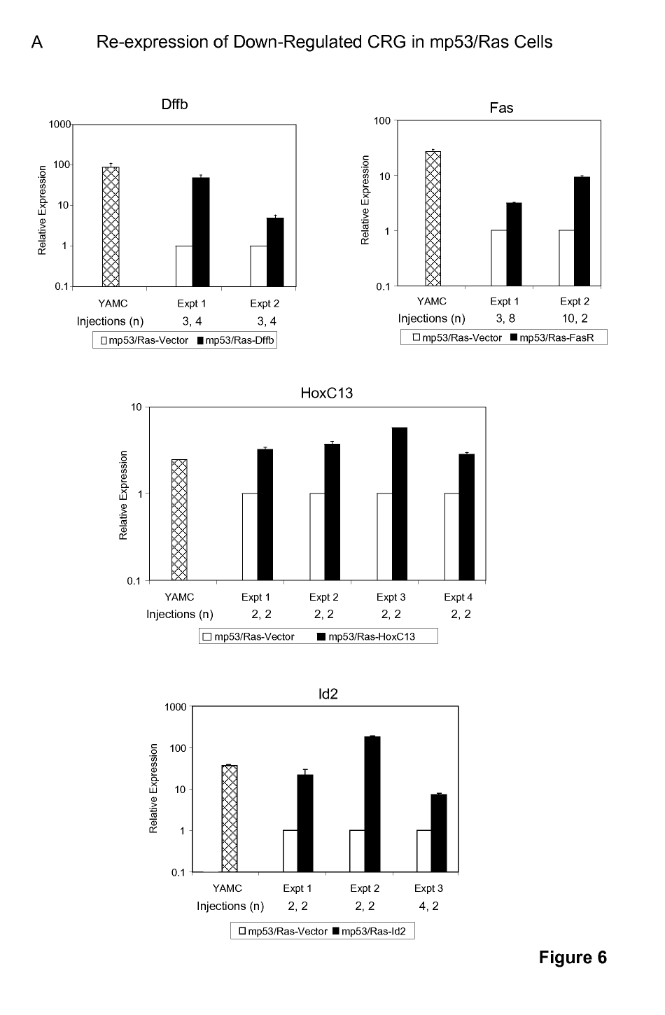

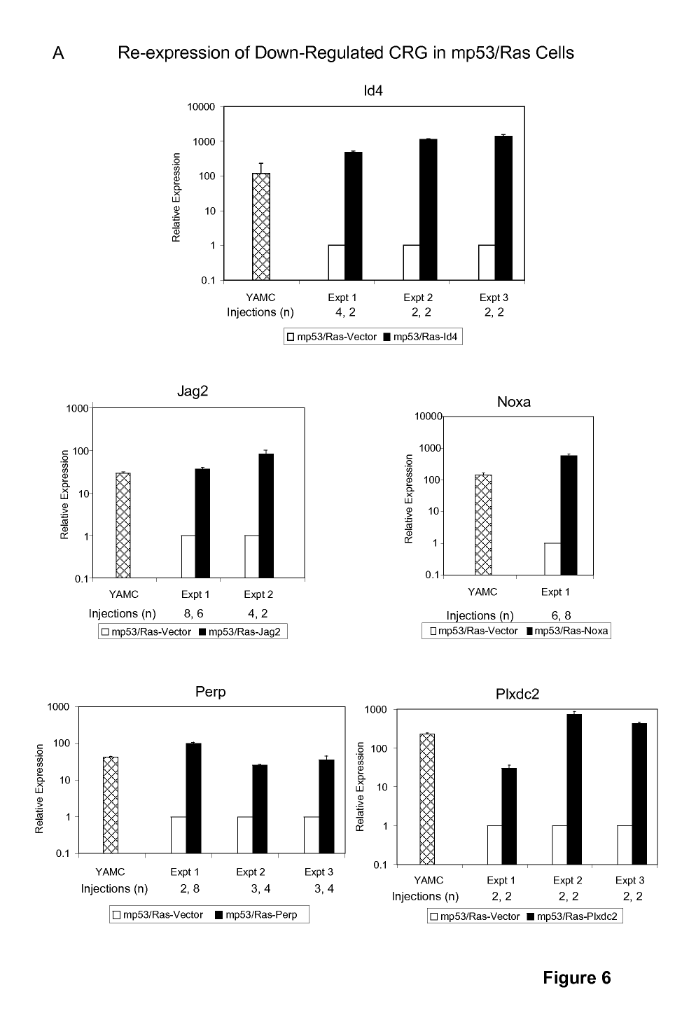

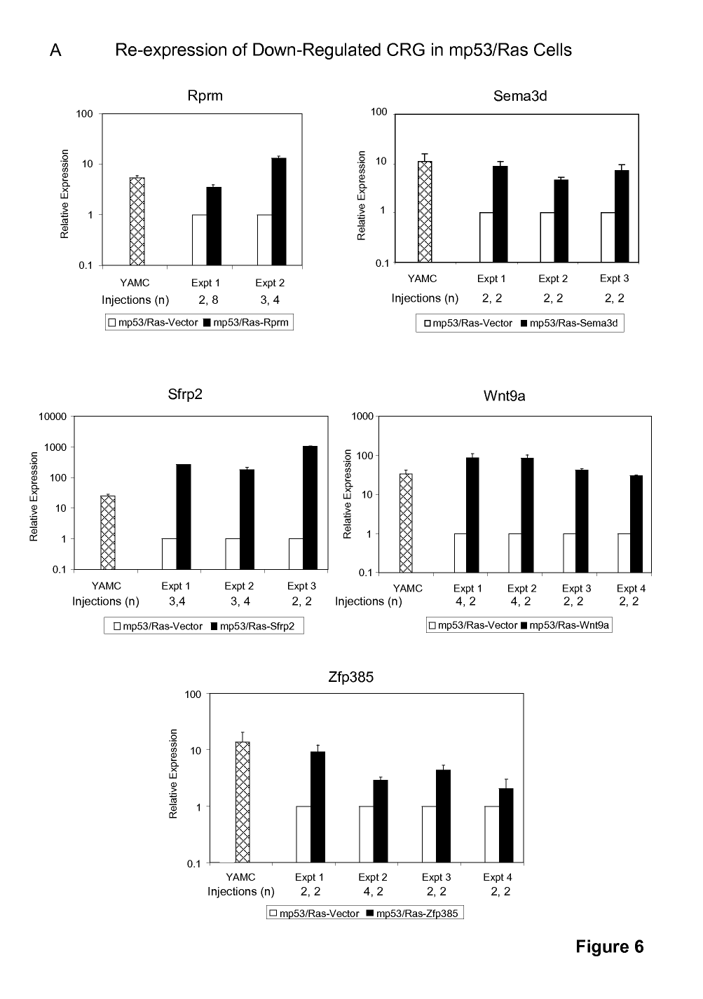

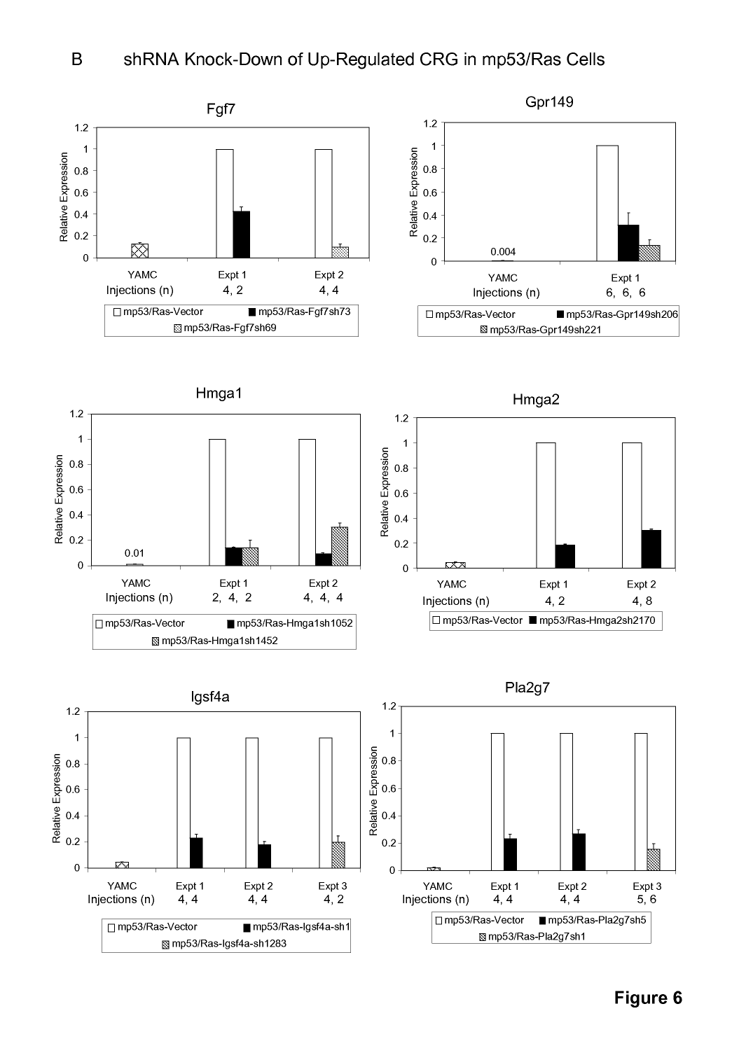

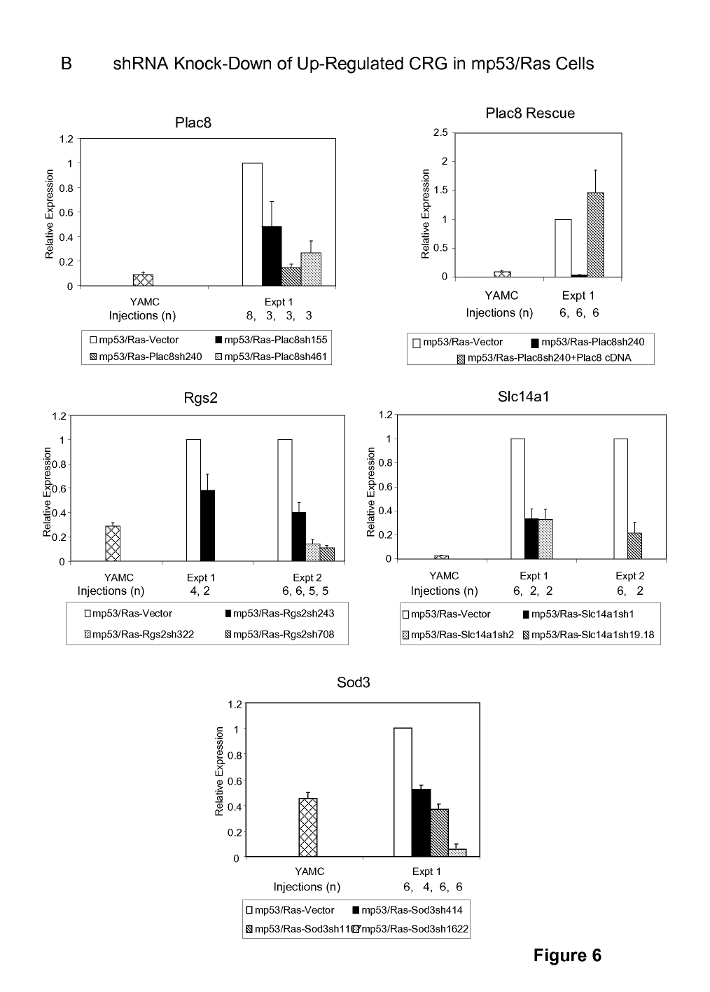

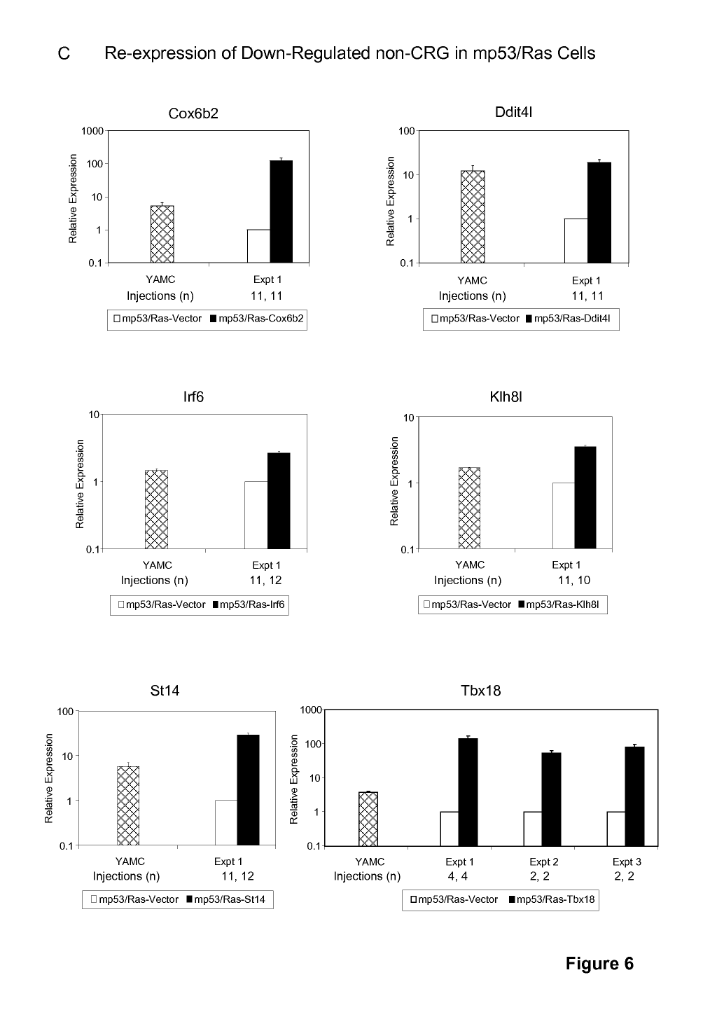

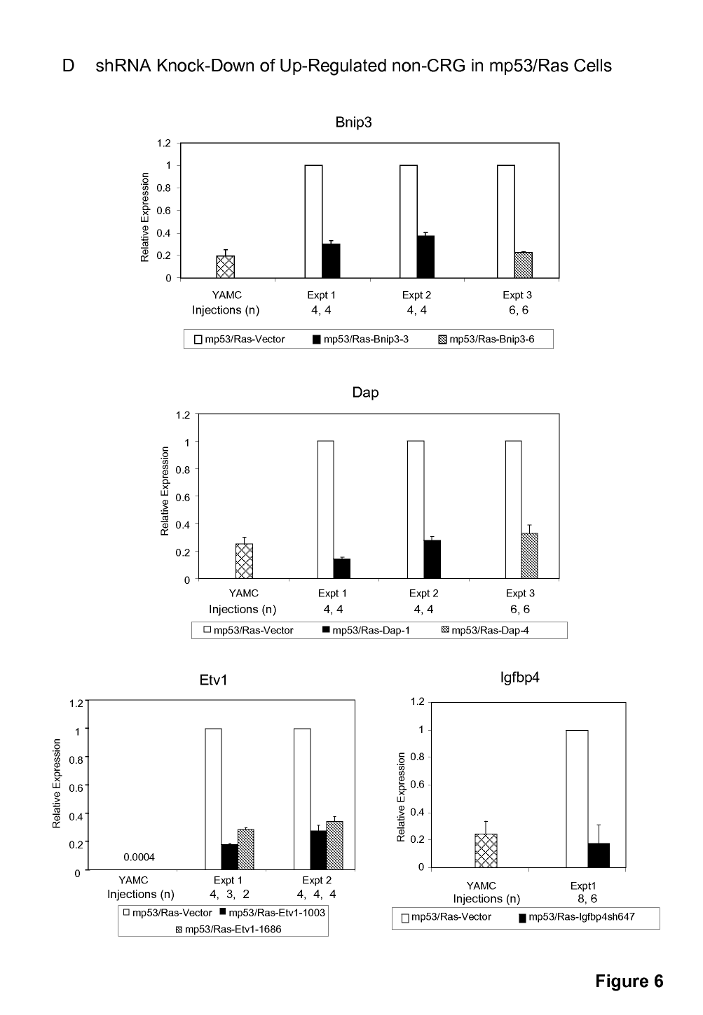

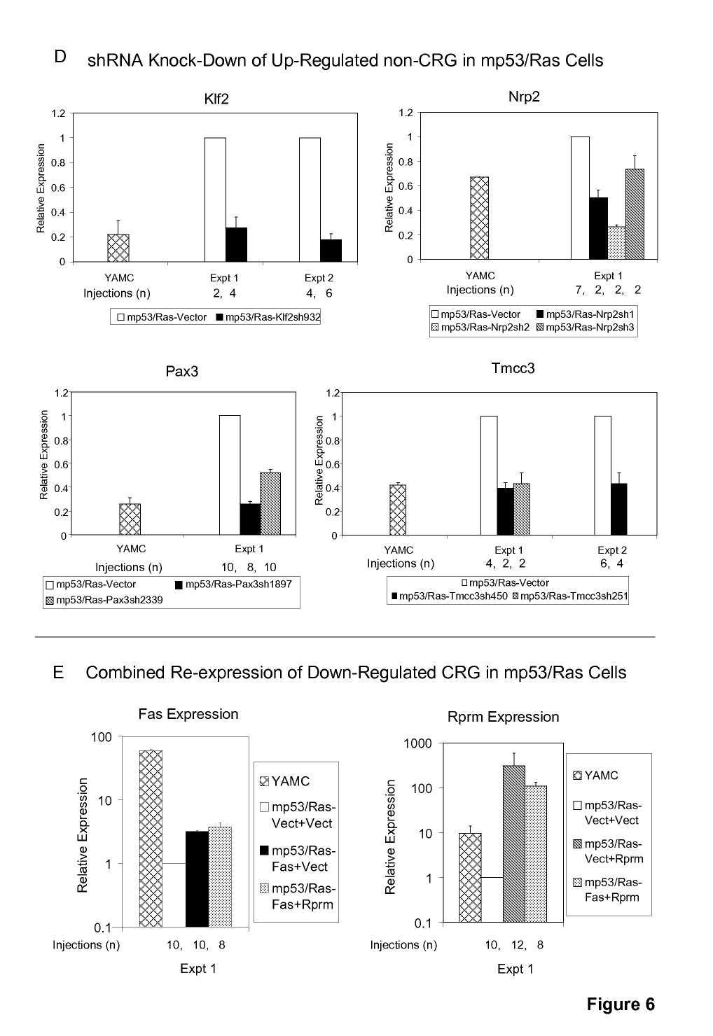

Genetic perturbation experiments were carried out utilizing retrovirus-mediated re-expression of corresponding cDNAs for down-regulated genes (Table 7) and shRNA-dependent stable knock-down using multiple independent targets for over-expressed genes (Table 8). In addition, Plac8 knock down was functionally rescued by expression of shRNA-resistant Plac8, confirming specificity of the Plac8 loss-of-function experiments. The extent of all gene perturbations was assessed by quantitative PCR (FIG. 6). As expected, the genetic perturbations disrupt tumor formation downstream of the initiating oncogenic mutations. Expression of both mutant p53 and activated Ras proteins was measured by Western blots for H-Ras, p53 and β-tubulin expression in matched vector and mp53/Ras cells and remained unaffected by all genetic manipulations that inhibit the formation of tumors. Moreover, gene perturbations distinguished tumor growth from in vitro cell proliferation, as they generally did not perceivably affect cell accumulation in tissue culture. Re-expression of the CRG Notch3, however, registered as a notable exception, resulting in cell growth inhibition in tissue culture, thus preventing tests of tumor formation in vivo in this case.

TABLE 7
cDNA clones used for gene re-expression perturbations

| | | IMAGE | | |
|---|---|---|---|---|
| | Gene Name | Clone ID | GenBank ID | Species |
| CRG | Jag2 | Gift of Dr. | NM_010588 | Mouse |
| | | L. Milner | | |
| (Critical) | HoxC13 | 6171228 | BC090850 | Human |
| | Dffb | 6403143 | BC053052 | Mouse |
| | Perp | 3985702 | BC021772 | Mouse |
| | Zfp385 | 4504518 | BC017644 | Mouse |
| | Wnt9a | 30435371 | BC066165 | Mouse |
| | Fas | 30302649 | BC061160 | Mouse |
| | Rprm | 1434823 | BC030065 | Mouse |
| CRG | Sfrp2 | 4487469 | BC014722 | Mouse |
| (Non-Critical) | Id2 | 2655173 | BC006921 | Mouse |
| | Noxa | 6517820 | BC050821 | Mouse |
| | Sema3d | 5272175 | BC029590 | Human |
| | Plxdc2 | 5349869 | BC057881 | Mouse |
| | Id4 | 4552357 | BC014941 | Human |
| Non-CRG (Critical) | Tbx18 | PCR cloned | NM_023814 | Mouse |
| Non-CRG | St14 | 3488059 | BC005496 | Mouse |
| (Non-Critical) | Klh18 | 30612176 | BC086802 | Mouse |
| | Irf6 | 3592582 | BC008515 | Mouse |
| | Ddit41 | 5254530 | BC038131 | Mouse |
| | Cox6b2 | 6773974 | BC048670 | Mouse |

TABLE 8
Gene knock-down perturbations

| | | | | Knock- | |
|---|---|---|---|---|---|
| | | | | Down | |
| | Gene | | Construct | Efficiency | |
| | Name | GenBank ID | Name | (%) | shRNA Target Sequence |
| CRG | Plac8 | NM_139198 | sh155 | 52 | CTGGCAGACCAGCCTGTGTTT (SEQ ID |
| (Critical) | | | | | NO: 1) |
| | | | sh240 | 86 | GTGGCAGCTGACATGAATGTT (SEQ ID |
| | | | | | NO: 2) |
| | | | sh461 | 74 | GCTCAACTCAGCACACACTTT (SEQ ID |
| | | | | | NO: 3) |
| | Sod3 | NM_011435 | sh414 | 50 | GGCGACACGCATGCCAAAG (SEQ ID NO: |
| | | | | | 4) |
| | | | sh1107 | 64 | GGCCTCTAGGCGTCCTAGA (SEQ ID NO: |
| | | | | | 5) |
| | | | sh1622 | 95 | GGCGCTCTGGGACCACTCT (SEQ ID NO: |
| | | | | | 6) |
| | Gpr149 | BC119599 | sh206 | 69 | TCCACGTAGTTTAGTAAGT (SEQ ID NO: |
| | | | | | 7) |
| | | | sh221 | 87 | GTGGTTCTGCTTGTCTTTC (SEQ ID NO: |
| | | | | | 8) |
| | Fgf7 | NM_008008 | sh73 | 60 | TGCCTGTACTGACTAATAT (SEQ ID NO: |
| | | | | | 9) |
| | | | sh69 | 90 | CATGCCTGTACTGACTAAT (SEQ ID NO: |
| | | | | | 10) |
| | Rgs2 | NM_009061 | sh243 | 42, 61 | GCGCAGCTCTGGGCAGAAG (SEQ ID NO: |
| | | | | | 11) |
| | | | sh322 | 86 | GTCCGAGTTCTGTGAAGAA (SEQ ID NO: |
| | | | | | 12) |
| | | | sh708 | 89 | GGCTGTGACCTGCCAGAAA (SEQ ID NO: |
| | | | | | 13) |
| | Pla2g7 | NM_013737 | sh1 | 85 | GGCCGTCAGTAATGTTTCA (SEQ ID NO: |
| | | | | | 14) |
| | | | sh5 | 74, 77 | GTGCGATTCTTGACATTGA (SEQ ID NO: |
| | | | | | 15) |
| CRG | Hmga2 | NM_178057 | sh2170 | 70, 82 | AAGGTTTGTACCTCAAATGAATT (SEQ |
| (Non- | | | | | ID NO: 16) |
| Critical) | Igsf4a | NM_018770 | sh1 | 77, 83 | GGAGAAGTGGCAACCATCATT (SEQ ID |
| | | | | | NO: 17) |
| | | | sh1283 | 80 | GACGCAGACACAGCTATAA (SEQ ID NO: |
| | | | | | 18) |
| | Hmga1 | NM_016660 | sh1052 | 86, 91 | CAAGGCTAACTTCCCATTTAGCC (SEQ |
| | | | | | ID NO: 19) |
| | | | sh1452 | 70, 86 | TACCGCCCATCTCCAGAGTAAGG (SEQ |
| | | | | | ID NO: 20) |
| | Slc14a1 | NM_028122 | sh1 | 66 | TCCTGATTCTGGTGGGACT (SEQ ID NO: |
| | | | | | 21) |
| | | | sh2 | 67 | ACTCTTCACACCTGTCAGC (SEQ ID NO: |
| | | | | | 22) |
| | | | sh19.18 | 79 | ATCCATGACAGTTGCAAAT (SEQ ID NO: |
| | | | | | 23) |
| Non- | Klf2 | NM_008452 | sh932 | 73, 83 | CAGGTGAGAAGCCTTATCATTGC (SEQ |
| CRG | | | | | ID NO: 24) |
| | Etv1 | NM_007960 | sh1003 | 73, 91 | AAGTGCCTAGCTGCCACTCCATT (SEQ |
| | | | | | ID NO: 25) |
| | | | sh1686 | 66, 67 | AAGATGCAGAGAATCACCGAATT (SEQ |
| | | | | | ID NO: 26) |
| | Igfbp4 | NM_010517 | sh647 | 83 | GGTGCCTGCAGAAGCATAT (SEQ ID NO: |
| | | | | | 27) |
| | Tmcc3 | NM_172051 | sh251 | 57 | CCCACTCCAACTTCTAAGT (SEQ ID NO: |
| | | | | | 28) |
| | | | sh450 | 60 | CACGGGAGACAGAGGTTTC (SEQ ID NO: |
| | | | | | 29) |
| | Pax3 | NM_008781 | sh1897 | 65, 74 | AAGCCTTTCATCCCAGTATCATT (SEQ ID |
| | | | | | NO: 30) |
| | | | sh2339 | 54, 50 | AACTGTCCACTTGGAGCCCTGTT (SEQ |
| | | | | | ID NO: 31) |
| | Dap | NM_146057 | sh1 | 72, 86 | GAGAGAGACAAGGATGACCTT (SEQ ID |
| | | | | | NO: 32) |
| | | | sh4 | 67 | TGCGGATTGTGCAGAAACA (SEQ ID NO: |
| | | | | | 33) |
| | Nrp2 | NM_010939 | sh1 | 50 | GACTGTGAAACACAAATTTTT (SEQ ID |
| | | | | | NO: 34) |
| | | | sh2 | 75 | TGGCAAGGACTGGGAATATTT (SEQ ID |
| | | | | | NO: 35) |
| | | | sh3 | 27 | GCTGGAAGTCAGCACAAATTT (SEQ ID |
| | | | | | NO: 36) |
| | Bnip3 | NM_009760 | sh3 | 63, 70 | GGTTACCCACGAACCCCACTT (SEQ ID |
| | | | | | NO: 37) |
| | | | sh6 | 77 | TGCGGTGTTCCTGAATTAG (SEQ ID NO: |
| | | | | | 38) |

Relative levels of gene expression were determined by SYBR Green qPCR. ShRNA knockdown efficiency values for independently derived replicate polyclonal cell populations are indicated, separated by comma. Perturbations with or without effects on tumor size average at 73% or 71.1% knockdown, respectively. In two instances, shRNA constructs producing less than 50% reduction in gene expression induced a decrease (Rgs2, 42% knockdown) or an increase (Nrp2, 27% knockdown) in tumor volume, consistent with results derived from more extensive perturbations by alternate shRNAs for each target.

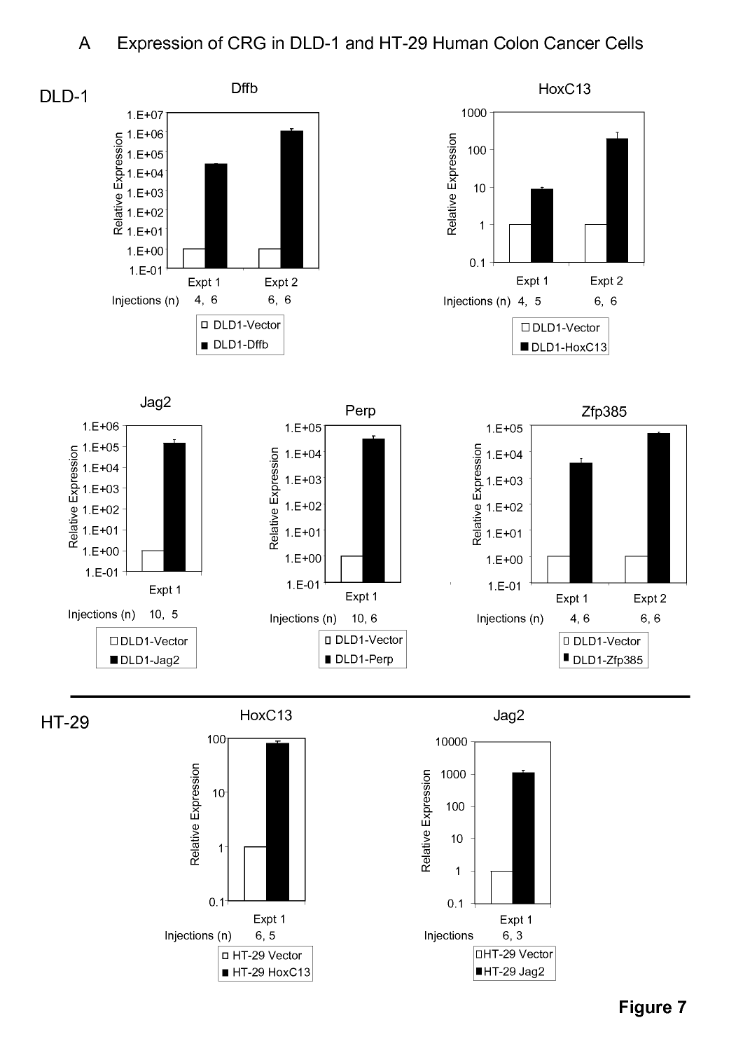

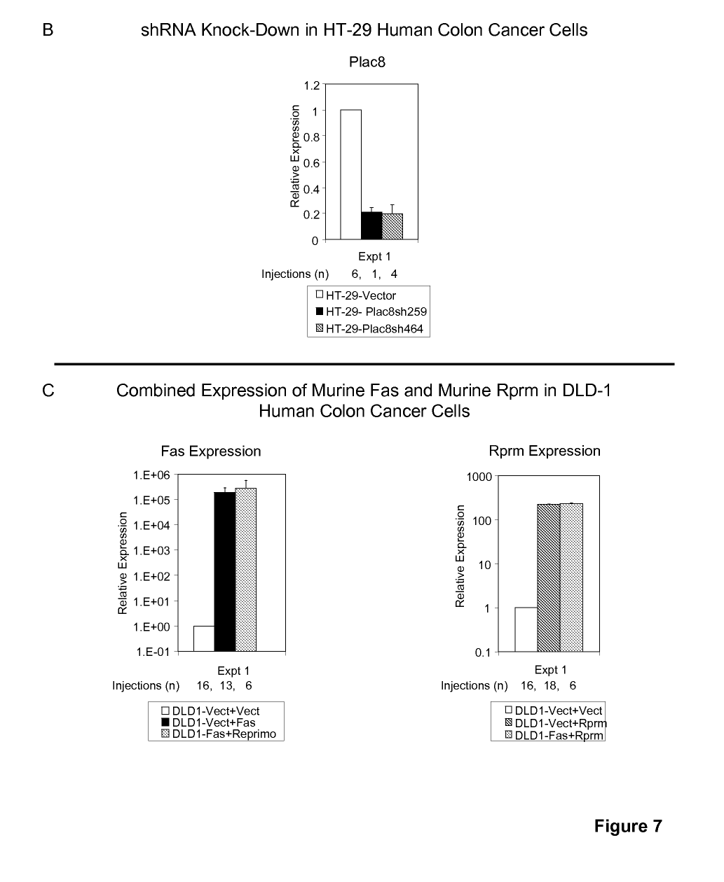

Perturbations of CRGs in human cancer cells (Tables 9 and 10) had similarly strong tumor inhibitory effects to those in the genetically tractable murine mp53/Ras cells, as assessed by xenografts in nude mice. Perturbations of both up- and down-regulated CRGs, i.e. Dffb, Fas, HoxC13, Jag2, Perp, Plac8, Rprm, Zfp385 and Fas+Rprm were performed in human DLD-1 or HT-29 colon cancer cell lines using retroviruses (FIG. 7, Tables 7 and 11) as described above. Similar to mp53/Ras cells, both human cancer cell lines have p53 mutations, whereas with K-Ras (DLD-1) and B-Raf (HT-29) mutations they express activated members of the Ras/Raf signaling pathway distinct from activated H-Ras in mp53/Ras cells. In addition, DLD-1 and HT29 cells carry further oncogenic lesions such as APC and PIK3CA mutations, with HT29 cells also exhibiting a mutation in Smad4. The genetic perturbations had no effect on mutant Ras/Raf or p53 protein expression levels in both DLD-1 and HT-29 cells was measured by Western blot, indicating disruption of the cancer phenotype downstream of oncogenic mutations. Taken together, these experiments indicate the relevance of CRG expression levels to cancer in a variety of backgrounds and genetic contexts.

TABLE 9
Tumor formation of human cancer cells following individual CRG
perturbations

| | | | % Change in | | |
|---|---|---|---|---|---|
| | | | Tumor Volume | | |
| Cell | Gene | Number of | (Perturbed | p Value | p Value |
| Type | Name | Injections (n) | vs. Control) | (Wilcoxn) | (t-Test) |
| DLD-1 | Perp | 6 | −75 | 0.0002 | 0.00001 |
| | Dffb | 12 | −69 | 0.00001 | 2 × 10⁻⁶ |
| | HoxC13 | 11 | −69 | 0.0002 | 2 × 10⁻⁶ |
| | Jag2 | 5 | −62 | 0.006 | 0.0006 |
| | Zfp385 | 12 | −49 | 0.002 | 0.008 |
| | Rprm | 18 | −47 | 0.01 | 0.005 |
| | Fas | 13 | −34 | 0.06 | 0.06 |
| HT-29 | Plac8 | 5 | −100.00 | 0.005 | 0.02 |
| | HoxC13 | 5 | −100.00 | 0.005 | 0.01 |
| | Jag2 | 3 | −81 | 0.09 | 0.03 |

For each gene perturbation, tumor volumes were compared to matched vector controls in the same experiment.
Corresponding to the number of injections performed with perturbed cells, matched vector tumors numbered between 6 and 18.

TABLE 10
Tumor formation of human cancer cells following dual CRG perturbations

| | | | % Change in | | | | |
|---|---|---|---|---|---|---|---|
| | | | Tumor Volume | p Value vs. Fas | p Value vs. | p Value vs. | p Value vs. |
| Cell | Gene | Number of | (Perturbed vs. | alone | Rprm alone | Fas alone (t- | Rprm alone |
| Type | Name | Injections (n) | Control) | (Wilcoxn) | (Wilcoxn) | test) | (t-test) |
| DLD-1 | Fas | 13 | −34 | | | | |
| | Rprm | 18 | −47 | | | | |
| | Fas + | 6 | −79 | 0.008 | 0.07 | 0.005 | 0.02 |
| | Rprm | | | | | | |

For each gene perturbation, tumor volumes were compared to matched vector controls in the same experiment. Corresponding to the number of injections performed with perturbed cells, matched vector tumors numbered between 6 and 18.

TABLE 11
Gene knock-down perturbations in human cells

| | | | Knock- | |
|---|---|---|---|---|
| | | | Down | |
| | | | Efficiency | |
| Gene Name | GenBank ID | Construct Name | (%) | shRNA Target Sequence |
| Plac8 | NM_016619.1 | sh259 | 80% | GTT GCA GCT GAT ATG AAT G (SEQ ID NO: 39) |
| | | sh464 | 85% | GCT CTT ACC GAA GCA ACA A (SEQ ID NO: 40) |

Relative levels of gene expression were determined by SYBR Green qPCR.

The data described here indicate that the cooperative nature of malignant cell transformation, to a considerable degree, depends on synergistic deregulation of downstream effector genes by multiple oncogenic mutations. The cooperation response genes (CRGs) identified here contain a strikingly large fraction of genes (14 out of 24) that are critical to the malignant phenotype, and that their perturbation, singly or in combination, can inhibit formation of tumors containing multiple oncogenic lesions, including p53 deficiency. In contrast, few of the genes differentially expressed in a non-synergistic manner (1 out of 14) significantly reduced tumor growth upon perturbation. Synergistic behavior found in gene expression data thus appears highly informative for identification of genes critically involved in malignant cell transformation (FIG. 2B) and provides a rational path to discovery of both cancer cell-specific vulnerabilities and targets for intervention in cancer cells harboring multiple mutations, including p53 loss-of-function.

CRGs represent a set of 95 annotated cellular genes, many of which have been associated with human cancer by virtue of altered gene expression (FIG. 1C, Table 1). They are involved in the regulation of cell signaling, transcription, apoptosis and metabolism, and based on the data represent key control points in many facets of cancer cell behavior. Thus CRGs are critical nodes in gene networks underlying the malignant phenotype, providing an attractive rationale to explain why several features of cancer cells emerge simultaneously out of the interaction of a few genetic lesions (Xia, M. & Land, H. (2007) Nat Struct Mol Biol 14, 215-23).

Among CRGs and other differentially expressed effector genes examples were also identified that when perturbed produce significantly larger tumors (FIG. 2, Tables 3 and 6). This is consistent with the notion that oncogenic mutations can induce strongly anti-proliferative cellular stress responses (Ridley, A. J., et al. (1998) Embo J 7, 1635-45; Hirakawa, T. & Ruley, H. E. (1988) Proc Natl Acad Sci USA 85, 1519-23; Fanidi, A., et al. (1992) Nature 359, 554-6; Denoyelle, C. et al. (2006) Nat Cell Biol 8, 1053-63). The existence of genes that while responding to oncogenic mutations restrict tumor formation provides direct evidence to support the idea that the state of malignant transformation arises as the result of a finely tuned balance between opposing signals generated by oncogenic mutations (Xia, M. & Land, H. (2007) Nat Struct Mol Biol 14, 215-23; Fanidi, A., et al. (1992) Nature 359, 554-6; Lloyd, A. C. et al. (1997) Genes Dev 11, 663-77; Serrano, M., et al. (1997) Cell 88, 593-602; Sewing, A., et al. (1997) Mol Cell Biol 17, 5588-97; Lowe, S. W., et al. (2004) Nature 432, 307-15). It is thus reasonable to speculate that tumor suppression via perturbation of CRGs, as shown here, disrupts this delicate balance. In fact, such targeted disruption downstream of oncogenic mutations can allow for selective cancer cell deconstruction yielding intervention strategies with high specificity for cancer cells.

For many of the 14 tumor-inhibitory CRGs identified, a clear causal role in tumor formation has been shown here for the first time. Moreover, the data indicate that both gene extinctions (eight genes) and gene inductions (six genes) play important roles in this process. For example, re-expression of the down-regulated CRGs Jag2, a Notch ligand, or of HoxC13, a homeobox transcription factor, as well as shRNA-dependent knock down of Plac8 gene expression are each strongly tumor inhibitory in p53 defective murine and human cancer cells. Both Notch signaling (Houde, C. et al. (2004) Blood 104, 3697-704) and HoxC13 (Panagopoulos, I. et al. (2003) Genes Chromosomes Cancer 36, 107-12) can play oncogenic roles in haematopoietic malignancies, but are involved in promoting differentiation of epithelial cells (Nicolas, M. et al. (2003) Nat Genet. 33, 416-21; Godwin, A. R. & Capecchi, M. R. (1998) Genes Dev 12, 11-20) consistent with the tumor-inhibitory function of Jag2 and HoxC13 in the context of the solid tumor models investigated here. Plac8 is a little investigated gene encoding a cysteine-rich highly conserved peptide expressed in placenta, haematopoietic and epithelial cells that is non-essential for mouse development (Ledford, J. G., et al. (2007) J Immunol 178, 5132-43). When over-expressed, Plac8 can suppress p53 (Rogulski, K. et al. (2005) Oncogene 24, 7524-41). Its essential role for tumor formation of p53-deficient cancer cells, however, is novel and unexpected. Among the eight down-regulated CRGs is Zfp385, another gene of unknown function. Moreover, there is a considerable number of pro-apoptotic/anti-proliferative genes such as Perp, Rprm, Fas, Dffb and Wnt9a, indicating that Ras activation and p53 deficiency cooperate to extinguish the expression of multiple growth inhibitory genes, each of which contributes significantly to restricting tumor growth in the YAMC model when re-expressed. Out of these genes, Perp, Rprm, and Fas previously have been identified as direct p53 targets, indicating that their regulation by p53 is highly conditional on Ras activity (Table 1). Most of the up-regulated CRGs contributing to tumor growth affect signal transduction. This involves Fgf7, Rgs2, Gpr149, an uncharacterized orphan seven-trans-membrane receptor, and Sod3, which acts on signaling via modulation of metabolites (Fattman, C. L., et al. (2003) Free Radic Biol Med 35, 236-56). For all of these genes including Pla2g7 a role in promoting tumor growth is reported here for the first time.

Notably, the efficacy of CRG perturbations performed in human colon cancer cells was comparable to that in the murine colon cell transformation model, indicating dependence of the malignant state on a similar set of genes in both backgrounds. This is remarkable in light of the fact that these human cancer cells carry oncogenic mutations in genes in addition to Ras or Raf and p53 and indicates that CRGs play key roles in the generation and maintenance of the cancer cell phenotype in a variety of contexts. CRGs thus provide a valuable source for identification of much sought ‘Achilles heels’ in human cancer by rational means.

a) Methods

(1) Cells:

Four polyclonal cell populations, control (Bleo/Neo), mp53 (p53175H/Neo), Ras (Bleo/RasV12) and mp53/Ras (p53175H/RasV12) were derived by retroviral infection of low-passage polyclonal young adult mouse colon (YAMC) cells (Xia, M. & Land, H. (2007) Nat Struct Mol Biol 14, 215-23). YAMC cells (a gift from R. Whitehead and A. W. Burgess) derived from the Immorto-mouse (aka H-2 Kb/tsA58 transgenic mouse) expressing temperature-sensitive simian virus 40 large T (tsA58) under the control of an interferon γ-inducible promoter (Whitehead, R. H., et al. (1993) Proc Natl Acad Sci USA 90, 587-91; Jat, P. S. et al. (1991) Proc Natl Acad Sci USA 88, 5096-100) were maintained at the permissive temperature (33° C.) for large T in the presence of interferon γ to support conditional immortalization in vitro. This permits expansion of the cells in tissue culture. In contrast, exposure of YAMC cells to the non-permissive temperature for large T (39° C.) in the absence of interferon γ leads to growth arrest followed by cell death (Whitehead, R. H., et al. (1993) Proc Natl Acad Sci USA 90, 587-91; D'Abaco, G. M., et al. (1996) Mol Cell Biol 16, 884-91), indicating the absence of spontaneous immortalizing mutations in the cell population. The cells were cultured on Collagen IV-coated dishes (1 μg/cm2 for 1.5 hr at room temp; Sigma) in RPMI 1640 medium (Invitrogen) containing 10% (v/v) fetal bovine serum (FBS) (Hyclone), 1×ITS-A (Invitrogen), 2.5 μg/ml gentamycin (Invitrogen), and 5 U/ml interferon γ (R&D Systems). All experiments testing the effects of RasV12 and p53175H were carried out at the non-permissive temperature for large T function (39° C.) and in the absence of interferon γ.

Human colon cancer cells HT-29, which harbor p53, B-Raf, APC, PIK3CA and Smad4 mutations (Ikediobi, 0. N. et al. (2006) Mol Cancer Ther 5, 2606-12), were obtained from the ATCC. DLD-1 cells were provided by Dr. J. Filmus. They carry p53 (Rodrigues, N. R. et al. (1990) Proc Natl Acad Sci USA 87, 7555-9), K-Ras (Shirasawa, S., et al. (1993) Science 260, 85-8), APC (Rubinfeld, B. et al. (1993) Science 262, 1731-4) and PIK3CA (Samuels, Y. et al. (2005) Cancer Cell 7, 561-73) mutations. Both cell lines were maintained at 37° C. in DMEM medium (Invitrogen) containing 10% FBS (Hyclone) and 2.5 μg/ml gentamycin (Invitrogen).

b) Microarray Experiments:

Polysomal RNA was harvested from YAMC, bleo/neo, mp53/neo, bleo/Ras and mp53/Ras cells to obtain gene expression profiles reflective of protein synthesis rates. RNA was harvested from ten replicates for each cell population grown in non-permissive conditions for 48 hr, followed by 24 hr in media with 0% FBS to maximize the contribution of oncogenic signaling to gene expression. RNA was collected while cells were sub-confluent and all cell populations were actively cycling. Cells were lysed in Extraction Buffer (50 mM MOPS, 15 mM MgC1, 150 mM NaCl, 0.5% Triton X-100 with 100 μg/mL cycloheximide, 1 mg/mL heparin, 200 U RNAsin (2 μL/mL of buffer), 2 mM PMSF). Supernatants were applied to 10-50% sucrose gradients, centrifuged at 36,000 rpm for 2 hr at 4° C. and fractions were collected using an ISCO gradient fractionator reading absorbance at 254 nm. Polysome containing fractions were pooled and RNA was purified using the RNeasy Mini Kit (Qiagen) following the standard protocol for animal cells, except that sucrose fractions were mixed with 3.5 volumes Buffer RLT before binding to the RNeasy column. RNA was DNase digested following the on-column digestion as part of the RNeasy RNA extraction protocol.

Five micrograms of RNA was reverse transcribed and labeled using the mAMP kit (Ambion), with the 1× amplification protocol. The cRNA yield was fragmented and hybridization cocktails were prepared using Affymetrix standard protocol for eukaryotic target hybridization. Targets were hybridized to Affymetrix Mouse Genome 430 2.0 Expression Arrays at 45° C. for 16 hours, washed and stained using Affymetrix Fluidics protocol EukGE-WS2v4_{—}450 in the Fluidics Station 450. Arrays were scanned with the Affymetrix GeneChip Scanner 3000.

c) TLDA QPCR:

The TaqMan Low-Density Array (Applied Biosystems) consists of TaqMan qPCR reactions targeting the cooperation response genes available (76 genes, listed in Table 2) and control genes (18S rRNA, GAPDH) in a microfluidic card. TLDA were used to independently test gene expression differences observed by Affymetrix arrays. To generate cDNA for qPCR analysis, quadruplicate samples of polysomal RNA from YAMC, mp53/neo, bleo/Ras and mp53/Ras cells isolated under conditions described above (10 μg/sample) were mixed with 1× SuperScript II reverse transcriptase buffer, 10 mM DTT, 400 μM dNTP mixture, 0.3 ng random hexamer primer, 2 μL RNaseOUT RNase inhibitor and 2 μL of SuperScript II reverse transcriptase in a 100 μL reaction (all components from Invitrogen). RT reactions were carried out by denaturing RNA at 70° C. for 10 minutes, plunging RNA on to ice, adding other components, incubating at 42° C. for 1 hour and heat inactivating the RT enzyme by a final incubation at 70° C. for 10 minutes.

For each sample, 82 μL of cDNA was combined with 328 μl of nuclease free water (Invitrogen) and an equal volume of TaqMan Universal PCR Master Mix No AmpErase UNG (Applied Biosystems). The mixture was loaded into each of 8 ports on the card at 100 μL per port. Each reaction contained forward and reverse primer at a final concentration of 900 nM and a TaqMan MGB probe (6-FAM) at 250 nM final concentration. The cards were sealed with a TaqMan Low-Density Array Sealer (Applied Biosystems) to prevent cross-contamination. The real-time RT-PCR amplifications were run on an ABI Prism 7900HT Sequence Detection System (Applied Biosystems) with a TaqMan Low Density Array Upgrade. Thermal cycling conditions were as follows: 2 min at 50° C., 10 min at 94.5° C., 40 cycles of 97° C. for 30 seconds, and annealing and extension at 59.7° C. for 1 minute. Each individual replicate cDNA sample was processed on a separate card.

Gene expression values were derived using SDS 2.0 software package (Applied Biosystems). Differential gene expression was calculated by the ΔΔCt method. Briefly, using threshold cycle (Ct) for each gene, change in gene expression was calculated for each sample comparison by the formulae:

ΔCt_{(test sample)}=Ct_{(target gene, test sample)}−Ct_{(reference gene, test sample)} 1.

ΔCt_{(control sample)}=Ct_{(target gene, control sample)}−Ct_{(reference gene, control sample)} 2.

ΔΔCt=ΔCt₍ₜₑₛₜ₎−ΔCt_{(calibrator)} 3.

d) Statistical Analysis and CRG Identification:

Expression values from the 50 microarrays processed were obtained using the RMA procedure in Bioconductor. Differentially expressed genes were identified by the step-down Westfall-Young procedure (Westfall, P. H. & Young, S. S. Resampling-based multiple testing: examples and methods for P-value adjustment (Wiley, New York, 1993)) in conjunction with the permutation N-test (Klebanov, L., et al. (2006) Computational Statistics & Data Analysis 50, 3619-3628). The latter test is nonparametric and does not require log-expression levels to be normally distributed. The family-wise error rate (FWER) was controlled at a level of 0.01. Gene expression values derived from mp53/Ras RNA samples were compared to those from two control cell populations, YAMC and bleo/neo cells, and differentially expressed genes within the intersection of both comparisons were selected for further analysis (p value of mp53/Ras vs. YAMC <0.01∩p value of mp53/Ras vs. Bleo/Neo <0.01). This selection process was executed in parallel using both raw and quantile normalized expression values, with the genes forming the union of both procedures being selected for further analysis (Raw∪Normalized). All ESTs and “Transcribed loci” were rejected from the set of genes thus selected.

The following procedure was applied for further sub-selection of genes with a synergistic response to mutant p53 and activated Ras. Let a be the mean expression level of a given gene in mp53, b represent the mean expression level of a gene in Ras and d represent the mean expression in mp53/Ras. Then, the selection criterion defines CRGs as (a+b)÷d≦0.9 for genes over-expressed in mp53/Ras and as (d÷a)+(d÷b)≦0.9 for genes under-expressed in mp53/Ras. Unlike a similar criterion based on the general isobol equation (Berenbaum, M. C. (1989) Pharmacol Rev 41, 93-141), this criterion has no rigorous theoretical justification. However, it is heuristically appealing and served well for the purposes of the study.

e) Genetic Perturbation of Gene Expression:

(1) Re-Expression of Down-Regulated Genes:

For stable gene re-expression, cDNA clones were obtained from the IMAGE consortium collection, distributed by Open Biosystems (Table 4), except for murine Jag2 (gift of Dr. L. Milner), and murine Tbx18, which was PCR-cloned from YAMC cDNA using sequence-specific primers. All cDNAs were sequence-verified prior to use and were cloned into the retroviral vector pBabe-puro (Morgenstern, J. P. & Land, H. (1990) Nucleic Acids Res 18, 3587-96). For combined perturbation of Fas+Rprm, cDNA for Fas was sub-cloned into the pBabe-hygro retroviral vector, allowing for consecutive selection for each gene introduced. Retroviruses for infection of mp53/Ras cells were produced following transient transfection of ΦNX-eco cells (ATCC). For production of pseudotyped, human cell infectious retrovirus, pBabe retroviral vectors were co-transfected with the VSV-G gene driven by the CMV promoter into ΦNX-gp cells (ATCC). Infections were carried out in media with 8 μg/mL polybrene at 33° C. for mp53/Ras cells and at 37° C. for DLD-1 cells. Selection with 5 μg/mL puromycin, and where applicable, 200 μg/mL hygromycin B, was used to generate polyclonal populations of cells stably expressing the indicated cDNAs. Polyclonal cell populations expressing each cDNA were generated. To test reproducibility of the highly frequent effects of CRG gene perturbations on tumor formation 2-4 independent replicates of such cell populations were derived (FIG. 6A). No significant effects on tumor formation were found upon testing cell populations each expressing one of five non-CRG cDNAs. The tumor-inhibitory effect of non-CRG cDNA Tbx18 was confirmed by multiple independent replicates (FIG. 6C). As expected, the magnitude of perturbation varies between cDNAs and replicates, and falls into the following groups. For tumor-inhibitory CRGs, all replicates express cDNAs at levels below, at or moderately above YAMC mRNA expression levels. For non-tumor-inhibitory CRGs and for non-CRGs, cDNA expression levels were found at or above the levels of the corresponding YAMC mRNAs (FIG. 6).

(2) Knock Down of Up-Regulated Genes:

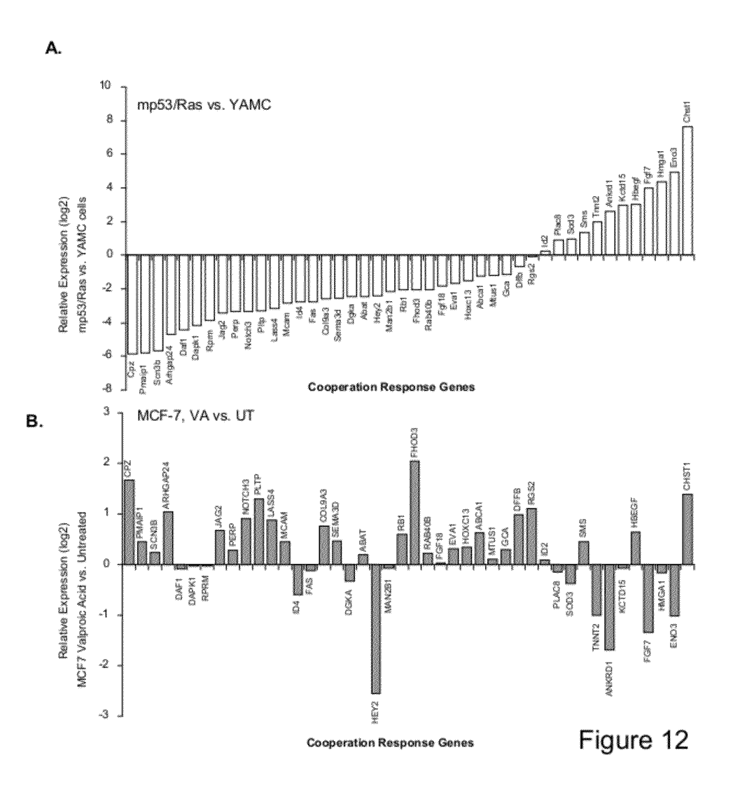

For stable gene knock-down, shRNA molecules were designed using an algorithm (Yuan, B., et al. (2004) Nucleic Acids Res 32, W130-4). Target sequences (Table 8) were synthesized as forward and reverse oligonucleotides (IDT), which were annealed and cloned into the pSuper-retro vector (Brummelkamp, T. R., et al. (2002) Science 296, 550-3) (Oligoengine). For each up-regulated gene, two or three independent shRNA target sequences were identified yielding at least 50% reduction in gene expression with the goal to guard against off-target effects (Table 8 and FIG. 12B, D). For this purpose between four and six shRNA targets for each gene were tested. In three cases, only one shRNA target sequence yielded appropriate levels of knock-down, reducing levels of gene expression comparable to those in YAMC cells (Hmga2, Igfbp4, and Klf2) (FIG. 12D). Retroviral infection of target cells was carried out as described above, except that infections of mp53/Ras cells were performed at 39° C. to maximize shRNA-mediated gene knockdown. HT-29 cells were infected at 37° C. ShRNA experiments with DLD1 and HT-29 cells were constrained by low efficiencies of mRNA knock down and instability of knock down maintenance during tumor formation.

The specificity of Plac8 knock-down was independently confirmed by expression of Plac8 cDNA rendered shRNA-resistant by introduction of appropriate silent mutations (FIG. 6B). This shRNA resistant cDNA was cloned (Genbank ID: NM_{—}139198, Wild Type sequence: 239-AAGTGGCAGCTGACATGAATG-259 (SEQ ID NO: 41), Mutated Sequence: 239-AGGTCGCCGCGGACATGAACG-259 (SEQ ID NO: 42)) into the pBabe-hygro retroviral vector and introduced into mp53/Ras cells harboring Plac8sh240 shRNA using the methods described above.

(3) Quantitation of Gene Perturbation:

The efficiency of gene perturbations was tested by comparison of RNA expression levels in empty vector-infected mp53/Ras cells and cells subjected to gene perturbation. Re-expression or knock-down was also compared with the respective levels of RNA expression in YAMC control cells. For collection of RNA, mp53/Ras cells were grown at the 39° C. for 2 days, followed by serum withdrawal for 24 hr. For quantitation of gene perturbations in HT-29 and DLD-1 cells, genetically manipulated cell populations and respective vector controls were grown in the absence of serum for 24 hr prior to harvesting RNA. Total RNA was extracted from cells following the standard RNeasy Mini Kit protocol for animal cells, with on-column DNase digestion (Qiagen).

SYBR Green-based quantitative PCR was run using cDNA produced as described above for TLDA, with 1× Bio-Rad iQ SYBR Green master mix, 0.2 μM forward and reverse primer mix, with gene-specific qPCR primers for each gene tested. Reactions were run on the iCycler (Bio-Rad), as follows: 5 mM at 95° C., 45 cycles of 95° C. for 30 seconds, 58 to 61° C. for 30 seconds, 68 to 72° C. for 45 seconds to amplify products, followed by 40 cycles of 94° C. with 1° C. step-down for 30 seconds to produce melt curves. Primers were identified using the Primer Bank database (Wang, X. & Seed, B. (2003) Nucleic Acids Res 31, e154) or designed using the IDT PrimerQuest tool. Differential gene expression was calculated by the ΔΔCt method, described above.

f) Western Blotting:

mp53/Ras cells were grown at 39° C. for 2 days prior to lysis for Western blots. HT-29 and DLD-1 cells were grown in standard conditions, described above. Cell pellets were lysed for 20 min at 4° C. with rotation in RIPA buffer (50 mM Tris-HCL, pH 7.4, 150 mM NaCL, 1% NP-40, 5 mM EDTA, 0.1% SDS, 0.5% deoxycholic acid, protease inhibitor cocktail tablet). Lysates were clarified by centrifugation at 13,000 g for 10 min at 4° C. and quantitated using Bradford protein assay (Bio-Rad). 25 μg of protein lysate was separated by SDS-PAGE and transferred to PVDF membrane (Millipore) Immunoblots were blocked in 5% non-fat dry milk in PBS with 0.2% Tween-20 for 1 hour at RT, probed with antibodies against p53 (FL-393, Santa Cruz) for all cell lines, H-Ras (C-20, Santa Cruz) for mp53/Ras cells, Raf (F-7, Santa Cruz) for HT-29 cells, Ras (Ab-1, Calbiochem) for DLD-1 cells, and tubulin (H-235, Santa Cruz) for all cell lines. Bands were visualized using the ECL+kit (Amersham).

g) Xenograft Assays:

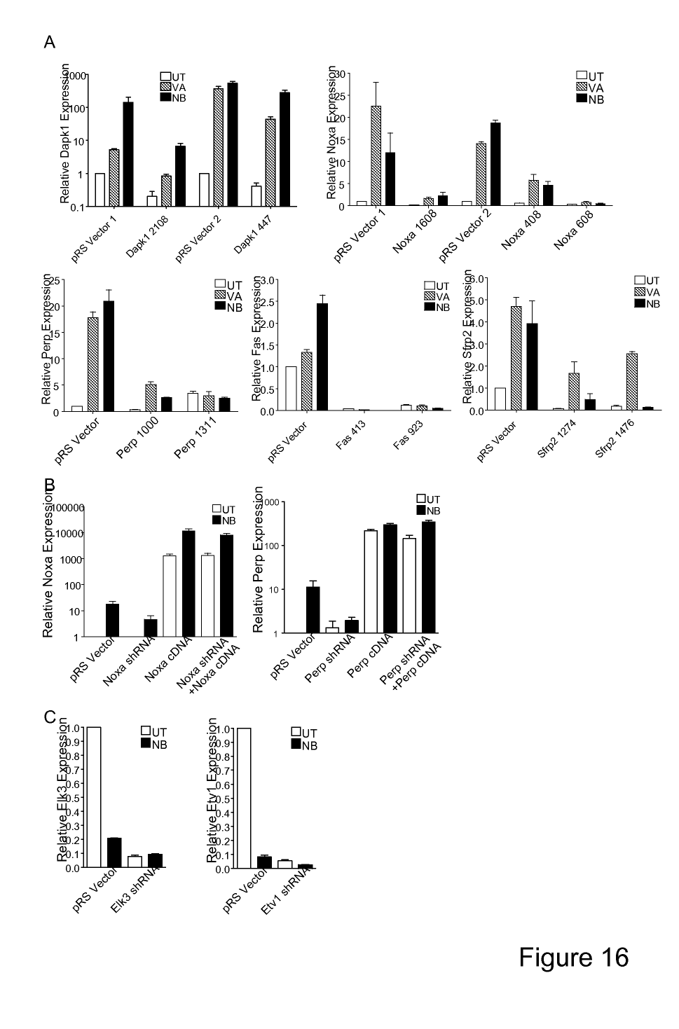

Murine mp53/Ras cells were grown at 39° C. for 2 days prior to injection. Human HT-29 and DLD-1 cells were grown in standard conditions, described above. Tumor formation was assessed by sub-cutaneous injection of 5×10⁵ cells (mp53/Ras and DLD-1 cells) or 1.25×105 cells (HT-29) into CD-1 nude mice (Crl:CD-1-Foxnlnu, Charles River Laboratories) in appropriate media (RPMI 1640 or DMEM) with no additives. For each replicate of all gene perturbations, 2-12 injections were performed for perturbed cells and vector controls, as indicated in FIGS. 12 and 16. Tumor size was measured by caliper at 2, 3 and 4 weeks post-injection. Tumor volume was calculated by the formula volume=(4/3)πr3, using the average of two radius measurements. Tumor reduction was calculated based on the average tumor volume following each gene perturbation as compared to the directly matched vector control tumors. Statistical significance of difference in tumor size was calculated by the Wilcoxn signed-rank test (Hollander, M. & Wolfe, D. A. Nonparametric Statistical Methods (Wiley-Interscience, Hoboken, N.J., 1998)), comparing tumors derived from perturbed cells with tumors induced by directly matching vector control cells.

### 2. Example 2

### Significance and Selection of Cooperation Response Genes

a) Results

In order to further assess the extent of CRG involvement in malignant transformation, perturbation of an additional 10 CRGs has been performed, revealing 6 new genes with an essential role in tumor formation. Substantial CRG co-regulation in human pancreatic and prostate cancer, which commonly contain p53 and Ras pathway mutations was also found. Finally, a number of aspects of the original process for identifying CRGs were examined and found that there are multiple paths to find this critically important gene set. Taken together, these results confirm the essential role for CRGs in malignant cell transformation, and indicate that CRGs play a role in other cancers with p53 and Ras pathway alterations. This class of genes provide new opportunities for therapeutic intervention in multiple human cancers.

(1) Cooperation Response Genes Contain High Proportion of Tumor Regulatory Genes

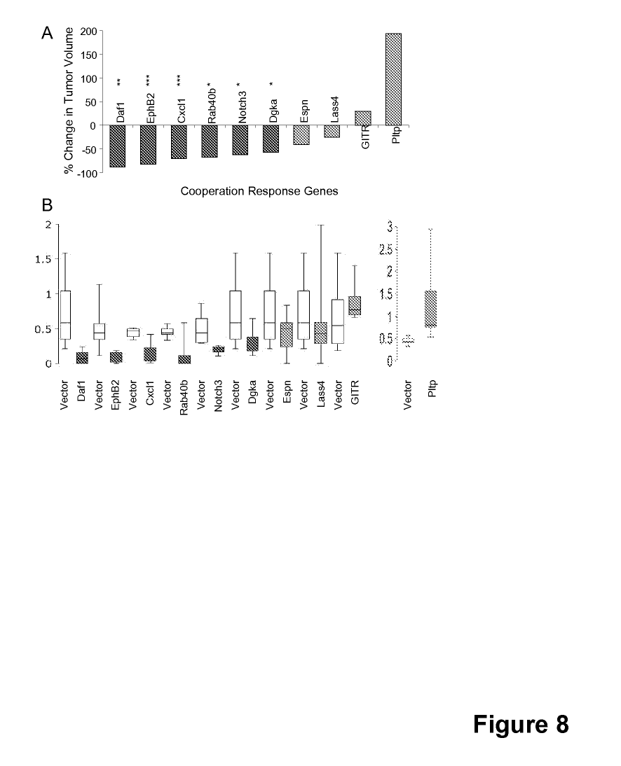

Because a subset of CRGs has been shown to play an essential role in tumor formation, additional CRGs were assessed to determine if they have a similar role in malignant transformation. To test this, an additional 10 CRGs were perturbed and found that a high proportion, 6 out of 10, are essential to tumor formation, producing significant reductions in tumor volume as compared to matched, empty vector-expressing cells (FIGS. 8A and B). Disclosed herein above, perturbation of 14 out of 24 CRGs produced a significant decrease in tumor formation upon xenograft in nude mice. The similar proportion of tumor inhibitory CRGs found here reinforces the observation that the CRG set contains many genes that regulate tumor formation capacity of cancer cells.

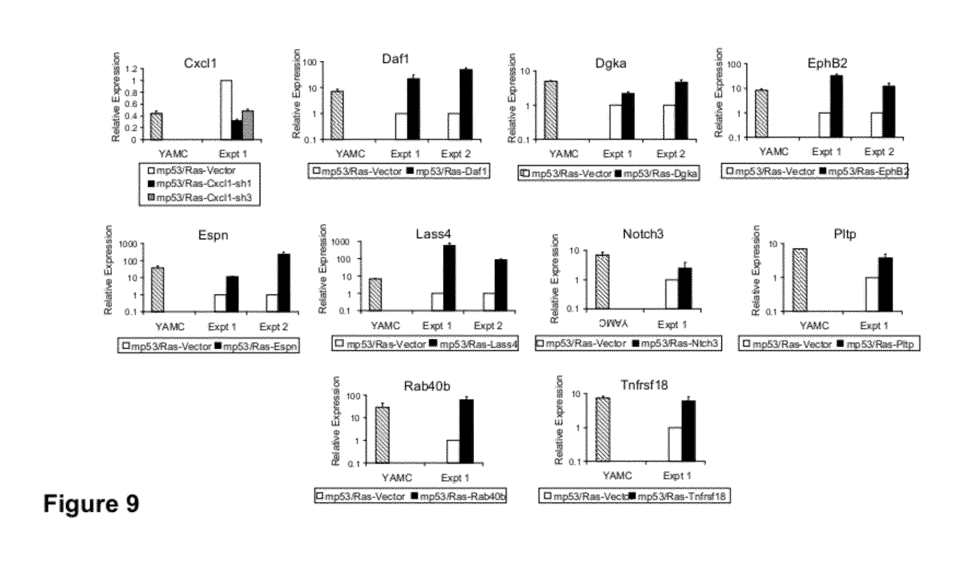

CRG perturbations were made by retroviral introduction of cDNA, encoding each target gene, or shRNA, targeting each gene for mRNA knock-down, using multiple independent shRNA targets to control for potential off-target effects. Murine colon cells (YAMC) transformed by co-expression of mutant p53^{175H} (mp53) and Ras^{v12} (Ras) were perturbed by infection with retroviral constructs containing appropriate shRNA or cDNA molecules. The extent of gene perturbation was controlled at the level of mRNA expression. Perturbed cells were compared to vector-infected mp53/Ras cells, as well as normal YAMC cells, to assess whether gene expression was in the range of normal cell expression or vastly different. Perturbation of all genes was at or about the level of expression in YAMC cells, with the exception of the Lass4 gene (FIG. 9). This cDNA appears to express to a substantially higher level than normal cells, but despite this, fails to show a biological effect on tumor formation capacity of cells. Polyclonal cell populations stably expressing these constructs were selected and implanted sub-cutaneously on nude mice. Tumor formation was assessed at four weeks post injection, with tumor volume measured by caliper.

(2) Crgs are Co-Regulated in Pancreatic and Prostate Cancer

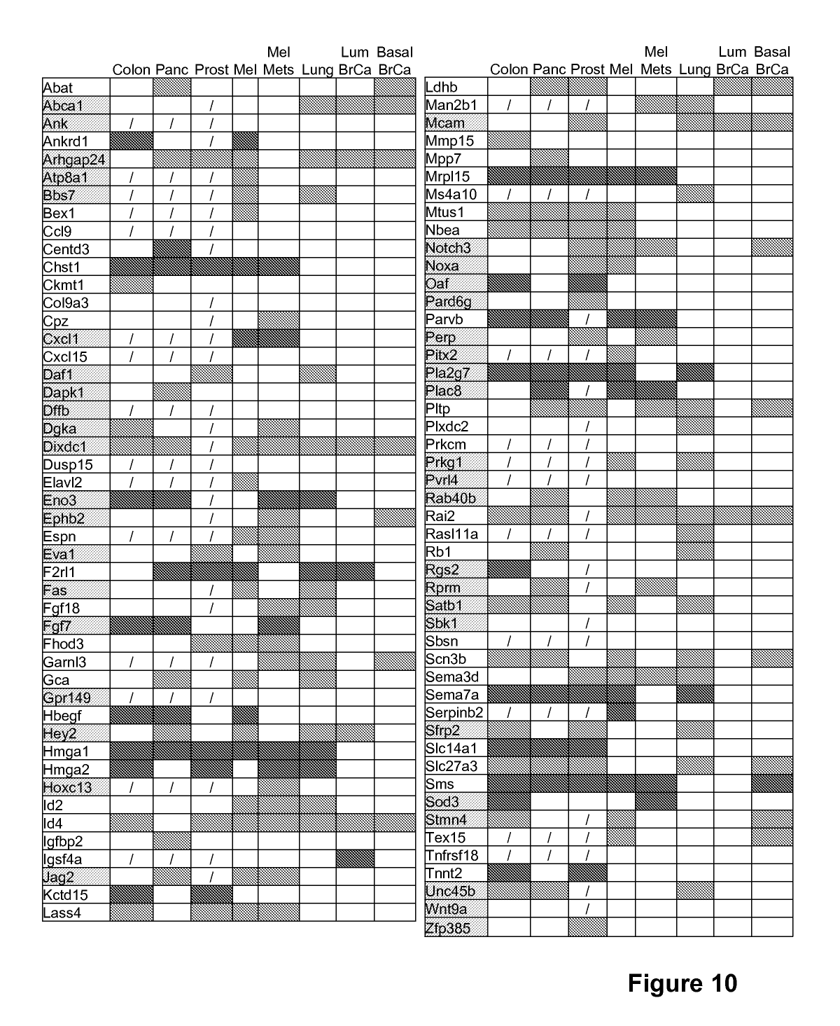

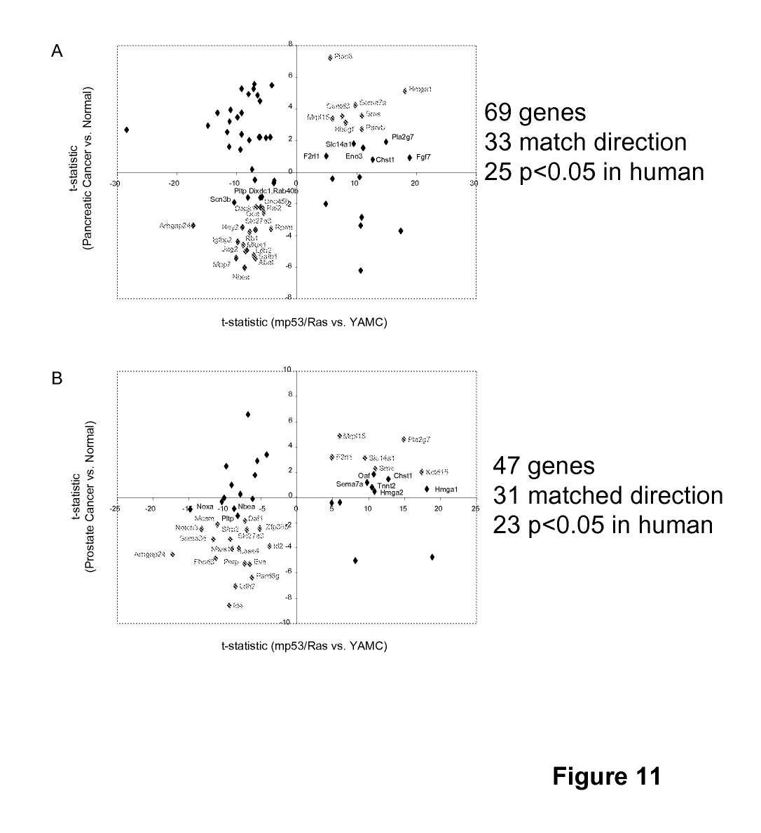

If CRGs represent the synergistic response of cells to cooperating oncogenic mutations, this gene signature may appear disregulated in cancers with a similar spectrum of mutations as the murine model. Thus, CRG expression patterns were examined in human pancreatic cancer, which frequently has mutations in the p53 and Ras genes (Hruban et al., 2000; Rozenblum et al., 1997), and prostate cancer, frequently characterized by p53 and PTEN mutation (Isaacs and Kainu, 2001). The results show that a substantial proportion of CRGs are co-regulated in both pancreatic and prostate cancer, in addition to colon cancer (FIG. 10). Specifically, of 69 CRGs represented in the pancreatic tumor data set, 33 appear co-regulated, with similar disregulation in pancreatic cancer as in the murine model system (FIG. 11A). Of these 33 genes, 25 are significantly differentially expressed in pancreatic cancer. For human prostate cancer, of 47 CRGs represented on the arrays, 31 appear co-regulated, with significant differences between cancer and normal samples for 23 of these genes (FIG. 11B). Notably, there is a substantial overlap between these cancers and colon cancer, with 9 genes similarly disregulated in all three cancers and the murine model. For these comparisons, publicly available data sets were used to compare cancer samples with normal controls for pancreatic (Lowe et al., 2007) and prostate (Lapointe et al., 2004) cancer. Differential expression in human tumor material was plotted against the differential expression pattern in mp53/Ras cells, relative to YAMC cells. These results show that CRGs are disregulated in cancers other than colon cancer, and indicates that CRGs have a similar biological role in pancreatic and prostate cancer cells.

(3) Oncogene Cooperation Limits Extracellular Cues' Contribution to Gene Expression

Identification of CRGs was done using RNA from cells grown in the absence of serum prior to harvesting, with the intent to reduce the contribution of growth and survival factors to gene expression patterns. The presence of extracellular signals from serum alters substantially the gene expression pattern in cells expressing mp53 or Ras alone. Interestingly, while gene expression in these cells is highly conditional on external signals, the mp53/Ras gene expression pattern is largely independent of external cues contributed by serum. In order to assess this, CRG expression profiles from cells grown in the presence or absence of serum for 24 hours were compared, using TaqMan Low-Density Arrays (TLDA), with four replicates of RNA from normal YAMC cells, cells expressing mp53 alone or Ras alone, and mp53/Ras cells. Gene expression is shown as expression in mp53, Ras or mp53/Ras cells relative to YAMC cells under the same growth condition. Thus, by removing serum from the cells prior to RNA extraction, the contribution of the individual oncogenes were separated from the noise of serum-derived external signals. Because CRG identification uses the gene expression values in mp53, Ras and mp53/Ras cells in a ratio, termed the synergy score, noise in the expression values of mp53 or Ras cells might have obscured synergistically regulated genes. In addition, the observation that individual oncogene effects are highly conditional, while cells with multiple mutations control gene expression regardless of their environment, may begin to explain how tumor cells gain independence from extracellular signals in the transformation process (Hanahan and Weinberg, 2000). Such independence can be driven by cooperating oncogenic lesions.

(4) N-Test is more selective of CRGs than T-Test

In order to identify CRGs, a newly developed statistical test, the N-test (Klebanov et al., 2006), was used to identify genes differentially expressed in mp53/Ras cells, as compared to two sets of control cells, YAMC, and YAMC infected with empty retroviral vectors (bleo/Neo). In order to determine whether this procedure detected a gene set that would otherwise have been obscured, the original microarray data was re-analyzed, comparing the gene list resulting from the N-test with that derived by using the more commonly applied t-test (Welch's t-test), each done with Westfall-Young adjustment. Both procedures identify a common set of 1127 genes with p-values<0.05 as compared to both normal cell controls (YAMC and empty vector-expressing bleo/Neo), but while the N-test only declares an additional 154 genes as differentially expressed, the t-test calls an additional 988 genes differentially expressed. Interestingly, using the synergy score criterion to identify CRGs produces similar lists of synergistically regulated genes, regardless of the statistical test used to identify differentially expressed genes, with the N-test list containing only 19 more CRGs than the t-test. Thus, CRGs can be found by multiple statistical methods. However, for the original purpose of comparing the biological roles of synergistically regulated genes to those regulated in a non-synergistic manner, while using the t-test produces a similar list of CRGs, the t-test also yields a substantially longer list of non-CRGs, which complicates the process of choosing such genes for perturbation.

(5) Synergy can be Found in Multiple Ways

Based on previous studies of changes in gene expression in response to single oncogenic mutations in cells, there might be hundreds or even thousands of genes that respond to the activity of a single oncogene (Fernandez et al., 2003; Huang et al., 2003). Therefore, a strategy was employed to sort the relevant changes, those on which tumor formation depends, from those that are not essential for tumor formation. Synergistic responses were utilized to cooperating oncogenes because of the substantial evidence that such cooperation induces transformation (Fanidi et al., 1992; Hahn et al., 1999; Hirakawa and Ruley, 1988; Land et al.). The synergy score metric was derived to identify genes whose expression showed a greater than additive change in mp53/Ras cells, as compared to

mp53 or Ras alone. One can define synergistic changes those that show a greater than multiplicative relationship, rather than the greater than additive relationship that was utilized in the original analysis. Alternatively, simply identifying genes with a unique expression pattern in mp53/Ras cells, as compared to cells with mp53 alone and Ras alone, indentifies tumor inhibitory genes in similar numbers.

In order to test such methods for segregating essential genes from non-essential, the results of the original additive synergy criterion was compared with a multiplicative synergy criterion, and with using the N-test to identify genes significantly differentially expressed in mp53/Ras cells as compared to mp53 or Ras alone. While the multiplicativity score and differential expression via the N-test identify somewhat different sets of genes than the additive synergy score, all three methods perform similarly at isolating genes critical to tumor formation from non-essential genes. The multiplicativity score has the drawback of generating a longer list of genes that meet the test, which increases the number of false positives, genes included on the list that do not contribute to tumor formation capacity of transformed cells. The use of differential expression in mp53/Ras vs. mp53 and Ras alone via the N-test generates a list of candidate genes similar in length to the additive synergy score list (˜100 genes), but this criterion fails to capture 5 genes that are critical to tumor formation, and which are identified as synergistic by the additive synergy score. Thus, for the purpose of using genomic data to identify functionally significant genes, the greater than additive synergistic expression criterion originally used provides the most robust separation of genes essential to tumor formation than do other criteria, but there are clearly multiple paths to identify genes required for malignant transformation.

b) Discussion

Identification of the genome-wide set of genes synergistically regulated by p53 loss-of-function and constitutive Ras activation, provides a roadmap to find downstream targets of critical importance to the cancer cell. Characterization of this gene set reveals additional genes essential for transformation, with an overall proportion of ˜60% of CRGs critical to malignant transformation individually.

Because the CRGs effectively inhibit tumor formation of p53-deficient cells, they can represent targets of great interest in colon, pancreatic and prostate cancer, for which the prognosis is poor once p53 mutations are acquired. This appears more likely given the substantial overlap in CRG disregulation between these 3 types of cancer. If CRG dependence is similar in pancreatic and prostate cancer, then targeting CRGs in other cancer cells can yield similar results as in colon cancer cells, and ultimately lead to additional therapeutic opportunities in pancreatic and prostate cancer.

In order to identify CRGs, appropriate methods must be used. If synergistic regulation is obscured by noise in the data generated, valuable information may be lost. Based on analysis of the methodology, there are multiple paths to finding CRGs, with the limitations of each taken into consideration. In particular, the choice to remove serum from cells prior to harvesting RNA appears to have greatly reduced the context-dependent noise in the single oncogene expressing cells' RNA populations. While the gene expression pattern in the mp53/Ras cells is largely independent of extracellular cues, gene expression in cells with mp53 or Ras alone show greater integration of the oncogenic and extracellular signals. This feature relates to the biological capacity of tumor cells to ignore normal extracellular cues to cease proliferation, commit suicide or remain within a confined tissue context (Hanahan and Weinberg, 2000). It is likely that cancer cells must become independent of extracellular cues in order to progress to full malignancy, and this appears to be a consequence of oncogene cooperation.

The statistical methodology used for the original analysis was important to the comparison of CRGs with non-synergistically regulated genes. The N-test produces a shorter list of differentially expressed genes, facilitating identification and perturbation of an appropriate number of non-CRGs. By using the t-test, the list of non-CRGs is substantially longer, and requires perturbation of many more non-CRGs. Because the number of synergistically regulated genes in the whole genome is independent of statistical differentials, having a longer list of non-synergistically regulated genes as a starting point is a significant barrier. For simple identification of CRGs, however, both tests perform similarly.

In terms of finding synergistically regulated genes, the synergy score appears to perform the best in terms of segregating tumor inhibitory perturbations from those which do not alter tumor formation capacity of cells. Identification of genes by a greater than multiplicative relationship in mp53/Ras cells, as compared to mp53 and Ras alone, includes the same number of tumor-regulatory CRGs, but has the limitation of generating a longer list. This increases the false-positive rate among the so-called CRGs. By choosing to find genes differentially expressed in mp53/Ras cells, as compared to mp53 and Ras alone, a similar number of CRGs were identified, but lose a subset of genes essential to transformation. Thus, the synergy score is a slightly better measure for identification of CRGs, which are enriched for tumor inhibitory genes. Clearly, other criteria for finding such genes also enrich the proportion of genes that play an essential role in malignant transformation.

The results demonstrate a means by which to discern functionally important features in genomic scale gene expression data. Genes regulated by the cooperation between oncogenic mutations represent an enriched set of targets with the capacity to control tumor formation of transformed cells, both mouse and human. Such “cooperation response addiction” opens up a wide range of potential cancer therapeutic targets from among these genes. Therapies that act downstream of initiating oncogenic lesions have the potential to ablate tumor formation despite the persistence of these oncogenes. Importantly, CRG perturbation can reduce or ablate tumor formation on a background of loss of p53 function, which currently confounds most chemotherapeutic strategies. The data indicates that restoring p53 function is not essential for disrupting tumor formation but can be replaced by targeting p53-negative tumors at the level of CRGs downstream of oncogenic mutations.

c) Materials and Methods

(1) Cells

Four polyclonal cell populations, control (Bleo/Neo), mp53 (p53175H/Neo), Ras (Bleo/RasV12) and mp53/Ras (p53175H/RasV12) were derived by retroviral infection of low-passage polyclonal young adult mouse colon (YAMC) cells (Xia and Land, 2007). YAMC cells (a gift from R. Whitehead and A. W. Burgess) derived from the Immorto-mouse (Jat et al., 1991; Whitehead et al., 1993) (aka H-2 Kb/tsA58 transgenic mouse) expressing temperature-sensitive simian virus 40 large T (tsA58) under the control of an interferon γ-inducible promoter were maintained at the permissive temperature (33° C.) for large T in the presence of interferon γ to support conditional immortalization in vitro. This permits expansion of the cells in tissue culture. In contrast, exposure of YAMC cells to the non-permissive temperature for large T (39° C.) in the absence of interferon leads to growth arrest followed by cell death, indicating the absence of spontaneous immortalizing mutations in the cell population. The cells were cultured on Collagen IV-coated dishes (1 μg/cm2 for 1.5 hr at room temp; Sigma) in RPMI 1640 medium (Invitrogen) containing 10% (v/v) fetal bovine serum (FBS) (Hyclone), 1×ITS-A (Invitrogen), 2.5 μg/ml gentamycin (Invitrogen), and 5 U/ml interferon γ (R&D Systems). All experiments testing the effects of RasV 12 and p53175H were carried out at the non-permissive temperature for large T function (39° C.) and in the absence of interferon γ.

(2) Genetic Perturbation of Gene Expression

Re-expression of down-regulated genes: For stable gene re-expression, cDNA for each gene was cloned into the pBabe retroviral vector, which was used to produce ecotropic or pseudotyped retrovirus for infection of mp53/Ras, HT-29 or DLD-1 cells. Cells were drug selected to derive polyclonal cell populations for xenograft assays.

Knock down of up-regulated genes: For stable gene knock-down, shRNA targeting each gene was cloned into the pSuper-retro retroviral vector, which was used as pBabe vectors above. The specificity of Plac8 knock-down was independently confirmed by expression of Plac8 cDNA rendered shRNA-resistant by introduction of appropriate silent mutations. This shRNA resistant cDNA was cloned into the pBabe-hygro retroviral vector and introduced into mp53/Ras cells harboring Plac8sh240 shRNA.

Quantitation of gene perturbation: The efficiency of gene perturbations was tested by comparison of RNA expression levels in empty vector-infected mp53/Ras cells and cells subjected to gene perturbation via SYBR Green qPCR with gene-specific primers. Re-expression or knock-down was also compared with the respective levels of RNA expression in YAMC control cells.

(3) Xenograft Assays

Tumor formation was assessed by sub-cutaneous injection of cells into CD-1 nude mice (Cr1: CD-1-Foxn1^{nu}, Charles River Laboratories). Tumor size was measured by caliper at 2, 3 and 4 weeks post-injection. Significance of difference in tumor size was calculated by the Wilcoxn signed-rank test and by the t-test using directly matching vector control cells for each perturbation.

Comparison of CRG expression in human colon cancer and mp53/Ras cells: Expression values from microarrays examining primary human cancer samples and normal tissue samples were obtained from the Stanford Microarray database. Representative probe sets were identified on the cDNA microarrays for 69 of the CRGs in colon and pancreatic samples and 47 of the CRGs for prostate samples. T-statistics and unadjusted p-values were calculated by Welch's t-test, comparing the expression values for these probe sets in human cancer samples, compared to normal tissue samples, and for mp53/Ras compared to YAMC samples.

(4) TLDA QPCR

The TaqMan Low-Density Array (Applied Biosystems) consists of TaqMan qPCR reactions targeting the cooperation response genes available (76 genes, listed in Table 2) and control genes (18S rRNA, GAPDH) in a microfluidic card. To generate cDNA for qPCR analysis, quadruplicate samples of total RNA (10 μg/sample) from YAMC, mp53/neo, bleo/Ras and mp53/Ras cells isolated from cells grown in the presence or absence of serum were mixed with 1× SuperScript II reverse transcriptase buffer, 10 mM DTT, 400 μM dNTP mixture, 0.3 ng random hexamer primer, 2 μL RNaseOUT RNase inhibitor and 2 μL of SuperScript II reverse transcriptase in a 100 μL reaction (all components from Invitrogen). RT reactions were carried out by denaturing RNA at 70° C. for 10 minutes, plunging RNA on to ice, adding other components, incubating at 42° C. for 1 hour and heat inactivating the RT enzyme by a final incubation at 70° C. for 10 minutes.

For each sample, 82 μL of cDNA was combined with 328 μl of nuclease free water (Invitrogen) and an equal volume of TaqMan Universal PCR Master Mix No AmpErase UNG (Applied Biosystems). The mixture was loaded into each of 8 ports on the card at 100 μL per port. Each reaction contained forward and reverse primer at a final concentration of 900 nM and a TaqMan MGB probe (6-FAM) at 250 nM final concentration. The cards were sealed with a TaqMan Low-Density Array Sealer (Applied Biosystems) to prevent cross-contamination. The real-time RT-PCR amplifications were run on an ABI Prism 7900HT Sequence Detection System (Applied Biosystems) with a TaqMan Low Density Array Upgrade. Thermal cycling conditions were as follows: 2 min at 50° C., 10 min at 94.5° C., 40 cycles of 97° C. for 30 seconds, and annealing and extension at 59.7° C. for 1 minute. Each individual replicate cDNA sample was processed on a separate card.

Gene expression values were derived using SDS 2.0 software package (Applied Biosystems). Differential gene expression was calculated by the ΔΔCt method. Briefly, using threshold cycle (Ct) for each gene, change in gene expression was calculated for each sample comparison by the formulae:

ΔCt_{(test sample)}=Ct_{(target gene, test sample)}−Ct_{(reference gene, test sample)} 1.

ΔCt_{(control sample)}=Ct_{(target gene, control sample)}−Ct_{(reference gene, control sample)} 2.

ΔΔCt=ΔCt₍ₜₑₛₜ₎−ΔCt_{(calibrator)} 3.

(5) Statistical Analysis and CRG Identification

Expression values from the 50 microarrays processed were obtained using the RMA procedure with background correction in Bioconductor. Differentially expressed genes were identified by the step-down Westfall-Young procedure in conjunction with the permutation N-test, or with Welch's t-test. The family-wise error rate (FWER) was controlled at a level of 0.05. Gene expression values derived from mp53/Ras RNA samples were compared to those from two control cell populations, YAMC and bleo/neo cells, and differentially expressed genes within the intersection of both comparisons were selected for further analysis, {p value of mp53/Ras vs. YAMC <0.05} AND {p value of mp53/Ras vs. Bleo/Neo <0.05}. This selection process was executed in parallel using both raw and quantile normalized expression values, with the genes forming the union of both procedures being selected for further analysis, {Raw} OR {Normalized}. ESTs and “Transcribed loci” were rejected from the set of genes thus selected.

Genes that respond synergistically to the combination of mutant p53 and activated Ras, i.e. with a fold-change larger than the sum of fold-changes induced by mutant p53 and activated Ras individually, were termed CRGs. The following procedure was applied in parallel to mean values of raw and quantile normalized expression measurements, with the genes forming the union of both procedures being selected as CRGs for further analysis, {CRG Raw} OR {CRG Normalized}. Let a be the mean expression value for a given gene in mp53 cells, b represent the mean expression value for the same gene in Ras cells and d represent the mean expression value for this gene in mp53/Ras cells. Then, the selection criterion defines CRGs as

$$\frac{a + b}{d} \leq 0.9$$

for genes over-expressed in mp53/Ras cells and as

$$\frac{d}{a} + \frac{d}{b} \leq 0.9$$

for genes under-expressed in mp53/Ras cells, as compared to controls.

The multiplicativity score was calculated as (a*b)/d≦0.9 for genes over-expressed in mp53/Ras cells and as (d/a)*(d/b)≦0.9 for genes under-expressed in mp53/Ras cells, as compared to controls.

### 3. Example 3

### Cooperation Response Genes as Targets for Anti-Tumor Agents

Genomic analysis of tumor gene expression has identified gene signatures that can predict tumor behavior (Alizadeh et al., 2000; Ramaswamy et al., 2003; van de Vijver et al., 2002) and drug sensitivity (Bild et al., 2006; Hassane et al., 2008; Lamb et al., 2006; Stegmaier et al., 2004), to aid cancer diagnosis and treatment decisions (Nevins et al., 2003; Nevins and Potti, 2007; van't Veer and Bernards, 2008). Numerous studies indicate the utility of gene expression-based strategies for identifying drugs that mimic or reverse biological states across different cell types and species (Hassane et al., 2008; Hieronymus et al., 2006; Hughes et al., 2000; Lamb et al., 2006; Stegmaier et al., 2004; Stegmaier et al., 2007; Wei et al., 2006). To facilitate such comparisons, the Connectivity Map (CMap) was created (Lamb et al., 2006). The CMap is a compendium of gene expression signatures from human cancer cells treated with pharmacologic agents, which uses a pattern-matching strategy to connect query gene expression signatures with reference profiles (Lamb et al., 2006). Positive connectivity can identify common biological effects of compounds (Lamb et al., 2006). The CMap can also identify antagonists of disease states, via negative connectivity, including novel putative inhibitors of Alzheimer's disease, dexamethasone-resistant acute lymphoblastic leukemia and acute myeloid leukemia stem cells (Hassane et al., 2008; Lamb et al., 2006; Wei et al., 2006).

The CMap was utilized to identify instances of negative connectivity to the CRG signature, in order to find pharmacologic agents that reverse the CRG signature and function to inhibit malignant transformation. This identified histone deacetylase inhibitors (HDACi) among the most negatively connected compounds in multiple instances. A variety of natural and synthetic compounds function as HDACi (Minucci and Pelicci, 2006) and induce cell cycle arrest, differentiation, and apoptosis in human cancer cell lines in vitro (Butler et al., 2000; Gottlicher et al., 2001; Hague et al., 1993; Heerdt et al., 1994). These drugs inhibit the function of the histone deacetylase enzymes (HDACs), which remove acetyl groups from lysine residues on histone tails, condensing chromatin structure and preventing transcription factor binding (Marks et al., 2000), associated with heterochromatin formation and transcriptional silencing (Iizuka and Smith, 2003; Jenuwein and Allis, 2001). Gene expression is highly dependent upon chromatin structure that is regulated by the opposing activities of histone acetyltransferases (HATs) and HDACs (Marks et al., 2000). HDACi are currently under clinical evaluation as single agents (Carducci et al., 2001; Gilbert et al., 2001; Gore et al., 2002; Kelly et al., 2005; Kelly et al., 2003; Patnaik et al., 2002) or in combination with existing chemotherapeutic agents (Kuendgen et al., 2006).

HDACi appeared to be an attractive test case for the idea that pharmacologically-induced reversion of CRG expression can mediate tumor inhibitory activity for several reasons: first, because of the large number of HDACi hits associated with reversal of CRG expression in the CMap search; second, the observation that expression of most CRGs are suppressed in the transformation process, and third, because of the potential clinical utility of HDACi in cancer intervention. Accordingly, whether HDACi reverses the CRG signature was tested in the system in which CRGs were identified, young adult mouse colon cells transformed by mutant p53 and activated Ras (mp53/Ras cells). Exposure to either of two HDACi, valproic acid (VA) or sodium butyrate (NB), induces an extensive reversal of the CRG expression signature, significantly altering ˜55% of CRGs. This includes five down-regulated genes that promote apoptosis, Dapk, Fas, Noxa, Perp, and Sfrp2. Gene perturbation experiments in mp53/Ras cells show that inhibiting HDACi-mediated induction of three of these five CRGs reduces death sensitivity and permits tumor formation by HDACi-treated cells. This indicates that the anti-tumor effects of HDACi are dependent upon restoring expression of the CRGs tested. A similar causal relationship between the anti-tumor effects of HDACi and induction of CRG expression was found in the human colon cancer cell line, SW480. Taken together, the data shows that changes in the CRG signature underlie HDACi sensitivity in both murine and human cancer cells, demonstrating a direct relationship between drug effects on gene expression and biological behavior of treated cells. Thus, reversion of the CRG signature can serve as an attractive tool set for the identification of new anti-cancer drugs.

a) Results

(1) Identification of Compounds that Reverse the CRG Signature

The CRG signature represents the malignant state of cells transformed by the cooperative effects of mp53 and Ras. Reversion of individual CRG expression by genetic means has been shown to abrogate tumor formation capacity of perturbed cells. Given that CRG reversal inhibits tumor formation, reversal of the CRG signature by pharmacologic means similarly compromises the transformed state of cancer cells. The CMap was utilized to identify compounds that reverse CRG expression in the human cancer cells tested, by searching for highly negatively connected instances from among the hundreds of CMap gene profiles (Hassane et al., 2008; Lamb et al., 2006). Among the most negatively connected compounds were multiple instances of HDACi, including valproic acid (VA), which reverses much of the CRG expression pattern, according to the gene profiles contained in the CMap (FIG. 12). Connectivity scores for the top 20 hits from the CMap (build 1) are shown in Table 12. Although the most negatively connected compound is the PI3-Kinase pathway inhibitor, LY-294002, experimental validation was focused on HDACi because of their translational value, multiple instances of identification and strong negative connectivity scores.

TABLE 12
Results of Connectivity Map comparison with CRG expression signature

| CMAP | | | | Connectivity | | |
|---|---|---|---|---|---|---|
| Instance | Perturbagen | Concentration | Cells | Score | ESup | ESdown |
| 258 | LY-294002 | .00001M | MCF7 | −1 | −0.38 | 0.18 |
| 433 | valproic acid | .001M | PC3 | −0.96 | −0.34 | 0.21 |
| 448 | trichostatin A | .0000001M | PC3 | −0.96 | −0.16 | 0.38 |
| 409 | valproic acid | .001M | HL60 | −0.95 | −0.36 | 0.18 |
| 1024 | haloperidol | .00001M | MCF7 | −0.94 | −0.28 | 0.25 |
| 327 | arachidonyltrifluoromethane | .00001M | MCF7 | −0.91 | −0.42 | 0.09 |
| 1014 | trichostatin A | .000001M | MCF7 | −0.90 | −0.23 | 0.28 |
| 901 | 5114445 | .00001M | MCF7 | −0.90 | −0.39 | 0.12 |
| 421 | trifluoperazine | .00001M | MCF7 | −0.89 | −0.35 | 0.15 |
| 869 | wortmannin | .000001M | MCF7 | −0.89 | −0.19 | 0.31 |
| 255 | dexamethasone | .000001M | MCF7 | −0.86 | −0.24 | 0.25 |
| 915 | topiramate | .000003M | MCF7 | −0.86 | −0.34 | 0.14 |
| 1022 | sirolimus | .0000001M | MCF7 | −0.86 | −0.30 | 0.18 |
| 1113 | doxycycline | .0000144M | MCF7 | −0.84 | −0.33 | 0.14 |
| 833 | 5255229 | .000013M | MCF7 | −0.81 | −0.32 | 0.13 |
| 603 | nifedipine | .00001M | MCF7 | −0.81 | −0.29 | 0.16 |
| 308 | sulindac sulfide | .00005M | MCF7 | −0.80 | −0.33 | 0.12 |
| 543 | 1,5-isoquinolinediol | .0001M | HL60 | −0.80 | −0.20 | 0.25 |
| 458 | valproic acid | .001M | PC3 | −0.79 | −0.29 | 0.16 |
| 332 | trichostatin A | .0000001M | MCF7 | −0.78 | −0.26 | 0.19 |

(2) HDAC Inhibitors Antagonize the Transformed Phenotype

To investigate whether and how HDACi affected the transformed phenotype, young adult mouse colon (YAMC) cells and their derivatives transformed mutant p53 and activated H-Ras (mp53/Ras) (Xia and Land, 2007) were exposed to either sodium butyrate (NB) or valproic acid (VA), two carboxylic acid HDACi that inhibit the activity of both class I and class II HDACs (Villar-Garea and Esteller, 2004). Transformed cells treated with 5 mM NB for three days in 10% FBS medium underwent a dramatic morphological change, where the treated cells became larger, less refractile, and reached confluence at a lower cell density, while YAMC cell morphology appeared unaffected. HDACi treatment also inhibited Mp53/Ras cell proliferation over a range of concentrations, where the maximal effects of NB and VA were reached at 1 to 2.5 mM and 2.5 to 5 mM, respectively. These compounds affect human cancer cell line behavior in vitro in the millimolar range and even higher concentrations are required in vivo (Villar-Garea and Esteller, 2004). Therefore mp53/Ras or YAMC cells were treated with 2.5 mM NB or VA to examine the effects of these compounds on cell proliferation over time. mp53/Ras cell proliferation was completely inhibited by NB or VA treatment, indicating that HDACi induce cell cycle arrest, apoptosis, or both in mp53/Ras cells. In contrast, YAMC cells did not proliferate under these conditions, and HDACi treatment did not alter this behavior.

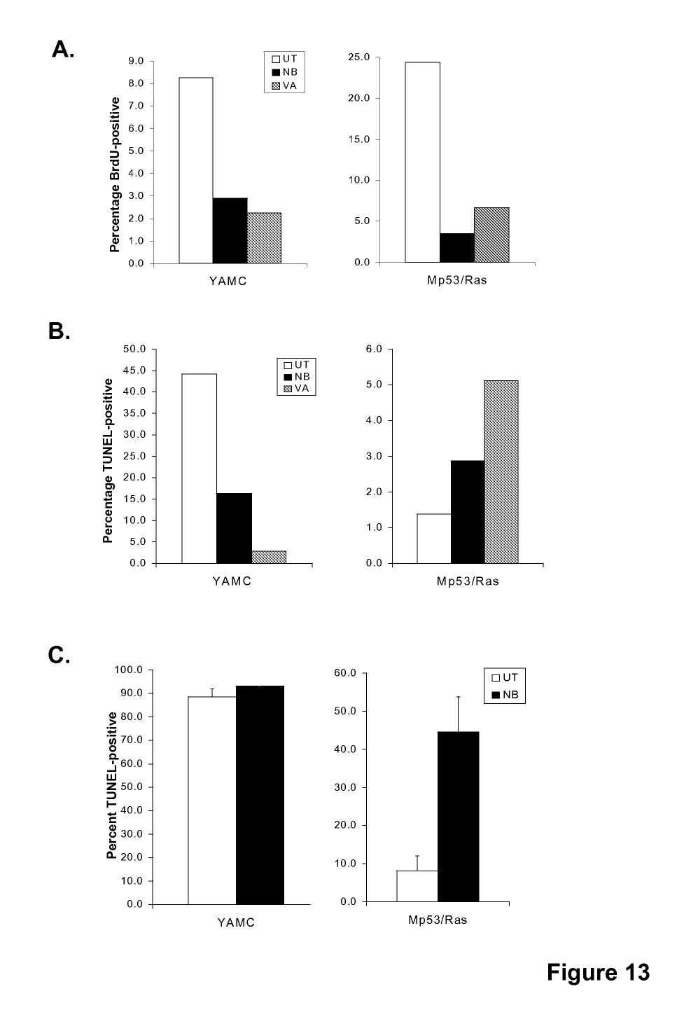

The dramatic anti-proliferative effects of HDACi on mp53/Ras cells indicated that these compounds inhibit critical properties of transformed cells, such as growth factor-independent proliferation, resistance to growth-inhibitory signals, or decreased sensitivity to pro-apoptotic signals (Hanahan and Weinberg, 2000). HDACi was investigated to determine if it abrogated the transformed phenotype by performing two cell transformation assays, in vitro colony formation in soft agar and in vivo tumor formation in immuno-compromised (nude) mice. HDACi treatment completely inhibited the ability of mp53/Ras cells to form colonies in soft agar, and tumors in nude mice, indicating that HDACi antagonize the transformed phenotype of mp53/Ras cells. To directly investigate whether HDACi-treated mp53/Ras cells lost the ability to divide or resist detachment-induced cell death under these conditions, HDACi-treated mp53/Ras or YAMC cells were suspended in methylcellose, either in the presence or absence of 10% FBS and ITS-A. In methylcellulose supplemented with 10% FBS and ITS-A, the proliferation of both mp53/Ras and YAMC cells, as measured by BrdU incorporation, was reduced by HDACi treatment (FIG. 13A). HDACi treatment also induced cell death in mp53/Ras cells under these conditions, as measured by TUNEL staining, while the percentage of apoptotic YAMC cells decreased (FIG. 13B), indicating that HDACi can selectively restore sensitivity to detachment-induced cell death, or anoikis, in transformed cells. In methylcellose without FBS or ITS-A, NB induced a greater than five-fold increase in cell death in mp53/Ras cells (FIG. 13C). Under these culture conditions, NB did not decrease apoptosis in YAMC cells, which had lost viability to approximately 90% regardless of HDACi treatment.

(3) HDACi Reverse Cooperation Response Gene Signature in mp53/Ras Cells

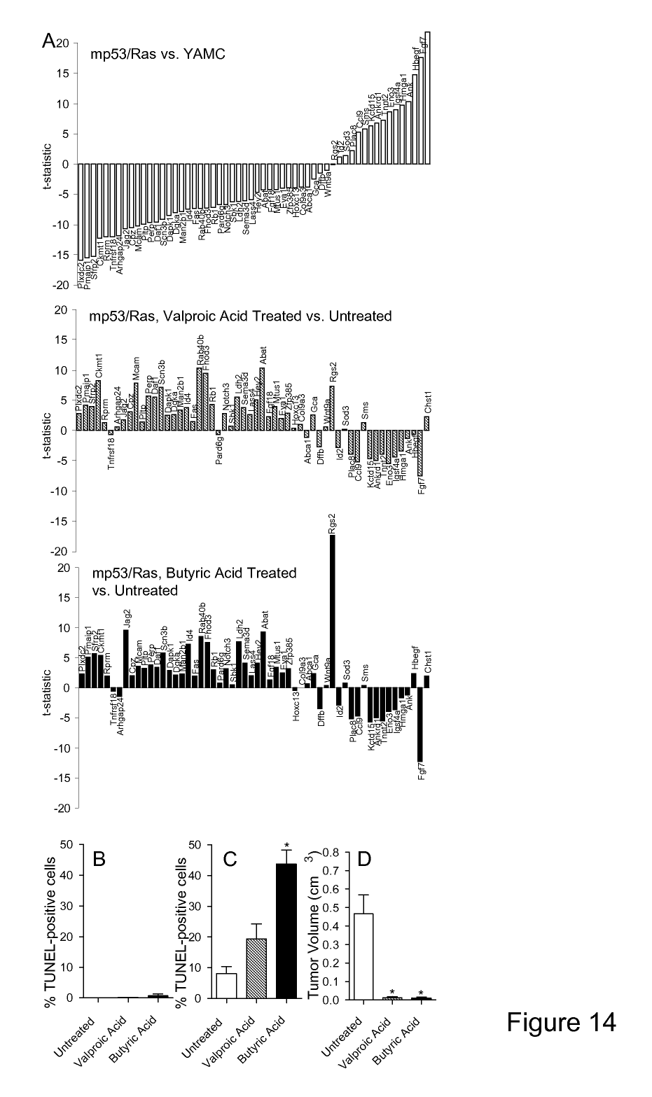

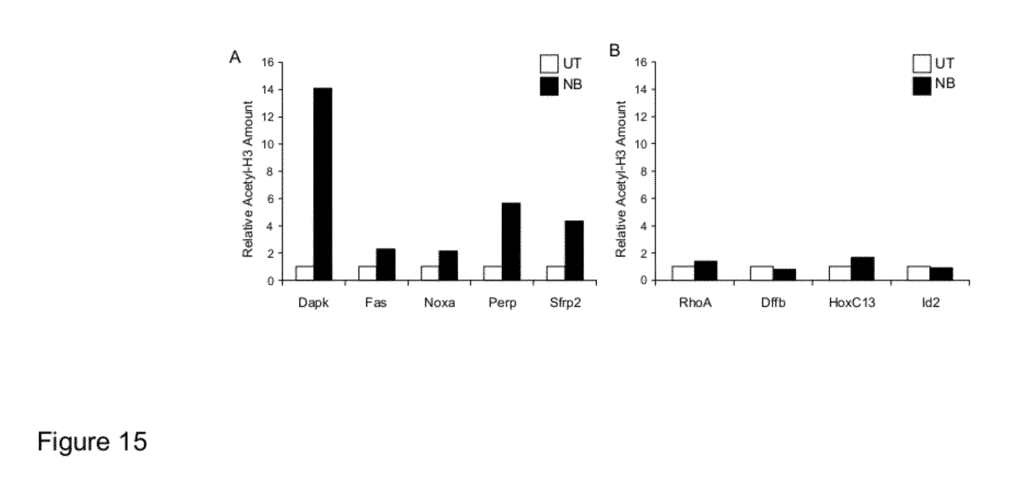

Although the CMap identifies HDACi as antagonizing the CRG signature in the human cancer cells included in the database, the effect of these drugs on CRG expression in genetically tractable cell transformation systems has not been tested. Thus, the response of 56 CRGs in mp53/Ras cells to treatment with VA or sodium butyrate (NB) was examined to determine whether these compounds have similar effects on CRG expression in cells where CRG expression is known to be essential for tumor formation. Gene expression profiles were examined using TaqMan Low-Density Arrays (TLDA) with probes to all available CRGs, comparing gene expression in mp53/Ras cells treated with VA or NB to untreated controls. Notably, the expression of about 55% of the 56 CRGs tested responded to HDACi exposure with a clear trend towards reversion of the expression pattern (FIG. 14A). The responses to both VA, identified by the CMap as a negatively connected compound, and NB, a related HDACi, were highly similar, with 31/32 regulated genes in common between the two drugs. As expected, increased expression of HDACi-induced genes correlated with an increase in histone acetylation at these gene promoters, while genes whose expression was unaffected by HDACi treatment show little difference in promoter acetylation upon drug treatment (FIG. 15).

The antagonism of CRG expression correlates with a reversion in phenotypes associated with cell transformation. HDACi treatment sensitized cells to anoikis, suspension-induced apoptosis, without causing an increase in apoptosis when cells were cultured on substratum (FIGS. 14B and C). Cells, pre-treated with VA or NB, were suspended in methylcellulose to induce cell death, which was measured by TUNEL staining. Importantly, reversion of the CRG signature also correlated with strong tumor inhibitory activity of both HDACi (FIG. 14D). Pre-treatment of cells with either VA or NB in vitro, followed by xenografting HDACi-treated cells into nude mice, produced significantly smaller tumors than those caused by untreated control cells. In this context, HDACi apparently act downstream of the oncogenic proteins, mp53 and Ras, as their levels remain unaltered and the GTP-binding activity of mutant Ras remains unaffected. These data indicate that HDACi antagonize both the CRG expression signature and malignant transformation in mp53/Ras cells downstream of the cooperating oncogenic mutations.

(4) Suppression of CRG induction by HDACi

Among the many changes in CRG expression induced by HDACi, a number of pro-apoptotic genes, including Dapk (Deiss et al., 1995; Raveh et al., 2001), Fas (Muschen et al., 2000), Noxa (Chen et al., 2005; Oda et al., 2000; Shibue et al., 2003; Villunger et al., 2003), Perp (Attardi et al., 2000; Ihrie et al., 2003), and Sfrp2 (Lee et al., 2006), show increased expression. A causal role for reversion of the Fas gene in the pro-apoptotic and anti-tumor effects of HDACi was established in a murine model of leukemia (Insing a et al., 2005). To test whether such alterations in gene expression contribute to the biological effects of HDACi treatment in the system, cells were established in which gene induction in the context of HDACi treatment was blocked or significantly inhibited. To do this, polyclonal cell populations of mp53/Ras cells stably expressing shRNA molecules targeting CRGs of interest were generated (Table 13). Cell populations exhibited a reduction in CRG expression in mp53/Ras cells without HDACi treatment. Importantly, upon HDACi treatment, CRG expression was induced in control cells, but in shRNA-expressing cells, this induction was diminished or, in the case of Fas, completely blocked. Similar effects were observed with multiple, independent shRNA targeting sequences, utilized to control for off-target effects of each shRNA (FIG. 16). In addition, the reduction in Noxa or Perp expression was rescued by expression of a shRNA-resistant form of the cDNA for each of these genes (FIG. 16). Finally, neither HDACi treatment by itself, nor interference with CRG re-expression upon HDACi treatment affected the expression of the mp53 or Ras oncogenes, demonstrating that RNA interference with HDACi-mediated gene induction operates downstream of the initiating oncogenic mutations. Taken together, these data show that the response of CRG expression to HDACi can be strongly inhibited. Moreover, the expression of four other pro-apoptotic genes that are not down-regulated in mp53/Ras vis-a-vis YAMC cells, i.e. Bad, Bakl, Bax, and Bid, was unaffected by HDACi treatment. The data thus indicates that HDACi revert the CRG expression signature in mp53/Ras cells with some degree of selectivity.

TABLE 13
Short interfering hairpin RNA constructs generated to interfere with HDACi-
induced gene expression.

| Gene | Target Region | Oligonucleotide Sequences |
|---|---|---|
| Dapk1 | 447 | Forward: 5′-GATCCCCGAGGAGGCAACGGAATTCCTTCAAGA |
| | | GAG GAA TTC CGT TGC CTC CTC TTT TTGGAA A-3′ (SEQ ID NO: 43) |
| | | Reverse: 5′-AGCTTTTCCAAAAAGAGGAGGCAACGGAATTCC |
| | | TCTCTTGAAGGAATTCCGTTGCCTCCTCGGG-3′ (SEQ ID NO: 44) |
| | 2108 | Forward: 5′-GATCCCCGGACACACACCGAGGACTCT TCAAGA |
| | | GAGAGTCCTCGGTGTGTGTCCTTTTTGGAAA-3′ (SEQ ID NO: 45) |
| | | Reverse: 5′-AGCTTTTCCAAAAAGGACACACACCGAGGACTC |
| | | TCTCTTGAAGAGTCCTCGGTGTGTGTCCGGG-3′ (SEQ ID NO: 46) |
| Elk3 | 1774 | Forward: 5′-GATCCCCTCTAGATGTATGTTAGCATTTCAAGAG |
| | | AATGCTAACATACATCTAGATTTTTGGAAA-3′ (SEQ ID NO: 103) |
| | | Reverse: 5′-AGCTTTTCCAAAAATCTAGATGTATGTTAGCATTC |
| | | TCTTGAAATGCTAAC TACATCTAGAGGG-3′ (SEQ ID NO: 104) |
| Etv1 | 1003 | Forward: 5′-GATCCCCGTGCCTAGCTGCCACTCCATTCAAGAG |
| | | ATGGAGTGGCAGCTAGGCACTTTTTGGAAA-3′ (SEQ ID NO: 105) |
| | | Reverse: 5′-AGCTTTTCCAAAAAGTGCCTAGCTGCCACTCCAT |
| | | CTCTTGAATGGAGTGGCAGCTAGGCACGGG-3′ (SEQ ID NO: 106) |
| Fas | 413 | Forward: 5′-GATCCCCGTGCAAGTGCAAACCAGACTTCAAGA |
| | | GAGTCTGGTTTGCACTTGCACTTTTTGGAAA-3′ (SEQ ID NO: 47) |
| | | Reverse: 5′-AGCTTTTCCAAAAAGTGCAAGTGCAAACCAGAC |
| | | TCTCTTGAAGTCTGGTTTGCACTTGCACGGG-3′ (SEQ ID NO: 48) |
| | 923 | Forward: 5′-GAT CCCAGCCGAATGTCGCAGAACCTTCAAGA |
| | | GAGGTTCTGCGACATTCGGCTTTTTTGGAAA-3′ (SEQ ID NO: 49) |
| | | Reverse: 5′-AGCTTTTCCAAAAAAGCCGAATGTCGCAGAACC |
| | | TCTCTTGAAGGTTCTGCGACATTCGGCTGGG-3′ (SEQ ID NO: 50) |
| Noxa | 408 | Forward: 5′-GATCCCCGTGAATTTACGGCAGAAACTTCAAGA |
| | | GAGTTTCTGCCGTAAATTCACTTTTTGGAAA-3′ (SEQ ID NO: 51) |
| | | Reverse: 5′-AGCTTTTCCAAAAAGTGAATTTACGGCAGAAAC |
| | | CTCTTGAAGTTTCTGCCGTAAATTCACGGG-3′ (SEQ ID NO: 52) |
| | 608 | Forward: 5′-GATCCCCGGAGATAGGAATGAGTTTCTTCAAGA |
| | | GAGAAACTCATTCCTATCTCCTTTTTGGAAA-3′ (SEQ ID NO: 53) |
| | | Reverse: 5′-AGCTTTTCCAAAAAGGAGATAGGAATGAGTTTC |
| | | TCTCTTGAAGAAACTCATTCCTATCTCCGGG-3′ (SEQ ID NO: 54) |
| | 1608 | Forward: 5′-GATCCCCCACGCAGAGTAAGGACTTTTTCAAGA |
| | | GAAAAGTCCTTACTCTGCGTGTTTTTGGAAA-3′ (SEQ ID NO: 55) |
| | | Reverse: 5′-AGCTTTTCCAAAAACACGCAGAGTAAGGACTTT |
| | | TCTCTTGAAAAAGTCCTTACTCTGCGTGGGG-3′ (SEQ ID NO: 56) |
| Perp | 1000 | Forward: 5′-GATCCCCGCAGCCTCTCATTTAATAATTCAA |
| | | GATTATTAAATGAGAGGCTGCTTTTTGGAAA-3 (SEQ ID NO: 57) |
| | | Reverse: 5′-AGCTTTTCCAAAAAGCAGCCTCTCATTTAATAA |
| | | TCTCTTGAATTATTAAATGAGAGGCTGCGGG-3′ (SEQ ID NO: 58) |
| | 1311 | Forward: 5′-GATCCCCGCCGCTGTCACTACTGAAATTCAAGA |
| | | GATTTCAGTAGTGACAGCGGCTTTTTGGAAA-3 (SEQ ID NO: 59) |
| | | Reverse: 5′-AGCTTTTCCAAAAAGCCGCTGTCACTACTGAAA |
| | | TCTCTTGAATTTCAGTAGTGACAGCGGCGGG-3′ (SEQ ID NO: 60) |
| Sfrp2 | 1274 | Forward: 5′-GATCCCCCCTAACATGTCCTGAGTTATATTCAA |
| | | GAGATATAACTCAGGACATGTTAGGTTTTTGGAAA-3′(SEQ ID NO: 61) |
| | | Reverse: 5′-AGCTTTTCCAAAAACCTAACATGTCCTGAGTTA |
| | | TATCTCTTGAATATAACTCAGGACATGTTAGGGGG-3′(SEQ ID NO: 62) |
| | 1476 | Forward: 5′-GATCCCCTGGTCAGTCTGTTGGCTTATATTCAA |
| | | GAGATATAAGCCAACAGACTGACCATTTTTGGAAA-3′(SEQ ID NO: 63) |
| | | Reverse: 5′-AGCTTTTCCAAAAATGGTCAGTCTGTTGGCTTA |
| | | TATCTCTTGAATATAAGCCAACAGACTGACCAGGG-3′(SEQ ID NO: 64) |
| Zac1 | 48 | Forward: 5′-GATCCCCTATCTGCCTCACAGCTGGCTTCAAGA |
| | | GAGCCAGCTGTGAGGCAGATATTTTTGGAAA-3′ (SEQ ID NO: 65) |
| | | Reverse: 5′-AGATTTTCCAAAAATATCTGCCTCACAGCTGGC |
| | | TCTCTTGAAGCCAGCTGTGAGGCAGATAGGG-3′ (SEQ ID NO: 66) |
| | 3164 | Forward: 5′-GATCCCCGAAGAATCAATCAAAGTGTTTCAAGA |
| | | GAACACTTTGATTGATTCTTCTTTTTGGAAA-3′ (SEQ ID NO: 67) |
| | | Reverse: 5′-AGCTTTTCCAAAAAGAAGAATCAATCAAAGTGT |
| | | TCTCTTGAAACACTTTGATTGATTCTTCGGG-3′ (SEQ ID NO: 68) |
| | 3745 | Forward: 5′-GATCCCCCAGCATATATCTCCTAATCTTCAAGA |
| | | GAGATTAGGAGATATATGCTGTTTTTGGAAA-3′ (SEQ ID NO: 69) |
| | | Reverse: 5′-AGCTTTTCCAAAAACAGCATATATCTCCTAATC |
| | | TCTCTTGAAGATTAGGAGATATATGCTGGGG-3′ (SEQ ID NO: 70) |

Specific shRNA molecules were designed using the Whitehead siRNA algorithm. The shRNA oligonucleotides were produced by Integrated DNA Technologies, annealed, and ligated into pRetroSuper. Gene names, target region/identifier and oligonucleotide sequences are indicated.

(5) HDACi Act Downstream of Ras

In transformed liver cells, the induction of apoptosis by NB has been reported to be associated with decreased farnesylated Ras expression and ERK1/2 phosphorylation (Jung et al., 2005). To determine whether the pro-apoptotic and anti-tumorigenic effects of HDACi on mp53/Ras cells correlates with decreased Ras expression, the expression of exogenous mutant H-Ras was examined in NB-treated Ras, and mp53/Ras cells. The data show that the expression levels of the exogenous mutant H-Ras protein were unaffected by NB treatment. In addition, expression levels of p21Cip1, a cyclin-dependent kinase inhibitor that is reportedly up-regulated by HDACi treatment (Archer et al., 1998; Gui et al., 2004; Jung et al., 2005; Richon et al., 2000), were also determined in NB-treated YAMC, mp53, Ras, and mp53/Ras cells. Notably, NB did not affect p21Cip1 expression in any of the cell lines tested. HDACi thus appears to antagonize the cancer phenotype downstream of activated Ras and independent of p21Cip1.

(6) Interference with CRG Induction by HDACi Mediates Anoikis Resistance

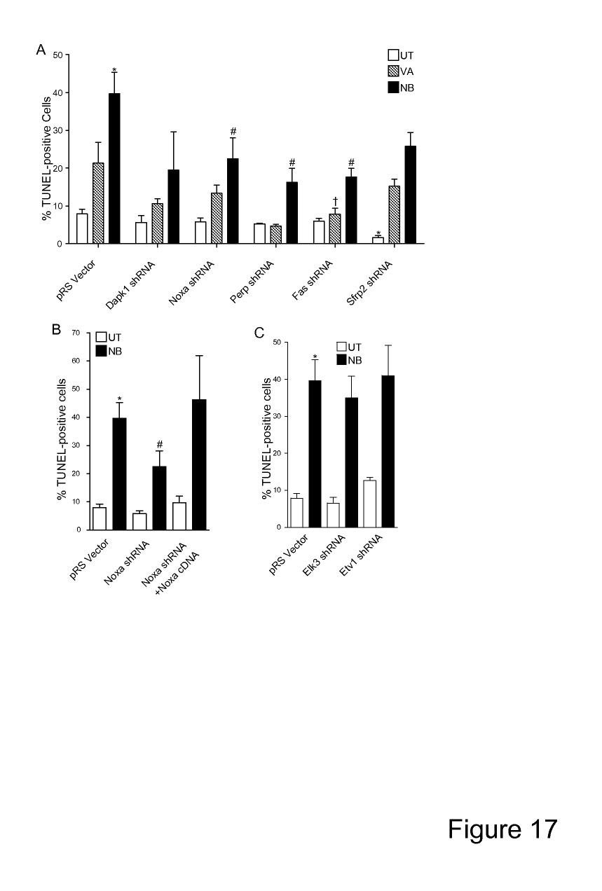

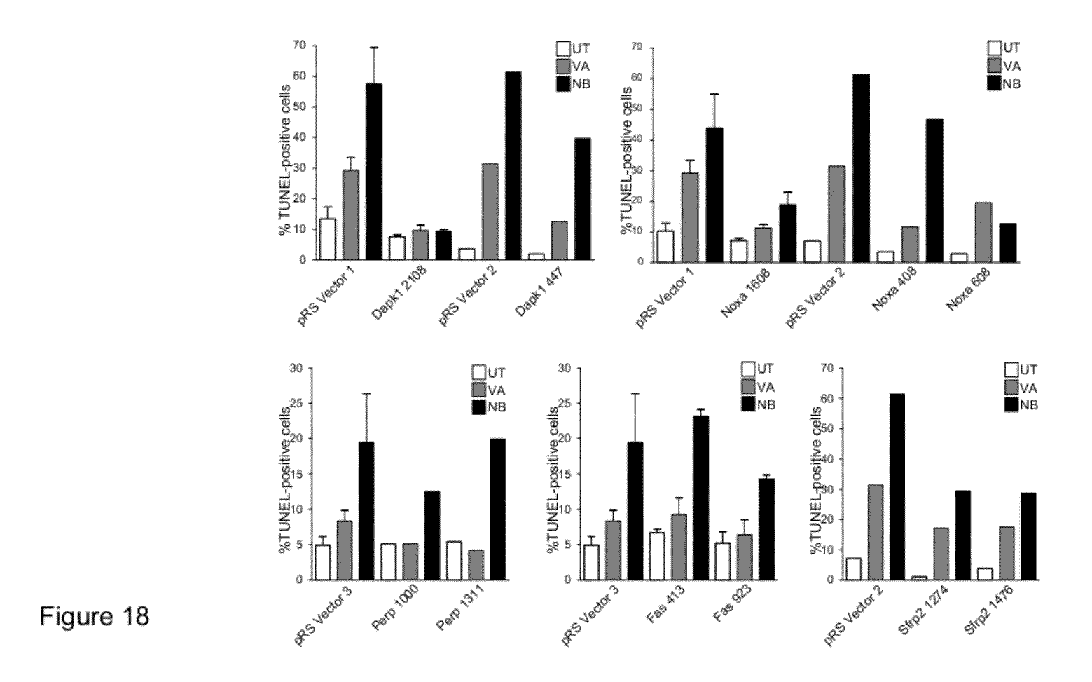

Because CRG induction by HDACi correlates with increased sensitivity to anoikis, the contribution of pro-apoptotic CRGs to this response was investigated. Anoikis was induced by cell suspension in methylcellulose after pre-treatment of cells with HDACi. Interference with Dapk, Fas, Noxa, Perp and Sfrp2 induction reduced anoikis in HDACi-treated mp53/Ras cells (FIG. 17A), demonstrating that HDACi-induced death sensitization depends on the induction of these CRGs. Only Sfrp2 reduction altered death sensitivity in untreated cells, indicating this gene controls apoptosis in an HDACi-independent manner. Similar results were observed with multiple, independent shRNA targeting molecules, indicating that the effects are specific to the targeted genes (FIG. 18). To further control for shRNA-mediated off-target effects, genetic rescue experiments were performed. Cells expressing shRNA-resistant Noxa cDNA were assayed for death sensitization by HDACi. The protective effects of Noxa reduction were reversed by restoration of Noxa expression (FIG. 17B and FIG. 16B), showing that HDACi-induced death sensitivity is Noxa dependent. In addition, to control for interference between HDACi effects and shRNA expression in general, cells with shRNA knock down of the CRGs Elk3 or Etv1 (FIG. 16C), which are not induced by HDACi treatment, did not influence HDACi-induced anoikis (FIG. 17C). Taken together, these results indicate that HDACi-induced anoikis sensitization is dependent upon the re-expression of the CRGs Dapk, Fas, Noxa, and Perp, while Sfrp2 controls cell death in an HDACi-independent manner.

(7) CRG Induction is Essential for Tumor Inhibition by HDACi

To determine whether the tumor inhibitory effects of HDACi are also dependent on CRG induction, control and shRNA expressing mp53/Ras cells were pre-treated with HDACi, and tested the tumor formation capacity of these cells in xenograft assays in nude mice. Because both HDACi VA and NB show similar effects on CRG expression (FIG. 14), and NB is a stronger death sensitizing agent (FIG. 16A), animal experiments were restricted to NB treatment to minimize animal use. Interference with Dapk, Fas, Noxa, Perp, and Sfrp2 induction destroyed tumor inhibition by HDACi, with multiple, independent shRNA targets producing similar results, demonstrating a role for these genes in HDACi-mediated tumor inhibition. However, untreated cells with reduced expression of Fas or Sfrp2 formed significantly larger tumors than controls, indicating that these genes control tumor formation in general, rather than in an HDACi-dependent manner. To again control for off-target effects of shRNAs, tumor formation capacity of cells expressing shRNA-resistant Noxa or Perp in combination with shRNA targeting these genes was compared to cells expressing only shRNA targeting these genes (FIG. 16B). Rescue of Noxa or Perp gene expression restored HDACi sensitivity to these cells, reducing tumor formation by HDACi-treated cells with high levels of Noxa or Perp expression. Moreover, interference with Elk3 or Etv1 expression did not alter tumor formation in HDACi-treated mp53/Ras cells, demonstrating that tumor formation is not altered by shRNA expression per se. Thus, while Fas and Sfrp2 control tumor formation capacity of cells in an HDACi-independent manner, the CRGs Dapk, Noxa and Perp appear to mediate the tumor inhibitory effects of HDACi.

Interference with Dapk1, Fas, Noxa, Perp, Sfrp2 or Zac1 re-expression also rescued the ability of HDACi-treated mp53/Ras cells to form tumors in vivo, indicating that the anti-tumorigenic effects of HDACi also depend on the restored expression of all six cooperation response genes. The rescued tumor formation in HDACi-treated mp53/Ras cells expressing Noxa or Zac1 shRNAs was reversed by introduction of shRNA-resistant Noxa or Zac1 cDNAs, respectively (Table 14). Moreover, interference with Elk3 or Etv1 expression did not rescue tumor formation in HDACi-treated mp53/Ras cells (Table 14). The ability of the shRNAs to rescue tumor formation in HDACi-treated mp53/Ras cells is therefore due to specifically interfering with the re-expression of Dapk1, Fas, Noxa, Perp, Sfrp2, or Zac1. HDACi thus compromise the malignant phenotype of cancer cells through antagonizing the regulation of cooperation response genes essential to the transformation process downstream of cooperating oncogenic mutations.

TABLE 14
Interference with cooperation response gene re-expression rescues
tumor formation in HDACi-treated Mp53/Ras cells.

| | UT | NB |
|---|---|---|
| Cell Line | Tumors | Tumors |
| Vector | 16/16 | 1/16 |
| Dapk1 shRNA | 4/4 | 4/4 |
| Fas shRNA | 4/4 | 4/4 |
| Perp shRNA | 4/4 | 4/4 |
| Sfrp2 shRNA | 4/4 | 4/4 |
| Noxa shRNA | 8/8 | 7/8 |
| Noxa | 4/4 | 1/4 |
| Noxa shRNA/Noxa | 4/4 | 0/4 |
| Zac1 shRNA | 10/10 | 8/10 |
| Zac1 | 2/2 | 0/2 |
| Zac1 shRNA/Zac1 | 2/2 | 0/2 |
| Elk3 shRNA | 4/4 | 0/4 |
| Etv1 shRNA | 4/4 | 0/4 |

mp53/Ras cells infected with shRNA constructs against Dapk1, Elk3, Etv1, Fas, Noxa, Sfrp2, and Zac1 were plated at 458,000 cells per 15 cm collagen IV-coated dish and treated with 2.5 mM NB for three days in 10% FBS medium for three days.
The cells were then re-suspended in additive-free medium and injected subcutaneously into the flanks of CD1 nude mice at 500,000 cells per 150 μL.
Tumor volume was measured using electronic Vernier calipers after four weeks.
The results for multiple independent shRNA constructs for Dapk1, Fas, Noxa, Perp, Sfrp2, and Zac1 are shown, including cells expressing shRNA-resistant Noxa or Zac1 cDNAs.

(8) CRG Induction Mediates HDACi Sensitivity in Human Cancer Cells

While the murine model system allows a high degree of genetic control, it is critical to determine whether similar gene dependencies exist in human cancer cells. In order to test whether the dependence of HDACi on CRG induction is similar in human colon cancer cells, the SW480 cell line was used because it harbors mutations in p53 and Ras, among a number of oncogenic mutations (McCoy et al., 1984; Rodrigues et al., 1990). HDACi treatment of these cells significantly increases expression of the CRGs Dapk, Fas, Noxa, Perp and Sfrp2, as measured by SYBR Green QPCR with gene specific primers. Because Dapk is the gene most strongly induced by NB treatment of SW480 cells, and because it mediates the anti-tumor effect of NB in mp53/Ras cells in an HDACi-dependent manner, this gene was chosen to test for CRG dependence of HDACi in human cells. RNA interference reduced the levels of Dapk in untreated SW480 cells by ˜80%, and interfered with the induction of Dapk by HDACi, suppressing Dapk levels to less than half that of cells without shRNA. Interference with Dapk induction by HDACi restored tumor formation in nude mice of HDACi-treated SW480 cells with minimal effects on untreated tumor size, demonstrating the dependence of HDACi on expression of the CRG Dapk in human cancer cells. Again, multiple independent shRNA targets were used to inhibit Dapk induction by HDACi, to control for off-target effects of shRNA molecules, with similar effects on Dapk expression and tumor formation. In addition, levels of the oncogenic p53 and Ras proteins are unaffected by either HDACi treatment or Dapk knock-down in SW480 cells, showing that the effects of HDACi and Dapk shRNA are downstream of the initiating oncogenic mutations. Therefore, the anti-tumor effects of HDACi appear to depend on CRG induction in both murine and human cancer cells.

b) Discussion

Synergistic regulation of gene expression by cooperating oncogenic mutations is a key feature of malignant transformation, demonstrated by the dependence on CRG levels in control of tumor formation capacity of transformed cells. Reversion of the CRG signature by pharmacologic means likewise antagonizes the transformed state. Here, is disclosed that the CRG signature can be pharmacologically reversed by HDACi, and importantly, that the anti-tumor activity of HDACi is mediated via induction of CRG expression. Treatment of mp53/Ras cells with VA or NB, two carboxylic acid HDACi, reversed the expression of about 55% of the 56 CRGs tested. Among the regulated CRGs are a number of pro-apoptotic genes that are repressed in cancer cells and reactivated by HDACi. These include the CRGs Dapk, Fas, Noxa, Perp, and Sfrp2, whose induction contributes to the cell death sensitivity and tumor formation capacity of cells in two modes. Dapk, Noxa and Perp underlie the apoptosis-inducing and tumor-inhibitory activities of HDACi in a specific manner. Fas and Sfrp2 act to control these behaviors in a more general way, thus blocking HDACi effects in a non-specific fashion. The consistent dependence of HDACi on CRGs in both murine mp53/Ras-transformed cells and in human colon cancer cells with similar mutations indicates that this is a general relationship, extending beyond the genetically tractable murine model system. Dependence of the biological effects of HDACi on the restored expression of CRGs demonstrates that HDACi antagonize the transformed phenotype, at least in part, by reversing oncogene-dependent repression of gene expression.

In addition to establishing a role for CRGs underlying the activity of these pharmacologic agents, the data shown here reveal a role for three additional CRGs not previously found to be essential in transformation. These genes, Sfrp2, Dapk, and Noxa, appear to act in two separate ways to control tumor formation. Because reduced expression of Sfrp2 leads to reduced apoptosis and formation of larger tumors in both untreated and HDACi treated cells, Sfrp2 expression appears to act as a restriction point in transformation, despite the fact that Sfrp2 over-expression in mp53/Ras cells fails to reduce the tumor formation capacity of these cells. A role for Sfrp2 in malignant transformation is consistent with the observation that expression of this gene is frequently lost in human cancer (Qi et al., 2006; Zou et al., 2005). While the CRGs Dapk (Chu et al., 2006; Kong et al., 2005; Kong et al., 2006; Kuester et al., 2007; Schildhaus et al., 2005) and Noxa (Mestre-Escorihuela et al., 2007) can also be lost in human cancer, they appear to play a different type of role in malignant transformation. Their importance is only revealed in the context of HDACi-induced changes in cell behavior, with no observed difference in cell death potential or tumor formation when these genes are perturbed individually (FIGS. 17A and B). This indicates the necessity for changes in other CRGs in addition to Dapk or Noxa levels in order for the effects of Dapk or Noxa to be apparent, consistent with the idea that CRGs can act together to more effectively control malignant transformation.

One critical finding here is the ease with which transformed cells can escape cell death and tumor inhibition by HDACi. The loss of any of 5 CRGs tested can reduce or prevent the biological effects of HDACi treatment. This indicates simple and parallel paths for tumors to evade the effects of HDACi, a feature that does not extend to other pharmacological agents. Nevertheless, the reletive ease with which HDACi resistance can be achieved reaffirms the importance of multi-drug combinations, with different modes of action or target sets of genes, in order to restrict the ability of tumor cells to avoid drug effects. The complexity of the CRG signature allow for identification and testing of compounds alone and in combination that affect non-overlapping sub-groups of CRGs.

Finally, the observation that reversion of the CRG signature underlies the tumor inhibitory activity of HDACi, which depend on altered CRG expression for their effects, has important practical implications. The responsiveness of the CRG signature to pharmacologic agents is expected to function as a diagnostic indicator to predict tumor sensitivity to such agents. Moreover, because the CRGs are known to be essential regulators of cancer, the mechanism of action of drugs that reverse the CRG signature can work through such changes in gene expression. The significance of CRG reversion in the response of cancer cells to pharmacological agents, such as HDACi, provides proof of principle that the CRG signature can be used as a powerful tool for anti-cancer drug screening. This is an exciting prospect for the identification of new small molecular drugs with potential for cancer therapy.

c) Materials and Methods

(1) Connectivity Map Query:

To facilitate rapid cross-species queries, a local version of the CMap database was created in which the CMap dataset was downloaded from GEO (accession# GSE5258) and treatment-control instances for each drug were generated using annotation provided in Lamb et al. (Lamb et al., 2006). Since Affymetrix IDs are human-specific in the CMap, Affymetrix IDs for each drug treatment instance were mapped to gene symbols. The median expression difference of multiple Affymetrix IDs was used when a many-to-one relationship existed between Affymetrix IDs and unique gene symbols. This local gene symbol-based version of the CMap performed similarly to the Affymetrix ID-based version originally described by Lamb et al. (Hassane and Jordan, unpublished).

The query signature consisted of 19 up-regulated CRGs and 39 down-regulated CRGs for which gene symbol annotation was present in the CMap data set. The Kolmogorov-Smirnov-based gene set enrichment analysis (GSEA) algorithm (Subramanian et al., 2005) was used to obtain enrichment scores (ES) for both up-regulated (ESᵤₚ) and down-regulated (ES_{down}) CRGs for each CMap drug treatment instance. The values of ESᵤₚ and ES_{down} were combined to generate a CMap “connectivity score” as described (Lamb et al., 2006). Drugs that mimic the CRG signature attain a positive connectivity score whereas drugs that oppose the CRG signature (and thereby are predicted as potential anti-cancer drugs) attain a negative connectivity score.

(2) Cell Culture, Anoikis and Tumor Formation Assays:

The YAMC cell system (Jat et al., 1991; Whitehead et al., 1993) and transformation of these cells by mp53/Ras are described elsewhere (Xia and Land, 2007). YAMC and mp53/Ras cells were cultured for two days at 39° C. in RPMI with 10% FBS without interferon-γ on collagen IV-coated dishes. Cells were then re-plated on collagen IV-coated dishes into the same medium containing either 2.5 mM NB, 2.5 mM VA, or no drug for 72 hours at a density of 4.58×10⁵ cells per 15-cm dish. Cells were harvested for RNA isolation at this point, or used for biological assays as described below.

For anoikis assays, cells were then trypsinized, counted and suspended in methylcellulose at a density of 1.5×10⁵ cells/mL for an additional 72 hours in the absence of HDACi. Suspended cells were pelleted, washed and fixed in 4% paraformaldehyde for TUNEL staining.

For tumor formation studies, cells were treated with HDACi as indicated above, then trypsinized, counted and injected sub-cutaneously into the flanks of CD-1 nude mice at a multiplicity of 5×10⁵ cells per injection. Mice were observed and tumors measured for 4 weeks post-injection by caliper.

SW480 cells were grown at 37° C. in DMEM with 10% FBS and antibiotics. For HDACi treatment of SW480, cells were plated into medium containing either 2.5 mM NB, 2.5 mM VA or no drug for 72 hours at a density of 1.37×10⁶ cells per 15-cm dish. Cells were then harvested for RNA isolation, or used for tumor formation studies as described above, except that SW480 cells were injected at a multiplicity of 5×10⁶ cells per injection.

(3) TLDA QPCR:

The TaqMan Low-Density Array (Applied Biosystems) consists of TaqMan qPCR reactions targeting the cooperation response genes available and control genes (18S rRNA, GAPDH) in a microfluidic card. TLDA were used to independently test gene expression differences observed in the CMap database which used Affymetrix arrays. To generate cDNA for qPCR analysis, quadruplicate samples of RNA was isolated from untreated YAMC cells or mp53/Ras cells treated with either 2.5 mM VA, 2.5 mM NB or no drug for 72 hours, using the RNeasy and Qiashredder kits (Qiagen). Ten μg of RNA per sample were mixed with 1× SuperScript II First Strand buffer, 10 mM DTT, 400 μM dNTP mixture, 0.3 ng random hexamer primer, 2 μL RNaseOUT RNase inhibitor and 2 μL of SuperScript II reverse transcriptase in a 100 μL reaction (all components from Invitrogen). RT reactions were carried out by denaturing RNA at 70° C. for 10 minutes, plunging RNA on to ice, adding other components, incubating at 42° C. for 1 hour and heat inactivating the RT enzyme by a final incubation at 70° C. for 10 minutes.

For each sample, 82 μL of cDNA was combined with 328 μl of nuclease free water (Invitrogen) and an equal volume of TaqMan Universal PCR Master Mix No AmpErase UNG (Applied Biosystems). The mixture was loaded into each of 8 ports on the card at 100 μL per port. Each reaction contained forward and reverse primer at a final concentration of 900 nM and a TaqMan MGB probe (6-FAM) at 250 nM final concentration. The cards were sealed with a TaqMan Low-Density Array Sealer (Applied Biosystems) to prevent cross-contamination. The real-time RT-PCR amplifications were run on an ABI Prism 7900HT Sequence Detection System (Applied Biosystems) with a TaqMan Low Density Array Upgrade. Thermal cycling conditions were as follows: 2 min at 50° C., 10 min at 94.5° C., 40 cycles of 97° C. for 30 seconds, and annealing and extension at 59.7° C. for 1 minute. Each individual replicate cDNA sample was processed on a separate card.

Gene expression values were derived using SDS 2.2 software package (Applied Biosystems). Differential gene expression was calculated by the ΔΔCt method. Briefly, using threshold cycle (Ct) for each gene, change in gene expression was calculated for each sample comparison by the formulae:

ΔCt_{(test sample)}=Ct_{(target gene, test sample)}−Ct_{(reference gene, test sample)} 1.

ΔCt_{(control sample)}=Ct_{(target gene, control sample)}−Ct_{(reference gene, control sample)} 2.

ΔΔCt=ΔCt₍ₜₑₛₜ₎−ΔCt_{(calibrator)} 3.

(4) Semi-Quantitative PCR

Cells were cultured for two days at 39° C. in 10% FBS medium w/o interferon-γ on collagen IV-coated 15 cm dishes. Then, the cells were washed twice in PBS and cultured for an additional day w/o serum at 39° C. Cells were plated at the following densities: YAMC—321,430, Mp53/Ras—250,000, and Mp53/Ras derivatives—250,000. Cells were then trypsinized, pelleted down at 1,500 rpm for 5 minutes at 4° C., snap-frozen in liquid N₂ and stored at −80° C. Total RNA was extracted using Qiashredder and RNeasy Mini RNA extraction kits (Qiagen). Five μg of total RNA was used for reverse transcription reactions. The RNA was first mixed with 10 μL 5× First strand buffer, 5 μL 0.1 M dithiothrietol, 5 μL 10 pmol/μL random hexamers (Invitrogen) and 2 μL 10 mM dNTPs (Invitrogen) and denatured for 10 minutes at 70° C. After a quick chill on ice, 1 μL of Single Strand II reverse transcriptase (Invitrogen) and 1 μL of RNaseOUT (Invitrogen) were added to each reaction. Reverse transcription reactions were then incubated at 42° C. for one hour. Semi-quantitative PCR reactions were performed using 1 μL cDNA, 5 μL 10× Taq Polymerase buffer (−MgCl₂), 1.5 μL MgCl₂, 1.5 μL 10 μmol/μL forward and reverse primers, 2 μL DMSO, 1 μL 10 mM dNTPs, and 0.5 μL Taq Polymerase (Invitrogen). All primers used an annealing temperature of 58° C. All cDNAs were amplified for 32 cycles with the exception of GAPDH, which was amplified for 28 cycles.

SemiQuantitative RT-PCR primers used
mouse Dapk:
Forward:
(SEQ ID NO: 71)
5′-GGA GAC ACC AAG CAA GAA A-3′
Reverse:
(SEQ ID NO: 72)
5′-ACA AGG AGC CCA GGA GAT-3′
human Dapk1:
Forward:
(SEQ ID NO: 107)
5′-GGG TGT TTC GTC GAT TAT CAA GA-3′
Reverse:
(SEQ ID NO: 108)
5′-TCG CCC ATA CTT GTT GGA GAT-3′
mouse Dffb:
Forward:
(SEQ ID NO: 73)
5′-ACC CAA ATG CGT CAA GTT-3′
Reverse:
(SEQ ID NO: 74)
5′-GCT GCT TCA TCC ACC ATA-3′
mouse Elk3: (Same as SQ RT-PCR)
Forward:
(SEQ ID NO: 89)
5′-TCC TCA CGC GGT AGA GAT CAG-3′
Reverse:
(SEQ ID NO: 90)
5′-GTG GAG GTA CTC GTT GCG G-3′
mouse Etv1:
Forward:
(SEQ ID NO: 91)
5′-GCA AGT GCC TTA CGT GGT CA-3′
Reverse:
(SEQ ID NO: 92)
5′-GCT TCA GCA AGC CAT GTT TCT T-3′
mouse Fas receptor:
Forward:
(SEQ ID NO: 75)
5′-CCG AGA GTT TAA AGC TGA GG-3′
Reverse:
(SEQ ID NO: 76)
5′-CCA GGA GAA TCG CAG TAG AAG TCT GG-3′
human Fas receptor:
Forward:
(SEQ ID NO: 109)
5′-TAT CAC CAC TAT TGC TGG AGT CA-3′
Reverse:
(SEQ ID NO: 110)
5′-ACG AAG CAG TTG AAC TTT CTG TT-3′
mouse GAPDH:
Forward:
(SEQ ID NO: 77)
5′-ACC ACA GTC CAT GCC ATC AC-3′
Reverse:
(SEQ ID NO: 78)
5′-TCC ACC ACC CTG TTG CTG TA-3′
mouse Noxa:
Forward:
(SEQ ID NO: 79)
5′-TGA GTT CGC AGC TCA ACT C-3′
Reverse:
(SEQ ID NO: 80)
5′-TCA GGT TAC TAA ATT GAA GAG CTT GGA AAT C-3′
human Noxa:
Forward:
(SEQ ID NO: 111)
5′-TCT CAG GAG GTG CAC GTT TCA TCA-3′
Reverse:
(SEQ ID NO: 112)
5′-ATT CCA TCT TCC GTT TCC AAG GGC-3′
mouse Perp:
Forward:
(SEQ ID NO: 81)
5′-CCA CAT CCA GAC ATC GTC-3′
Reverse:
(SEQ ID NO: 82)
5′-TAC CAG GGA GAT GAT CTG G-3′
human Perp:
Forward:
(SEQ ID NO: 113)
5′-TGG TTG CAG TCT ACG GAC C-3′
Reverse:
(SEQ ID NO: 114)
5′-TCA GGA AGA CAA GCA TCT GGG-3′
mouse Reprimo:
Forward:
(SEQ ID NO: 83)
5′-TGA ATT CAG TGC TGG GC-3′
Reverse:
(SEQ ID NO: 84)
5′-CAC TGC CTC CAC CTC TTT AG-3′
mouse Sfrp2:
Forward:
(SEQ ID NO: 85)
5′-ATG ATG ATG ACA ACG ACA TAA TG-3′
Reverse:
(SEQ ID NO: 86)
5′-GAT GAC AAC GAC ATA ATG GAA ACG-3′
human Sfrp2:
Forward:
(SEQ ID NO: 115)
5′-ATG ACC TAG ACG AGA CCA TCC-3′
Reverse:
(SEQ ID NO: 116)
5′-GTC GCA CTC AAG CAT GTC G-3′
mouse Zac1:
Forward:
(SEQ ID NO: 87)
5′-ATC CTG TTC CTA CCT CAT ATG C-3′
Reverse:
(SEQ ID NO: 88)
5′-CTG GAT CTG CAA CTG AAA CT-3′

(5) Real-Time Quantitative PCR:

Total RNA was extracted using the RNeasy and Qiashredder kits (Qiagen). Five μg of RNA was mixed with 1× SuperScript II First Strand buffer, 10 mM DTT, 400 1 μM dNTP mixture, 0.15 ng random hexamer primer, 1 μL RNaseOUT RNase inhibitor and 1 μL of SuperScript II reverse transcriptase in a 50 μL reaction (all components from Invitrogen). RT reactions were carried out by denaturing RNA at 70° C. for 10 minutes, plunging RNA on to ice, adding other components, incubating at 42° C. for 1 hour and heat inactivating the RT enzyme by a final incubation at 70° C. for 10 minutes.

PCR reactions were prepared in triplicate using (per reaction) 1 μL cDNA (diluted 1:10), 1×SYBR Green Universal Master Mix (Bio-Rad), and 5 μmol forward and reverse primers in a 25 uL reaction volume. All primers sets, listed in Table 13, used an annealing temperature of 58° C. PCR reactions were run on an iCycler (Bio-Rad). Fluorescence intensity values were analyzed by the ΔΔCt method to generate relative fold expression values.

Real-time PCR primers used
mouse Dapk1: (Same as SQ RT-PCR)
Forward:
(SEQ ID NO: 71)
5′-GGA GAC ACC AAG CAA GAA A-3′
Reverse:
(SEQ ID NO: 72)
5′-ACA AGG AGC CCA GGA GAT-3′
mouse Dffb: (Same as SQ RT-PCR)
Forward:
(SEQ ID NO: 73)
5′-ACC CAA ATG CGT CAA GTT-3′
Reverse:
(SEQ ID NO: 74)
5′-GCT GCT TCA TCC ACC ATA-3′
mouse Elk3: (Same as SQ RT-PCR)
Forward:
(SEQ ID NO: 89)
5′-TCC TCA CGC GGT AGA GAT CAG-3′
Reverse:
(SEQ ID NO: 90)
5′-GTG GAG GTA CTC GTT GCG G-3′
mouse Etv1:
Forward:
(SEQ ID NO: 91)
5′-GCA AGT GCC TTA CGT GGT CA-3′
Reverse:
(SEQ ID NO: 92)
5′-GCT TCA GCA AGC CAT GTT TCT T-3′
mouse Fas receptor: (Same as SQ RT-PCR)
Forward:
(SEQ ID NO: 75)
5′-CCG AGA GTT TAA AGC TGA GG-3′
Reverse:
(SEQ ID NO: 76)
5′-CCA GGA GAA TCG CAG TAG AAG TCT GG-3′
mouse Noxa: (Same as SQ RT-PCR)
Forward:
(SEQ ID NO: 79)
5′-TGA GTT CGC AGC TCA ACT C-3′
Reverse:
(SEQ ID NO: 80)
5′-TCA GGT TAC TAA ATT GAA GAG CTT GGA AAT C-3′
mouse Perp:
Forward:
(SEQ ID NO: 93)
5′-ATG GAG TAC GCA TGG GGA C-3′
Reverse:
(SEQ ID NO: 94)
5′-GAT TAC CAG GGA GAT GAT CTG GA-3′
mouse Reprimo:
Forward:
(SEQ ID NO: 95)
5′-GTG TGG TGC AGA TCG CAG T-3′
Reverse:
(SEQ ID NO: 96)
5′-ATC ATG CCT TCG GAC TTG ATG-3′
mouse RhoA:
Forward:
(SEQ ID NO: 97)
5′-AGC TTG TGG TAA GAC ATG CTT G-3′
Reverse:
(SEQ ID NO: 98)
5′-GTG TCC CAT AAA GCC AAC TCT AC-3′
mouse Sfrp2:
Forward:
(SEQ ID NO: 99)
5′-CAT CGA GTA CCA GAA CAT GCG-3′
Reverse:
(SEQ ID NO: 100)
5′-GAA GAG CGA GCA CAG GAA CT-3′
mouse Zac1:
Forward:
(SEQ ID NO: 101)
5′-ACC TCA AGT CTC ACG CGG AAG AAA-3′
Reverse:
(SEQ ID NO: 102)
5′-TGA CAC AGG AAG TCC TTG CAT CCT-3′

(6) TUNEL Assay and Flow Cytometry Analysis:

Paraformaldehyde-fixed cells were pelleted and washed with PBS containing 0.1% BSA. Cells were permeabilized in 0.1% sodium citrate, 0.1% Triton X-100 for 2 minutes on ice. Cells were washed and re-suspended in 50 μL, of TUNEL enzyme and labeling solution (Roche) or 50 μL, of labeling solution alone as a negative control for one hour at 37° C. The positive control sample was first incubated for 10 minutes at room temperature with DNase enzyme (Invitrogen), washed and then re-suspended in 50 μL, of TUNEL enzyme with labeling solution. Following TUNEL labeling, cells were washed and re-suspended in PBS. TUNEL-stained cells were analyzed by flow cytometry using a FACScalibur (Becton Dickinson). The percentage of TUNEL-positive cells was analyzed using ModFit LT for Mac v2.0.

(7) Chromatin Immunoprecipitation and Promoter QPCR:

Cells were incubated at 37° C. for 15 minutes in the presence of 1% formaldehyde. This reaction was stopped with the addition of glycine to a final concentration of 0.125M and incubation at room temperature for five minutes. Cells were then washed 2 times with ice-cold PBS. Cells were scraped off of the dishes, pelleted and stored at −80° C. until ready for lysis and sonication. An Acetyl-Histone H3 Immunoprecipitation (ChIP) Assay Kit (Millipore) was then used according to the manufacturer's protocol. SYBR Green-based quantitative PCR was run using 1× Bio-Rad iQ SYBR Green master mix, 0.2 mM forward and reverse primer mix, with gene-specific qPCR primers for each gene tested. Reactions were run on the iCycler (Bio-Rad), as follows: 5 min at 95° C., 45 cycles of 95° C. for 30 seconds, 60° C. for 30 seconds, 72° C. for 45 seconds to amplify products, followed by 40 cycles of 94° C. with 1° C. step-down for 30 seconds to produce melt curves.

(8) Western Blotting:

mp53/Ras cells were grown at 39° C. for 2 days, followed by plating into 2.5 mM VA or NB for 3 days prior to lysis for Western blots. SW480 cells were grown in standard conditions, then plated into 2.5 mM VA or NB for 3 days prior to Western analysis. Cell pellets were lysed for 20 min at 4° C. with rotation in RIPA buffer (50 mM Tris-HCL, pH 7.4, 150 mM NaCL, 1% NP-40, 5 mM EDTA, 0.1% SDS, 0.5% deoxycholic acid, protease inhibitor cocktail tablet). Lysates were clarified by centrifugation at 13,000 g for 10 min at 4° C. and quantitated using Bradford protein assay (Bio-Rad). 25 μg of protein lysate was separated by SDS-PAGE and transferred to PVDF membrane (Millipore). Immunoblots were blocked in 5% non-fat dry milk in PBS with 0.2% Tween-20 for 1 hour at RT, probed with antibodies against p53 (FL-393, Santa Cruz) for all cell lines, H-Ras (C-20, Santa Cruz) for mp53/Ras cells, Raf (F-7, Santa Cruz) for HT-29 cells, Ras (Ab-1, Calbiochem) for DLD-1 cells, and tubulin (H-235, Santa Cruz) for all cell lines. Bands were visualized using the ECL+kit (Amersham).

(9) BrdU Labeling and Staining

Cells were cultured for two days at 39° C. in 10% FBS in the absence of interferon-γ on collagen IV-coated 10 cm dishes. Cells were then washed twice in PBS and cultured for an additional day at 39° C. without FBS or interferon-γ. Cells were finally labeled for 90 minutes with 10 μM bromodeoxyuridine (BrdU). Note: a separate plate of unlabeled cells served as a negative control. Cells were then trypsinized and washed in PBS. After the final spin, all but 200 μL of the PBS was aspirated and with gentle vortexing, 2 mL of cold 80% ethanol was added to each sample. Ethanol-fixed samples were then stored at 4° C. For BrdU/propidium iodide (PI) staining, cells were first spun out of ethanol at 2,500 rpm for 5 minutes, washed twice in PBS w/0.1% BSA and then incubated at room temperature for 30 minutes in 2M HCl with occasional vortexing. All subsequent spins were at 1,500 rpm, for 5 minutes at 4° C. Cells were again washed twice in PBS w/0.1% BSA and then permeabilized for 10 minutes at room temperature in PBS w/0.1% BSA, 0.1% Tween 20 (PBS-T) with occasional vortexing. Permeabilized cells were then incubated in a 1:10 dilution of monoclonal anti-BrdU antibody (Becton Dickinson) in a total volume of 100 μL of PBS-T for 20 minutes at room temperature. Cells were then washed twice in PBS-T and then incubated in 100 μL of PBS-T with 1.125 μL of anti-mouse Alexa Fluor 488 (Molecular Probes) for 20 minutes at room temperature. Cells were then washed twice in PBS and incubated for 15 minutes at room temperature in 100 μL of 100 μg/mL RNase in ddH₂O. Finally, cells were re-suspended in PBS with 10 μg/mL PI (Sigma). BrdU/PI-stained cells were analyzed by flow cytometry using the FLT-1 channel of a FASCalibur to measure anti-BrdU fluorescence intensity and the FLT-3 channel to measure PI fluorescence intensity. Cellquest software was used to analyze flow cytometry data.

### 4. Example 4

### Identification of Compounds Inhibiting Tumor Growth

a) Use of CRGs to Query the Connectivity Map Identifies Drugs that Abrogate the Malignant Phenotype.

The malignant phenotype is diminished by antagonism of individual or combinations of CRGs using either molecular genetic perturbations or treatment with histone deacetylase inhibitors (HDACi). Based on these observations, it is known that an important general characteristic of efficacious anti-cancer drugs is the ability to reverse the expression pattern of CRGs that results upon transformation. Since numerous studies indicate the utility of the gene expression-based strategies for identifying drugs that mimic or reverse biological states across different cell types and species (Hassane et al., 2008; Hieronymus et al., 2006; Hughes et al., 2000; Lamb et al., 2006; Stegmaier et al., 2004; Stegmaier et al., 2007; Wei et al., 2006), the CMap database (build 2.0) was queried for drug signatures that reverse the CRG signature.

b) Query of the Connectivity Map Database.

To facilitate rapid cross-species queries using human-specific Affymetrix IDs contained in the CMap, murine Affymetrix IDs for CRGs were mapped to gene symbols, which were then mapped to Affymetrix IDs contained within the CMap. All available probe sets were used when a many-to-one relationship existed between Affymetrix IDs and unique gene symbols. The query signature consisted of 23 up-regulated CRGs and 59 down-regulated CRGs for which gene symbol annotation was present in the CMap data set. Using the web-based Connectivity Map, the Kolmogorov-Smirnov-based gene set enrichment analysis (GSEA) algorithm (Subramanian et al., 2005) was used to obtain enrichment scores (ES) for both up-regulated (ESₚ) and down-regulated (ES_{down}) CRGs for each CMap drug treatment instance. The values of ESᵤₚ and ES_{down} are combined to generate a CMap “connectivity score” as described (Lamb et al., 2006). Drugs that mimic the CRG signature attain a positive connectivity score whereas drugs that oppose the CRG signature (and thereby are predicted as potential anti-cancer drugs) attain a negative connectivity score. Highly negatively connected drugs, with connectivity scores <−0.5 are indicated in Table 15. These compounds generally target both the up- and down-regulated CRG sets.

TABLE 15
Compounds predicted to reverse the overall CRG signature,
identified by the Connectivity Map

| Rank | Batch | CMap Name | Dose | | Cell | Score | ESup | ESdown | Instance_ID |
|---|---|---|---|---|---|---|---|---|---|
| 6100 | 692 | trichostatin A | 100 | nM | PC3 | −1 | −0.29 | 0.383 | 4184 |
| 6099 | 1009 | trichostatin A | 1 | μM | PC3 | −0.955 | −0.327 | 0.315 | 5950 |
| 6098 | 703 | rifabutin | 5 | μM | PC3 | −0.953 | −0.237 | 0.404 | 4527 |
| 6097 | 683 | trichostatin A | 100 | nM | PC3 | −0.933 | −0.307 | 0.321 | 3791 |
| 6096 | 689 | trichostatin A | 100 | nM | PC3 | −0.923 | −0.274 | 0.347 | 4072 |
| 6095 | 727 | trichostatin A | 1 | μM | PC3 | −0.876 | −0.352 | 0.238 | 4458 |
| 6094 | 754 | trichostatin A | 100 | nM | PC3 | −0.855 | −0.258 | 0.318 | 6340 |
| 6093 | 715 | trichostatin A | 100 | nM | PC3 | −0.838 | −0.245 | 0.319 | 6736 |
| 6092 | 56 | valproic acid | 1 | mM | PC3 | −0.821 | −0.355 | 0.197 | 433 |
| 6091 | 693 | trichostatin A | 100 | nM | PC3 | −0.808 | −0.244 | 0.3 | 4237 |
| 6090 | 728 | piretanide | 11 | μM | PC3 | −0.807 | −0.413 | 0.13 | 4490 |
| 6089 | 702 | trichostatin A | 100 | nM | PC3 | −0.804 | −0.225 | 0.316 | 4344 |
| 6088 | 727 | vorinostat | 10 | μM | PC3 | −0.784 | −0.265 | 0.263 | 4444 |
| 6087 | 1001 | trichostatin A | 1 | μM | PC3 | −0.783 | −0.252 | 0.275 | 5908 |
| 6086 | 1071 | trichostatin A | 1 | μM | PC3 | −0.778 | −0.207 | 0.317 | 7073 |
| 6085 | 750 | vorinostat | 10 | μM | HL60 | −0.773 | −0.334 | 0.186 | 6179 |
| 6084 | 1095 | trichostatin A | 1 | μM | PC3 | −0.765 | −0.274 | 0.241 | 7555 |
| 6083 | 648 | butirosin | 5 | μM | HL60 | −0.751 | −0.349 | 0.157 | 2518 |
| 6082 | 1032 | trichostatin A | 1 | μM | PC3 | −0.75 | −0.23 | 0.275 | 6546 |
| 6081 | 727 | trichostatin A | 100 | nM | PC3 | −0.738 | −0.223 | 0.274 | 4436 |
| 6080 | 1031 | trichostatin A | 1 | μM | PC3 | −0.736 | −0.17 | 0.325 | 6439 |
| 6079 | 713 | trichostatin A | 100 | nM | PC3 | −0.733 | −0.183 | 0.31 | 4665 |
| 6078 | 709 | trichostatin A | 100 | nM | PC3 | −0.731 | −0.208 | 0.284 | 6609 |
| 6077 | 688 | trichostatin A | 100 | nM | PC3 | −0.73 | −0.18 | 0.311 | 3993 |
| 6076 | 681 | trichostatin A | 100 | nM | PC3 | −0.729 | −0.111 | 0.38 | 3746 |
| 6074 | 710 | trichostatin A | 100 | nM | PC3 | −0.724 | −0.149 | 0.338 | 6671 |
| 6075 | 741 | lansoprazole | 11 | μM | MCF7 | −0.724 | −0.362 | 0.126 | 6009 |
| 6072 | 727 | valproic acid | 200 | μM | PC3 | −0.718 | −0.174 | 0.308 | 4438 |
| 6073 | 1007 | trichostatin A | 1 | μM | PC3 | −0.718 | −0.197 | 0.286 | 5940 |
| 6071 | 603 | valproic acid | 1 | mM | PC3 | −0.715 | −0.213 | 0.269 | 1209 |
| 6070 | 762 | trichostatin A | 100 | nM | PC3 | −0.705 | −0.202 | 0.272 | 7285 |
| 6069 | 1083 | trichostatin A | 1 | μM | PC3 | −0.703 | −0.219 | 0.254 | 7503 |
| 6068 | 753 | trichostatin A | 100 | nM | PC3 | −0.697 | −0.136 | 0.333 | 6316 |
| 6067 | 701 | trichostatin A | 100 | nM | PC3 | −0.696 | −0.24 | 0.228 | 4302 |
| 6066 | 1003 | PF-00562151-00 | 10 | μM | PC3 | −0.691 | −0.299 | 0.166 | 5922 |
| 6065 | 683 | spiradoline | 1 | μM | PC3 | −0.684 | −0.324 | 0.136 | 3818 |
| 6064 | 63 | valproic acid | 1 | mM | PC3 | −0.683 | −0.288 | 0.172 | 458 |
| 6063 | 55 | troglitazone | 10 | μM | PC3 | −0.682 | −0.344 | 0.115 | 431 |
| 6062 | 603 | valproic acid | 500 | μM | PC3 | −0.68 | −0.142 | 0.315 | 1240 |
| 6061 | 1062 | scriptaid | 10 | μM | PC3 | −0.679 | −0.229 | 0.227 | 6919 |
| 6060 | 733 | ticarcillin | 9 | μM | PC3 | −0.678 | −0.259 | 0.197 | 5829 |
| 6059 | 648 | napelline | 11 | μM | HL60 | −0.677 | −0.216 | 0.24 | 2522 |
| 6058 | 1065 | trichostatin A | 1 | μM | PC3 | −0.675 | −0.192 | 0.262 | 7047 |
| 6057 | 1052 | trichostatin A | 1 | μM | PC3 | −0.673 | −0.252 | 0.201 | 6886 |
| 6056 | 704 | trichostatin A | 100 | nM | PC3 | −0.672 | −0.117 | 0.335 | 4565 |
| 6054 | 658 | beclometasone | 8 | μM | HL60 | −0.669 | −0.194 | 0.256 | 3001 |
| 6055 | 1073 | trichostatin A | 1 | μM | PC3 | −0.669 | −0.216 | 0.234 | 7077 |
| 6053 | 650 | trichostatin A | 1 | μM | HL60 | −0.667 | −0.233 | 0.216 | 2694 |
| 6052 | 615 | trichostatin A | 100 | nM | HL60 | −0.667 | −0.258 | 0.191 | 1421 |
| 6050 | 648 | estropipate | 9 | μM | HL60 | −0.666 | −0.17 | 0.278 | 2506 |
| 6051 | 650 | vorinostat | 10 | μM | HL60 | −0.666 | −0.251 | 0.197 | 2680 |
| 6049 | 650 | chlorpromazine | 1 | μM | HL60 | −0.659 | −0.235 | 0.208 | 2677 |
| 6048 | 683 | CP-690334-01 | 10 | μM | PC3 | −0.659 | −0.267 | 0.176 | 3823 |
| 6047 | 612 | hexamethonium | 10 | μM | HL60 | −0.658 | −0.263 | 0.18 | 1982 |
| | | bromide | | | | | | | |
| 6046 | 750 | trichostatin A | 1 | μM | HL60 | −0.656 | −0.267 | 0.174 | 6193 |
| 6045 | 761 | trichostatin A | 100 | nM | PC3 | −0.655 | −0.169 | 0.272 | 7245 |
| 6044 | 750 | LY-294002 | 10 | μM | HL60 | −0.655 | −0.337 | 0.103 | 6186 |
| 6043 | 750 | alpha-estradiol | 10 | nM | HL60 | −0.654 | −0.257 | 0.182 | 6169 |
| 6042 | 665 | trichostatin A | 100 | nM | HL60 | −0.652 | −0.16 | 0.278 | 2949 |
| 6039 | 614 | nalbuphine | 10 | μM | HL60 | −0.65 | −0.216 | 0.221 | 1379 |
| 6040 | 613 | trichostatin A | 100 | nM | HL60 | −0.65 | −0.223 | 0.215 | 2035 |
| 6041 | 602 | trichostatin A | 1 | μM | HL60 | −0.65 | −0.263 | 0.175 | 1175 |
| 6038 | 646 | terbutaline | 7 | μM | MCF7 | −0.646 | −0.315 | 0.12 | 3202 |
| 6037 | 664 | sitosterol | 10 | μM | HL60 | −0.645 | −0.192 | 0.242 | 2912 |
| 6036 | 623 | trichostatin A | 100 | nM | HL60 | −0.643 | −0.22 | 0.213 | 1612 |
| 6035 | 693 | carcinine | 22 | μM | PC3 | −0.643 | −0.278 | 0.154 | 4225 |
| 6034 | 661 | protriptyline | 13 | μM | HL60 | −0.642 | −0.233 | 0.199 | 3119 |
| 6033 | 767 | sirolimus | 100 | nM | MCF7 | −0.641 | −0.345 | 0.087 | 6958 |
| 6032 | 719 | trichostatin A | 100 | nM | PC3 | −0.64 | −0.178 | 0.253 | 5086 |
| 6031 | 714 | trichostatin A | 100 | nM | PC3 | −0.638 | −0.158 | 0.271 | 6709 |
| 6030 | 615 | meclofenamic acid | 12 | μM | HL60 | −0.637 | −0.193 | 0.235 | 1445 |
| 6029 | 683 | diethylstilbestrol | 15 | μM | PC3 | −0.636 | −0.253 | 0.175 | 3812 |
| 6028 | 758 | biperiden | 11 | μM | MCF7 | −0.635 | −0.227 | 0.2 | 5644 |
| 6027 | 645 | famprofazone | 11 | μM | HL60 | −0.633 | −0.159 | 0.268 | 2174 |
| 6025 | 660 | trichostatin A | 100 | nM | HL60 | −0.632 | −0.086 | 0.339 | 3077 |
| 6026 | 741 | thalidomide | 15 | μM | MCF7 | −0.632 | −0.257 | 0.168 | 5990 |
| 6024 | 612 | idoxuridine | 11 | μM | HL60 | −0.628 | −0.263 | 0.16 | 1980 |
| 6023 | 615 | alverine | 8 | μM | HL60 | −0.627 | −0.247 | 0.175 | 1426 |
| 6022 | 646 | bambuterol | 10 | μM | MCF7 | −0.627 | −0.261 | 0.16 | 3199 |
| 6020 | 617 | nimesulide | 13 | μM | PC3 | −0.626 | −0.236 | 0.185 | 2112 |
| 6021 | 650 | LY-294002 | 10 | μM | HL60 | −0.626 | −0.275 | 0.147 | 2696 |
| 6019 | 1079 | trichostatin A | 1 | μM | PC3 | −0.623 | −0.191 | 0.229 | 7105 |
| 6018 | 750 | trifluoperazine | 10 | μM | HL60 | −0.623 | −0.257 | 0.163 | 6183 |
| 6017 | 35 | trichostatin A | 100 | nM | HL60 | −0.619 | −0.213 | 0.204 | 364 |
| 6015 | 737 | gemfibrozil | 16 | μM | MCF7 | −0.619 | −0.281 | 0.136 | 5488 |
| 6016 | 686 | indapamide | 11 | μM | MCF7 | −0.619 | −0.307 | 0.11 | 3859 |
| 6014 | 632 | 4-hydroxyphenazone | 20 | μM | MCF7 | −0.618 | −0.29 | 0.126 | 1497 |
| 6012 | 698 | trichostatin A | 100 | nM | PC3 | −0.617 | −0.145 | 0.27 | 7387 |
| 6013 | 630 | buspirone | 9 | μM | HL60 | −0.617 | −0.259 | 0.156 | 1282 |
| 6011 | 731 | trichostatin A | 100 | nM | PC3 | −0.616 | −0.131 | 0.283 | 5745 |
| 6010 | 632 | naphazoline | 16 | μM | MCF7 | −0.615 | −0.285 | 0.128 | 1466 |
| 6009 | 750 | alvespimycin | 100 | nM | HL60 | −0.614 | −0.201 | 0.212 | 6172 |
| 6008 | 762 | iobenguane | 11 | μM | PC3 | −0.614 | −0.229 | 0.184 | 7299 |
| 6007 | 651 | methazolamide | 17 | μM | HL60 | −0.613 | −0.225 | 0.187 | 2733 |
| 6006 | 771 | pinacidil | 16 | μM | MCF7 | −0.612 | −0.308 | 0.104 | 7437 |
| 6005 | 629 | trichostatin A | 100 | nM | HL60 | −0.611 | −0.128 | 0.283 | 1835 |
| 6004 | 692 | probenecid | 14 | μM | PC3 | −0.61 | −0.316 | 0.095 | 4185 |
| 6002 | 728 | trichostatin A | 100 | nM | PC3 | −0.609 | −0.165 | 0.245 | 4483 |
| 6003 | 750 | valproic acid | 500 | μM | HL60 | −0.609 | −0.217 | 0.193 | 6199 |
| 6001 | 623 | vanoxerine | 8 | μM | HL60 | −0.608 | −0.2 | 0.209 | 1625 |
| 6000 | 623 | methyldopa | 19 | μM | HL60 | −0.607 | −0.185 | 0.224 | 1619 |
| 5999 | 612 | naphazoline | 16 | μM | HL60 | −0.606 | −0.223 | 0.185 | 1966 |
| 5998 | 733 | trichostatin A | 100 | nM | PC3 | −0.605 | −0.136 | 0.271 | 5822 |
| 5997 | 630 | flupentixol | 8 | μM | HL60 | −0.605 | −0.138 | 0.269 | 1288 |
| 5994 | 650 | valproic acid | 1 | mM | HL60 | −0.602 | −0.247 | 0.158 | 2669 |
| 5996 | 692 | naftopidil | 9 | μM | PC3 | −0.602 | −0.304 | 0.101 | 4193 |
| 5995 | 705 | ethionamide | 24 | μM | MCF7 | −0.602 | −0.32 | 0.085 | 4418 |
| 5993 | 631 | bacampicillin | 8 | μM | HL60 | −0.601 | −0.191 | 0.213 | 1337 |
| 5992 | 19 | LY-294002 | 10 | μM | MCF7 | −0.601 | −0.287 | 0.117 | 258 |
| 5991 | 650 | valproic acid | 500 | μM | HL60 | −0.599 | −0.218 | 0.185 | 2700 |
| 5989 | 734 | vidarabine | 15 | μM | PC3 | −0.598 | −0.234 | 0.168 | 5850 |
| 5990 | 654 | SR-95531 | 11 | μM | MCF7 | −0.598 | −0.282 | 0.12 | 3253 |
| 5988 | 660 | tyloxapol | 4 | μM | HL60 | −0.597 | −0.196 | 0.206 | 3074 |
| 5985 | 762 | epirizole | 17 | μM | PC3 | −0.596 | −0.197 | 0.204 | 7292 |
| 5986 | 1054 | scriptaid | 10 | μM | PC3 | −0.596 | −0.247 | 0.154 | 6896 |
| 5987 | 715 | lynestrenol | 14 | μM | PC3 | −0.596 | −0.295 | 0.106 | 6756 |
| 5984 | 603 | trichostatin A | 100 | nM | PC3 | −0.594 | −0.128 | 0.272 | 1212 |
| 5982 | 734 | trichostatin A | 100 | nM | PC3 | −0.594 | −0.153 | 0.247 | 5882 |
| 5980 | 641 | cinchonidine | 14 | μM | HL60 | −0.594 | −0.186 | 0.213 | 1780 |
| 5983 | 703 | 2,6-dimethylpiperidine | 27 | μM | PC3 | −0.594 | −0.254 | 0.146 | 4543 |
| 5979 | 44 | valproic acid | 10 | mM | HL60 | −0.594 | −0.274 | 0.126 | 410 |
| 5981 | 610 | pheniramine | 11 | μM | PC3 | −0.594 | −0.318 | 0.081 | 1910 |
| 5978 | 650 | trichostatin A | 100 | nM | HL60 | −0.593 | −0.163 | 0.236 | 2672 |
| 5977 | 771 | niflumic acid | 14 | μM | MCF7 | −0.593 | −0.304 | 0.095 | 7430 |
| 5976 | 751 | diphenylpyraline | 13 | μM | MCF7 | −0.591 | −0.254 | 0.144 | 6061 |
| 5975 | 602 | vorinostat | 10 | μM | HL60 | −0.591 | −0.253 | 0.144 | 1161 |
| 5974 | 736 | piribedil | 12 | μM | MCF7 | −0.59 | −0.286 | 0.111 | 5434 |
| 5973 | 640 | laudanosine | 11 | μM | HL60 | −0.589 | −0.152 | 0.245 | 1741 |
| 5972 | 622 | ketotifen | 9 | μM | HL60 | −0.589 | −0.169 | 0.227 | 1583 |
| 5971 | 659 | trichostatin A | 100 | nM | HL60 | −0.589 | −0.212 | 0.184 | 3058 |
| 5970 | 646 | mepacrine | 8 | μM | MCF7 | −0.586 | −0.16 | 0.234 | 3179 |
| 5969 | 513 | fulvestrant | 10 | nM | MCF7 | −0.585 | −0.27 | 0.124 | 1076 |
| 5968 | 513 | wortmannin | 10 | nM | MCF7 | −0.584 | −0.256 | 0.137 | 1081 |
| 5965 | 644 | solanine | 5 | μM | HL60 | −0.582 | −0.18 | 0.211 | 2152 |
| 5967 | 699 | atractyloside | 5 | μM | MCF7 | −0.582 | −0.22 | 0.172 | 4717 |
| 5966 | 690 | canadine | 12 | μM | MCF7 | −0.582 | −0.264 | 0.128 | 4138 |
| 5964 | 1015 | trichostatin A | 1 | μM | PC3 | −0.581 | −0.197 | 0.195 | 5981 |
| 5963 | 614 | trichostatin A | 100 | nM | HL60 | −0.581 | −0.252 | 0.139 | 1400 |
| 5961 | 683 | pramocaine | 12 | μM | PC3 | −0.58 | −0.192 | 0.198 | 3811 |
| 5962 | 762 | ketorolac | 11 | μM | PC3 | −0.58 | −0.235 | 0.155 | 7286 |
| 5960 | 612 | diflunisal | 16 | μM | HL60 | −0.58 | −0.236 | 0.154 | 1990 |
| 5959 | 618 | metoclopramide | 12 | μM | HL60 | −0.579 | −0.221 | 0.168 | 2353 |
| 5957 | 712 | trichostatin A | 100 | nM | PC3 | −0.578 | −0.133 | 0.256 | 4632 |
| 5958 | 612 | lidocaine | 15 | μM | HL60 | −0.578 | −0.18 | 0.209 | 1999 |
| 5956 | 701 | PNU-0230031 | 1 | μM | PC3 | −0.578 | −0.322 | 0.067 | 4291 |
| 5955 | 505 | 5186223 | 12 | μM | MCF7 | −0.577 | −0.256 | 0.132 | 885 |
| 5953 | 614 | dihydroergotamine | 3 | μM | HL60 | −0.575 | −0.197 | 0.19 | 1398 |
| 5951 | 640 | mometasone | 8 | μM | HL60 | −0.575 | −0.2 | 0.186 | 1746 |
| 5954 | 641 | calycanthine | 12 | μM | HL60 | −0.575 | −0.248 | 0.139 | 1771 |
| 5952 | 671 | iopromide | 5 | μM | MCF7 | −0.575 | −0.298 | 0.089 | 3481 |
| 5950 | 762 | gliquidone | 8 | μM | PC3 | −0.574 | −0.194 | 0.192 | 7301 |
| 5949 | 698 | monensin | 6 | μM | PC3 | −0.574 | −0.317 | 0.069 | 7402 |
| 5948 | 650 | trifluoperazine | 10 | μM | HL60 | −0.573 | −0.195 | 0.19 | 2684 |
| 5947 | 694 | gabexate | 10 | μM | MCF7 | −0.573 | −0.238 | 0.148 | 4804 |
| 5946 | 642 | vincamine | 11 | μM | MCF7 | −0.572 | −0.227 | 0.158 | 2327 |
| 5945 | 719 | bufexamac | 18 | μM | PC3 | −0.571 | −0.185 | 0.199 | 5090 |
| 5944 | 1004 | fulvestrant | 1 | μM | MCF7 | −0.571 | −0.221 | 0.164 | 5926 |
| 5942 | 703 | Prestwick-1100 | 9 | μM | PC3 | −0.571 | −0.272 | 0.112 | 4534 |
| 5943 | 767 | wortmannin | 10 | nM | MCF7 | −0.571 | −0.274 | 0.11 | 6959 |
| 5940 | 736 | iopanoic acid | 7 | μM | MCF7 | −0.57 | −0.253 | 0.13 | 5448 |
| 5941 | 710 | famotidine | 12 | μM | PC3 | −0.57 | −0.308 | 0.076 | 6665 |
| 5939 | 748 | trichostatin A | 100 | nM | MCF7 | −0.569 | −0.247 | 0.136 | 7236 |
| 5937 | 644 | trichostatin A | 100 | nM | HL60 | −0.568 | −0.176 | 0.206 | 2137 |
| 5938 | 765 | valproic acid | 500 | μM | MCF7 | −0.568 | −0.258 | 0.125 | 6999 |
| 5936 | 754 | isradipine | 11 | μM | PC3 | −0.568 | −0.271 | 0.111 | 6347 |
| 5935 | 714 | propofol | 22 | μM | PC3 | −0.567 | −0.279 | 0.103 | 6707 |
| 5932 | 1033 | trichostatin A | 1 | μM | MCF7 | −0.566 | −0.143 | 0.237 | 6551 |
| 5934 | 690 | cinchonine | 14 | μM | MCF7 | −0.566 | −0.203 | 0.178 | 4107 |
| 5933 | 741 | chenodeoxycholic acid | 10 | μM | MCF7 | −0.566 | −0.247 | 0.134 | 6012 |
| 5928 | 617 | trichostatin A | 100 | nM | PC3 | −0.565 | −0.13 | 0.25 | 2105 |
| 5930 | 659 | phthalylsulfathiazole | 10 | μM | HL60 | −0.565 | −0.145 | 0.236 | 3033 |
| 5931 | 632 | dicycloverine | 12 | μM | MCF7 | −0.565 | −0.293 | 0.087 | 1483 |
| 5929 | 766 | thiamphenicol | 11 | μM | MCF7 | −0.565 | −0.297 | 0.083 | 7033 |
| 5925 | 622 | tremorine | 15 | μM | HL60 | −0.564 | −0.15 | 0.229 | 1579 |
| 5926 | 612 | ticlopidine | 13 | μM | HL60 | −0.564 | −0.217 | 0.162 | 1975 |
| 5927 | 727 | haloperidol | 10 | μM | PC3 | −0.564 | −0.251 | 0.129 | 4468 |
| 5924 | 612 | trichostatin A | 100 | nM | HL60 | −0.562 | −0.243 | 0.135 | 1971 |
| 5923 | 715 | zidovudine | 15 | μM | PC3 | −0.562 | −0.254 | 0.124 | 6733 |
| 5922 | 651 | mevalolactone | 31 | μM | HL60 | −0.559 | −0.142 | 0.234 | 2718 |
| 5921 | 603 | valproic acid | 200 | μM | PC3 | −0.559 | −0.173 | 0.203 | 1214 |
| 5920 | 649 | eucatropine | 12 | μM | HL60 | −0.559 | −0.18 | 0.195 | 2556 |
| 5917 | 718 | flufenamic acid | 14 | μM | PC3 | −0.558 | −0.222 | 0.153 | 5059 |
| 5919 | 665 | etomidate | 16 | μM | HL60 | −0.558 | −0.255 | 0.121 | 2958 |
| 5918 | 701 | 0179445-0000 | 1 | μM | PC3 | −0.558 | −0.299 | 0.077 | 4292 |
| 5915 | 661 | trichostatin A | 100 | nM | HL60 | −0.556 | −0.155 | 0.219 | 3114 |
| 5914 | 602 | valproic acid | 500 | μM | HL60 | −0.556 | −0.184 | 0.19 | 1181 |
| 5912 | 641 | 1,4-chrysenequinone | 15 | μM | HL60 | −0.556 | −0.185 | 0.189 | 1773 |
| 5913 | 623 | methylergometrine | 9 | μM | HL60 | −0.556 | −0.204 | 0.17 | 1607 |
| 5916 | 689 | betulinic acid | 9 | μM | PC3 | −0.556 | −0.293 | 0.081 | 4101 |
| 5905 | 661 | scopoletin | 21 | μM | HL60 | −0.555 | −0.172 | 0.201 | 3131 |
| 5910 | 749 | benzylpenicillin | 11 | μM | HL60 | −0.555 | −0.174 | 0.2 | 6155 |
| 5911 | 762 | phenindione | 18 | μM | PC3 | −0.555 | −0.187 | 0.187 | 7289 |
| 5906 | 771 | lisinopril | 9 | μM | MCF7 | −0.555 | −0.207 | 0.166 | 7403 |
| 5909 | 692 | isoxsuprine | 12 | μM | PC3 | −0.555 | −0.212 | 0.161 | 4205 |
| 5907 | 670 | atractyloside | 5 | μM | MCF7 | −0.555 | −0.255 | 0.119 | 3435 |
| 5908 | 692 | epitiostanol | 13 | μM | PC3 | −0.555 | −0.29 | 0.083 | 4204 |
| 5900 | 641 | yohimbine | 10 | μM | HL60 | −0.554 | −0.169 | 0.204 | 1763 |
| 5901 | 750 | fluphenazine | 10 | μM | HL60 | −0.554 | −0.24 | 0.133 | 6196 |
| 5899 | 735 | carbimazole | 21 | μM | MCF7 | −0.554 | −0.249 | 0.124 | 5399 |
| 5903 | 693 | seneciphylline | 12 | μM | PC3 | −0.554 | −0.26 | 0.113 | 4238 |
| 5902 | 750 | 15-delta prostaglandin | 10 | μM | HL60 | −0.554 | −0.281 | 0.092 | 6190 |
| | | J2 | | | | | | | |
| 5904 | 702 | indapamide | 11 | μM | PC3 | −0.554 | −0.281 | 0.092 | 4335 |
| 5898 | 690 | chlorogenic acid | 11 | μM | MCF7 | −0.553 | −0.216 | 0.156 | 4142 |
| 5896 | 645 | diphenylpyraline | 13 | μM | HL60 | −0.552 | −0.254 | 0.118 | 2205 |
| 5897 | 692 | galantamine | 11 | μM | PC3 | −0.552 | −0.269 | 0.102 | 4186 |
| 5895 | 602 | LY-294002 | 10 | μM | HL60 | −0.552 | −0.279 | 0.092 | 1180 |
| 5894 | 659 | fluvastatin | 9 | μM | HL60 | −0.551 | −0.102 | 0.269 | 3032 |
| 5893 | 702 | proglumide | 12 | μM | PC3 | −0.551 | −0.27 | 0.101 | 4337 |
| 5892 | 626 | LY-294002 | 10 | μM | MCF7 | −0.55 | −0.244 | 0.127 | 1652 |
| 5891 | 692 | idoxuridine | 11 | μM | PC3 | −0.549 | −0.221 | 0.149 | 4200 |
| 5890 | 623 | methapyrilene | 13 | μM | HL60 | −0.549 | −0.224 | 0.145 | 1588 |
| 5889 | 1048 | SC-560 | 10 | μM | PC3 | −0.549 | −0.299 | 0.071 | 6865 |
| 5888 | 658 | roxithromycin | 5 | μM | HL60 | −0.548 | −0.127 | 0.242 | 2992 |
| 5887 | 725 | vorinostat | 10 | μM | MCF7 | −0.548 | −0.141 | 0.227 | 5217 |
| 5886 | 612 | thioridazine | 10 | μM | HL60 | −0.547 | −0.212 | 0.156 | 1986 |
| 5885 | 1032 | dinoprostone | 10 | μM | PC3 | −0.546 | −0.225 | 0.142 | 6547 |
| 5883 | 641 | (+)-chelidonine | 11 | μM | HL60 | −0.546 | −0.248 | 0.119 | 1786 |
| 5884 | 1068 | SB-203580 | 1 | μM | MCF7 | −0.546 | −0.285 | 0.083 | 7061 |
| 5882 | 650 | LY-294002 | 10 | μM | HL60 | −0.545 | −0.243 | 0.123 | 2687 |
| 5881 | 632 | sulfathiazole | 16 | μM | MCF7 | −0.544 | −0.259 | 0.106 | 1463 |
| 5880 | 505 | wortmannin | 10 | nM | MCF7 | −0.544 | −0.267 | 0.099 | 911 |
| 5878 | 645 | halcinonide | 9 | μM | HL60 | −0.543 | −0.162 | 0.204 | 2185 |
| 5877 | 747 | cinchonidine | 14 | μM | MCF7 | −0.543 | −0.233 | 0.132 | 7190 |
| 5879 | 712 | droperidol | 11 | μM | PC3 | −0.543 | −0.258 | 0.107 | 4629 |
| 5876 | 654 | SR-95639A | 10 | μM | MCF7 | −0.542 | −0.275 | 0.089 | 3272 |
| 5875 | 622 | fendiline | 11 | μM | HL60 | −0.541 | −0.227 | 0.137 | 1573 |
| 5874 | 648 | altizide | 10 | μM | HL60 | −0.54 | −0.177 | 0.186 | 2527 |
| 5869 | 615 | oxolinic acid | 15 | μM | HL60 | −0.539 | −0.188 | 0.174 | 1419 |
| 5870 | 610 | levodopa | 20 | μM | PC3 | −0.539 | −0.214 | 0.149 | 1892 |
| 5871 | 689 | carbenoxolone | 7 | μM | PC3 | −0.539 | −0.22 | 0.142 | 4093 |
| 5873 | 750 | prochlorperazine | 10 | μM | HL60 | −0.539 | −0.222 | 0.141 | 6174 |
| 5872 | 767 | fulvestrant | 10 | nM | MCF7 | −0.539 | −0.253 | 0.109 | 6955 |
| 5867 | 1089 | pioglitazone | 10 | μM | PC3 | −0.538 | −0.184 | 0.178 | 7528 |
| 5865 | 623 | amikacin | 7 | μM | HL60 | −0.538 | −0.185 | 0.176 | 1618 |
| 5866 | 612 | sulfaguanidine | 19 | μM | HL60 | −0.538 | −0.234 | 0.127 | 1995 |
| 5864 | 712 | betaxolol | 12 | μM | PC3 | −0.538 | −0.283 | 0.078 | 4608 |
| 5868 | 617 | tiratricol | 6 | μM | PC3 | −0.538 | −0.298 | 0.065 | 2096 |
| 5862 | 641 | dacarbazine | 22 | μM | HL60 | −0.537 | −0.136 | 0.225 | 1762 |
| 5863 | 56 | sodium phenylbutyrate | 1 | mM | PC3 | −0.537 | −0.17 | 0.191 | 434 |
| 5859 | 750 | monorden | 100 | nM | HL60 | −0.536 | −0.219 | 0.142 | 6178 |
| 5861 | 686 | fludrocortisone | 9 | μM | MCF7 | −0.536 | −0.243 | 0.118 | 3866 |
| 5860 | 744 | ampyrone | 20 | μM | MCF7 | −0.536 | −0.252 | 0.108 | 6845 |
| 5858 | 602 | thioridazine | 10 | μM | HL60 | −0.535 | −0.193 | 0.166 | 1171 |
| 5857 | 617 | norfloxacin | 13 | μM | PC3 | −0.535 | −0.245 | 0.115 | 2090 |
| 5856 | 700 | gossypol | 8 | μM | MCF7 | −0.535 | −0.276 | 0.084 | 4762 |
| 5855 | 614 | naltrexone | 10 | μM | HL60 | −0.534 | −0.203 | 0.157 | 1363 |
| 5854 | 513 | LY-294002 | 10 | μM | MCF7 | −0.534 | −0.273 | 0.086 | 1065 |
| 5853 | 734 | praziquantel | 13 | μM | PC3 | −0.534 | −0.275 | 0.084 | 5874 |
| 5851 | 665 | rimexolone | 11 | μM | HL60 | −0.533 | −0.136 | 0.223 | 2955 |
| 5846 | 750 | sirolimus | 100 | nM | HL60 | −0.533 | −0.193 | 0.166 | 6201 |
| 5847 | 1094 | trichostatin A | 1 | μM | MCF7 | −0.533 | −0.194 | 0.164 | 7550 |
| 5848 | 654 | piperine | 14 | μM | MCF7 | −0.533 | −0.219 | 0.14 | 3263 |
| 5849 | 756 | pirlindole | 12 | μM | MCF7 | −0.533 | −0.234 | 0.125 | 6519 |
| 5850 | 610 | prednisone | 11 | μM | PC3 | −0.533 | −0.241 | 0.118 | 1897 |
| 5852 | 692 | pepstatin | 6 | μM | PC3 | −0.533 | −0.241 | 0.117 | 4206 |
| 5845 | 750 | valproic acid | 200 | μM | HL60 | −0.532 | −0.18 | 0.178 | 6173 |
| 5844 | 1059 | trichostatin A | 1 | μM | MCF7 | −0.532 | −0.185 | 0.173 | 6910 |
| 5843 | 698 | clemizole | 11 | μM | PC3 | −0.531 | −0.182 | 0.175 | 7371 |
| 5842 | 1050 | trichostatin A | 1 | μM | PC3 | −0.53 | −0.172 | 0.184 | 6874 |
| 5841 | 681 | demeclocycline | 8 | μM | PC3 | −0.53 | −0.191 | 0.165 | 3706 |
| 5838 | 661 | ursodeoxycholic acid | 10 | μM | HL60 | −0.529 | −0.162 | 0.193 | 3105 |
| 5840 | 642 | orphenadrine | 13 | μM | MCF7 | −0.529 | −0.204 | 0.152 | 2318 |
| 5839 | 682 | proglumide | 12 | μM | PC3 | −0.529 | −0.241 | 0.115 | 3780 |
| 5837 | 21 | genistein | 1 | μM | MCF7 | −0.529 | −0.299 | 0.056 | 267 |
| 5835 | 693 | amprolium | 13 | μM | PC3 | −0.528 | −0.241 | 0.114 | 4241 |
| 5836 | 698 | pentolonium | 7 | μM | PC3 | −0.528 | −0.258 | 0.097 | 7375 |
| 5834 | 614 | acenocoumarol | 11 | μM | HL60 | −0.527 | −0.168 | 0.187 | 1394 |
| 5833 | 86 | fisetin | 50 | μM | PC3 | −0.527 | −0.174 | 0.18 | 579 |
| 5832 | 720 | thiamazole | 35 | μM | MCF7 | −0.527 | −0.239 | 0.115 | 4372 |
| 5831 | 682 | lanatoside C | 4 | μM | PC3 | −0.526 | −0.203 | 0.151 | 3771 |
| 5828 | 648 | cefalotin | 10 | μM | HL60 | −0.525 | −0.12 | 0.233 | 2517 |
| 5829 | 634 | naringin | 7 | μM | HL60 | −0.525 | −0.124 | 0.23 | 2425 |
| 5830 | 749 | trichostatin A | 100 | nM | HL60 | −0.525 | −0.222 | 0.131 | 6143 |
| 5827 | 664 | fluticasone | 8 | μM | HL60 | −0.524 | −0.096 | 0.257 | 2928 |
| 5826 | 602 | tanespimycin | 1 | μM | HL60 | −0.524 | −0.125 | 0.228 | 1159 |
| 5825 | 757 | sirolimus | 100 | nM | MCF7 | −0.524 | −0.17 | 0.182 | 5602 |
| 5823 | 1061 | trichostatin A | 1 | μM | MCF7 | −0.522 | −0.182 | 0.169 | 6916 |
| 5824 | 753 | amoxicillin | 11 | μM | PC3 | −0.522 | −0.187 | 0.164 | 6285 |
| 5822 | 753 | terguride | 12 | μM | PC3 | −0.521 | −0.241 | 0.11 | 6299 |
| 5821 | 734 | glibenclamide | 8 | μM | PC3 | −0.521 | −0.292 | 0.058 | 5849 |
| 5820 | 749 | oxprenolol | 13 | μM | HL60 | −0.519 | −0.158 | 0.191 | 6145 |
| 5817 | 689 | co-dergocrine mesilate | 6 | μM | PC3 | −0.519 | −0.222 | 0.127 | 4071 |
| 5818 | 613 | baclofen | 19 | μM | HL60 | −0.519 | −0.237 | 0.112 | 2036 |
| 5819 | 26b | arachidonyltrifluoromethane | 10 | μM | MCF7 | −0.519 | −0.258 | 0.092 | 327 |
| 5816 | 612 | niclosamide | 12 | μM | HL60 | −0.518 | −0.134 | 0.215 | 1998 |
| 5815 | 658 | fosfosal | 18 | μM | HL60 | −0.518 | −0.134 | 0.214 | 2997 |
| 5811 | 690 | boldine | 12 | μM | MCF7 | −0.517 | −0.234 | 0.114 | 4122 |
| 5813 | 772 | esculetin | 22 | μM | MCF7 | −0.517 | −0.237 | 0.111 | 7459 |
| 5810 | 709 | liothyronine | 6 | μM | PC3 | −0.517 | −0.237 | 0.111 | 6602 |
| 5812 | 710 | lisuride | 12 | μM | PC3 | −0.517 | −0.245 | 0.103 | 6682 |
| 5814 | 699 | guanadrel | 8 | μM | MCF7 | −0.517 | −0.249 | 0.099 | 4720 |
| 5809 | 649 | medrysone | 12 | μM | HL60 | −0.516 | −0.094 | 0.253 | 2544 |
| 5808 | 614 | mefloquine | 10 | μM | HL60 | −0.516 | −0.18 | 0.167 | 1364 |
| 5806 | 1078 | 0198306-0000 | 10 | μM | MCF7 | −0.516 | −0.223 | 0.125 | 7099 |
| 5805 | 732 | azlocillin | 8 | μM | PC3 | −0.516 | −0.241 | 0.106 | 5788 |
| 5807 | 692 | spectinomycin | 10 | μM | PC3 | −0.516 | −0.259 | 0.088 | 4187 |
| 5804 | 762 | homochlorcyclizine | 10 | μM | PC3 | −0.516 | −0.262 | 0.085 | 7295 |
| 5800 | 622 | chlortalidone | 12 | μM | HL60 | −0.515 | −0.131 | 0.215 | 1581 |
| 5801 | 688 | carbarsone | 15 | μM | PC3 | −0.515 | −0.203 | 0.143 | 3991 |
| 5802 | 682 | sulfadimidine | 13 | μM | PC3 | −0.515 | −0.216 | 0.131 | 3765 |
| 5803 | 714 | estradiol | 15 | μM | PC3 | −0.515 | −0.239 | 0.108 | 6718 |
| 5799 | 664 | harpagoside | 8 | μM | HL60 | −0.514 | −0.114 | 0.232 | 2935 |
| 5798 | 683 | 2,6-dimethylpiperidine | 27 | μM | PC3 | −0.514 | −0.225 | 0.121 | 3806 |
| 5797 | 602 | 15-delta prostaglandin | 10 | μM | HL60 | −0.514 | −0.229 | 0.117 | 1172 |
| | | J2 | | | | | | | |
| 5795 | 735 | chlorhexidine | 8 | μM | MCF7 | −0.514 | −0.248 | 0.098 | 5403 |
| 5796 | 745 | racecadotril | 10 | μM | MCF7 | −0.514 | −0.26 | 0.086 | 6231 |
| 5793 | 664 | etofenamate | 11 | μM | HL60 | −0.513 | −0.139 | 0.207 | 2907 |
| 5792 | 661 | Prestwick-981 | 11 | μM | HL60 | −0.513 | −0.181 | 0.164 | 3125 |
| 5791 | 661 | esculetin | 22 | μM | HL60 | −0.513 | −0.217 | 0.128 | 3120 |
| 5794 | 650 | tanespimycin | 1 | μM | HL60 | −0.513 | −0.236 | 0.11 | 2686 |
| 5790 | 613 | hydroxyzine | 9 | μM | HL60 | −0.512 | −0.154 | 0.191 | 2024 |
| 5787 | 750 | LY-294002 | 100 | nM | HL60 | −0.512 | −0.16 | 0.184 | 6175 |
| 5786 | 644 | diflorasone | 8 | μM | HL60 | −0.512 | −0.161 | 0.183 | 2142 |
| 5788 | 650 | sirolimus | 100 | nM | HL60 | −0.512 | −0.199 | 0.145 | 2681 |
| 5789 | 617 | antimycin A | 7 | μM | PC3 | −0.512 | −0.209 | 0.136 | 2098 |
| 5784 | 733 | isoetarine | 12 | μM | PC3 | −0.511 | −0.182 | 0.162 | 5812 |
| 5782 | 746 | ifosfamide | 15 | μM | MCF7 | −0.511 | −0.183 | 0.16 | 6279 |
| 5783 | 771 | trifluoperazine | 8 | μM | MCF7 | −0.511 | −0.203 | 0.141 | 7420 |
| 5781 | 708 | bromocriptine | 5 | μM | MCF7 | −0.511 | −0.249 | 0.094 | 5665 |
| 5785 | 726 | azathioprine | 14 | μM | MCF7 | −0.511 | −0.272 | 0.072 | 5262 |
| 5778 | 618 | trichostatin A | 100 | nM | HL60 | −0.51 | −0.091 | 0.252 | 2370 |
| 5777 | 695 | doxylamine | 10 | μM | MCF7 | −0.51 | −0.164 | 0.179 | 4819 |
| 5776 | 650 | alpha-estradiol | 10 | nM | HL60 | −0.51 | −0.178 | 0.165 | 2670 |
| 5780 | 640 | ceftazidime | 6 | μM | HL60 | −0.51 | −0.201 | 0.143 | 1721 |
| 5779 | 683 | santonin | 16 | μM | PC3 | −0.51 | −0.225 | 0.119 | 3795 |
| 5775 | 1030 | trichostatin A | 1 | μM | MCF7 | −0.509 | −0.159 | 0.183 | 6434 |
| 5774 | 655 | cephaeline | 6 | μM | MCF7 | −0.509 | −0.244 | 0.098 | 3290 |
| 5772 | 699 | levomepromazine | 9 | μM | MCF7 | −0.508 | −0.194 | 0.148 | 4723 |
| 5771 | 755 | dexibuprofen | 19 | μM | MCF7 | −0.508 | −0.209 | 0.133 | 6471 |
| 5770 | 758 | haloperidol | 11 | μM | MCF7 | −0.508 | −0.231 | 0.111 | 5638 |
| 5773 | 703 | tinidazole | 16 | μM | PC3 | −0.508 | −0.232 | 0.11 | 4548 |
| 5766 | 751 | trichostatin A | 100 | nM | MCF7 | −0.507 | −0.119 | 0.222 | 6064 |
| 5769 | 664 | letrozole | 14 | μM | HL60 | −0.507 | −0.138 | 0.203 | 2916 |
| 5765 | 729 | glycocholic acid | 9 | μM | MCF7 | −0.507 | −0.173 | 0.167 | 5316 |
| 5767 | 651 | sulfanilamide | 23 | μM | HL60 | −0.507 | −0.208 | 0.133 | 2709 |
| 5768 | 707 | diloxanide | 12 | μM | MCF7 | −0.507 | −0.28 | 0.061 | 5025 |
| 5762 | 745 | cefepime | 7 | μM | MCF7 | −0.506 | −0.165 | 0.176 | 6237 |
| 5764 | 688 | 6-azathymine | 31 | μM | PC3 | −0.506 | −0.178 | 0.163 | 3987 |
| 5763 | 728 | riboflavin | 11 | μM | PC3 | −0.506 | −0.232 | 0.108 | 4485 |
| 5760 | 681 | meclofenoxate | 14 | μM | PC3 | −0.505 | −0.177 | 0.163 | 3707 |
| 5761 | 629 | noretynodrel | 13 | μM | HL60 | −0.505 | −0.191 | 0.149 | 1860 |
| 5758 | 41 | estradiol | 10 | nM | HL60 | −0.505 | −0.204 | 0.135 | 387 |
| 5757 | 753 | dextromethorphan | 11 | μM | PC3 | −0.505 | −0.222 | 0.117 | 6300 |
| 5759 | 736 | tolfenamic acid | 15 | μM | MCF7 | −0.505 | −0.225 | 0.115 | 5454 |
| 5755 | 688 | gramine | 23 | μM | PC3 | −0.504 | −0.162 | 0.177 | 3999 |
| 5753 | 660 | aminohippuric acid | 21 | μM | HL60 | −0.504 | −0.172 | 0.167 | 3076 |
| 5756 | 613 | perphenazine | 10 | μM | HL60 | −0.504 | −0.188 | 0.152 | 2040 |
| 5754 | 644 | canavanine | 14 | μM | HL60 | −0.504 | −0.199 | 0.14 | 2141 |
| 5751 | 687 | phenelzine | 17 | μM | MCF7 | −0.504 | −0.218 | 0.121 | 3884 |
| 5752 | 1061 | carmustine | 100 | μM | MCF7 | −0.504 | −0.254 | 0.085 | 6914 |
| 5750 | 641 | papaverine | 11 | μM | HL60 | −0.503 | −0.121 | 0.218 | 1755 |
| 5747 | 658 | trichostatin A | 100 | nM | HL60 | −0.503 | −0.145 | 0.194 | 2993 |
| 5748 | 632 | diphemanil metilsulfate | 10 | μM | MCF7 | −0.503 | −0.2 | 0.139 | 1494 |
| 5749 | 753 | pralidoxime | 23 | μM | PC3 | −0.503 | −0.239 | 0.1 | 6283 |
| 5744 | 513 | vorinostat | 10 | μM | MCF7 | −0.502 | −0.128 | 0.209 | 1058 |
| 5746 | 736 | trichostatin A | 100 | nM | MCF7 | −0.502 | −0.15 | 0.188 | 5441 |
| 5745 | 671 | butacaine | 13 | μM | MCF7 | −0.502 | −0.245 | 0.093 | 3469 |
| 5742 | 689 | yohimbic acid | 11 | μM | PC3 | −0.501 | −0.196 | 0.141 | 4082 |
| 5743 | 720 | CP-320650-01 | 10 | μM | MCF7 | −0.501 | −0.24 | 0.097 | 4379 |
| 5741 | 734 | nomifensine | 11 | μM | PC3 | −0.5 | −0.208 | 0.128 | 5863 |
| 5740 | 26b | monorden | 100 | nM | MCF7 | −0.5 | −0.232 | 0.105 | 325 |

c) Drugs with Negative Connectivity Scores that Reverse CRG Expression Suppress the Malignant Phenotype.

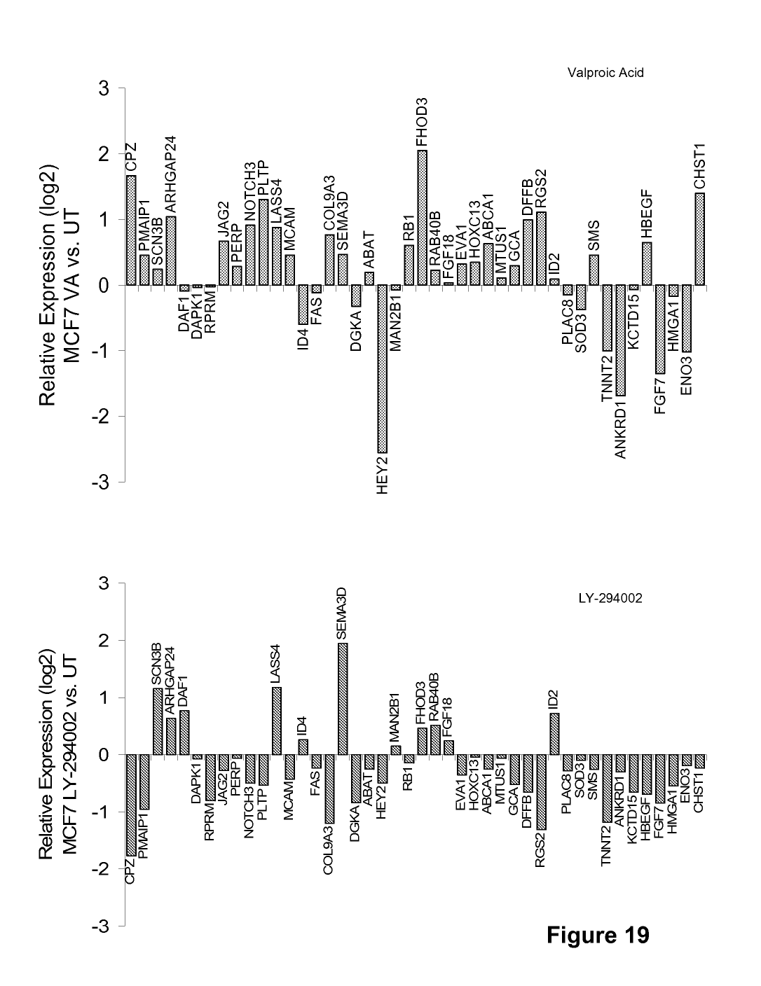

The general utility of the CRGs in identifying anti-cancer agents was immediately validated by the query results, which indicate that the list of negatively-connected drugs contains a variety of HDACi, such as valproic acid, which was previously shown be effective in reversing CRG expression and abrogating the malignant phenotype, as well as others e.g., trichostatin A and vorinostat. In addition to HDACi, the CRG-based query revealed several negatively-connected compounds, such as LY-294002, wortmannin, and sirolimus (rapamycin), acting along the PI3K pathway, a well-known mediator of cancer survival, progression, and resistance to chemotherapy (Tokunaga et al., 2008; Zhang et al., 2007). To investigate whether HDAC1 and PI3K pathway inhibitors demonstrating strong negative connectivity antagonized similar or complementary subsets of CRGs, the gene expression changes of individual CRGs for these drugs were extracted and compared. This comparison revealed that the subsets of CRGs modulated by the two drug classes were distinct, consistent with their different mechanisms of action. (FIG. 19).

d) Drugs which Preferentially Target Up- or Down-Regulated CRGs can Interact to Inhibit Malignant Transformation

Further analysis of the CMap data shows that many drugs preferentially target either up- or down-regulated CRGs (Tables 16 and 17). Because only part of the overall signature is targeted, such compounds do not attain a negative connectivity score, but they clearly reverse a proportion of the CRG signature. Based on the CRG perturbation experiments, these compounds have tumor-inhibitory efficacy on their own and in combination with other compounds that affect expression of complementary sets of CRGs. For example, this includes combinations of any of the compounds targeting up-regulated CRGs shown in Table 16 with any of the compounds that target down-regulated CRGs shown in Table 17.

TABLE 16
Compounds predicted to increase the expression of down-regulated CRGs with minimal
effect on up-regulated CRGs, identified by the Connectivity Map

| Rank | Batch | CMap Name | Dose | | Cell | Score | ESup | ESdown | Instance_ID |
|---|---|---|---|---|---|---|---|---|---|
| 2333 | 682 | trichostatin A | 100 | nM | PC3 | 0 | 0.18 | 0.379 | 3787 |
| 3239 | 727 | valproic acid | 500 | μM | PC3 | 0 | 0.103 | 0.372 | 4464 |
| 3124 | 718 | trichostatin A | 100 | nM | PC3 | 0 | 0.118 | 0.339 | 5065 |
| 3070 | 732 | trichostatin A | 100 | nM | PC3 | 0 | 0.122 | 0.318 | 5802 |
| 2248 | 637 | trichostatin A | 100 | nM | MCF7 | 0 | 0.187 | 0.313 | 2268 |
| 3211 | 603 | vorinostat | 10 | μM | PC3 | 0 | 0.106 | 0.288 | 1220 |
| 2232 | 603 | trichostatin A | 1 | μM | PC3 | 0 | 0.188 | 0.284 | 1234 |
| 1514 | 744 | trichostatin A | 100 | nM | MCF7 | 0 | 0.259 | 0.281 | 6820 |
| 3137 | 680 | trichostatin A | 100 | nM | PC3 | 0 | 0.116 | 0.28 | 3688 |
| 2314 | 671 | pipenzolate bromide | 9 | μM | MCF7 | 0 | 0.182 | 0.28 | 3460 |
| 2767 | 659 | ioversol | 5 | μM | HL60 | 0 | 0.145 | 0.278 | 3026 |
| 2697 | 686 | trichostatin A | 100 | nM | MCF7 | 0 | 0.151 | 0.276 | 3868 |
| 3173 | 658 | mestranol | 13 | μM | HL60 | 0 | 0.112 | 0.273 | 3008 |
| 3306 | 664 | pronetalol | 15 | μM | HL60 | 0 | 0.09 | 0.271 | 2902 |
| 2999 | 636 | trichostatin A | 100 | nM | MCF7 | 0 | 0.128 | 0.271 | 2247 |
| 2812 | 706 | trichostatin A | 100 | nM | MCF7 | 0 | 0.142 | 0.271 | 4954 |
| 2649 | 60 | trichostatin A | 100 | nM | PC3 | 0 | 0.155 | 0.271 | 448 |
| 1427 | 663 | trichostatin A | 100 | nM | MCF7 | 0 | 0.273 | 0.27 | 2794 |
| 2686 | 648 | trichostatin A | 100 | nM | HL60 | 0 | 0.152 | 0.269 | 2523 |
| 2138 | 685 | trichostatin A | 100 | nM | MCF7 | 0 | 0.195 | 0.269 | 3643 |
| 2494 | 671 | trichostatin A | 100 | nM | MCF7 | 0 | 0.167 | 0.268 | 3462 |
| 2472 | 725 | trichostatin A | 100 | nM | MCF7 | 0 | 0.169 | 0.266 | 5209 |
| 3062 | 660 | desoxycortone | 12 | μM | HL60 | 0 | 0.123 | 0.264 | 3099 |
| 3298 | 634 | dicloxacillin | 8 | μM | HL60 | 0 | 0.091 | 0.262 | 2445 |
| 1916 | 654 | trichostatin A | 100 | nM | MCF7 | 0 | 0.213 | 0.261 | 3243 |
| 1641 | 694 | trichostatin A | 100 | nM | MCF7 | 0 | 0.241 | 0.26 | 4770 |
| 3313 | 629 | allantoin | 25 | μM | HL60 | 0 | 0.088 | 0.258 | 1842 |
| 3222 | 659 | rolitetracycline | 8 | μM | HL60 | 0 | 0.105 | 0.258 | 3031 |
| 2108 | 33 | valproic acid | 2 | mM | MCF7 | 0 | 0.197 | 0.258 | 346 |
| 2961 | 687 | rifabutin | 5 | μM | MCF7 | 0 | 0.131 | 0.255 | 3873 |
| 2745 | 616 | trichostatin A | 100 | nM | PC3 | 0 | 0.147 | 0.255 | 2084 |
| 2432 | 729 | trichostatin A | 100 | nM | MCF7 | 0 | 0.172 | 0.253 | 5308 |
| 1699 | 611 | trichostatin A | 100 | nM | PC3 | 0 | 0.234 | 0.252 | 1951 |
| 3276 | 648 | metoprolol | 6 | μM | HL60 | 0 | 0.097 | 0.251 | 2543 |
| 1968 | 700 | metoclopramide | 12 | μM | MCF7 | 0 | 0.209 | 0.25 | 4750 |
| 1832 | 730 | trichostatin A | 100 | nM | MCF7 | 0 | 0.22 | 0.25 | 5336 |
| 3036 | 645 | benfotiamine | 9 | μM | HL60 | 0 | 0.125 | 0.249 | 2177 |
| 3231 | 645 | trichostatin A | 100 | nM | HL60 | 0 | 0.104 | 0.248 | 2208 |
| 1458 | 653 | procainamide | 15 | μM | MCF7 | 0 | 0.268 | 0.247 | 2618 |
| 2941 | 618 | 6-benzylaminopurine | 18 | μM | HL60 | 0 | 0.133 | 0.246 | 2351 |
| 2876 | 743 | trichostatin A | 100 | nM | MCF7 | 0 | 0.137 | 0.246 | 6784 |
| 2995 | 700 | trichostatin A | 100 | nM | MCF7 | 0 | 0.128 | 0.244 | 4768 |
| 3348 | 629 | sulfaphenazole | 13 | μM | HL60 | 0 | 0.064 | 0.243 | 1836 |
| 1871 | 626 | trichostatin A | 100 | nM | MCF7 | 0 | 0.218 | 0.243 | 1637 |
| 1799 | 695 | trichostatin A | 100 | nM | MCF7 | 0 | 0.223 | 0.243 | 4821 |
| 1679 | 752 | trichostatin A | 100 | nM | MCF7 | 0 | 0.236 | 0.243 | 6085 |
| 3152 | 628 | trichostatin A | 100 | nM | PC3 | 0 | 0.114 | 0.242 | 1793 |
| 3346 | 629 | chloramphenicol | 12 | μM | HL60 | 0 | 0.069 | 0.241 | 1837 |
| 3037 | 610 | trichostatin A | 100 | nM | PC3 | 0 | 0.125 | 0.24 | 1891 |
| 2857 | 629 | 8-azaguanine | 26 | μM | HL60 | 0 | 0.139 | 0.24 | 1833 |
| 2101 | 640 | propafenone | 11 | μM | HL60 | 0 | 0.197 | 0.239 | 1722 |
| 1771 | 764 | trichostatin A | 100 | nM | PC3 | 0 | 0.225 | 0.238 | 7136 |
| 2881 | 629 | morantel | 11 | μM | HL60 | 0 | 0.137 | 0.237 | 1840 |
| 2886 | 641 | ipratropium bromide | 10 | μM | HL60 | 0 | 0.136 | 0.236 | 1769 |
| 2775 | 659 | carbachol | 22 | μM | HL60 | 0 | 0.145 | 0.235 | 3042 |
| 2436 | 665 | pyrvinium | 3 | μM | HL60 | 0 | 0.172 | 0.235 | 2957 |
| 2193 | 660 | cantharidin | 20 | μM | HL60 | 0 | 0.191 | 0.235 | 3075 |
| 2153 | 732 | alpha-yohimbine | 10 | μM | PC3 | 0 | 0.194 | 0.235 | 5800 |
| 3201 | 640 | triflusal | 16 | μM | HL60 | 0 | 0.108 | 0.233 | 1717 |
| 3006 | 648 | skimmianine | 15 | μM | HL60 | 0 | 0.127 | 0.233 | 2504 |
| 2386 | 735 | trichostatin A | 100 | nM | MCF7 | 0 | 0.176 | 0.233 | 5417 |
| 2024 | 738 | trichostatin A | 100 | nM | MCF7 | 0 | 0.204 | 0.233 | 5511 |
| 1902 | 630 | suloctidil | 12 | μM | HL60 | 0 | 0.214 | 0.233 | 1297 |
| 3321 | 749 | trifluridine | 14 | μM | HL60 | 0 | 0.086 | 0.231 | 6136 |
| 3081 | 659 | bemegride | 26 | μM | HL60 | 0 | 0.121 | 0.231 | 3051 |
| 3267 | 720 | rifabutin | 5 | μM | MCF7 | 0 | 0.098 | 0.23 | 4349 |
| 3016 | 658 | propantheline bromide | 9 | μM | HL60 | 0 | 0.127 | 0.23 | 3013 |
| 1917 | 630 | thioguanosine | 13 | μM | HL60 | 0 | 0.213 | 0.23 | 1264 |
| 3270 | 612 | isoxsuprine | 12 | μM | HL60 | 0 | 0.098 | 0.229 | 1985 |
| 3177 | 708 | trichostatin A | 100 | nM | MCF7 | 0 | 0.112 | 0.229 | 5693 |
| 2834 | 645 | ethotoin | 20 | μM | HL60 | 0 | 0.14 | 0.228 | 2196 |
| 2744 | 699 | trichostatin A | 100 | nM | MCF7 | 0 | 0.147 | 0.226 | 4710 |
| 2090 | 630 | benfluorex | 10 | μM | HL60 | 0 | 0.198 | 0.226 | 1266 |
| 2448 | 613 | metolazone | 11 | μM | HL60 | 0 | 0.171 | 0.225 | 2014 |
| 2388 | 647 | trichostatin A | 100 | nM | MCF7 | 0 | 0.176 | 0.225 | 3227 |
| 2004 | 602 | geldanamycin | 1 | μM | HL60 | 0 | 0.205 | 0.225 | 1169 |
| 1775 | 45 | trichostatin A | 100 | nM | ssMCF7 | 0 | 0.224 | 0.225 | 413 |
| 1624 | 676 | trichostatin A | 100 | nM | MCF7 | 0 | 0.242 | 0.225 | 7324 |
| 3078 | 1043 | trichostatin A | 1 | μM | MCF7 | 0 | 0.122 | 0.223 | 6579 |
| 2557 | 705 | trichostatin A | 100 | nM | MCF7 | 0 | 0.161 | 0.223 | 4388 |
| 1896 | 618 | phenelzine | 17 | μM | HL60 | 0 | 0.215 | 0.223 | 2357 |
| 2977 | 1014 | trichostatin A | 1 | μM | MCF7 | 0 | 0.129 | 0.222 | 5976 |
| 1567 | 671 | vidarabine | 15 | μM | MCF7 | 0 | 0.249 | 0.222 | 3445 |
| 3317 | 630 | tacrine | 16 | μM | HL60 | 0 | 0.087 | 0.221 | 1278 |
| 2378 | 655 | trichostatin A | 100 | nM | MCF7 | 0 | 0.177 | 0.221 | 3312 |
| 3147 | 737 | trichostatin A | 100 | nM | MCF7 | 0 | 0.115 | 0.22 | 5484 |
| 3020 | 644 | picrotoxinin | 14 | μM | HL60 | 0 | 0.126 | 0.22 | 2161 |
| 2730 | 664 | epitiostanol | 13 | μM | HL60 | 0 | 0.148 | 0.22 | 2922 |
| 1959 | 640 | trichostatin A | 100 | nM | HL60 | 0 | 0.209 | 0.219 | 1732 |
| 2002 | 767 | trichostatin A | 100 | nM | MCF7 | 0 | 0.206 | 0.218 | 6932 |
| 3223 | 615 | etofylline | 18 | μM | HL60 | 0 | 0.105 | 0.217 | 1409 |
| 3063 | 648 | fluorometholone | 11 | μM | HL60 | 0 | 0.123 | 0.217 | 2509 |
| 2840 | 514 | trichostatin A | 100 | nM | MCF7 | 0 | 0.14 | 0.217 | 1112 |
| 2152 | 659 | ethaverine | 9 | μM | HL60 | 0 | 0.194 | 0.217 | 3037 |
| 3323 | 664 | sanguinarine | 12 | μM | HL60 | 0 | 0.085 | 0.216 | 2927 |
| 3030 | 662 | trichostatin A | 100 | nM | MCF7 | 0 | 0.125 | 0.216 | 2777 |
| 2231 | 660 | etynodiol | 10 | μM | HL60 | 0 | 0.188 | 0.215 | 3102 |
| 2025 | 1084 | daunorubicin | 1 | μM | MCF7 | 0 | 0.204 | 0.215 | 7507 |
| 1683 | 691 | trichostatin A | 100 | nM | MCF7 | 0 | 0.236 | 0.215 | 4153 |
| 1700 | 757 | vorinostat | 10 | μM | MCF7 | 0 | 0.234 | 0.214 | 5580 |
| 3213 | 659 | sulconazole | 9 | μM | HL60 | 0 | 0.106 | 0.213 | 3035 |
| 3117 | 642 | trichostatin A | 100 | nM | MCF7 | 0 | 0.118 | 0.213 | 2330 |
| 3022 | 645 | bromopride | 12 | μM | HL60 | 0 | 0.126 | 0.213 | 2182 |
| 2776 | 750 | acetylsalicylic acid | 100 | μM | HL60 | 0 | 0.144 | 0.213 | 6164 |
| 3079 | 602 | tanespimycin | 1 | μM | HL60 | 0 | 0.122 | 0.211 | 1147 |
| 2820 | 649 | meclofenoxate | 14 | μM | HL60 | 0 | 0.141 | 0.211 | 2546 |
| 2624 | 634 | neostigmine bromide | 13 | μM | HL60 | 0 | 0.157 | 0.211 | 2432 |
| 2416 | 618 | mebendazole | 14 | μM | HL60 | 0 | 0.174 | 0.211 | 2338 |
| 1828 | 670 | fenoprofen | 7 | μM | MCF7 | 0 | 0.221 | 0.211 | 3412 |
| 1585 | 613 | hesperetin | 13 | μM | HL60 | 0 | 0.247 | 0.211 | 2031 |
| 1444 | 646 | quinidine | 11 | μM | MCF7 | 0 | 0.271 | 0.21 | 3191 |
| 3214 | 752 | napelline | 11 | μM | MCF7 | 0 | 0.106 | 0.209 | 6084 |
| 2968 | 758 | trichostatin A | 100 | nM | MCF7 | 0 | 0.131 | 0.209 | 5625 |
| 2527 | 664 | tracazolate | 12 | μM | HL60 | 0 | 0.164 | 0.209 | 2919 |
| 2159 | 737 | trimetazidine | 12 | μM | MCF7 | 0 | 0.194 | 0.209 | 5479 |
| 3051 | 634 | iohexol | 5 | μM | HL60 | 0 | 0.124 | 0.208 | 2461 |
| 2442 | 757 | trichostatin A | 100 | nM | MCF7 | 0 | 0.172 | 0.208 | 5572 |
| 2266 | 665 | S-propranolol | 14 | μM | HL60 | 0 | 0.186 | 0.208 | 2961 |
| 2085 | 731 | trioxysalen | 18 | μM | PC3 | 0 | 0.198 | 0.208 | 5736 |
| 1295 | 1071 | MS-275 | 10 | μM | PC3 | 0 | 0.317 | 0.208 | 7074 |
| 3227 | 651 | azlocillin | 8 | μM | HL60 | 0 | 0.104 | 0.207 | 2727 |
| 3172 | 631 | ginkgolide A | 10 | μM | HL60 | 0 | 0.112 | 0.207 | 1324 |
| 1535 | 738 | lisinopril | 9 | μM | MCF7 | 0 | 0.255 | 0.207 | 5504 |
| 3091 | 612 | pyrimethamine | 16 | μM | HL60 | 0 | 0.121 | 0.206 | 1974 |
| 1644 | 651 | sulfametoxydiazine | 14 | μM | HL60 | 0 | 0.24 | 0.206 | 2712 |
| 2987 | 641 | syrosingopine | 6 | μM | HL60 | 0 | 0.128 | 0.205 | 1761 |
| 2921 | 629 | meticrane | 15 | μM | HL60 | 0 | 0.134 | 0.205 | 1834 |
| 2435 | 502 | trichostatin A | 1 | μM | MCF7 | 0 | 0.172 | 0.205 | 981 |
| 2523 | 711 | trichostatin A | 100 | nM | MCF7 | 0 | 0.165 | 0.204 | 3979 |
| 2116 | 635 | tolazamide | 13 | μM | HL60 | 0 | 0.196 | 0.204 | 2482 |
| 1792 | 645 | citiolone | 25 | μM | HL60 | 0 | 0.223 | 0.204 | 2176 |
| 3071 | 755 | trichostatin A | 100 | nM | MCF7 | 0 | 0.122 | 0.203 | 6454 |
| 2893 | 690 | trichostatin A | 100 | nM | MCF7 | 0 | 0.136 | 0.203 | 4112 |
| 1309 | 642 | mephenesin | 22 | μM | MCF7 | 0 | 0.313 | 0.203 | 2304 |
| 2493 | 619 | pimethixene | 10 | μM | HL60 | 0 | 0.167 | 0.202 | 2395 |
| 1418 | 765 | trichostatin A | 100 | nM | MCF7 | 0 | 0.275 | 0.202 | 6972 |
| 3192 | 741 | dosulepin | 12 | μM | MCF7 | 0 | 0.109 | 0.201 | 5986 |
| 2980 | 651 | cinoxacin | 15 | μM | HL60 | 0 | 0.129 | 0.201 | 2722 |
| 3046 | 641 | berberine | 11 | μM | HL60 | 0 | 0.124 | 0.2 | 1778 |
| 2573 | 756 | trichostatin A | 100 | nM | MCF7 | 0 | 0.16 | 0.2 | 6493 |
| 2418 | 649 | fenoprofen | 7 | μM | HL60 | 0 | 0.174 | 0.2 | 2553 |
| 2348 | 665 | ioxaglic acid | 3 | μM | HL60 | 0 | 0.179 | 0.2 | 2966 |

Reversal of down-regulated CRG expression is indicated by a positive ES score for the down-regulated genes. Drugs are considered to target the down-regulated genes if the ESdown value is greater than 0.2. A lack of reversal of up-regulated genes is indicated by a positive ES score for this segment of the CRG signature.

TABLE 17
Compounds predicted to decrease the expression of up-regulated CRGs with minimal
effect on down-regulated CRGs, identified by the Connectivity Map

| Rank | Batch | CMap Name | Dose | | Cell | Score | ESup | ESdown | Instance_ID |
|---|---|---|---|---|---|---|---|---|---|
| 4652 | 766 | pergolide | 10 | μM | MCF7 | 0 | −0.386 | −0.109 | 7031 |
| 4651 | 683 | withaferin A | 1 | μM | PC3 | 0 | −0.371 | −0.141 | 3819 |
| 4650 | 676 | alprostadil | 11 | μM | MCF7 | 0 | −0.365 | −0.128 | 7358 |
| 4649 | 715 | betamethasone | 10 | μM | PC3 | 0 | −0.358 | −0.121 | 6728 |
| 4648 | 1048 | fulvestrant | 1 | μM | PC3 | 0 | −0.357 | −0.137 | 6867 |
| 4647 | 747 | doxycycline | 8 | μM | MCF7 | 0 | −0.354 | −0.109 | 7195 |
| 4646 | 627 | atracurium besilate | 3 | μM | MCF7 | 0 | −0.349 | −0.083 | 1702 |
| 4645 | 632 | metronidazole | 23 | μM | MCF7 | 0 | −0.347 | −0.115 | 1503 |
| 4644 | 746 | demecarium bromide | 6 | μM | MCF7 | 0 | −0.346 | −0.149 | 6269 |
| 4643 | 676 | harpagoside | 8 | μM | MCF7 | 0 | −0.343 | −0.127 | 7355 |
| 4642 | 728 | securinine | 18 | μM | PC3 | 0 | −0.341 | −0.284 | 4493 |
| 4641 | 626 | fulvestrant | 10 | nM | MCF7 | 0 | −0.339 | −0.098 | 1663 |
| 4640 | 748 | bambuterol | 10 | μM | MCF7 | 0 | −0.338 | −0.097 | 7239 |
| 4639 | 660 | terguride | 12 | μM | HL60 | 0 | −0.334 | −0.143 | 3082 |
| 4638 | 703 | withaferin A | 1 | μM | PC3 | 0 | −0.33 | −0.088 | 4554 |
| 4637 | 504 | tretinoin | 1 | μM | MCF7 | 0 | −0.324 | −0.135 | 849 |
| 4636 | 514 | minocycline | 11 | μM | MCF7 | 0 | −0.324 | −0.117 | 1135 |
| 4635 | 745 | tranexamic acid | 25 | μM | MCF7 | 0 | −0.322 | −0.169 | 6238 |
| 4634 | 692 | molindone | 13 | μM | PC3 | 0 | −0.319 | −0.082 | 4199 |
| 4632 | 662 | yohimbine | 10 | μM | MCF7 | 0 | −0.316 | −0.176 | 2755 |
| 4633 | 766 | meclofenamic acid | 12 | μM | MCF7 | 0 | −0.316 | −0.09 | 7038 |
| 4631 | 714 | mimosine | 20 | μM | PC3 | 0 | −0.315 | −0.143 | 6703 |
| 4630 | 701 | foliosidine | 13 | μM | PC3 | 0 | −0.313 | −0.083 | 4295 |
| 4629 | 1041 | alprostadil | 10 | μM | MCF7 | 0 | −0.311 | −0.128 | 6576 |
| 4628 | 505 | 5186324 | 2 | μM | MCF7 | 0 | −0.31 | −0.118 | 900 |
| 4627 | 671 | raloxifene | 8 | μM | MCF7 | 0 | −0.309 | −0.136 | 3480 |
| 4626 | 670 | merbromin | 5 | μM | MCF7 | 0 | −0.307 | −0.129 | 3439 |
| 4625 | 772 | halofantrine | 7 | μM | MCF7 | 0 | −0.306 | −0.091 | 7469 |
| 4624 | 734 | vinpocetine | 11 | μM | PC3 | 0 | −0.305 | −0.086 | 5859 |
| 4623 | 729 | fluvastatin | 9 | μM | MCF7 | 0 | −0.304 | −0.075 | 5290 |
| 4622 | 656 | probenecid | 14 | μM | MCF7 | 0 | −0.304 | −0.065 | 2825 |
| 4620 | 710 | fluspirilene | 8 | μM | PC3 | 0 | −0.303 | −0.174 | 6662 |
| 4621 | 743 | cefoxitin | 9 | μM | MCF7 | 0 | −0.303 | −0.159 | 6796 |
| 4619 | 771 | diethylcarbamazine | 10 | μM | MCF7 | 0 | −0.303 | −0.103 | 7425 |
| 4618 | 693 | simvastatin | 10 | μM | PC3 | 0 | −0.302 | −0.105 | 4244 |
| 4617 | 718 | tridihexethyl | 11 | μM | PC3 | 0 | −0.301 | −0.07 | 5067 |
| 4615 | 692 | atovaquone | 11 | μM | PC3 | 0 | −0.3 | −0.136 | 4201 |
| 4616 | 725 | rosiglitazone | 10 | μM | MCF7 | 0 | −0.3 | −0.113 | 5230 |
| 4614 | 615 | aztreonam | 9 | μM | HL60 | 0 | −0.299 | −0.121 | 1435 |
| 4612 | 632 | tolnaftate | 13 | μM | MCF7 | 0 | −0.298 | −0.144 | 1501 |
| 4613 | 683 | alpha-ergocryptine | 7 | μM | PC3 | 0 | −0.298 | −0.128 | 3817 |
| 4611 | 764 | yohimbine | 10 | μM | PC3 | 0 | −0.297 | −0.067 | 7130 |
| 4609 | 627 | heptaminol | 22 | μM | MCF7 | 0 | −0.296 | −0.249 | 1703 |
| 4610 | 735 | nizatidine | 12 | μM | MCF7 | 0 | −0.296 | −0.041 | 5406 |
| 4608 | 686 | 0317956-0000 | 10 | μM | MCF7 | 0 | −0.295 | −0.092 | 3855 |
| 4606 | 688 | levobunolol | 12 | μM | PC3 | 0 | −0.294 | −0.126 | 4016 |
| 4607 | 632 | cimetidine | 16 | μM | MCF7 | 0 | −0.294 | −0.107 | 1464 |
| 4605 | 702 | sulfachlorpyridazine | 14 | μM | PC3 | 0 | −0.294 | −0.061 | 4326 |
| 4604 | 701 | PNU-0230031 | 10 | μM | PC3 | 0 | −0.293 | −0.144 | 4288 |
| 4603 | 726 | clozapine | 12 | μM | MCF7 | 0 | −0.293 | −0.093 | 5265 |
| 4599 | 1029 | F0447-0125 | 10 | μM | PC3 | 0 | −0.292 | −0.157 | 6429 |
| 4601 | 654 | carteolol | 12 | μM | MCF7 | 0 | −0.292 | −0.121 | 3276 |
| 4600 | 1047 | PHA-00767505E | 10 | μM | MCF7 | 0 | −0.292 | −0.101 | 6596 |
| 4602 | 656 | rifampicin | 5 | μM | MCF7 | 0 | −0.292 | −0.076 | 2847 |
| 4594 | 728 | acepromazine | 9 | μM | PC3 | 0 | −0.291 | −0.156 | 4494 |
| 4597 | 706 | khellin | 15 | μM | MCF7 | 0 | −0.291 | −0.149 | 4987 |
| 4595 | 734 | atropine | 6 | μM | PC3 | 0 | −0.291 | −0.112 | 5865 |
| 4596 | 766 | dihydroergocristine | 6 | μM | MCF7 | 0 | −0.291 | −0.097 | 7034 |
| 4598 | 706 | methyldopate | 15 | μM | MCF7 | 0 | −0.291 | −0.093 | 4986 |
| 4593 | 676 | fursultiamine | 9 | μM | MCF7 | 0 | −0.289 | −0.156 | 7349 |
| 4589 | 767 | rosiglitazone | 10 | μM | MCF7 | 0 | −0.289 | −0.101 | 6950 |
| 4592 | 692 | lumicolchicine | 10 | μM | PC3 | 0 | −0.289 | −0.076 | 4195 |
| 4591 | 725 | LY-294002 | 10 | μM | MCF7 | 0 | −0.289 | −0.061 | 5236 |
| 4590 | 725 | troglitazone | 10 | μM | MCF7 | 0 | −0.289 | −0.058 | 5229 |
| 4588 | 743 | isopropamide iodide | 8 | μM | MCF7 | 0 | −0.288 | −0.064 | 6781 |
| 4587 | 745 | tetracycline | 8 | μM | MCF7 | 0 | −0.287 | −0.131 | 6233 |
| 4586 | 1094 | meteneprost | 10 | μM | MCF7 | 0 | −0.286 | −0.12 | 7552 |
| 4585 | 1032 | 5155877 | 10 | μM | PC3 | 0 | −0.285 | −0.122 | 6544 |
| 4581 | 633 | lisuride | 12 | μM | MCF7 | 0 | −0.284 | −0.181 | 1545 |
| 4582 | 690 | levobunolol | 12 | μM | MCF7 | 0 | −0.284 | −0.128 | 4134 |
| 4583 | 771 | bumetanide | 11 | μM | MCF7 | 0 | −0.284 | −0.121 | 7440 |
| 4584 | 727 | 15-delta prostaglandin J2 | 10 | μM | PC3 | 0 | −0.284 | −0.101 | 4455 |
| 4580 | 750 | LY-294002 | 10 | μM | HL60 | 0 | −0.283 | −0.137 | 6195 |
| 4579 | 678 | mesalazine | 26 | μM | MCF7 | 0 | −0.283 | −0.126 | 3584 |
| 4576 | 676 | oxamniquine | 14 | μM | MCF7 | 0 | −0.282 | −0.106 | 7344 |
| 4578 | 646 | alprenolol | 14 | μM | MCF7 | 0 | −0.282 | −0.105 | 3188 |
| 4577 | 707 | benzbromarone | 9 | μM | MCF7 | 0 | −0.282 | −0.1 | 5015 |
| 4575 | 1061 | SB-203580 | 1 | μM | MCF7 | 0 | −0.281 | −0.067 | 6915 |
| 4573 | 710 | (−)-MK-801 | 12 | μM | PC3 | 0 | −0.28 | −0.109 | 6657 |
| 4574 | 743 | tetryzoline | 17 | μM | MCF7 | 0 | −0.28 | −0.101 | 6769 |
| 4572 | 617 | chlorphenesin | 16 | μM | PC3 | 0 | −0.28 | −0.064 | 2115 |
| 4569 | 660 | estrone | 15 | μM | HL60 | 0 | −0.279 | −0.163 | 3071 |
| 4571 | 640 | lobelanidine | 11 | μM | HL60 | 0 | −0.279 | −0.143 | 1747 |
| 4570 | 640 | prenylamine | 10 | μM | HL60 | 0 | −0.279 | −0.129 | 1737 |
| 4566 | 710 | bemegride | 26 | μM | PC3 | 0 | −0.278 | −0.115 | 6668 |
| 4568 | 1041 | Gly-His-Lys | 1 | μM | MCF7 | 0 | −0.278 | −0.108 | 6575 |
| 4567 | 693 | oxetacaine | 9 | μM | PC3 | 0 | −0.278 | −0.105 | 4246 |
| 4565 | 745 | pheneticillin | 10 | μM | MCF7 | 0 | −0.278 | −0.071 | 6239 |
| 4562 | 654 | myricetin | 13 | μM | MCF7 | 0 | −0.277 | −0.136 | 3270 |
| 4563 | 116 | monastrol | 100 | μM | PC3 | 0 | −0.277 | −0.09 | 668 |
| 4564 | 671 | iopamidol | 5 | μM | MCF7 | 0 | −0.277 | −0.072 | 3473 |
| 4561 | 772 | clemastine | 9 | μM | MCF7 | 0 | −0.276 | −0.092 | 7485 |
| 4560 | 689 | sotalol | 13 | μM | PC3 | 0 | −0.276 | −0.081 | 4079 |
| 4559 | 682 | dicoumarol | 12 | μM | PC3 | 0 | −0.273 | −0.135 | 3766 |
| 4558 | 683 | phenelzine | 17 | μM | PC3 | 0 | −0.273 | −0.118 | 3802 |
| 4557 | 747 | terazosin | 9 | μM | MCF7 | 0 | −0.272 | −0.173 | 7187 |
| 4556 | 745 | mefloquine | 10 | μM | MCF7 | 0 | −0.272 | −0.092 | 6205 |
| 4555 | 702 | methylbenzethonium | 9 | μM | PC3 | 0 | −0.271 | −0.138 | 4325 |
| | | chloride | | | | | | | |
| 4553 | 746 | cefuroxime | 9 | μM | MCF7 | 0 | −0.271 | −0.084 | 6261 |
| 4554 | 748 | gentamicin | 3 | μM | MCF7 | 0 | −0.271 | −0.074 | 7237 |
| 4552 | 713 | phenoxybenzamine | 12 | μM | PC3 | 0 | −0.27 | −0.077 | 4652 |
| 4550 | 751 | finasteride | 11 | μM | MCF7 | 0 | −0.269 | −0.135 | 6062 |
| 4551 | 729 | ambroxol | 10 | μM | MCF7 | 0 | −0.269 | −0.122 | 5319 |
| 4549 | 1094 | CP-863187 | 10 | μM | MCF7 | 0 | −0.268 | −0.136 | 7553 |
| 4548 | 728 | epivincamine | 11 | μM | PC3 | 0 | −0.268 | −0.122 | 4500 |
| 4544 | 623 | zaprinast | 15 | μM | HL60 | 0 | −0.267 | −0.19 | 1611 |
| 4545 | 631 | myricetin | 13 | μM | HL60 | 0 | −0.267 | −0.182 | 1334 |
| 4547 | 720 | PHA-00745360 | 10 | μM | MCF7 | 0 | −0.267 | −0.117 | 4381 |
| 4546 | 741 | pivmecillinam | 8 | μM | MCF7 | 0 | −0.267 | −0.096 | 6014 |
| 4543 | 676 | methyldopate | 15 | μM | MCF7 | 0 | −0.266 | −0.105 | 7360 |
| 4539 | 672 | (+/−)-catechin | 14 | μM | MCF7 | 0 | −0.265 | −0.119 | 3351 |
| 4542 | 693 | fosfosal | 18 | μM | PC3 | 0 | −0.265 | −0.119 | 4239 |
| 4541 | 626 | haloperidol | 10 | μM | MCF7 | 0 | −0.265 | −0.102 | 1669 |
| 4540 | 728 | hydrocotarnine | 13 | μM | PC3 | 0 | −0.265 | −0.075 | 4489 |
| 4536 | 617 | flufenamic acid | 14 | μM | PC3 | 0 | −0.264 | −0.113 | 2104 |
| 4535 | 692 | sulfathiazole | 16 | μM | PC3 | 0 | −0.264 | −0.102 | 4183 |
| 4534 | 750 | nordihydroguaiaretic acid | 1 | μM | HL60 | 0 | −0.264 | −0.098 | 6182 |
| 4537 | 676 | fluvoxamine | 9 | μM | MCF7 | 0 | −0.264 | −0.071 | 7333 |
| 4538 | 733 | hecogenin | 9 | μM | PC3 | 0 | −0.264 | −0.068 | 5818 |
| 4533 | 1040 | 5155877 | 10 | μM | PC3 | 0 | −0.263 | −0.104 | 6569 |
| 4531 | 710 | estrone | 15 | μM | PC3 | 0 | −0.263 | −0.093 | 6647 |
| 4532 | 715 | rolitetracycline | 8 | μM | PC3 | 0 | −0.263 | −0.073 | 6731 |
| 4530 | 656 | R-atenolol | 15 | μM | MCF7 | 0 | −0.262 | −0.151 | 2855 |
| 4527 | 706 | naphazoline | 16 | μM | MCF7 | 0 | −0.262 | −0.144 | 4949 |
| 4526 | 676 | sotalol | 13 | μM | MCF7 | 0 | −0.262 | −0.131 | 7338 |
| 4529 | 514 | tyrphostin AG-1478 | 32 | μM | MCF7 | 0 | −0.262 | −0.119 | 1141 |
| 4528 | 734 | bergenin | 12 | μM | PC3 | 0 | −0.262 | −0.116 | 5870 |
| 4525 | 715 | carbachol | 22 | μM | PC3 | 0 | −0.262 | −0.08 | 6742 |
| 4524 | 714 | methylergometrine | 9 | μM | PC3 | 0 | −0.261 | −0.09 | 6704 |
| 4523 | 693 | 7-aminocephalosporanic | 15 | μM | PC3 | 0 | −0.261 | −0.084 | 4242 |
| | | acid | | | | | | | |
| 4522 | 1069 | SB-203580 | 1 | μM | PC3 | 0 | −0.26 | −0.083 | 7066 |
| 4520 | 504 | geldanamycin | 1 | μM | MCF7 | 0 | −0.259 | −0.189 | 864 |
| 4521 | 676 | etilefrine | 18 | μM | MCF7 | 0 | −0.259 | −0.146 | 7350 |
| 4519 | 750 | LY-294002 | 10 | μM | HL60 | 0 | −0.259 | −0.098 | 6198 |
| 4518 | 692 | norcyclobenzaprine | 15 | μM | PC3 | 0 | −0.259 | −0.078 | 4190 |
| 4517 | 622 | vinpocetine | 11 | μM | HL60 | 0 | −0.258 | −0.178 | 1557 |
| 4514 | 766 | adiphenine | 11 | μM | MCF7 | 0 | −0.258 | −0.152 | 7037 |
| 4516 | 756 | Prestwick-983 | 17 | μM | MCF7 | 0 | −0.258 | −0.136 | 6520 |
| 4515 | 627 | diphenhydramine | 14 | μM | MCF7 | 0 | −0.258 | −0.103 | 1708 |
| 4512 | 663 | benzocaine | 24 | μM | MCF7 | 0 | −0.257 | −0.173 | 2822 |
| 4513 | 614 | cefotaxime | 8 | μM | HL60 | 0 | −0.257 | −0.158 | 1389 |
| 4511 | 657 | clorsulon | 11 | μM | MCF7 | 0 | −0.257 | −0.153 | 2884 |
| 4509 | 701 | diphenylpyraline | 13 | μM | PC3 | 0 | −0.256 | −0.092 | 4299 |
| 4510 | 734 | fluphenazine | 8 | μM | PC3 | 0 | −0.256 | −0.06 | 5880 |
| 4507 | 654 | dl-alpha tocopherol | 9 | μM | MCF7 | 0 | −0.255 | −0.113 | 3256 |
| 4505 | 736 | nomegestrol | 11 | μM | MCF7 | 0 | −0.255 | −0.108 | 5461 |
| 4504 | 751 | Prestwick-675 | 10 | μM | MCF7 | 0 | −0.255 | −0.104 | 6042 |
| 4506 | 694 | diflunisal | 16 | μM | MCF7 | 0 | −0.255 | −0.1 | 4794 |
| 4508 | 26b | LY-294002 | 10 | μM | MCF7 | 0 | −0.255 | −0.098 | 328 |
| 4503 | 1041 | PNU-0293363 | 10 | μM | MCF7 | 0 | −0.255 | −0.087 | 6573 |
| 4502 | 1094 | BCB000040 | 10 | μM | MCF7 | 0 | −0.255 | −0.081 | 7554 |
| 4499 | 513 | genistein | 10 | μM | MCF7 | 0 | −0.254 | −0.136 | 1073 |
| 4500 | 1033 | dinoprostone | 10 | μM | MCF7 | 0 | −0.254 | −0.116 | 6552 |
| 4501 | 680 | Prestwick-685 | 11 | μM | PC3 | 0 | −0.254 | −0.087 | 3683 |
| 4498 | 767 | haloperidol | 10 | μM | MCF7 | 0 | −0.253 | −0.209 | 6960 |
| 4496 | 612 | amiloride | 13 | μM | HL60 | 0 | −0.253 | −0.143 | 1970 |
| 4495 | 730 | ceforanide | 8 | μM | MCF7 | 0 | −0.253 | −0.113 | 5351 |
| 4497 | 1054 | pioglitazone | 10 | μM | PC3 | 0 | −0.253 | −0.061 | 6893 |
| 4494 | 623 | metergoline | 10 | μM | HL60 | 0 | −0.252 | −0.193 | 1606 |
| 4492 | 747 | isoniazid | 29 | μM | MCF7 | 0 | −0.252 | −0.162 | 7197 |
| 4493 | 701 | ketoprofen | 16 | μM | PC3 | 0 | −0.252 | −0.112 | 4286 |
| 4491 | 734 | abamectin | 5 | μM | PC3 | 0 | −0.252 | −0.108 | 5864 |
| 4485 | 1078 | thapsigargin | 100 | nM | MCF7 | 0 | −0.251 | −0.243 | 7100 |
| 4487 | 706 | arcaine | 15 | μM | MCF7 | 0 | −0.251 | −0.135 | 4974 |
| 4489 | 513 | valproic acid | 500 | μM | MCF7 | 0 | −0.251 | −0.126 | 1078 |
| 4490 | 701 | benzamil | 11 | μM | PC3 | 0 | −0.251 | −0.104 | 4294 |
| 4486 | 617 | oxymetazoline | 13 | μM | PC3 | 0 | −0.251 | −0.099 | 2114 |
| 4488 | 56 | fasudil | 10 | μM | PC3 | 0 | −0.251 | −0.071 | 436 |
| 4482 | 656 | colistin | 3 | μM | MCF7 | 0 | −0.25 | −0.1 | 2851 |
| 4483 | 733 | terazosin | 9 | μM | PC3 | 0 | −0.25 | −0.073 | 5831 |
| 4484 | 734 | sulfadoxine | 13 | μM | PC3 | 0 | −0.25 | −0.07 | 5852 |
| 4481 | 702 | helveticoside | 7 | μM | PC3 | 0 | −0.25 | −0.068 | 4327 |
| 4480 | 727 | troglitazone | 10 | μM | PC3 | 0 | −0.249 | −0.081 | 4456 |
| 4477 | 706 | cefaclor | 10 | μM | MCF7 | 0 | −0.248 | −0.134 | 4967 |
| 4476 | 720 | CP-690334-01 | 10 | μM | MCF7 | 0 | −0.248 | −0.116 | 4380 |
| 4475 | 646 | oxybutynin | 10 | μM | MCF7 | 0 | −0.248 | −0.099 | 3168 |
| 4479 | 764 | methylprednisolone | 11 | μM | PC3 | 0 | −0.248 | −0.094 | 7137 |
| 4473 | 772 | methocarbamol | 17 | μM | MCF7 | 0 | −0.248 | −0.092 | 7467 |
| 4474 | 704 | thiostrepton | 2 | μM | PC3 | 0 | −0.248 | −0.09 | 4563 |
| 4478 | 626 | sirolimus | 100 | nM | MCF7 | 0 | −0.248 | −0.085 | 1667 |
| 4467 | 663 | yohimbic acid | 11 | μM | MCF7 | 0 | −0.247 | −0.141 | 2803 |
| 4469 | 1004 | pioglitazone | 10 | μM | MCF7 | 0 | −0.247 | −0.105 | 5925 |
| 4471 | 673 | felbinac | 19 | μM | MCF7 | 0 | −0.247 | −0.102 | 3398 |
| 4472 | 754 | propafenone | 11 | μM | PC3 | 0 | −0.247 | −0.097 | 6336 |
| 4468 | 633 | edrophonium chloride | 20 | μM | MCF7 | 0 | −0.247 | −0.096 | 1519 |
| 4470 | 743 | naproxen | 16 | μM | MCF7 | 0 | −0.247 | −0.088 | 6794 |
| 4465 | 1041 | 5155877 | 10 | μM | MCF7 | 0 | −0.246 | −0.185 | 6574 |
| 4463 | 663 | Prestwick-642 | 14 | μM | MCF7 | 0 | −0.246 | −0.094 | 2815 |
| 4464 | 735 | dobutamine | 12 | μM | MCF7 | 0 | −0.246 | −0.066 | 5386 |
| 4466 | 610 | minoxidil | 19 | μM | PC3 | 0 | −0.246 | −0.057 | 1914 |
| 4462 | 662 | cinchonidine | 14 | μM | MCF7 | 0 | −0.245 | −0.176 | 2772 |
| 4456 | 659 | 2- | 23 | μM | HL60 | 0 | −0.245 | −0.149 | 3063 |
| | | aminobenzenesulfonamide | | | | | | | |
| 4459 | 728 | stachydrine | 22 | μM | PC3 | 0 | −0.245 | −0.101 | 4469 |
| 4460 | 632 | minaprine | 11 | μM | MCF7 | 0 | −0.245 | −0.091 | 1468 |
| 4461 | 506 | LY-294002 | 10 | μM | MCF7 | 0 | −0.245 | −0.089 | 1016 |
| 4457 | 733 | doxycycline | 8 | μM | PC3 | 0 | −0.245 | −0.086 | 5838 |
| 4458 | 683 | ethotoin | 20 | μM | PC3 | 0 | −0.245 | −0.084 | 3809 |
| 4455 | 765 | haloperidol | 10 | μM | MCF7 | 0 | −0.244 | −0.112 | 7003 |
| 4453 | 693 | cefalonium | 9 | μM | PC3 | 0 | −0.244 | −0.108 | 4245 |
| 4452 | 506 | clozapine | 10 | μM | MCF7 | 0 | −0.244 | −0.104 | 1009 |
| 4454 | 728 | furosemide | 12 | μM | PC3 | 0 | −0.244 | −0.102 | 4503 |
| 4451 | 683 | oxaprozin | 14 | μM | PC3 | 0 | −0.243 | −0.151 | 3794 |
| 4450 | 735 | dinoprost | 8 | μM | MCF7 | 0 | −0.243 | −0.114 | 5409 |
| 4449 | 767 | tanespimycin | 1 | μM | MCF7 | 0 | −0.242 | −0.11 | 6943 |
| 4448 | 662 | diclofenac | 13 | μM | MCF7 | 0 | −0.242 | −0.073 | 2756 |
| 4446 | 747 | diazoxide | 17 | μM | MCF7 | 0 | −0.241 | −0.13 | 7168 |
| 4447 | 655 | dicloxacillin | 8 | μM | MCF7 | 0 | −0.241 | −0.111 | 3307 |
| 4444 | 1062 | H-89 | 500 | nM | PC3 | 0 | −0.241 | −0.101 | 6921 |
| 4443 | 771 | fenofibrate | 11 | μM | MCF7 | 0 | −0.241 | −0.09 | 7432 |
| 4445 | 673 | capsaicin | 13 | μM | MCF7 | 0 | −0.241 | −0.08 | 3372 |
| 4442 | 728 | sertaconazole | 8 | μM | PC3 | 0 | −0.241 | −0.07 | 4475 |
| 4440 | 734 | neomycin | 4 | μM | PC3 | 0 | −0.24 | −0.148 | 5867 |
| 4436 | 735 | coralyne | 10 | μM | MCF7 | 0 | −0.24 | −0.137 | 5418 |
| 4438 | 754 | pinacidil | 16 | μM | PC3 | 0 | −0.24 | −0.13 | 6356 |
| 4441 | 676 | fluticasone | 8 | μM | MCF7 | 0 | −0.24 | −0.125 | 7348 |
| 4437 | 626 | LY-294002 | 10 | μM | MCF7 | 0 | −0.24 | −0.097 | 1664 |
| 4439 | 663 | cinchonine | 14 | μM | MCF7 | 0 | −0.24 | −0.094 | 2789 |
| 4428 | 747 | sulfamonomethoxine | 14 | μM | MCF7 | 0 | −0.239 | −0.199 | 7200 |
| 4431 | 706 | SR-95639A | 10 | μM | MCF7 | 0 | −0.239 | −0.185 | 4977 |
| 4432 | 648 | abamectin | 5 | μM | HL60 | 0 | −0.239 | −0.157 | 2519 |
| 4429 | 747 | cefotaxime | 8 | μM | MCF7 | 0 | −0.239 | −0.135 | 7186 |
| 4434 | 615 | oxymetazoline | 13 | μM | HL60 | 0 | −0.239 | −0.13 | 1431 |
| 4427 | 710 | ketanserin | 7 | μM | PC3 | 0 | −0.239 | −0.125 | 6649 |
| 4426 | 1094 | vinblastine | 100 | nM | MCF7 | 0 | −0.239 | −0.118 | 7551 |
| 4433 | 506 | LY-294002 | 10 | μM | MCF7 | 0 | −0.239 | −0.098 | 1019 |
| 4430 | 734 | estriol | 14 | μM | PC3 | 0 | −0.239 | −0.086 | 5866 |
| 4435 | 702 | PHA-00851261E | 10 | μM | PC3 | 0 | −0.239 | −0.086 | 4330 |
| 4424 | 632 | levodopa | 20 | μM | MCF7 | 0 | −0.238 | −0.135 | 1472 |
| 4420 | 689 | trimethadione | 28 | μM | PC3 | 0 | −0.238 | −0.127 | 4086 |
| 4422 | 646 | chlortalidone | 12 | μM | MCF7 | 0 | −0.238 | −0.118 | 3198 |
| 4423 | 676 | gabexate | 10 | μM | MCF7 | 0 | −0.238 | −0.097 | 7357 |
| 4425 | 506 | estradiol | 10 | nM | MCF7 | 0 | −0.238 | −0.084 | 1021 |
| 4421 | 71 | sodium phenylbutyrate | 200 | μM | SKMEL5 | 0 | −0.238 | −0.073 | 502 |
| 4419 | 747 | tetrandrine | 6 | μM | MCF7 | 0 | −0.237 | −0.233 | 7178 |
| 4417 | 725 | sirolimus | 100 | nM | MCF7 | 0 | −0.237 | −0.125 | 5239 |
| 4418 | 690 | fluticasone | 8 | μM | MCF7 | 0 | −0.237 | −0.113 | 4129 |
| 4415 | 655 | iohexol | 5 | μM | MCF7 | 0 | −0.237 | −0.112 | 3322 |
| 4414 | 617 | chlorzoxazone | 24 | μM | PC3 | 0 | −0.237 | −0.103 | 2100 |
| 4416 | 701 | metoclopramide | 12 | μM | PC3 | 0 | −0.237 | −0.084 | 4285 |
| 4410 | 747 | ursolic acid | 9 | μM | MCF7 | 0 | −0.236 | −0.143 | 7181 |
| 4413 | 661 | nabumetone | 18 | μM | HL60 | 0 | −0.236 | −0.125 | 3108 |
| 4411 | 735 | clebopride | 8 | μM | MCF7 | 0 | −0.236 | −0.12 | 5412 |
| 4412 | 1065 | AH-6809 | 1 | μM | PC3 | 0 | −0.236 | −0.087 | 7049 |
| 4407 | 680 | halcinonide | 9 | μM | PC3 | 0 | −0.235 | −0.087 | 3680 |
| 4409 | 655 | methoxsalen | 19 | μM | MCF7 | 0 | −0.235 | −0.086 | 3302 |
| 4408 | 708 | guanabenz | 14 | μM | MCF7 | 0 | −0.235 | −0.079 | 5703 |
| 4406 | 743 | ribostamycin | 7 | μM | MCF7 | 0 | −0.235 | −0.054 | 6765 |
| 4400 | 623 | betamethasone | 10 | μM | HL60 | 0 | −0.234 | −0.153 | 1590 |
| 4404 | 614 | disulfiram | 13 | μM | HL60 | 0 | −0.234 | −0.152 | 1369 |
| 4405 | 703 | orphenadrine | 13 | μM | PC3 | 0 | −0.234 | −0.136 | 4537 |
| 4401 | 699 | PNU-0251126 | 1 | μM | MCF7 | 0 | −0.234 | −0.134 | 4714 |
| 4403 | 1021 | orlistat | 10 | μM | PC3 | 0 | −0.234 | −0.112 | 6388 |
| 4399 | 720 | spiradoline | 1 | μM | MCF7 | 0 | −0.234 | −0.108 | 4375 |
| 4402 | 690 | nadolol | 13 | μM | MCF7 | 0 | −0.234 | −0.083 | 4139 |
| 4396 | 691 | alprostadil | 11 | μM | MCF7 | 0 | −0.233 | −0.098 | 4179 |
| 4398 | 690 | nafcillin | 9 | μM | MCF7 | 0 | −0.233 | −0.096 | 4103 |
| 4397 | 681 | sulfamethoxypyridazine | 14 | μM | PC3 | 0 | −0.233 | −0.087 | 3711 |
| 4393 | 680 | kawain | 17 | μM | PC3 | 0 | −0.232 | −0.156 | 3670 |
| 4392 | 771 | isotretinoin | 13 | μM | MCF7 | 0 | −0.232 | −0.124 | 7438 |
| 4395 | 734 | quipazine | 9 | μM | PC3 | 0 | −0.232 | −0.116 | 5887 |
| 4391 | 736 | S-propranolol | 14 | μM | MCF7 | 0 | −0.232 | −0.115 | 5444 |
| 4394 | 705 | dicycloverine | 12 | μM | MCF7 | 0 | −0.232 | −0.101 | 4405 |
| 4389 | 633 | ampicillin | 10 | μM | MCF7 | 0 | −0.231 | −0.13 | 1530 |
| 4390 | 1010 | tanespimycin | 1 | μM | MCF7 | 0 | −0.231 | −0.101 | 5953 |
| 4387 | 757 | trifluoperazine | 10 | μM | MCF7 | 0 | −0.23 | −0.225 | 5584 |
| 4388 | 659 | propranolol | 14 | μM | HL60 | 0 | −0.23 | −0.152 | 3059 |
| 4386 | 757 | wortmannin | 10 | nM | MCF7 | 0 | −0.23 | −0.087 | 5603 |
| 4384 | 663 | palmatine | 10 | μM | MCF7 | 0 | −0.229 | −0.119 | 2795 |
| 4383 | 746 | hydroquinine | 9 | μM | MCF7 | 0 | −0.229 | −0.1 | 6263 |
| 4385 | 676 | zardaverine | 15 | μM | MCF7 | 0 | −0.229 | −0.085 | 7347 |
| 4379 | 702 | mexiletine | 19 | μM | PC3 | 0 | −0.228 | −0.127 | 4338 |
| 4376 | 730 | metanephrine | 17 | μM | MCF7 | 0 | −0.228 | −0.12 | 5334 |
| 4381 | 502 | rottlerin | 10 | μM | MCF7 | 0 | −0.228 | −0.118 | 941 |
| 4378 | 732 | methazolamide | 17 | μM | PC3 | 0 | −0.228 | −0.115 | 5794 |
| 4377 | 701 | betonicine | 25 | μM | PC3 | 0 | −0.228 | −0.097 | 4301 |
| 4380 | 711 | mexiletine | 19 | μM | MCF7 | 0 | −0.228 | −0.088 | 3973 |
| 4382 | 677 | penbutolol | 6 | μM | MCF7 | 0 | −0.228 | −0.075 | 3534 |
| 4374 | 632 | khellin | 15 | μM | MCF7 | 0 | −0.227 | −0.104 | 1504 |
| 4375 | 757 | genistein | 10 | μM | MCF7 | 0 | −0.227 | −0.098 | 5595 |
| 4369 | 695 | zuclopenthixol | 9 | μM | MCF7 | 0 | −0.226 | −0.18 | 4843 |
| 4368 | 654 | lactobionic acid | 11 | μM | MCF7 | 0 | −0.226 | −0.13 | 3246 |
| 4371 | 680 | dilazep | 6 | μM | PC3 | 0 | −0.226 | −0.102 | 3665 |
| 4373 | 53 | trifluoperazine | 10 | μM | MCF7 | 0 | −0.226 | −0.097 | 421 |
| 4370 | 713 | loperamide | 8 | μM | PC3 | 0 | −0.226 | −0.095 | 4672 |
| 4367 | 706 | Prestwick-857 | 12 | μM | MCF7 | 0 | −0.226 | −0.091 | 4980 |
| 4372 | 726 | haloperidol | 11 | μM | MCF7 | 0 | −0.226 | −0.086 | 5273 |
| 4362 | 702 | vincamine | 11 | μM | PC3 | 0 | −0.225 | −0.134 | 4341 |
| 4365 | 611 | lisuride | 12 | μM | PC3 | 0 | −0.225 | −0.117 | 1962 |
| 4361 | 632 | phenazone | 21 | μM | MCF7 | 0 | −0.225 | −0.102 | 1489 |
| 4366 | 681 | sulfamerazine | 15 | μM | PC3 | 0 | −0.225 | −0.072 | 3718 |
| 4364 | 738 | dropropizine | 17 | μM | MCF7 | 0 | −0.225 | −0.068 | 5531 |
| 4363 | 767 | estradiol | 10 | nM | MCF7 | 0 | −0.225 | −0.046 | 6957 |
| 4360 | 623 | ascorbic acid | 22 | μM | HL60 | 0 | −0.224 | −0.167 | 1610 |
| 4356 | 728 | diperodon | 9 | μM | PC3 | 0 | −0.224 | −0.117 | 4498 |
| 4359 | 707 | brinzolamide | 10 | μM | MCF7 | 0 | −0.224 | −0.116 | 5016 |
| 4354 | 710 | diloxanide | 12 | μM | PC3 | 0 | −0.224 | −0.104 | 6679 |
| 4355 | 673 | primidone | 18 | μM | MCF7 | 0 | −0.224 | −0.096 | 3402 |
| 4358 | 689 | moxonidine | 17 | μM | PC3 | 0 | −0.224 | −0.092 | 4084 |
| 4357 | 626 | tanespimycin | 1 | μM | MCF7 | 0 | −0.224 | −0.059 | 1650 |
| 4351 | 699 | monensin | 6 | μM | MCF7 | 0 | −0.223 | −0.143 | 4726 |
| 4347 | 713 | flurbiprofen | 16 | μM | PC3 | 0 | −0.223 | −0.129 | 4674 |
| 4352 | 685 | finasteride | 11 | μM | MCF7 | 0 | −0.223 | −0.124 | 3641 |
| 4353 | 654 | metrizamide | 5 | μM | MCF7 | 0 | −0.223 | −0.112 | 3255 |
| 4349 | 647 | metitepine | 8 | μM | MCF7 | 0 | −0.223 | −0.107 | 3231 |
| 4350 | 703 | ciclacillin | 12 | μM | PC3 | 0 | −0.223 | −0.105 | 4536 |
| 4348 | 116 | estradiol | 10 | nM | PC3 | 0 | −0.223 | −0.067 | 665 |
| 4342 | 743 | butirosin | 5 | μM | MCF7 | 0 | −0.222 | −0.143 | 6779 |
| 4341 | 708 | felbinac | 19 | μM | MCF7 | 0 | −0.222 | −0.127 | 5700 |
| 4336 | 648 | podophyllotoxin | 10 | μM | HL60 | 0 | −0.222 | −0.121 | 2540 |
| 4338 | 743 | tamoxifen | 7 | μM | MCF7 | 0 | −0.222 | −0.12 | 6768 |
| 4343 | 631 | carbarsone | 15 | μM | HL60 | 0 | −0.222 | −0.116 | 1313 |
| 4334 | 743 | pyrithyldione | 24 | μM | MCF7 | 0 | −0.222 | −0.109 | 6801 |
| 4345 | 698 | riluzole | 15 | μM | PC3 | 0 | −0.222 | −0.109 | 7365 |
| 4335 | 712 | colchicine | 10 | μM | PC3 | 0 | −0.222 | −0.103 | 4614 |
| 4339 | 772 | trapidil | 19 | μM | MCF7 | 0 | −0.222 | −0.091 | 7475 |
| 4340 | 90 | splitomicin | 20 | μM | PC3 | 0 | −0.222 | −0.088 | 661 |
| 4344 | 37 | rofecoxib | 10 | μM | HL60 | 0 | −0.222 | −0.083 | 371 |
| 4337 | 695 | tocainide | 17 | μM | MCF7 | 0 | −0.222 | −0.07 | 4838 |
| 4346 | 719 | parthenolide | 16 | μM | PC3 | 0 | −0.222 | −0.068 | 5105 |
| 4332 | 729 | tacrine | 16 | μM | MCF7 | 0 | −0.221 | −0.173 | 5297 |
| 4329 | 683 | tinidazole | 16 | μM | PC3 | 0 | −0.221 | −0.11 | 3813 |
| 4333 | 617 | pentetrazol | 29 | μM | PC3 | 0 | −0.221 | −0.081 | 2092 |
| 4330 | 734 | harmine | 16 | μM | PC3 | 0 | −0.221 | −0.078 | 5855 |
| 4328 | 713 | pirenperone | 10 | μM | PC3 | 0 | −0.221 | −0.076 | 4679 |
| 4331 | 626 | genistein | 10 | μM | MCF7 | 0 | −0.221 | −0.066 | 1660 |
| 4327 | 676 | decamethonium bromide | 10 | μM | MCF7 | 0 | −0.22 | −0.168 | 7353 |
| 4325 | 732 | dexamethasone | 9 | μM | PC3 | 0 | −0.22 | −0.158 | 5797 |
| 4324 | 109 | benserazide | 10 | μM | SKMEL5 | 0 | −0.22 | −0.141 | 631 |
| 4321 | 725 | LY-294002 | 10 | μM | MCF7 | 0 | −0.22 | −0.126 | 5233 |
| 4323 | 678 | ramipril | 10 | μM | MCF7 | 0 | −0.22 | −0.11 | 3572 |
| 4322 | 673 | aminophylline | 10 | μM | MCF7 | 0 | −0.22 | −0.099 | 3374 |
| 4326 | 71 | LY-294002 | 10 | μM | SKMEL5 | 0 | −0.22 | −0.087 | 501 |
| 4320 | 703 | fenbendazole | 13 | μM | PC3 | 0 | −0.219 | −0.132 | 4542 |
| 4318 | 1066 | colforsin | 500 | nM | MCF7 | 0 | −0.219 | −0.122 | 7055 |
| 4319 | 737 | tridihexethyl | 11 | μM | MCF7 | 0 | −0.219 | −0.092 | 5486 |
| 4316 | 754 | doxepin | 13 | μM | PC3 | 0 | −0.219 | −0.086 | 6337 |
| 4315 | 730 | erythromycin | 5 | μM | MCF7 | 0 | −0.219 | −0.082 | 5329 |
| 4317 | 505 | ikarugamycin | 2 | μM | MCF7 | 0 | −0.219 | −0.08 | 918 |
| 4314 | 712 | practolol | 15 | μM | PC3 | 0 | −0.219 | −0.066 | 4603 |
| 4313 | 706 | methoxamine | 16 | μM | MCF7 | 0 | −0.218 | −0.178 | 4972 |
| 4311 | 602 | fluphenazine | 10 | μM | HL60 | 0 | −0.218 | −0.173 | 1178 |
| 4312 | 725 | fluphenazine | 10 | μM | MCF7 | 0 | −0.218 | −0.084 | 5234 |
| 4310 | 718 | harmalol | 15 | μM | PC3 | 0 | −0.218 | −0.076 | 5076 |
| 4309 | 741 | lincomycin | 9 | μM | MCF7 | 0 | −0.218 | −0.069 | 5992 |
| 4304 | 1079 | thapsigargin | 100 | nM | PC3 | 0 | −0.217 | −0.185 | 7103 |
| 4308 | 725 | tanespimycin | 1 | μM | MCF7 | 0 | −0.217 | −0.146 | 5215 |
| 4307 | 701 | lomefloxacin | 10 | μM | PC3 | 0 | −0.217 | −0.124 | 4281 |
| 4306 | 1003 | rotenone | 1 | μM | PC3 | 0 | −0.217 | −0.119 | 5920 |
| 4301 | 702 | fluocinonide | 8 | μM | PC3 | 0 | −0.217 | −0.109 | 4314 |
| 4300 | 701 | Prestwick-674 | 14 | μM | PC3 | 0 | −0.217 | −0.104 | 4276 |
| 4296 | 772 | penbutolol | 6 | μM | MCF7 | 0 | −0.217 | −0.103 | 7476 |
| 4303 | 676 | zalcitabine | 19 | μM | MCF7 | 0 | −0.217 | −0.094 | 7352 |
| 4299 | 734 | mepyramine | 10 | μM | PC3 | 0 | −0.217 | −0.091 | 5869 |
| 4297 | 718 | pizotifen | 9 | μM | PC3 | 0 | −0.217 | −0.09 | 5072 |
| 4302 | 676 | 3-acetamidocoumarin | 20 | μM | MCF7 | 0 | −0.217 | −0.086 | 7361 |
| 4305 | 632 | acebutolol | 11 | μM | MCF7 | 0 | −0.217 | −0.069 | 1493 |
| 4298 | 611 | metolazone | 11 | μM | PC3 | 0 | −0.217 | −0.067 | 1932 |
| 4293 | 729 | naftidrofuryl | 8 | μM | MCF7 | 0 | −0.216 | −0.145 | 5287 |
| 4295 | 677 | naftifine | 12 | μM | MCF7 | 0 | −0.216 | −0.133 | 3536 |
| 4292 | 735 | nimodipine | 10 | μM | MCF7 | 0 | −0.216 | −0.108 | 5421 |
| 4294 | 745 | fluorocurarine | 12 | μM | MCF7 | 0 | −0.216 | −0.102 | 6219 |
| 4291 | 656 | tiaprofenic acid | 15 | μM | MCF7 | 0 | −0.215 | −0.107 | 2852 |
| 4290 | 671 | sulfamonomethoxine | 14 | μM | MCF7 | 0 | −0.215 | −0.099 | 3484 |
| 4289 | 626 | wortmannin | 10 | nM | MCF7 | 0 | −0.215 | −0.096 | 1668 |
| 4284 | 704 | vitexin | 9 | μM | PC3 | 0 | −0.214 | −0.187 | 4588 |
| 4286 | 747 | podophyllotoxin | 10 | μM | MCF7 | 0 | −0.214 | −0.183 | 7198 |
| 4285 | 772 | triflupromazine | 10 | μM | MCF7 | 0 | −0.214 | −0.171 | 7466 |
| 4282 | 670 | cefamandole | 8 | μM | MCF7 | 0 | −0.214 | −0.146 | 3436 |
| 4288 | 673 | esculin | 12 | μM | MCF7 | 0 | −0.214 | −0.107 | 3390 |
| 4287 | 758 | probucol | 8 | μM | MCF7 | 0 | −0.214 | −0.103 | 5626 |
| 4283 | 753 | nizatidine | 12 | μM | PC3 | 0 | −0.214 | −0.061 | 6305 |
| 4278 | 626 | estradiol | 10 | nM | MCF7 | 0 | −0.213 | −0.151 | 1666 |
| 4280 | 651 | securinine | 18 | μM | HL60 | 0 | −0.213 | −0.122 | 2729 |
| 4281 | 706 | acebutolol | 11 | μM | MCF7 | 0 | −0.213 | −0.113 | 4976 |
| 4277 | 714 | florfenicol | 11 | μM | PC3 | 0 | −0.213 | −0.103 | 6701 |
| 4279 | 663 | Prestwick-682 | 6 | μM | MCF7 | 0 | −0.213 | −0.067 | 2819 |
| 4272 | 730 | fluoxetine | 12 | μM | MCF7 | 0 | −0.212 | −0.132 | 5356 |
| 4274 | 714 | naftidrofuryl | 8 | μM | PC3 | 0 | −0.212 | −0.107 | 6687 |
| 4273 | 754 | scopolamine N-oxide | 10 | μM | PC3 | 0 | −0.212 | −0.104 | 6335 |
| 4276 | 734 | oxprenolol | 13 | μM | PC3 | 0 | −0.212 | −0.102 | 5871 |
| 4275 | 506 | prochlorperazine | 10 | μM | MCF7 | 0 | −0.212 | −0.091 | 995 |
| 4270 | 729 | nitrofural | 20 | μM | MCF7 | 0 | −0.211 | −0.083 | 5321 |
| 4271 | 734 | convolamine | 12 | μM | PC3 | 0 | −0.211 | −0.077 | 5876 |
| 4264 | 676 | tracazolate | 12 | μM | MCF7 | 0 | −0.21 | −0.134 | 7339 |
| 4269 | 602 | LY-294002 | 10 | μM | HL60 | 0 | −0.21 | −0.128 | 1177 |
| 4268 | 623 | alfuzosin | 9 | μM | HL60 | 0 | −0.21 | −0.122 | 1586 |
| 4265 | 602 | nordihydroguaiaretic acid | 1 | μM | HL60 | 0 | −0.21 | −0.111 | 1164 |
| 4266 | 672 | arcaine | 15 | μM | MCF7 | 0 | −0.21 | −0.083 | 3349 |
| 4267 | 1011 | estradiol | 10 | nM | PC3 | 0 | −0.21 | −0.079 | 5960 |
| 4261 | 514 | phentolamine | 12 | μM | MCF7 | 0 | −0.209 | −0.178 | 1138 |
| 4257 | 661 | tiletamine | 15 | μM | HL60 | 0 | −0.209 | −0.169 | 3137 |
| 4260 | 730 | neostigmine bromide | 13 | μM | MCF7 | 0 | −0.209 | −0.131 | 5335 |
| 4258 | 616 | dexamethasone | 9 | μM | PC3 | 0 | −0.209 | −0.128 | 2079 |
| 4263 | 646 | clotrimazole | 12 | μM | MCF7 | 0 | −0.209 | −0.111 | 3166 |
| 4255 | 700 | PNU-0230031 | 10 | μM | MCF7 | 0 | −0.209 | −0.111 | 4754 |
| 4254 | 686 | metamizole sodium | 12 | μM | MCF7 | 0 | −0.209 | −0.105 | 3835 |
| 4259 | 745 | trichostatin A | 100 | nM | MCF7 | 0 | −0.209 | −0.098 | 6222 |
| 4262 | 706 | harmaline | 14 | μM | MCF7 | 0 | −0.209 | −0.086 | 4968 |
| 4256 | 738 | metampicillin | 10 | μM | MCF7 | 0 | −0.209 | −0.07 | 5540 |
| 4249 | 707 | metixene | 12 | μM | MCF7 | 0 | −0.208 | −0.192 | 5018 |
| 4250 | 677 | tribenoside | 8 | μM | MCF7 | 0 | −0.208 | −0.15 | 3507 |
| 4251 | 662 | syrosingopine | 6 | μM | MCF7 | 0 | −0.208 | −0.125 | 2753 |
| 4252 | 750 | sirolimus | 100 | nM | HL60 | 0 | −0.208 | −0.09 | 6180 |
| 4253 | 1073 | AH-6809 | 1 | μM | PC3 | 0 | −0.208 | −0.089 | 7075 |
| 4248 | 658 | iodixanol | 3 | μM | HL60 | 0 | −0.207 | −0.166 | 3023 |
| 4244 | 658 | oxolamine | 9 | μM | HL60 | 0 | −0.207 | −0.143 | 3006 |
| 4240 | 686 | famprofazone | 11 | μM | MCF7 | 0 | −0.207 | −0.129 | 3834 |
| 4245 | 505 | topiramate | 3 | μM | MCF7 | 0 | −0.207 | −0.114 | 915 |
| 4243 | 771 | dyclonine | 12 | μM | MCF7 | 0 | −0.207 | −0.102 | 7423 |
| 4247 | 765 | estradiol | 10 | nM | MCF7 | 0 | −0.207 | −0.101 | 7000 |
| 4241 | 687 | thiamazole | 35 | μM | MCF7 | 0 | −0.207 | −0.094 | 3898 |
| 4242 | 506 | haloperidol | 10 | μM | MCF7 | 0 | −0.207 | −0.06 | 983 |
| 4246 | 693 | Prestwick-967 | 26 | μM | PC3 | 0 | −0.207 | −0.057 | 4250 |
| 4236 | 731 | cyclopentolate | 12 | μM | PC3 | 0 | −0.206 | −0.144 | 5734 |
| 4238 | 743 | anabasine | 25 | μM | MCF7 | 0 | −0.206 | −0.132 | 6774 |
| 4239 | 678 | kaempferol | 14 | μM | MCF7 | 0 | −0.206 | −0.129 | 3579 |
| 4234 | 771 | enalapril | 8 | μM | MCF7 | 0 | −0.206 | −0.117 | 7428 |
| 4235 | 741 | ribavirin | 16 | μM | MCF7 | 0 | −0.206 | −0.105 | 6018 |
| 4237 | 505 | decitabine | 100 | nM | MCF7 | 0 | −0.206 | −0.066 | 920 |
| 4227 | 514 | cytochalasin B | 21 | μM | MCF7 | 0 | −0.205 | −0.175 | 1122 |
| 4228 | 731 | alclometasone | 8 | μM | PC3 | 0 | −0.205 | −0.146 | 5752 |
| 4232 | 727 | rosiglitazone | 10 | μM | PC3 | 0 | −0.205 | −0.139 | 4457 |
| 4229 | 762 | dosulepin | 12 | μM | PC3 | 0 | −0.205 | −0.109 | 7284 |
| 4233 | 654 | cefixime | 9 | μM | MCF7 | 0 | −0.205 | −0.093 | 3247 |
| 4231 | 748 | fluphenazine | 8 | μM | MCF7 | 0 | −0.205 | −0.079 | 7234 |
| 4230 | 1014 | PF-00539745-00 | 10 | μM | MCF7 | 0 | −0.205 | −0.062 | 5974 |
| 4222 | 1047 | 5194442 | 20 | μM | MCF7 | 0 | −0.204 | −0.144 | 6599 |
| 4226 | 648 | benzethonium chloride | 9 | μM | HL60 | 0 | −0.204 | −0.112 | 2508 |
| 4221 | 1000 | estradiol | 10 | nM | MCF7 | 0 | −0.204 | −0.109 | 5905 |
| 4224 | 627 | benzonatate | 7 | μM | MCF7 | 0 | −0.204 | −0.104 | 1679 |
| 4225 | 657 | tubocurarine chloride | 5 | μM | MCF7 | 0 | −0.204 | −0.099 | 2887 |
| 4223 | 729 | loxapine | 9 | μM | MCF7 | 0 | −0.204 | −0.084 | 5293 |
| 4217 | 671 | bucladesine | 8 | μM | MCF7 | 0 | −0.203 | −0.152 | 3483 |
| 4216 | 676 | gibberellic acid | 12 | μM | MCF7 | 0 | −0.203 | −0.147 | 7330 |
| 4220 | 673 | bemegride | 26 | μM | MCF7 | 0 | −0.203 | −0.145 | 3389 |
| 4213 | 677 | bethanechol | 20 | μM | MCF7 | 0 | −0.203 | −0.128 | 3537 |
| 4214 | 514 | doxycycline | 14 | μM | MCF7 | 0 | −0.203 | −0.123 | 1113 |
| 4211 | 734 | diclofenac | 13 | μM | PC3 | 0 | −0.203 | −0.101 | 5861 |
| 4212 | 765 | fluphenazine | 10 | μM | MCF7 | 0 | −0.203 | −0.088 | 6996 |
| 4218 | 753 | zoxazolamine | 24 | μM | PC3 | 0 | −0.203 | −0.067 | 6290 |
| 4219 | 747 | benzydamine | 12 | μM | MCF7 | 0 | −0.203 | −0.065 | 7169 |
| 4215 | 738 | sulindac | 11 | μM | MCF7 | 0 | −0.203 | −0.064 | 5528 |
| 4207 | 766 | aceclofenac | 11 | μM | MCF7 | 0 | −0.202 | −0.148 | 7029 |
| 4208 | 747 | mifepristone | 9 | μM | MCF7 | 0 | −0.202 | −0.129 | 7183 |
| 4209 | 626 | valproic acid | 500 | μM | MCF7 | 0 | −0.202 | −0.129 | 1665 |
| 4210 | 719 | prednicarbate | 8 | μM | PC3 | 0 | −0.202 | −0.101 | 5119 |
| 4199 | 703 | santonin | 16 | μM | PC3 | 0 | −0.201 | −0.161 | 4531 |
| 4201 | 677 | risperidone | 10 | μM | MCF7 | 0 | −0.201 | −0.153 | 3508 |
| 4206 | 506 | wortmannin | 10 | nM | MCF7 | 0 | −0.201 | −0.085 | 1023 |
| 4204 | 703 | chlorcyclizine | 12 | μM | PC3 | 0 | −0.201 | −0.084 | 4546 |
| 4205 | 718 | allantoin | 25 | μM | PC3 | 0 | −0.201 | −0.076 | 5052 |
| 4200 | 1085 | daunorubicin | 1 | μM | PC3 | 0 | −0.201 | −0.066 | 7511 |
| 4203 | 715 | buspirone | 9 | μM | PC3 | 0 | −0.201 | −0.059 | 6743 |
| 4202 | 715 | ioversol | 5 | μM | PC3 | 0 | −0.201 | −0.051 | 6726 |
| 4191 | 703 | parbendazole | 16 | μM | PC3 | 0 | −0.2 | −0.165 | 4535 |
| 4197 | 627 | thiamphenicol | 11 | μM | MCF7 | 0 | −0.2 | −0.162 | 1704 |
| 4195 | 613 | josamycin | 5 | μM | HL60 | 0 | −0.2 | −0.16 | 2034 |
| 4193 | 725 | wortmannin | 10 | nM | MCF7 | 0 | −0.2 | −0.152 | 5240 |
| 4192 | 632 | trimethobenzamide | 9 | μM | MCF7 | 0 | −0.2 | −0.149 | 1502 |
| 4198 | 681 | heliotrine | 13 | μM | PC3 | 0 | −0.2 | −0.124 | 3717 |
| 4194 | 728 | clobetasol | 9 | μM | PC3 | 0 | −0.2 | −0.122 | 4497 |
| 4189 | 631 | meclocycline | 6 | μM | HL60 | 0 | −0.2 | −0.111 | 1341 |
| 4190 | 683 | flutamide | 14 | μM | PC3 | 0 | −0.2 | −0.105 | 3803 |
| 4196 | 694 | amantadine | 10 | μM | MCF7 | 0 | −0.2 | −0.056 | 4806 |

Reversal of up-regulated CRG expression is indicated by a negative ES score for the up-regulated genes. Drugs are considered to target the up-regulated genes if the ESup value is lower than −0.2. A lack of reversal of down-regulated genes is indicated by a negative ES score for this segment of the CRG signature.

### 5. Example 5

### System-Wide Control of Malignant Cell Transformation by Cooperating Oncogenic Mutations

a) Results

(1) Malignant Transformation Relies on Altered Expression of Cooperation Response Genes Implicated in Multiple Cell Biological Processes

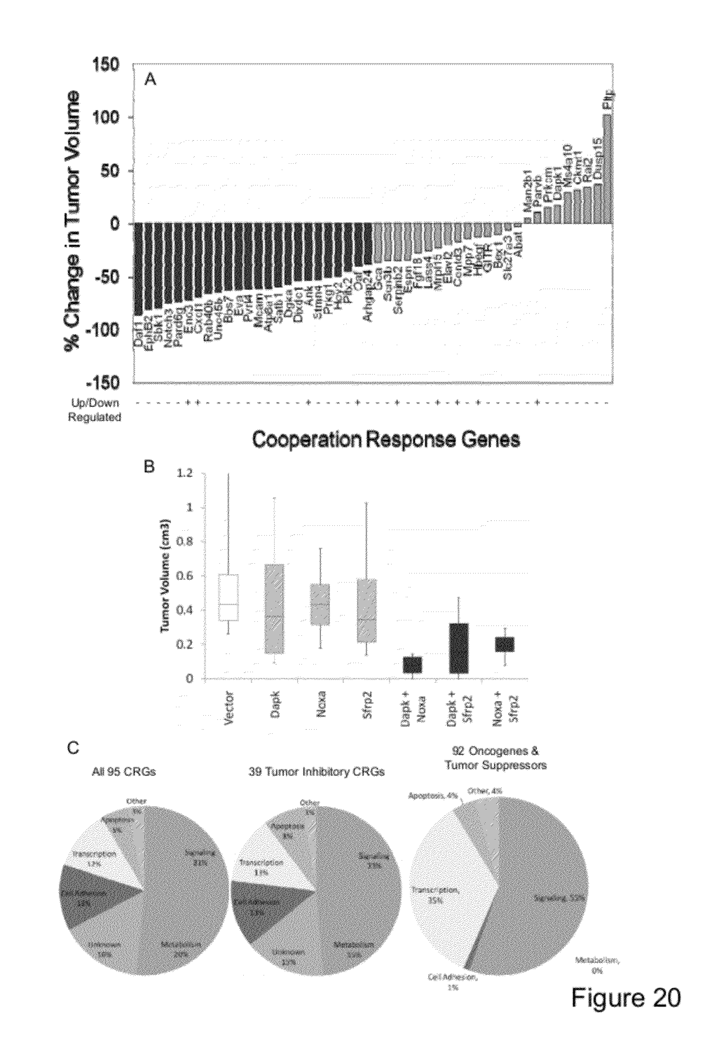

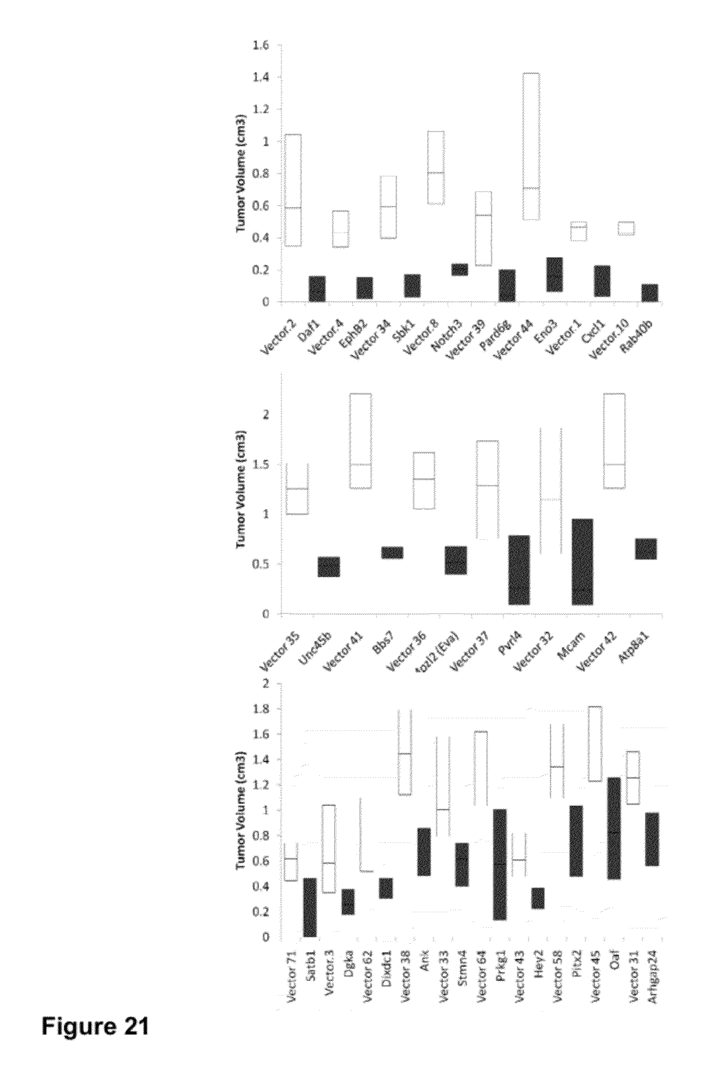

While a subset of CRGs has been shown to play an essential role in tumor formation, this set of perturbations was neither sufficient to test whether CRGs essential to the cancer cell regulate all or only specific biological processes, nor to assess the full extent to which members of the CRG set contribute to malignant transformation. To answer these questions, a novel set of 48 CRGs were perturbed in young adult mouse colon (YAMC) cells transformed by the combination of mutant p53^{175H} and Ras^{v12} (mp53/Ras cells), representing all the CRGs not previously tested and amenable to genetic manipulation with currently available tools. Among these 48 CRGs, a high proportion, 24 genes, is essential to the tumor formation capacity of mp53/Ras cells, with gene perturbation producing significant reductions in tumor volume at four weeks post-injection, as compared to matched, empty vector-expressing cells (FIG. 20A, FIG. 21, t-test, p<0.05). Disclosed in an earlier example herein, similar proportion of CRG perturbations (14/24 genes) produced a significant decrease in tumor formation upon xenograft in nude mice. Thus, more than 50% of the CRG set is comprised of genes that individually regulate the tumor formation capacity of cancer cells.

Although single perturbation of a large proportion of CRGs reveals an important role for these genes in transformation, among CRGs without a demonstrable effect on tumor formation were a number of genes with reported effects on cancer cells. Notably, genes such as Dapk1, a pro-apoptotic kinase and known tumor suppressor, Noxa, a p53 target gene and BH3-domain protein with a direct role in apoptotic control, and Sfrp2, a negative regulator of the Wnt signaling pathway whose expression is lost in many human colon cancers, has a causal role in cell transformation downstream of cooperating oncogenes. Because combined perturbation of weakly tumor inhibitory CRGs produced synergistic reductions in tumor size, combinations of CRG perturbations without significant effects individually were tested to determine if they could interact to inhibit tumors. Cells were engineered with each pair-wise combination of Dapk1, Noxa and Sfrp2, as well as cells re-expressing each of these genes individually and appropriate controls. Resetting expression of any of these CRG pairs produced significant tumor inhibition, while individual perturbation of these genes had little effect on tumor volume (FIG. 20B), demonstrating a role for Dapk1, Noxa and Sfrp2 in control of malignancy that could not be observed upon single gene perturbation.

CRG perturbations were made by retroviral introduction into mp53/Ras cells of cDNA encoding each target gene, or shRNA targeting each gene for mRNA knock-down, using multiple independent shRNA targets to control for potential off-target effects. The extent of gene perturbation was controlled at the RNA level (FIG. 9). Perturbed cells were compared to empty vector-infected mp53/Ras cells, as well as normal YAMC cells, to assess whether gene expression was reset in the range of normal cell expression. For tumor-inhibitory CRGs, replicates express cDNAs at levels below, at or moderately above YAMC mRNA expression levels, with the exception of the CRGs Pyr14, Rab40b, and Stmn4 (FIG. 9). For shRNA-mediated gene knockdown, two independent shRNA constructs were utilized for perturbation of all genes, with each construct achieving at least 50% knockdown of mRNA levels for the target gene. Polyclonal mp53/Ras cell populations stably expressing these constructs were implanted sub-cutaneously on nude mice and tumor formation was assessed over four weeks post injection. Effects on tumor formation capacity of mp53/Ras cells occur downstream of cooperating oncogenic mp53 and Ras proteins, as tumor inhibitory CRG perturbations do not alter the expression levels of either protein, assessed by Western blotting.

Based on comprehensive targeting of the CRG set, the contribution to tumor formation of genes involved in various cell processes was assessed. Overall, the CRG set contains a large number of genes involved in cell signaling and metabolism/transport, with relatively fewer genes regulating cell adhesion and motility, transcription and apoptosis (FIG. 20C), according to the Gene Ontology database biological process designations (GO). Remarkably, CRGs whose individual perturbation restricts tumor formation capacity of cells are drawn proportionally from each of these functional classes, demonstrating that oncogene cooperation induces a state change in the cancer cell via the CRG set, which control all the key functionalities required for cell transformation. The distribution of biological processes regulated by CRGs, especially cancer cell metabolism and adhesion/motility, is quite distinct from the functionalities of known cellular oncogenes, which are comprised almost exclusively of signaling molecules and transcription factors (FIG. 20C). The CRG set thereby can open access to a novel set of molecules, such as metabolic enzymes, critical to cancer cells, which are more readily targetable than classical oncogenes and tumor suppressors.

(2) Cooperative Control of Gene Expression at Transcriptional and Translational Levels

Cooperating oncogenes can alter the expression and/or activity of downstream targets, depending on the specific genes involved, indicating that the synergistic response to oncogenic mutations happens at multiple levels of cell regulation.

Original CRG expression profiles were derived from polysomal RNA, the mRNA fraction bound to ribosomes and actively being translated, in order to access genomic information that integrated the various levels of gene expression regulation in the cell, including transcriptional and translational. In order to test whether cooperative control of CRGs takes place at both levels of expression, CRG expression profiles derived from total RNA and polysomal RNA were compared using TaqMan Low Density Arrays (TLDA), QPCR-based arrays, which were customized to probe for the CRG set (56 CRGs represented based on probe set availability). Four replicates of total or polysomal RNA were analyzed for CRG expression patterns from young adult mouse colon cells (YAMC), YAMC cells expressing mp53 alone (mp53), YAMC cells expressing Ras alone (Ras) and YAMC cells expressing the combination of mp53 and Ras together.

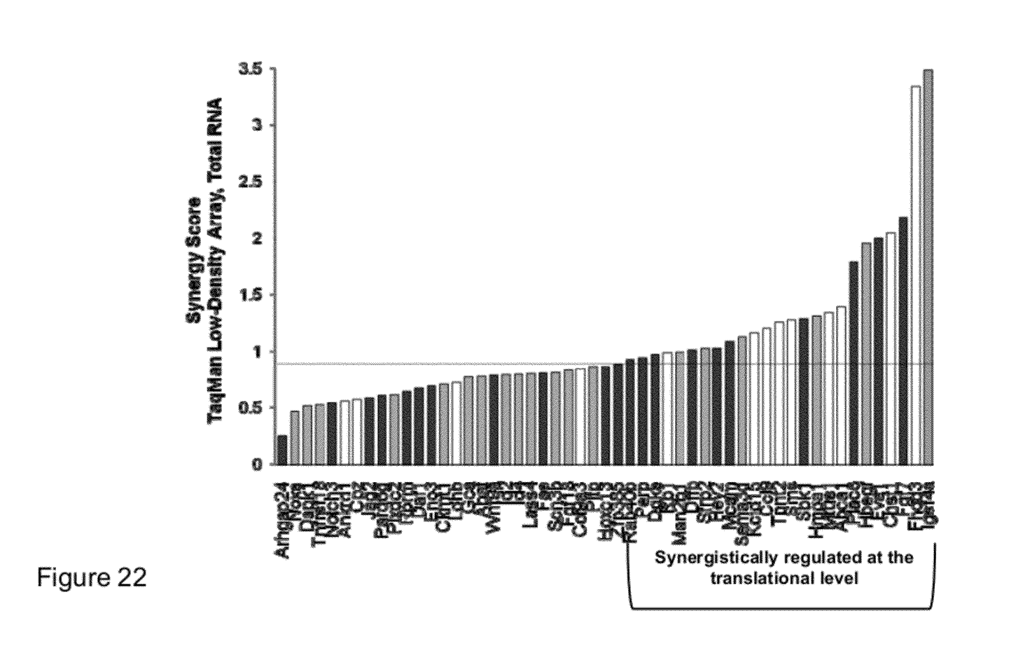

While all CRGs appear synergistically regulated in polysomal RNA, where they were originally identified, 25/56 CRGs examined do not appear synergistically regulated in total RNA (FIG. 22), demonstrating that the cooperative control of expression of these genes takes place at the translational, but not at the transcriptional, level. Notably, among the CRGs cooperatively regulated only in polysomal RNA are 10 genes with tumor inhibitory effects. Thus, oncogene cooperation driving cell transformation controls downstream targets at every level or regulation, including transcriptional, translational and post-translational levels.

(3) Oncogene Cooperation Overrides Extracellular Signals to Dictate Gene Expression Patterns

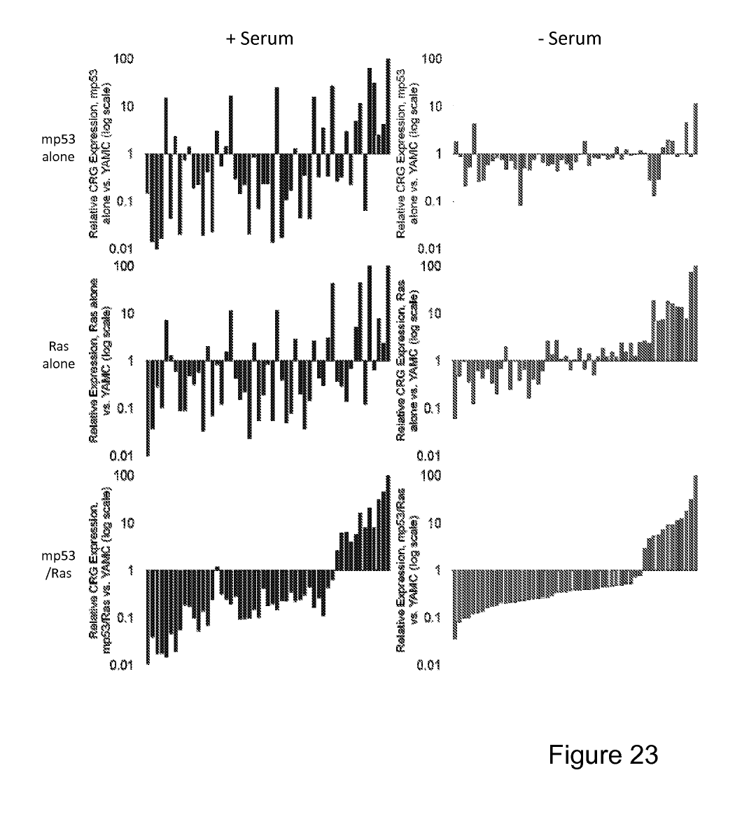

While normal cell behavior is dictated by cell responses to extracellular cues, cancer cells acquire the capability to ignore such signals, and grow or survive inappropriately. To test whether cooperating oncogenic mutations drive this aspect of the state change of the cancer cell, CRG expression profiles were compared from YAMC, mp53, Ras and mp53/Ras cells, grown in the presence or absence of serum for 24 hours prior to harvesting. While gene expression patterns in cells with mp53 alone or Ras alone is highly conditional on extracellular signals, the mp53/Ras gene expression pattern is largely independent of external cues from serum (FIG. 23). CRG expression patterns were compared using Taqman Low Density Arrays, with four replicates each of RNA from appropriate cell populations. Cooperating oncogenic mutations, thus, appear to dictate cellular responses to external stimuli as part of the comprehensive change in the state of the cell during transformation.

(4) CRGs Mediate Tumorigenicity of Pancreatic and Prostate Cancer Cells

As CRGs represent the synergistic response of cells to cooperating oncogenic mutations, disregulation of these genes is involved in malignant transformation in different types of human cancer with a similar spectrum of mutations as the murine colon cell model. Thus, it was determined whether CRGs play a role in the tumorigenicity of human pancreatic cancer, which frequently has mutations in the p53 and Ras genes, and prostate cancer, frequently characterized by p53 and PTEN mutation. CRGs disregulated in these tumors were identified by comparative genomics, based on publicly available microarray analysis of gene expression patterns in human pancreatic or prostate cancer samples. For the analysis, gene expression levels in human tumor samples were compared with normal controls, to identify CRGs disregulated in each human cancer type. The relative expression values from pancreatic or prostate cancer were compared to the relative expression values of each CRG in mp53/Ras cells as compared to YAMC cells. As in human colon cancer, the analysis shows that a substantial proportion of CRGs are disregulated in pancreatic and/or prostate cancer. Specifically, of 69 CRGs represented on the human arrays used for pancreatic samples, 33 appear similarly disregulated in pancreatic cancer as in the murine colon model system (FIG. 10, FIG. 11A). Of these 33 genes, 25 are significantly differentially expressed in pancreatic cancer. For human prostate cancer, of 47 CRGs represented on the arrays, 31 appear disregulated in the same direction as in the colon model system, with significant differences between cancer and normal samples for 23 of these genes (FIG. 10, FIG. 11B). Notably, there is a substantial overlap between these three cancers, with 10 CRGs disregulated in all three cancer types. These results show that CRGs are disregulated in cancers other than colon, and indicates that CRGs have a similar biological role in pancreatic and prostate cancer cells.

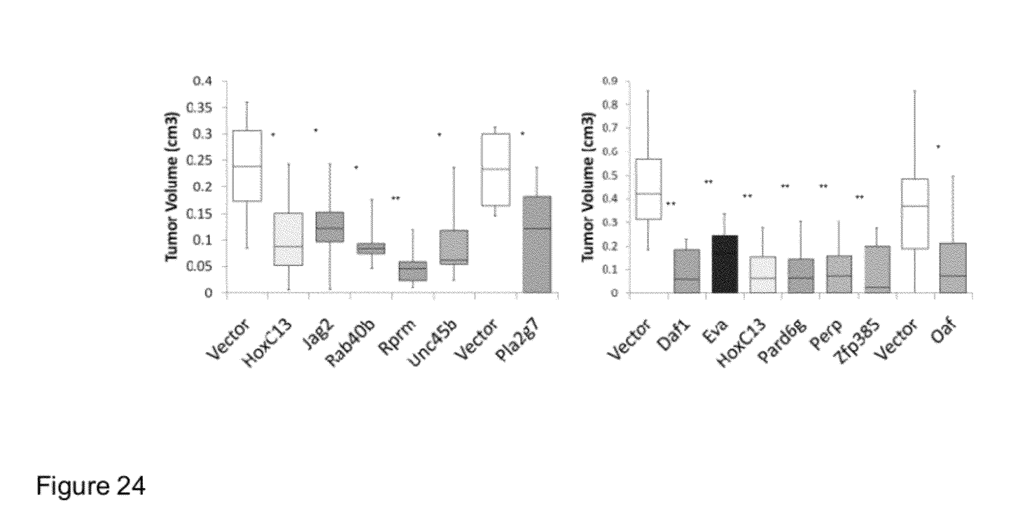

To directly test the whether CRGs control the tumor formation capacity of human pancreatic and prostate cancer cells, gene perturbation experiments were performed. A set of CRGs was perturbed in either CaPan-2 pancreatic cancer cells, which harbor p53 and Ras mutations, or in PC3 prostate cancer cells, which harbor p53 and PTEN mutations. In the case of both cancer cell lines, perturbation of CRG expression significantly inhibited the ability of cells to form tumors upon xenograft in nude mice (FIG. 24). These results indicate that the importance of CRGs is not limited to colon cancer cells, but extends to multiple human cancer types, providing a sizeable new target space in difficult to treat cancers, such as pancreatic cancer and androgen-independent prostate cancer.

b) Discussion

Taken together, the results show that genes whose expression is driven by the cooperation between oncogenes comprise a class essential for malignant transformation. Cooperating oncogenes appear to act through a limited set of downstream target genes to engender the properties of the cancer cell. Identification of the genome-wide set of genes synergistically regulated by p53 loss-of-function and constitutive Ras activation provides a roadmap to find these critically important downstream targets of cooperating oncogenes. Further characterization of this gene set reveals additional genes essential for transformation, with an overall proportion of >50% of CRGs critical to malignant transformation individually (FIG. 20). Genes regulated by the cooperation between oncogenic mutations represent an enriched set of control points in the tumor formation capacity of transformed cells, both mouse and human. Such “cooperation response addiction” opens up a wide range of cancer therapeutic targets from among these genes. Therapies that act downstream of initiating oncogenic lesions have the potential to ablate tumor formation despite the persistence of these oncogenes. Importantly, CRG perturbation can reduce or ablate tumor formation on a background of loss of p53 function, which currently confounds most chemotherapeutic strategies. The data indicates that restoring p53 function is not essential for disrupting tumor formation but can be replaced by targeting p53-negative tumors at the level of CRGs downstream of oncogenic mutations.

Among the 24 tumor inhibitory CRGs described here, a novel role in controlling malignant transformation was shown for 18 of these genes. Notably, a number of these CRGs are implicated in either regulation of cellular metabolism and transport, including Eno3, an isoform of enolase, a glycolytic enzyme normally expressed in muscle tissue, Atp8a1, a P-type ATPase/aminophospholipid translocase, and Ank (ANKH), a pyrophosphate transporter, or cell adhesion and/or cell motility, such as Mpzl2, an Ig super-family cell surface protein, Pyr14, encoding the cell adhesion molecule Nectin-4, Stmn4, a regulator of microtubule dynamics. These cellular processes are minimally represented among known oncogenes and tumor suppressors (FIG. 20C), revealing a novel target space for tumor inhibition via rational targeting of cancer cell metabolism, not previously observed.

In addition, the set of CRGs regulating carcinogenesis also includes a number of cell signaling molecules, such as Sbk1, an SH3 binding domain kinase, Prkg1, a cGMP-dependent protein kinase, and Arhgap24, a Rac and cdc42 GTPase activating protein. Several CRGs, including Dgka, a kinase involved in cell signaling by converting diacylglycerol to phosphatidic acid [29], Daf1/CD55, an inhibitor of the complement cascade, CxCl1, a chemokine receptor, and Pitx2, a homeobox-related transcription factor, show altered expression in human cancer, but have never before been shown to regulate tumorigenicity. Lastly, among CRGs with a newly identified causal role in carcinogenesis are five genes of unknown function, Bbs7, Oaf, Pard6g, Rab40b and Unc45b.

Several CRGs appear to play a distinct role in colon cell transformation by mp53 and Ras, as compared other cancers. For example, Satb1, a nuclear matrix attachment protein, is up-regulated in human breast cancers, and loss of this gene prevents breast cancer metastasis, while in mp53/Ras cells, Satb1 is down-regulated, and restoration of its expression suppresses tumor formation capacity of these cells. Moreover, Dixdc1, a positive regulator of the Wnt signaling pathway, and Mcam, a cell adhesion molecule implicated in melanoma metastasis, are down-regulated in colon cells transformed by mp53/Ras expression, and the re-expression of either of these genes significantly inhibits tumor growth from mp53/Ras cells.

Finally, the Notch signaling pathway plays a complex role in cancer progression, with context dependent effects in either promoting or suppressing tumorigenesis. Consistent with the previous results that re-expression of the Notch ligand, Jag2, was highly tumor suppressive in colon cancer cells, re-expression of the down-regulated CRGs Notch3, or the canonical Notch target gene, Hey2, are shown here to reduce tumor formation in mp53/Ras cells, supporting the idea that in colon cancer cells with multiple additional mutations, Notch signaling can antagonize tumor formation. Finally, the CRG EphB2, a member of the Ephrin family of cell guidance receptors, has a known role in suppressing colon cancer progression, consistent with the loss of EphB2 expression in the mp53/Ras transformation model and the tumor suppressive role reported here.

Synergistic regulation of gene expression appears to be controlled at multiple levels, including transcription and translation. The data disclosed herin shows synergistic regulation of protein activation, these results indicate that cooperating oncogenic lesions operate at multiple cellular levels to control the state of the cell. Identification of the first cancer synergome, the set of genes synergistically regulated by p53 loss-of-function and constitutive Ras activation, provides a roadmap to find downstream targets of critical importance to the cancer cell. This mp53-Ras synergome appears to represent a cancer causality signature required for maintenance of the malignant state, because reversal of individual CRG expression to normal cell levels can inhibit tumor formation by perturbed cells. Reversal of this state and its components represents a broad opportunity for new cancer therapeutic interventions.

Inhibiting or activating individual CRGs promotes tumor regression, as genetic perturbation of these genes inhibits tumor formation of both murine and human transformed cells in xenograft models. Reversal of the CRG signature is useful to identify compounds with the power to inhibit or reverse malignant transformation, similar to efforts made in leukemia and lymphoma. Since the CRG signature represents the transformed state, and is causally related to maintaining transformation, then compounds which can reverse this gene expression pattern have the power to inhibit tumor formation of cells dependent on CRGs. Also, since reversal of the CRG signature can predict therapeutic utility of chemotherapeutic compounds, it is important to identify the spectrum of cancers dependent on CRGs.

Finally, multiple instances were identified where CRGs interact to control cell transformation (FIG. 20B). Recent data indicates that inhibition of multiple initiating oncogenes is more effective at inducing tumor regression than inactivating a single oncogene. Like the initiating oncogenic lesions, which synergize to drive malignant transformation, CRGs can themselves interact to support this state. Thus, combined perturbation of CRGs can reduce tumor formation of transformed cells and reveal further interactions within the CRG set. Understanding the rules controlling the outcome of such interactions can reveal additional therapeutic opportunities.

The current results demonstrate the importance of non-oncogene addiction to synergistically regulated genes in cancer. Genes regulated by the cooperation between oncogenic mutations represent an enriched set of targets with the capacity to control tumor formation of transformed cells of distinct tissues. Therapies that act downstream of initiating oncogenic lesions have the potential to abrogate tumor formation despite the persistence of these oncogenes. Importantly, CRG perturbation can reduce or ablate tumor formation on a background of loss of p53 function, which currently confounds most chemotherapeutic strategies. The data indicates that restoring p53 function is not essential for disrupting tumor formation. It is possible to target p53-negative tumors downstream of p53 and inhibit tumor growth.

c) Materials and Methods

(1) Cells

Four polyclonal cell populations, control (Bleo/Neo), mp53 (p53^{175H}/Neo), Ras (Bleo/Ras^{v12}) and mp53/Ras (p53^{175H}/Ras^{v12}) were derived and used as previously described. Cells were cultured on collagen IV-coated dishes (1 μg/cm² for 1.5 hr at room temp; Sigma) in RPMI 1640 medium (Invitrogen) containing 10% (v/v) fetal bovine serum (FBS) (Hyclone), 1×ITS-A (Invitrogen), 2.5 μg/ml gentamycin (Invitrogen), and 5 U/ml interferon γ (R&D Systems). All experiments testing the effects of CRG perturbations were carried out at the non-permissive temperature for large T function (39° C.) and in the absence of interferon γ. Human cell lines CaPan-2 pancreatic cancer cells and PC3 prostate cancer cells were grown in RPMI 1640 medium (Invitrogen) containing 10% (v/v) fetal bovine serum (FBS) (Hyclone), and 2.5 μg/ml gentamycin (Invitrogen).

(2) Genetic Perturbation of Gene Expression

(a) Re-Expression of Down-Regulated Genes:

cDNA clones were obtained from the IMAGE consortium collection, distributed by Open Biosystems or PCR-cloned from YAMC cDNA using sequence-specific primers. All cDNAs were sequence-verified prior to use and were cloned into the retroviral vector pBabe-puro. For combined perturbation of Dapk, Noxa and Sfrp2, cDNA for Dapk or Noxa was sub-cloned into the pBabe-hygro retroviral vector, allowing for consecutive selection for each gene introduced. Retroviruses for infection of mp53/Ras cells were produced following transient transfection of ΦNX-eco cells (ATCC). For production of pseudotyped, human cell infectious retrovirus, pBabe retroviral vectors were co-transfected with the VSV-G gene driven by the CMV promoter into ΦNX-eco cells (ATCC). Infections were carried out in media with 8 μg/mL polybrene at 33° C. for mp53/Ras cells and at 37° C. for CaPan-2 and PC3 cells. Selection with 2.5-5 μg/mL puromycin, and where applicable, 100-200 μg/mL hygromycin B, was used to generate polyclonal populations of cells stably expressing the indicated cDNAs. To test reproducibility of the highly frequent effects of CRG gene perturbations on tumor formation, up to 4 independent replicates of such cell populations were derived. As expected, the magnitude of perturbation varies between cDNAs and replicates, and falls into the following groups. For tumor-inhibitory CRGs, all replicates express cDNAs at levels below, at or moderately above YAMC mRNA expression levels, except for Pyr14, Rab40b, and Stmn4.

(b) Knock Down of Up-Regulated Genes

shRNA molecules were designed using an algorithm. Target sequences were synthesized as forward and reverse oligonucleotides (IDT), which were annealed and cloned into the pSuper-retro vector (Oligoengine). For each up-regulated gene, two or three independent shRNA target sequences were identified yielding at least 50% reduction in gene expression with the goal to guard against off-target effects. For this purpose between four and six shRNA targets for each gene were tested. Where no effective shRNA target sequence was identified, pLKO-shRNA vectors were identified among the collection at Open Biosystems, and sets of these molecules were tested to identify appropriate knock-down constructs. For production of lentivirus, pLKO lentiviral constructs were co-transfected with the VSV-G gene and a packaging plasmid containing the gag, pol, and rev genes into 293TN cells. Retroviral or lentiviral infection of target cells was carried out as described above, except that infections and subsequent cell maintenance of mp53/Ras cells were performed at 39° C. to maximize shRNA-mediated gene knockdown. CaPan-2 and PC3 cells were infected at 37° C.

(c) Quantitation of Gene Perturbation

The efficiency of gene perturbations was tested by comparison of RNA expression levels in empty vector-infected mp53/Ras cells and cells subjected to gene perturbation. Re-expression or knock-down was also compared with the respective levels of RNA expression in YAMC control cells. For collection of RNA, mp53/Ras cells were grown at the 39° C. for 2 days, followed by serum withdrawal for 24 hr. For quantitation of gene perturbations in CaPan-2 and PC3 cells, genetically manipulated cell populations and respective vector controls were grown in the absence of serum for 24 hr prior to harvesting RNA. Total RNA was extracted from cells following the standard RNeasy Mini Kit protocol for animal cells, with on-column DNase digestion (Qiagen).

SYBR Green-based quantitative PCR was run using cDNA produced as described above for TLDA, with 1× Bio-Rad iQ SYBR Green master mix, 0.2 μM forward and reverse primer mix, with gene-specific qPCR primers for each gene tested. Primers were identified using the Primer Bank database or designed using the IDT PrimerQuest tool. Differential gene expression was calculated by the ΔΔCt method. Reactions were run on the iCycler (Bio-Rad), as follows: 5 min at 95° C., 45 cycles of 95° C. for 30 seconds, 58 to 61° C. for 30 seconds, 68 to 72° C. for 45 seconds to amplify products, followed by 40 cycles of 94° C. with 1° C. step-down for 30 seconds to produce melt curves.

(3) Xenograft Assays

Murine mp53/Ras cells were grown at 39° C. for 2 days prior to injection. Human CaPan-2 and PC3 cells were grown in standard conditions, described above. Tumor formation was assessed by sub-cutaneous injection of 5×10⁵ mp53/Ras cells or 7.5×10⁵ CaPan-2 or PC3 cells into CD-1 nude mice (Cr1: CD-1-Foxn1^{nu}, Charles River

Laboratories) in appropriate media (RPMI 1640 or DMEM) with no additives. For each replicate of all gene perturbations, 2-12 injections were performed for perturbed cells and vector controls. Tumor size was measured by caliper weekly for up to 6 weeks post-injection. Tumor volume was calculated by the formula volume=(4/3)Πr³, using the average of two radius measurements. Tumor reduction was calculated based on the average tumor volume following each gene perturbation as compared to the directly matched vector control tumors. Statistical significance of difference in tumor size was calculated by both the Wilcoxn signed-rank test and the t-test to assess consistency of significance calls, comparing tumors derived from perturbed cells with tumors induced by directly matching vector control cells.

(4) Western Blotting

mp53/Ras cells were grown at 39° C. for 2 days prior to lysis for Western blots. CaPan-2 and PC3 cells were grown in standard conditions, described above. Cell pellets were lysed for 20 min at 4° C. with rotation in RIPA buffer (50 mM Tris-HCL, pH 7.4, 150 mM NaCL, 1% NP-40, 5 mM EDTA, 0.1% SDS, 0.5% deoxycholic acid, protease inhibitor cocktail tablet). Lysates were clarified by centrifugation at 13,000 g for 10 min at 4° C. and quantitated using Bradford protein assay (Bio-Rad). 25 μg of protein lysate was separated by SDS-PAGE and transferred to PVDF membrane (Millipore) Immunoblots were blocked in 5% non-fat dry milk in PBS with 0.2% Tween-20 for 1 hour at RT, probed with antibodies against p53 (FL-393, Santa Cruz) for all cell lines, H-Ras (C-20, Santa Cruz) for mp53/Ras cells, Ras (Ab-1, Calbiochem) for CaPan-2 cells, phosphorylated and total Akt for PC3 cells (Cell Signaling, to assess downstream effects of PTEN loss), and tubulin (H-235, Santa Cruz) for all cell lines. Bands were visualized using the ECL+kit (Amersham).

(5) Biological Process Analysis of Gene Sets

Gene ontology classification of CRGs and oncogenes/tumor suppressors was assigned by mapping Affymetrix probe set IDs to G0 biological process categories for each gene via the Affymetrix NetAffx tool.

(6) RNA Isolation, RT and TLDA QPCR

Polysomal RNA was harvested as previously described from YAMC, bleo/neo, mp53/neo, bleo/Ras and mp53/Ras cells to obtain gene expression profiles reflective of protein synthesis rates. Total RNA was harvedted for each cell population as for assessment of gene perturbations described above. RNA samples of each type from four replicates of each cell line were used for reverse transcription reactions containing 10 μg RNA, 1× SuperScript II reverse transcriptase buffer, 10 mM DTT, 400 μM dNTP mixture, 0.3 ng random hexamer primer, 2 μL RNaseOUT RNase inhibitor and 2 μL of SuperScript II reverse transcriptase in a 100 μL reaction (all components from Invitrogen). RT reactions were carried out by denaturing RNA at 70° C. for 10 minutes, plunging RNA on to ice, adding other components, incubating at 42° C. for 1 hour and heat inactivating the RT enzyme by a final incubation at 70° C. for 10 minutes.

TaqMan Low-Density Arrays (Applied Biosystems), comprised of TaqMan qPCR reactions targeting the cooperation response genes available and control genes in a microfluidic card, were used as previously described. Briefly, for each sample, cDNA was combined with TaqMan Universal PCR Master Mix No AmpErase UNG (Applied Biosystems) and loaded into the card, which contains forward and reverse primer and a TaqMan MGB probe (6-FAM). Amplifications were done on an ABI Prism 7900HT Sequence Detection System (Applied Biosystems) with a TaqMan Low Density Array Upgrade. Thermal cycling conditions were as follows: 2 min at 50° C., 10 min at 94.5° C., 40 cycles of 97° C. for 30 seconds, and annealing and extension at 59.7° C. for 1 minute. Gene expression values were derived using SDS 2.2 software package (Applied Biosystems). Differential gene expression was calculated by the ΔΔCt method.

(7) Statistical Analysis and CRG Identification

Expression values from the TLDA were used to identify genes that respond synergistically to the combination of mutant p53 and activated Ras in total RNA samples. For each genes, a synergy score was calculated by the following metric, as previously described: Let a be the mean expression value for a given gene in mp53 cells, b represent the mean expression value for the same gene in Ras cells and d represent the mean expression value for this gene in mp53/Ras cells. Then, the selection criterion defines CRGs as

$$\frac{a + b}{d} \leq 0.9$$

for genes over-expressed in mp53/Ras cells and as

$$\frac{d}{a} + \frac{d}{b} \leq 0.9$$

for genes under-expressed in mp53/Ras cells, as compared to controls.

(8) Comparison of CRG Expression in Human Pancreatic and Prostate Cancer and mp53/Ras Cells

Publically available microarray datasets were mined for primary human cancer and normal tissue samples. Expression values from microarrays examining human primary pancreatic or prostate cancer samples and normal tissue samples of each type were obtained from the Stanford Microarray database. Representative probe sets were identified on the cDNA microarrays for 69 of the CRGs in the pancreatic cancer dataset and for 47 CRGs in the prostate cancer dataset, and used for comparison. T-statistics and unadjusted p-values were calculated by Welch's t-test, comparing the expression values for these probe sets in either pancreatic or prostate cancer compared to normal samples of the same tissue origin, and for mp53/Ras compared to YAMC samples.

### E. REFERENCES

Abdollahi, A., Pisarcik, D., Roberts, D., Weinstein, J., Cairns, P., and Hamilton, T. C. (2003). LOT1 (PLAGL1/ZAC1), the candidate tumor suppressor gene at chromosome 6q24-25, is epigenetically regulated in cancer. J Biol Chem 278, 6041-6049.
Adachi, K. et al. Identification of SCN3B as a novel p53-inducible proapoptotic gene. Oncogene 23, 7791-8 (2004).
Alizadeh, A. A., Eisen, M. B., Davis, R. E., Ma, C., Lossos, I. S., Rosenwald, A., Boldrick, J. C., Sabet, H., Tran, T., Yu, X., et al. (2000). Distinct types of diffuse large B-cell lymphoma identified by gene expression profiling. Nature 403, 503-511.
Archer, S. Y., Meng, S., Shei, A., and Hodin, R. A. (1998). p21 (WAF1) is required for butyrate-mediated growth inhibition of human colon cancer cells. Proc Natl Acad Sci USA 95, 6791-6796.
Ashburner, M. et al. Gene ontology: tool for the unification of biology. The Gene Ontology Consortium. Nat. Genet. 25, 25-9 (2000).
Attardi, L. D., Reczek, E. E., Cosmas, C., Demicco, E. G., McCurrach, M. E., Lowe, S. W., and Jacks, T. (2000). PERP, an apoptosis-associated target of p53, is a novel member of the PMP-22/gas3 family. Genes Dev 14, 704-718.
Baeg, G. H., Matsumine, A., Kuroda, T., Bhattacharjee, R. N., Miyashiro, I., Toyoshima,
K., and Akiyama, T. (1995). The tumour suppressor gene product APC blocks cell cycle progression from G0/G1 to S phase. Embo J 14, 5618-5625.
Batlle, E. et al. EphB receptor activity suppresses colorectal cancer progression. Nature 435, 1126-30 (2005).
Berenbaum, M. C. What is synergy? Pharmacol Rev 41, 93-141 (1989).
Berrar, D., et al., Survival trees for analyzing clinical outcome in lung adenocarcinomas based on gene expression profiles: identification of neogenin and diacylglycerol kinase alpha expression as critical factors. J Comput Biol, 2005. 12(5): p. 534-44.
Bild, A. H., Yao, G., Chang, J. T., Wang, Q., Potti, A., Chasse, D., Joshi, M. B., Harpole, D., Lancaster, J. M., Berchuck, A., et al. (2006). Oncogenic pathway signatures in human cancers as a guide to targeted therapies. Nature 439, 353-357.
Boiko, A. D., Porteous, S., Razorenova, 0. V., Krivokrysenko, V. I., Williams, B. R., and Gudkov, A. V. (2006). A systematic search for downstream mediators of tumor suppressor function of p53 reveals a major role of BTG2 in suppression of Ras-induced transformation. Genes Dev 20, 236-252.
Brummelkamp, T. R., Bernards, R. & Agami, R. A system for stable expression of short interfering RNAs in mammalian cells. Science 296, 550-3 (2002).
Brummelkamp, T. R., R. Bernards, and R. Agami, A system for stable expression of short interfering RNAs in mammalian cells. Science, 2002. 296(5567): p. 550-3.
Butler, L. M., Agus, D. B., Scher, H. I., Higgins, B., Rose, A., Cordon-Cardo, C., Thaler, H. T., Rifkind, R. A., Marks, P. A., and Richon, V. M. (2000). Suberoylanilide hydroxamic acid, an inhibitor of histone deacetylase, suppresses the growth of prostate cancer cells in vitro and in vivo. Cancer research 60, 5165-5170.
Carducci, M. A., Gilbert, J., Bowling, M. K., Noe, D., Eisenberger, M. A., Sinibaldi, V., Zabelina, Y., Chen, T. L., Grochow, L. B., and Donehower, R. C. (2001). A Phase I clinical and pharmacological evaluation of sodium phenylbutyrate on an 120-h infusion schedule. Clin Cancer Res 7, 3047-3055.
Certo, M., et al., Mitochondria primed by death signals determine cellular addiction to antiapoptotic BCL-2 family members. Cancer Cell, 2006. 9(5): p. 351-65.
Chen, L., et al., Differential targeting of prosurvival Bcl-2 proteins by their BH3-only ligands allows complementary apoptotic function. Mol Cell, 2005. 17(3): p. 393-403.
Chiba, T. et al. Identification and investigation of methylated genes in hepatoma. Eur J Cancer 41, 1185-94 (2005).
Chu, L. C., Eberhart, C. G., Grossman, S. A., and Herman, J. G. (2006). Epigenetic silencing of multiple genes in primary CNS lymphoma. Int J Cancer 119, 2487-2491.
D'Abaco, G. M., Whitehead, R. H., and Burgess, A. W. (1996). Synergy between Apc min and an activated ras mutation is sufficient to induce colon carcinomas. Mol Cell Biol 16, 884-891.
Deiss, L. P., et al., Identification of a novel serine/threonine kinase and a novel 15-kD protein as potential mediators of the gamma interferon-induced cell death. Genes Dev, 1995. 9(1): p. 15-30.
Demetri, G. D., et al., Efficacy and safety of imatinib mesylate in advanced gastrointestinal stromal tumors. N Engl J Med, 2002. 347(7): p. 472-80.
Denoyelle, C. et al. Anti-oncogenic role of the endoplasmic reticulum differentially activated by mutations in the MAPK pathway. Nat Cell Biol 8, 1053-63 (2006).
Downward, J. Targeting RAS signalling pathways in cancer therapy. Nat Rev Cancer 3, 11-22 (2003).
Elenbaas, B., et al., Human breast cancer cells generated by oncogenic transformation of primary mammary epithelial cells. Genes Dev, 2001. 15(1): p. 50-65.
Fanidi, A., E. A. Harrington, and G. I. Evan, Cooperative interaction between c-myc and bcl-2 proto-oncogenes. Nature, 1992. 359(6395): p. 554-6.
Fattman, C. L., Schaefer, L. M. & Oury, T. D. Extracellular superoxide dismutase in biology and medicine. Free Radic Biol Med 35, 236-56 (2003).
Fearon, E. R., and Vogelstein, B. (1990). A genetic model for colorectal tumorigenesis. Cell 61, 759-767.
Felsher, D. W., Oncogene addiction versus oncogene amnesia: perhaps more than just a bad habit? Cancer Res, 2008. 68(9): p. 3081-6; discussion 3086.
Fernandez, P. C., Frank, S. R., Wang, L., Schroeder, M., Liu, S., Greene, J., Cocito, A., and Amati, B. (2003). Genomic targets of the human c-Myc protein. Genes Dev 17, 1115-1129.
Fleming, J. B., et al., Molecular consequences of silencing mutant K-ras in pancreatic cancer cells: justification for K-ras-directed therapy. Mol Cancer Res, 2005. 3(7): p. 413-23.
Foltz, G. et al. Genome-wide analysis of epigenetic silencing identifies BEX1 and BEX2 as candidate tumor suppressor genes in malignant glioma. Cancer Res 66, 6665-74 (2006).
Fraga, M. F., Ballestar, E., Villar-Garea, A., Boix-Chornet, M., Espada, J., Schotta, G., Bonaldi, T., Haydon, C., Ropero, S., Petrie, K., et al. (2005). Loss of acetylation at Lys16 and trimethylation at Lys20 of histone H4 is a common hallmark of human cancer. Nat Genet. 37, 391-400.
Fukuchi, J. et al. Androgenic suppression of ATP-binding cassette transporter A1 expression in LNCaP human prostate cancer cells. Cancer Res 64, 7682-5 (2004).
Garraway, L. A., et al., Integrative genomic analyses identify MITF as a lineage survival oncogene amplified in malignant melanoma. Nature, 2005. 436(7047): p. 117-22.
Gilbert, J., Baker, S. D., Bowling, M. K., Grochow, L., Figg, W. D., Zabelina, Y., Donehower, R. C., and Carducci, M. A. (2001). A phase I dose escalation and bioavailability study of oral sodium phenylbutyrate in patients with refractory solid tumor malignancies. Clin Cancer Res 7, 2292-2300.
Giuriato, S., et al., Sustained regression of tumors upon MYC inactivation requires p53 or thrombospondin-1 to reverse the angiogenic switch. Proc Natl Acad Sci USA, 2006. 103(44): p. 16266-71.
Glaser, K. B., Stayer, M. J., Waring, J. F., Stender, J., Ulrich, R. G., and Davidsen, S. K. (2003). Gene expression profiling of multiple histone deacetylase (HDAC) inhibitors: defining a common gene set produced by HDAC inhibition in T24 and MDA carcinoma cell lines. Mol Cancer Ther 2, 151-163.
Godwin, A. R. & Capecchi, M. R. Hoxc13 mutant mice lack external hair. Genes Dev 12, 11-20 (1998).
Goodrich, D. W. The retinoblastoma tumor-suppressor gene, the exception that proves the rule. Oncogene 25, 5233-43 (2006).
Gore, S. D., Weng, L. J., Figg, W. D., Zhai, S., Donehower, R. C., Dover, G., Greyer, M. R., Griffin, C., Grochow, L. B., Hawkins, A., et al. (2002). Impact of prolonged infusions of the putative differentiating agent sodium phenylbutyrate on myelodysplastic syndromes and acute myeloid leukemia. Clin Cancer Res 8, 963-970.
Gottlicher, M., Minucci, S., Zhu, P., Kramer, 0. H., Schimpf, A., Giavara, S., Sleeman, J. P., Lo Coco, F., Nervi, C., Pelicci, P. G., and Heinzel, T. (2001). Valproic acid defines a novel class of HDAC inhibitors inducing differentiation of transformed cells. Embo J 20, 6969-6978.
Greulich, H., et al., Oncogenic transformation by inhibitor-sensitive and -resistant EGFR mutants. PLoS Med, 2005. 2(11): p. e313.
Gui, C. Y., Ngo, L., Xu, W. S., Richon, V. M., and Marks, P. A. (2004). Histone deacetylase (HDAC) inhibitor activation of p21WAF1 involves changes in promoter-associated proteins, including HDAC1. Proc Natl Acad Sci USA 101, 1241-1246.
Guo, D. L. et al. Reduced expression of EphB2 that parallels invasion and metastasis in colorectal tumours. Carcinogenesis 27, 454-64 (2006).
Hague, A., Manning, A. M., Hanlon, K. A., Huschtscha, L. I., Hart, D., and Paraskeva, C. (1993). Sodium butyrate induces apoptosis in human colonic tumour cell lines in a p53-independent pathway: implications for the possible role of dietary fibre in the prevention of large-bowel cancer. Int J Cancer 55, 498-505.
Hahn, W. C., et al., Creation of human tumour cells with defined genetic elements. Nature, 1999. 400(6743): p. 464-8.
Hamilton, J. P. et al. Reprimo methylation is a potential biomarker of Barrett's-Associated esophageal neoplastic progression. Clin Cancer Res 12, 6637-42 (2006).
Hanahan, D. and R. A. Weinberg, The hallmarks of cancer. Cell, 2000. 100(1): p. 57-70.
Hassane, D. C., Guzman, M. L., Corbett, C., Li, X., Abboud, R., Young, F., Liesveld, J. L., Carroll, M., and Jordan, C. T. (2008). Discovery of agents that eradicate leukemia stem cells using an in silico screen of public gene expression data. Blood.
Heerdt, B. G., Houston, M. A., and Augenlicht, L. H. (1994). Potentiation by specific short-chain fatty acids of differentiation and apoptosis in human colonic carcinoma cell lines. Cancer research 54, 3288-3293.
Hieronymus, H., Lamb, J., Ross, K. N., Peng, X. P., Clement, C., Rodina, A., Nieto, M., Du, J., Stegmaier, K., Raj, S. M., et al. (2006). Gene expression signature-based chemical genomic prediction identifies a novel class of HSP90 pathway modulators. Cancer Cell 10, 321-330.
Hildebrandt, T., van Dijk, M. C., van Muijen, G. N. & Weidle, U. H. Loss of heterozygosity of gene THW is frequently found in melanoma metastases. Anticancer Res 21, 1071-80 (2001).
Hirakawa, T. and H. E. Ruley, Rescue of cells from ras oncogene-induced growth arrest by a second, complementing, oncogene. Proc Natl Acad Sci USA, 1988. 85(5): p. 1519-23.
Hoeflich, A. et al. Insulin-like growth factor-binding protein 2 in tumorigenesis: protector or promoter? Cancer Res 61, 8601-10 (2001).
Hollander, M. and D. A. Wolfe, Nonparametric Statistical Methods. 2nd ed. 1998, Hoboken, N.J.: Wiley-Interscience. 816.
Houde, C. et al. Overexpression of the NOTCH ligand JAG2 in malignant plasma cells from multiple myeloma patients and cell lines. Blood 104, 3697-704 (2004).
Hruban, R. H., Goggins, M., Parsons, J., and Kern, S. E. (2000). Progression model for pancreatic cancer. Clin Cancer Res 6, 2969-2972.
Hruban, R. H., et al., Progression model for pancreatic cancer. Clin Cancer Res, 2000. 6(8): p. 2969-72.
Huang, E., Ishida, S., Pittman, J., Dressman, H., Bild, A., Kloos, M., D'Amico, M., Pestell, R. G., West, M., and Nevins, J. R. (2003). Gene expression phenotypic models that predict the activity of oncogenic pathways. Nat Genet. 34, 226-230.
Hughes, T. R., Marton, M. J., Jones, A. R., Roberts, C. J., Stoughton, R., Armour, C. D., Bennett, H. A., Coffey, E., Dai, H., He, Y. D., et al. (2000). Functional discovery via a compendium of expression profiles. Cell 102, 109-126.
Huusko, P. et al. Nonsense-mediated decay microarray analysis identifies mutations of EPHB2 in human prostate cancer. Nat Genet. 36, 979-83 (2004).
Iacobuzio-Donahue, C. A., et al., Exploration of global gene expression patterns in pancreatic adenocarcinoma using cDNA microarrays. Am J Pathol, 2003. 162(4): p. 1151-62.
Ihrie, R. A., Reczek, E., Horner, J. S., Khachatrian, L., Sage, J., Jacks, T., and Attardi, L. D. (2003). Perp is a mediator of p53-dependent apoptosis in diverse cell types. Curr Biol 13, 1985-1990.
Iizuka, M., and Smith, M. M. (2003). Functional consequences of histone modifications. Curr Opin Genet Dev 13, 154-160.
Ikediobi, 0. N. et al. Mutation analysis of 24 known cancer genes in the NCl-60 cell line set. Mol Cancer Ther 5, 2606-12 (2006).
Inbal, B., et al., DAP kinase links the control of apoptosis to metastasis. Nature, 1997. 390(6656): p. 180-4.
Insing a, A., Monestiroli, S., Ronzoni, S., Gelmetti, V., Marchesi, F., Viale, A., Altucci, L., Nervi, C., Minucci, S., and Pelicci, P. G. (2005). Inhibitors of histone deacetylases induce tumor-selective apoptosis through activation of the death receptor pathway. Nat Med 11, 71-76.
Isaacs, W. and T. Kainu, Oncogenes and tumor suppressor genes in prostate cancer. Epidemiol Rev, 2001. 23(1): p. 36-41.
Jat, P. S., Noble, M. D., Ataliotis, P., Tanaka, Y., Yannoutsos, N., Larsen, L., and Kioussis, D. (1991). Direct derivation of conditionally immortal cell lines from an H-2 Kb-tsA58 transgenic mouse. Proc Natl Acad Sci USA 88, 5096-5100.
Jenuwein, T., and Allis, C. D. (2001). Translating the histone code. Science 293, 1074-1080.
Jung, J. W., Cho, S. D., Ahn, N. S., Yang, S. R., Park, J. S., Jo, E. H., Hwang, J. W., Jung, J. Y., Kim, S. H., Kang, K. S., and Lee, Y. S. (2005). Ras/MAP kinase pathways are involved in Ras specific apoptosis induced by sodium butyrate. Cancer Lett 225, 199-206.
Kannangai, R., Vivekanandan, P., Martinez-Murillo, F., Choti, M. & Torbenson, M. Fibrolamellar carcinomas show overexpression of genes in the RAS, MAPK, PIK3, and xenobiotic degradation pathways. Hum Pathol 38, 639-44 (2007).
Kelly, W. K., O'Connor, O. A., Krug, L. M., Chiao, J. H., Heaney, M., Curley, T., MacGregore-Cortelli, B., Tong, W., Secrist, J. P., Schwartz, L., et al. (2005). Phase I study of an oral histone deacetylase inhibitor, suberoylanilide hydroxamic acid, in patients with advanced cancer. J Clin Oncol 23, 3923-3931.
Kelly, W. K., Richon, V. M., O'Connor, O., Curley, T., MacGregor-Curtelli, B., Tong, W., Klang, M., Schwartz, L., Richardson, S., Rosa, E., et al. (2003). Phase I clinical trial of histone deacetylase inhibitor: suberoylanilide hydroxamic acid administered intravenously. Clin Cancer Res 9, 3578-3588.
Klebanov, L., Gordon, A., Xiao, Y., Land, H., and Yakovlev, A. (2006). A permutation test motivated by microarray data analysis. Computational Statistics & Data Analysis 50, 3619-3628.
Kong, W. J., Zhang, S., Guo, C. K., Wang, Y. J., Chen, X., Zhang, S. L., Zhang, D., Liu, Z., and Kong, W. (2006). Effect of methylation-associated silencing of the death-associated protein kinase gene on nasopharyngeal carcinoma. Anticancer Drugs 17, 251-259.
Kong, W. J., Zhang, S., Guo, C., Zhang, S., Wang, Y., and Zhang, D. (2005). Methylation-associated silencing of death-associated protein kinase gene in laryngeal squamous cell cancer. Laryngoscope 115, 1395-1401.
Kuendgen, A., Schmid, M., Schlenk, R., Knipp, S., Hildebrandt, B., Steidl, C., Germing, U., Haas, R., Dohner, H., and Gattermann, N. (2006). The histone deacetylase (HDAC) inhibitor valproic acid as monotherapy or in combination with all-trans retinoic acid in patients with acute myeloid leukemia. Cancer 106, 112-119.
Kuester, D., Dar, A. A., Moskaluk, C. C., Krueger, S., Meyer, F., Hartig, R., Stolte, M., Malfertheiner, P., Lippert, H., Roessner, A., et al. (2007). Early involvement of death-associated protein kinase promoter hypermethylation in the carcinogenesis of Barrett's esophageal adenocarcinoma and its association with clinical progression. Neoplasia 9, 236-245.
Labbe, E., Lock, L., Letamendia, A., Gorska, A. E., Gryfe, R., Gallinger, S., Moses, H. L., and Attisano, L. (2007). Transcriptional cooperation between the transforming growth factor-beta and Wnt pathways in mammary and intestinal tumorigenesis. Cancer Res 67, 75-84.
Lagger, G., O'Carroll, D., Rembold, M., Khier, H., Tischler, J., Weitzer, G., Schuettengruber, B., Hauser, C., Brunmeir, R., Jenuwein, T., and Seiser, C. (2002). Essential function of histone deacetylase 1 in proliferation control and CDK inhibitor repression. Embo J 21, 2672-2681.
Lamb, J., et al., The Connectivity Map: using gene-expression signatures to connect small molecules, genes, and disease. Science, 2006. 313(5795): p. 1929-35.
Land, H., Parada, L. F., and Weinberg, R. A. (1983). Tumorigenic conversion of primary embryo fibroblasts requires at least two cooperating oncogenes. Nature 304, 596-602.
Lapointe, J., et al., Gene expression profiling identifies clinically relevant subtypes of prostate cancer. Proc Natl Acad Sci USA, 2004. 101(3): p. 811-6.
Ledford, J. G., Kovarova, M. & Koller, B. H. Impaired host defense in mice lacking ONZIN. J Immunol 178, 5132-43 (2007).
Lee, J. L., Chang, C. J., Chueh, L. L., and Lin, C. T. (2006). Secreted frizzled related protein 2 (sFRP2) decreases susceptibility to UV-induced apoptosis in primary culture of canine mammary gland tumors by NF-kappaB activation or JNK suppression. Breast Cancer Res Treat 100, 49-58.
Leiblich, A. et al. Lactate dehydrogenase-B is silenced by promoter hypermethylation in human prostate cancer. Oncogene 25, 2953-60 (2006).
Lim, K. H. and C. M. Counter, Reduction in the requirement of oncogenic Ras signaling to activation of PI3K/AKT pathway during tumor maintenance. Cancer Cell, 2005. 8(5): p. 381-92.
Lloyd, A. C., et al., Cooperating oncogenes converge to regulate cyclin/cdk complexes. Genes Dev, 1997. 11(5): p. 663-77.
Lowe, A. W., Olsen, M., Hao, Y., Lee, S. P., Taek Lee, K., Chen, X., van de Rijn, M., and Brown, P. O. (2007). Gene expression patterns in pancreatic tumors, cells and tissues. PLoS ONE 2, e323.
Lowe, S. W., Cepero, E. & Evan, G. Intrinsic tumour suppression. Nature 432, 307-15 (2004).
Lugli, A. et al. EphB2 expression across 138 human tumor types in a tissue microarray: high levels of expression in gastrointestinal cancers. Clin Cancer Res 11, 6450-8 (2005).
Luo, J., N. L. Solimini, and S. J. Elledge, Principles of cancer therapy: oncogene and non-oncogene addiction. Cell, 2009. 136(5): p. 823-37.
Lynch, T. J., et al., Activating mutations in the epidermal growth factor receptor underlying responsiveness of non-small-cell lung cancer to gefitinib. N Engl J Med, 2004. 350(21): p. 2129-39.
Madjd, Z. et al. Loss of CD55 is associated with aggressive breast tumors. Clin Cancer Res 10, 2797-803 (2004).
Marks, P. A., Richon, V. M., and Rifkind, R. A. (2000). Histone deacetylase inhibitors: inducers of differentiation or apoptosis of transformed cells. J Natl Cancer Inst 92, 1210-1216.
McCoy, M. S., Bargmann, C. I., and Weinberg, R. A. (1984). Human colon carcinoma Ki-ras2 oncogene and its corresponding proto-oncogene. Mol Cell Biol 4, 1577-1582.
McDonald, J. M. et al. Attenuated expression of DFFB is a hallmark of oligodendrogliomas with 1p-allelic loss. Mol Cancer 4, 35 (2005).
McMurray, H. R., et al., Synergistic response to oncogenic mutations defines gene class critical to cancer phenotype. Nature, 2008. 453(7198): p. 1112-6.
Mestre-Escorihuela, C., Rubio-Moscardo, F., Richter, J. A., Siebert, R., Climent, J., Fresquet, V., Beltran, E., Agirre, X., Marugan, I., Marin, M., et al. (2007). Homozygous deletions localize novel tumor suppressor genes in B-cell lymphomas. Blood 109, 271-280.
Mikesch, J. H., Buerger, H., Simon, R. & Brandt, B. Decay-accelerating factor (CD55): a versatile acting molecule in human malignancies. Biochim Biophys Acta 1766, 42-52 (2006).
Milyaysky, M., Tabach, Y., Shats, I., Erez, N., Cohen, Y., Tang, X., Kalis, M., Kogan, I., Buganim, Y., Goldfinger, N., et al. (2005). Transcriptional programs following genetic alterations in p53, INK4A, and H-Ras genes along defined stages of malignant transformation. Cancer research 65, 4530-4543.
Minn, A. J. et al. Genes that mediate breast cancer metastasis to lung. Nature 436, 518-24 (2005).
Minucci, S., and Pelicci, P. G. (2006). Histone deacetylase inhibitors and the promise of epigenetic (and more) treatments for cancer. Nat Rev Cancer 6, 38-51.
Minucci, S., Nervi, C., Lo Coco, F., and Pelicci, P. G. (2001). Histone deacetylases: a common molecular target for differentiation treatment of acute myeloid leukemias? Oncogene 20, 3110-3115.
Mitsiades, C. S., Mitsiades, N. S., McMullan, C. J., Poulaki, V., Shringarpure, R., Hideshima, T., Akiyama, M., Chauhan, D., Munshi, N., Gu, X., et al. (2004). Transcriptional signature of histone deacetylase inhibition in multiple myeloma: biological and clinical implications. Proc Natl Acad Sci USA 101, 540-545.
Morgenstern, J. P. & Land, H. Advanced mammalian gene transfer: high titre retroviral vectors with multiple drug selection markers and a complementary helper-free packaging cell line. Nucleic Acids Res 18, 3587-96 (1990).
Moustafa, M. A. et al. Comparative analysis of ATP-binding cassette (ABC) transporter gene expression levels in peripheral blood leukocytes and in liver with hepatocellular carcinoma. Cancer Sci 95, 530-6 (2004).
Muschen, M., Warskulat, U., and Beckmann, M. W. (2000). Defining CD95 as a tumor suppressor gene. J Mol Med 78, 312-325.
Narayan, G. et al. Gene dosage alterations revealed by cDNA microarray analysis in cervical cancer: identification of candidate amplified and overexpressed genes. Genes Chromosomes Cancer 46, 373-84 (2007).
Nebbioso, A., Clarke, N., Voltz, E., Germain, E., Ambrosino, C., Bontempo, P., Alvarez, R., Schiavone, E. M., Ferrara, F., Bresciani, F., et al. (2005). Tumor-selective action of HDAC inhibitors involves TRAIL induction in acute myeloid leukemia cells. Nat Med 11, 77-84.
Nevins, J. R., and Potti, A. (2007). Mining gene expression profiles: expression signatures as cancer phenotypes. Nat Rev Genet.
Nevins, J. R., Huang, E. S., Dressman, H., Pittman, J., Huang, A. T., and West, M. (2003).
Towards integrated clinico-genomic models for personalized medicine: combining gene expression signatures and clinical factors in breast cancer outcomes prediction. Hum Mol Genet. 12 Spec No 2, R153-157.
Nicolas, M. et al. Notchl functions as a tumor suppressor in mouse skin. Nat Genet. 33, 416-21 (2003).
Nishi, E. & Klagsbrun, M. Heparin-binding epidermal growth factor-like growth factor (HB-EGF) is a mediator of multiple physiological and pathological pathways. Growth Factors 22, 253-60 (2004).
Oda, E., et al., Noxa, a BH3-only member of the Bcl-2 family and candidate mediator of p53-induced apoptosis. Science, 2000. 288(5468): p. 1053-8.
Ohki, R. et al. Reprimo, a new candidate mediator of the p53-mediated cell cycle arrest at the G2 phase. J Biol Chem 275, 22627-30 (2000).
Ohno, R., Treatment of chronic myeloid leukemia with imatinib mesylate. Int J Clin Oncol, 2006. 11(3): p. 176-83.
Okada, F. et al. Impact of oncogenes in tumor angiogenesis: mutant K-ras up-regulation of vascular endothelial growth factor/vascular permeability factor is necessary, but not sufficient for tumorigenicity of human colorectal carcinoma Clark, E. A., Golub, T. R., Lander, E. S. & Hynes, R. O. Genomic analysis of metastasis reveals an essential role for RhoC. Nature 406, 532-5 (2000).
Onda, T. et al. Ubiquitous mitochondrial creatine kinase downregulated in oral squamous cell carcinoma. Br J Cancer 94, 698-709 (2006).
Panagopoulos, I. et al. Fusion of the NUP98 gene and the homeobox gene HOXC13 in acute myeloid leukemia with t(11;12)(p15;q13). Genes Chromosomes Cancer 36, 107-12 (2003).
Paraoan, L. et al. Expression of p53-induced apoptosis effector PERP in primary uveal melanomas: downregulation is associated with aggressive type. Exp Eye Res 83, 911-9 (2006).
Patnaik, A., Rowinsky, E. K., Villalona, M. A., Hammond, L. A., Britten, C. D., Siu, L. L., Goetz, A., Felton, S. A., Burton, S., Valone, F. H., and Eckhardt, S. G. (2002). A phase I study of pivaloyloxymethyl butyrate, a prodrug of the differentiating agent butyric acid, in patients with advanced solid malignancies. Clin Cancer Res 8, 2142-2148.
Peart, M. J., Smyth, G. K., van Laar, R. K., Bowtell, D. D., Richon, V. M., Marks, P. A., Holloway, A. J., and Johnstone, R. W. (2005). Identification and functional significance of genes regulated by structurally different histone deacetylase inhibitors. Proc Natl Acad Sci USA 102, 3697-3702.
Peters, D. G. et al. Comparative gene expression analysis of ovarian carcinoma and normal ovarian epithelium by serial analysis of gene expression. Cancer Epidemiol Biomarkers Prey 14, 1717-23 (2005).
Podsypanina, K., et al., Oncogene cooperation in tumor maintenance and tumor recurrence in mouse mammary tumors induced by Myc and mutant Kras. Proc Natl Acad Sci USA, 2008. 105(13): p. 5242-7.
Qi, J., Zhu, Y. Q., Luo, J., and Tao, W. H. (2006). Hypermethylation and expression regulation of secreted frizzled-related protein genes in colorectal tumor. World J Gastroenterol 12, 7113-7117.
Qin, J. Z., Stennett, L., Bacon, P., Bodner, B., Hendrix, M. J., Seftor, R. E., Seftor, E. A., Margaryan, N. V., Pollock, P. M., Curtis, A., et al. (2004). p53-independent NOXA induction overcomes apoptotic resistance of malignant melanomas. Mol Cancer Ther 3, 895-902.
Raab, G. & Klagsbrun, M. Heparin-binding EGF-like growth factor. Biochim Biophys Acta 1333, F179-99 (1997).
Radtke, F. and K. Raj, The role of Notch in tumorigenesis: oncogene or tumour suppressor? Nat Rev Cancer, 2003. 3(10): p. 756-67.
Ramaswamy, S., et al., Multiclass cancer diagnosis using tumor gene expression signatures. Proc Natl Acad Sci USA, 2001. 98(26): p. 15149-54.
Ramaswamy, S., Ross, K. N., Lander, E. S., and Golub, T. R. (2003). A molecular signature of metastasis in primary solid tumors. Nature genetics 33, 49-54.
Raveh, T., et al., DAP kinase activates a p19ARF/p53-mediated apoptotic checkpoint to suppress oncogenic transformation. Nat Cell Biol, 2001. 3(1): p. 1-7.
Rho, Y. S. et al. High mobility group HMGI(Y) protein expression in head and neck squamous cell carcinoma. Acta Otolaryngol 127, 76-81 (2007).
Richon, V. M., Sandhoff, T. W., Rifkind, R. A., and Marks, P. A. (2000). Histone deacetylase inhibitor selectively induces p21WAF1 expression and gene-associated histone acetylation. Proc Natl Acad Sci USA 97, 10014-10019.
Ridley, A. J., Paterson, H. F., Noble, M., and Land, H. (1988). Ras-mediated cell cycle arrest is altered by nuclear oncogenes to induce Schwann cell transformation. Embo J 7, 1635-1645.
Rodriguez, N. R., Rowan, A., Smith, M. E., Kerr, I. B., Bodmer, W. F., Gannon, J. V., and Lane, D. P. (1990). p53 mutations in colorectal cancer. Proc Natl Acad Sci USA 87, 7555-7559.
Rodriguez-Viciana, P. et al. Cancer targets in the Ras pathway. Cold Spring Harb Symp Quant Biol 70, 461-7 (2005).
Rogulski, K. et al. Onzin, a c-Myc-repressed target, promotes survival and transformation by modulating the Akt-Mdm2-p53 pathway. Oncogene 24, 7524-41 (2005).
Rozenblum, E., et al., Tumor-suppressive pathways in pancreatic carcinoma. Cancer Res, 1997. 57(9): p. 1731-4.
Rubinfeld, B. et al. Association of the APC gene product with beta-catenin. Science 262, 1731-4 (1993).
Saaf, A. M., et al., Parallels between global transcriptional programs of polarizing Caco-2 intestinal epithelial cells in vitro and gene expression programs in normal colon and colon cancer. Mol Biol Cell, 2007. 18(11): p. 4245-60.
Samuels, Y. et al. Mutant PIK3CA promotes cell growth and invasion of human cancer cells. Cancer Cell 7, 561-73 (2005).
Sarhadi, V. K. et al. Increased expression of high mobility group A proteins in lung cancer. J Pathol 209, 206-12 (2006).
Sato, N. et al. Aberrant methylation of Reprimo correlates with genetic instability and predicts poor prognosis in pancreatic ductal adenocarcinoma. Cancer 107, 251-7 (2006).
Schildhaus, H. U., Krockel, I., Lippert, H., Malfertheiner, P., Roessner, A., and Schneider-Stock, R. (2005). Promoter hypermethylation of p161NK4a, E-cadherin, O6-MGMT, DAPK and FHIT in adenocarcinomas of the esophagus, esophagogastric junction and proximal stomach. Int J Oncol 26, 1493-1500.
Schulze, A., Lehmann, K., Jefferies, H. B., McMahon, M. & Downward, J. Analysis of the transcriptional program induced by Raf in epithelial cells. Genes Dev 15, 981-94 (2001).
Seibold, S. et al. Identification of a new tumor suppressor gene located at chromosome 8p21.3-22. Faseb J 17, 1180-2 (2003).
Seligson, D. B., Horvath, S., Shi, T., Yu, H., Tze, S., Grunstein, M., and Kurdistani, S. K. (2005). Global histone modification patterns predict risk of prostate cancer recurrence. Nature 435, 1262-1266.
Serrano, M., Lin, A. W., McCurrach, M. E., Beach, D. & Lowe, S. W. Oncogenic ras provokes premature cell senescence associated with accumulation of p53 and p161NK4a. Cell 88, 593-602 (1997).
Sewing, A., et al., High-intensity Raf signal causes cell cycle arrest mediated by p21Cip1. Mol Cell Biol, 1997. 17(9): p. 5588-97.
Shaffer, A. L., et al., IRF4 addiction in multiple myeloma. Nature, 2008. 454(7201): p. 226-31.
Shaoul, R. et al. Elevated expression of FGF7 protein is common in human gastric diseases. Biochem Biophys Res Commun 350, 825-33 (2006).
Sharma, S. V. and J. Settleman, Exploiting the balance between life and death: targeted cancer therapy and “oncogenic shock”. Biochem Pharmacol, 2010. 80(5): p. 666-73.
Sharma, S. V. and J. Settleman, Oncogene addiction: setting the stage for molecularly targeted cancer therapy. Genes Dev, 2007. 21(24): p. 3214-31.
Sharma, S. V., et al., A common signaling cascade may underlie “addiction” to the Src, BCR-ABL, and EGF receptor oncogenes. Cancer Cell, 2006. 10(5): p. 425-35.
Shibue, T., et al., Integral role of Noxa in p53-mediated apoptotic response. Genes Dev, 2003. 17(18): p. 2233-8.
Shih, L. M., Hsu, M. Y., Palazzo, J. P. & Herlyn, M. The cell-cell adhesion receptor MeI-CAM acts as a tumor suppressor in breast carcinoma. Am J Pathol 151, 745-51 (1997).
Shirasawa, S., Furuse, M., Yokoyama, N. & Sasazuki, T. Altered growth of human colon cancer cell lines disrupted at activated Ki-ras. Science 260, 85-8 (1993).
Shu, J. et al. Silencing of bidirectional promoters by DNA methylation in tumorigenesis. Cancer Res 66, 5077-84 (2006).
Smith, M. W. et al. Identification of novel tumor markers in hepatitis C virus-associated hepatocellular carcinoma. Cancer Res 63, 859-64 (2003).
Smolen, G. A., et al., Amplification of MET may identify a subset of cancers with extreme sensitivity to the selective tyrosine kinase inhibitor PHA-665752. Proc Natl Acad Sci U S A, 2006. 103(7): p. 2316-21.
Solimini, N. L., J. Luo, and S. J. Elledge, Non-oncogene addiction and the stress phenotype of cancer cells. Cell, 2007. 130(6): p. 986-8.
Stegmaier, K., et al., Gene expression-based high-throughput screening (GE-HTS) and application to leukemia differentiation. Nat Genet, 2004. 36(3): p. 257-63.
Stegmaier, K., et al., Signature-based small molecule screening identifies cytosine arabinoside as an EWS/FLI modulator in Ewing sarcoma. PLoS Med, 2007. 4(4): p. e122.
Subramanian, A., Tamayo, P., Mootha, V. K., Mukherjee, S., Ebert, B. L., Gillette, M. A., Paulovich, A., Pomeroy, S. L., Golub, T. R., Lander, E. S., and Mesirov, J. P. (2005). Gene set enrichment analysis: a knowledge-based approach for interpreting genome-wide expression profiles. Proc Natl Acad Sci USA 102, 15545-15550.
Sugimoto, M., Gromley, A. & Sherr, C. J. Hzf, a p53-responsive gene, regulates maintenance of the G2 phase checkpoint induced by DNA damage. Mol Cell Biol 26, 502-12 (2006).
Suzuki, H., et al., Epigenetic inactivation of SFRP genes allows constitutive WNT signaling in colorectal cancer. Nat Genet, 2004. 36(4): p. 417-22.
Suzuki, H., Gabrielson, E., Chen, W., Anbazhagan, R., van Engeland, M., Weijenberg, M. P., Herman, J. G., and Baylin, S. B. (2002). A genomic screen for genes upregulated by demethylation and histone deacetylase inhibition in human colorectal cancer. Nat Genet. 31, 141-149.
Suzuki, M. et al. Aberrant methylation of Reprimo in lung cancer. Lung Cancer 47, 309-14 (2005).
Suzuki, M. et al. Methylation of apoptosis related genes in the pathogenesis and prognosis of prostate cancer. Cancer Lett 242, 222-30 (2006).
Takahashi, T. et al. Aberrant methylation of Reprimo in human malignancies. Int J Cancer 115, 503-10 (2005).
Tokunaga, E., Oki, E., Egashira, A., Sadanaga, N., Morita, M., Kakeji, Y., and Maehara, Y. (2008). Deregulation of the Akt pathway in human cancer. Curr Cancer Drug Targets 8, 27-36.
Tran, T. P., et al., Combined Inactivation of MYC and K-Ras oncogenes reverses tumorigenesis in lung adenocarcinomas and lymphomas. PLoS ONE, 2008. 3(5): p. e2125.
van de Vijver, M. J., He, Y. D., van't Veer, L. J., Dai, H., Hart, A. A., Voskuil, D. W., Schreiber, G. J., Peterse, J. L., Roberts, C., Marton, M. J., et al. (2002). A gene-expression signature as a predictor of survival in breast cancer. N Engl J Med 347, 1999-2009.
Van Lint, C., Emiliani, S., and Verdin, E. (1996). The expression of a small fraction of cellular genes is changed in response to histone hyperacetylation. Gene Expr 5, 245-253.
van't Veer, L. J., and Bernards, R. (2008). Enabling personalized cancer medicine through analysis of gene-expression patterns. Nature 452, 564-570.
Vaux, D. L., S. Cory, and J. M. Adams, Bcl-2 gene promotes haemopoietic cell survival and cooperates with c-myc to immortalize pre-B cells. Nature, 1988. 335(6189): p. 440-2.
Vega, R. B., Matsuda, K., Oh, J., Barbosa, A. C., Yang, X., Meadows, E., McAnally, J.,
Pomajzl, C., Shelton, J. M., Richardson, J. A., et al. (2004). Histone deacetylase 4 controls chondrocyte hypertrophy during skeletogenesis. Cell 119, 555-566.
Ventura, A., et al., Restoration of p53 function leads to tumour regression in vivo. Nature, 2007. 445(7128): p. 661-5.
Verdin, E., Dequiedt, F., and Kasler, H. G. (2003). Class II histone deacetylases: versatile regulators. Trends Genet. 19, 286-293.
Villar-Garea, A., and Esteller, M. (2004). Histone deacetylase inhibitors: understanding a new wave of anticancer agents. Int J Cancer 112, 171-178.
Villunger, A., et al., p53- and drug-induced apoptotic responses mediated by BH3-only proteins puma and noxa. Science, 2003. 302(5647): p. 1036-8.
Vogelstein, B., and Kinzler, K. W. (1993). The multistep nature of cancer. Trends Genet. 9, 138-141.
Vogelstein, B., Lane, D. & Levine, A. J. Surfing the p53 network. Nature 408, 307-10 (2000).
Vousden, K. H. & Lu, X. Live or let die: the cell's response to p53. Nat Rev Cancer 2, 594-604 (2002).
Wakeling, A. E., Inhibitors of growth factor signalling. Endocr Relat Cancer, 2005. 12 Suppl 1: p. S183-7.
Wang, W., F. Rastinejad, and W. S. El-Deiry, Restoring p53-dependent tumor suppression. Cancer Biol Ther, 2003. 2(4 Suppl 1): p. S55-63.
Wang, X. and B. Seed, A PCR primer bank for quantitative gene expression analysis. Nucleic Acids Res, 2003. 31(24): p. e154.
Wei, G., Twomey, D., Lamb, J., Schlis, K., Agarwal, J., Stam, R. W., Opferman, J. T., Sallan, S. E., den Boer, M. L., Pieters, R., et al. (2006). Gene expression-based chemical genomics identifies rapamycin as a modulator of MCL1 and glucocorticoid resistance. Cancer Cell 10, 331-342.
Weinstein, I. B. and A. Joe, Oncogene addiction. Cancer Res, 2008. 68(9): p. 3077-80.
Weinstein, I. B., Cancer. Addiction to oncogenes—the Achilles heal of cancer. Science, 2002. 297(5578): p. 63-4.
Westfall, P. H. & Young, S. S. Resampling-based multiple testing: examples and methods for P-value adjustment (Wiley, New York, 1993).
Whitehead, R. H., VanEeden, P. E., Noble, M. D., Ataliotis, P., and Jat, P. S. (1993). Establishment of conditionally immortalized epithelial cell lines from both colon and small intestine of adult H-2 Kb-tsA58 transgenic mice. Proc Natl Acad Sci USA 90, 587-591.
Wong, T. S., Kwong, D. L., Sham, J. S., Wei, W. I. & Yuen, A. P. Methylation status of Reprimo in head and neck carcinomas. Int J Cancer 117, 697 (2005).
Wu, C. H., et al., Cellular senescence is an important mechanism of tumor regression upon c-Myc inactivation. Proc Natl Acad Sci USA, 2007. 104(32): p. 13028-33.
Xia, M. and H. Land, Tumor suppressor p53 restricts Ras stimulation of RhoA and cancer cell motility. Nat Struct Mol Biol, 2007. 14(3): p. 215-23.
Xiang, Y., Lin, G., Zhang, Q., Tan, Y. & Lu, G. Knocking down Wnt9a mRNA levels increases cellular proliferation. Mol Biol Rep (2007).
Yamayoshi, T. et al. Expression of keratinocyte growth factor/fibroblast growth factor-7 and its receptor in human lung cancer: correlation with tumour proliferative activity and patient prognosis. J Pathol 204, 110-8 (2004).
Yang, J. et al. Twist, a master regulator of morphogenesis, plays an essential role in tumor metastasis. Cell 117, 927-39 (2004).
Yasuhara, T. et al. FGF7-like gene is associated with pericentric inversion of chromosome 9, and FGF7 is involved in the development of ovarian cancer. Int J Oncol 26, 1209-16 (2005).
Yu, J. et al. Identification and classification of p53-regulated genes. Proc Natl Acad Sci U S A 96, 14517-22 (1999).
Yuan, B., Latek, R., Hossbach, M., Tuschl, T. & Lewitter, F. siRNA Selection Server: an automated siRNA oligonucleotide prediction server. Nucleic Acids Res 32, W130-4 (2004).
Zang, X. P., Lerner, M. R., Dunn, S. T., Brackett, D. J. & Pento, J. T. Antisense KGFR oligonucleotide inhibition of KGF-induced motility in breast cancer cells. Anticancer Res 23, 4913-9 (2003).
Zhang, C. L., McKinsey, T. A., Chang, S., Antos, C. L., Hill, J. A., and Olson, E. N. (2002). Class II histone deacetylases act as signal-responsive repressors of cardiac hypertrophy. Cell 110, 479-488.
Zhang, X., Jin, B., and Huang, C. (2007). The PI3K/Akt pathway and its downstream transcriptional factors as targets for chemoprevention. Curr Cancer Drug Targets 7, 305-316.
Zhao, R. et al. Analysis of p53-regulated gene expression patterns using oligonucleotide arrays. Genes Dev 14, 981-93 (2000).
Zhu, P., Martin, E., Mengwasser, J., Schlag, P., Janssen, K. P., and Gottlicher, M. (2004). Induction of HDAC2 expression upon loss of APC in colorectal tumorigenesis. Cancer Cell 5, 455-463.
Zou, H., Molina, J. R., Harrington, J. J., Osborn, N. K., Klatt, K. K., Romero, Y., Burgart, L. J., and Ahlquist, D. A. (2005). Aberrant methylation of secreted frizzled-related protein genes in esophageal adenocarcinoma and Barrett's esophagus. Int J Cancer 116, 584-591.

What is claimed is:

1. A method for identifying targets for the treatment of a cancer comprising performing an assay that measures differential expression of a gene or protein and identifying those genes, proteins, or micro RNAs that respond synergistically to the combination of two or more cancer genes.

2. The method of claim 1, wherein the cancer genes are selected from the group consisting of ABL1, ABL2, AF15Q14, AF1Q, AF3p21, AF5q31, AKT, AKT2, ALK, ALO17, AML1, AP1, APC, ARHGEF, ARHH, ARNT, ASPSCR1, ATIC, ATM, AXL, BCL10, BCL11A, BCL11B, BCL2, BCL3, BCL5, BCL6, BCL7A, BCL9, BCR, BHD, BIRC3, BLM, BMPR1A, BRCA1, BRCA2, BRD4, BTG1, CBFA2T1, CBFA2T3, CBFB, CBL, CCND1, c-fbs, CDH1, c-jun, CDK4, c-kit, CDKN2A-p14^{ARF}, cDKN2A. p16^{INK4A}, CDX2, CEBPA, CEP1, CHEK2, CHIC2, CHN1, CLTC, c-met, c-myc, COL1A1, COPEB, COX6C, CREBBP, c-ret, CTNNB1, CYLD, D10S170, DDB2, DDIT3, DDX10, DEK, EGFR, EIF4A2, ELKS, ELL, EP300, EPS15, erbB, ERBB2, ERCC2, ERCC3, ERCC4, ERCC5, ERG, ETV1, ETV4, ETV6, EVI1, EWSR1, EXT1, EXT2, FACL6, FANCA, FANCC, FANCD2, FANCE, FANCF, FANCG, FEV, FGFR1, FGFR1OP, FGFR2, FGFR3, FH, FIP1L1, FLI1, FLT3, FLT4, FMS, FNBP1, FOXO1A, FOXO3A, FPS, FSTL3, FUS, GAS7, GATA1, GIP, GMPS, GNAS, GOLGA5, GPC3, GPHN, GRAF, HEI10, HER3, HIP1, HIST1H4I, HLF, HMGA2, HOXA11, HOXAI3, HOXA9, HOXC13, HOXD11, HOXD13, HRAS, HRPT2, HSPCA, HSPCB, hTERT, IGHα, IGKα, IGLα, IL21R, IRF4, IRTA1, JAK2, KIT, KRAS2, LAF4, LASP1, LCK, LCP1, LCX, LHFP, LMO1, LMO2, LPP, LYL1, MADH4, MALT1, MAML2, MAP2K4, MDM2, MECT1, MEN1, MET, MHC2TA, MLF1, MLH1, MLL, MLLT1, MLLT10, MLLT2, MLLT3, MLLT4, MLLT6, MLLT7, MLM, MN1, MSF, MSH2, MSH6, MSN, MTS1, MUTYH, MYC, MYCL1, MYCN, MYH11, MYH9, MYST4, NACA, NBS1, NCOA2, NCOA4, NF1, NF2, NOTCH1, NPM1, NR4A3, NRAS, NSD1, NTRK1, NTRK3, NUMA1, NUP214, NUP98, NUT, OLIG2, p53, p27, p57, p16, p21, p73, PAX3, PAR5, PAX7, PAX8, PBX1, PCM1, PDGFB, PDGFRA, PDGFRB, PICALM, PIM1, PML, PMS1, PMS2, PMX1, PNUTL1, POU2AF1, PPARG, PRAD-1, PRCC, PRKAR1A, PRO1073, PSIP2, PTCH, PTEN, PTPN11, RAB5EP, RAD51L1, RAF, RAP1GDS1, RARA, RAS, Rb, RB1, RECQL4, REL, RET, RPL22, RUNX1, RUNXBP2, SBDS, SDHB, SDHC, SDHD, SEPT6, SET, SFPQ, SH3GL1, SIS, SMAD2, SMAD3, SMAD4, SMARCB1, SMO, SRC, SS18, SS18L1, SSH3BP1, SSX1, SSX2, SSX4, Stathmin, STK11, STL, SUFU, TAF15, TAL1, TAL2, TCF1, TCF12, TCF3, TCL1A, TEC, TCF12, TFE3, TFEB, TFG, TFPT, TFRC, TIF1, TLX1, TLX3, TNFRSF6, TOP1, TP53, TPM3, TPM4, TPR, TRAα, TRBα, TRDα, TRIM33, TRIP11, TRK, TSC1, TSC2, TSHR, VHL, WAS, WHSC1L8, WRN, WT1, XPA, XPC, ZNF145, ZNF198, ZNF278, ZNF384, and ZNFN1A1.

3. The method of claim 1, wherein the cancer genes comprise an oncogene and loss of function of a tumor suppressor gene.

4. The method of claim 3, wherein the oncogene is selected from the list of oncogenes consisting of ras, raf, Bcl-2, Akt, Sis, src, Notch, Stathmin, mdm2, abl, hTERT, c-fos, c-jun, c-myc, erbB, HER2/Neu, HER3, c-kit, c-met, c-ret, flt3, AP1, AML1, axl, alk, fms, fps, gip, lck, MLM, PRAD-1, and trk.

5. The method of claim 3, wherein the tumor suppressor gene is selected from the list of tumor suppressor genes consisting of p53, Rb, PTEN, BRCA-1, BRCA-2, APC, p57, p27, p16, p21, p73, p14^{ARF}, Chek2, NF1, NF2, VHL, WRN, WT1, MEN1, MTS1, SMAD2, SMAD3, and SMAD4.

6. The method of claims 1, wherein in the assay measures differential gene expression.

7. The method of claim 6, wherein the assay is selected from the group of assays consisting of, Northern analysis, RNAse protection assay, PCR, QPCR, genome microarray, low density PCR array, oligo array, SAGE and high throughput sequencing.

8. The method of claims 1, wherein in the assay measures differential protein expression.

9. The method of claim 8, wherein the assay is selected from the group of assays consisting of protein microarray, antibody-based or protein activity-based assays and mass spectrometry.

10. The method of claim 1, further comprising measuring the effect of the targets on neoplastic cell transformation in vitro, in vitro cell death, in vitro survival, in vivo cell death, in vivo survival, in vitro angiogenesis, in vivo tumor angiogenesis, tumor formation, tumor maintenance, or tumor proliferation.

11. The method of claim 10, wherein the effect of the targets is measured through the perturbation of one or more targets and assaying for a change in the tumor or cancer cells relative to a control wherein a difference in the tumor or cancer cells relative to a control indicates a target that affects the tumor.

12. A method for screening for one or more agents that treat a cancer comprising contacting the agent with a target identified by the method of claim 1, wherein an agent that modulates the target such that tumor activity is inhibited is an agent that treats cancer.

13. The method of claim 12, wherein the target is a cooperation response gene selected from the list of cooperation response genes consisting of Abat, Abca1, Ank, Ankrd1, Arhgap24, Atp8a1, Bbs7, Bex1, Ccl9, Centd3, Chst1, Ckmt1, Col9a3, Cpz, Cxcl1, Cxcl15, Daf1, Dapk1, Dffb, Dgka, Dixdc, Dusp15, Elav12, Eno3, Ephb2, Espn, Eva1, Fas, F2r11, Fgf18, Fgf7, Fhod3, FHOS2, Garn13, Gca, Gpr149, Hbegf, Hey2, Hmga1, Hmga2, Hoxc13, Id2, Id4, Igfbp2, Igsf4a, Jag2, Kctd15, Lass4, Ldhb, Man2b1, Mcam, Mmp15, Mpp7, Mrpl15, Mrpplf4, Ms4a10, Mtus1, Nbea, Notch3, Noxa, Oaf, Parvb, Pard6g, Perp, Plac8, Pla2g7, Pitx2, Pltp, Plxdc2, Prkcm, Prkg, Prss22, Pvrl4, Rab40b, Rai2, Rasl11a, Rb1, Rgs2, Rprm, Rspo3, Satb1, Sbk1, Sbsn, Scn3b, Sema3d, Sema7a, Serpinb2, Sfrp2, Slc14a1, Slc27a3, Sms, Sod3, Stmn4, Tex15, Tnfrsf18, Tnnt2, Unc45b, Wnt9a, Zac1, and Zfp385.

14. The method of claim 13, wherein the target is a cooperation response gene selected from the group of cooperation response genes consisting of Abca1, Ank, Arhgap24, Atp8a1, Bbs7, Bnip3, Cox6b2, Cxcl1, Daf1, Dap, Dapk1, Dffb, Dgka, Dixdc, Eno3, Ephb2, Eva1, Fas, Fgf7, Gpr149, Hbegf, Hey2, Hmga1, Hoxc13, Id2, Id4, Igsf4a, Jag2, Mcam, Notch3, Noxa, Nrp2, Oaf, Pard6g, Perp, Pitx2, Plac8, Pla2g7, Pltp, Plxdc2, Prkg, Pvr14, Rab40b, Rb1, Rgs2, Rprm, Satb1, Sbk1, Sema3d, Sfrp2, Slc14a1, Sod3, Stmn4, Unc45b, Wnt9a, Zac1, and Zfp385.

15. A method for screening for one or more agents that treats cancer comprising contacting the agent with the one or more targets, wherein the agent modulates the activity of the target in a manner such that tumor proliferation is inhibited, and wherein the targets are selected from the group of targets consisting of Abat, Abca1, Ank, Ankrd1, Arhgap24, Atp8a1, Bbs7, Bex1, Ccl9, Centd3, Chst1, Ckmt1, Col9a3, Cpz, Cxcl1, Cxcl15, Daf1, Dapk1, Dffb, Dgka, Dixdc, Dusp15, Elav12, Eno3, Ephb2, Espn, Eva1, Fas, F2r11, Fgf18, Fgf7, Fhod3, FHOS2, Garn13, Gca, Gpr149, Hbegf, Hey2, Hmga1, Hmga2, Hoxc13, Id2, Id4, Igfbp2, Igsf4a, Jag2, Kctd15, Lass4, Ldhb, Man2b1, Mcam, Mmp15, Mpp7, Mrpl15, Mrpplf4, Ms4a10, Mtus1, Nbea, Notch3, Noxa, Oaf, Parvb, Pard6g, Perp, Plac8, Pla2g7, Pitx2, Pltp, Plxdc2, Prkcm, Prkg, Prss22, Pvr14, Rab40b, Rai2, Rasl11a, Rb1, Rgs2, Rprm, Rspo3, Satb1, Sbk1, Sbsn, Scn3b, Sema3d, Sema7a, Serpinb2, Sfrp2, Slc14a1, Slc27a3, Sms, Sod3, Stmn4, Tex15, Tnfrsf18, Tnnt2, Unc45b, Wnt9a, Zac1, and Zfp385.

16. The method of claim 15, wherein the targets are selected from the group of targets consisting of Abca1, Ank, Arhgap24, Atp8a1, Bbs7, Bnip3, Cox6b2, Cxcl1, Daf1, Dap, Dapk1, Dffb, Dgka, Dixdc, Eno3, Ephb2, Eva1, Fas, Fgf7, Gpr149, Hbegf, Hey2, Hmga1, Hoxc13, Id2, Id4, Igsf4a, Jag2, Mcam, Notch3, Noxa, Nrp2, Oaf, Pard6g, Perp, Pitx2, Plac8, Pla2g7, Pltp, Plxdc2, Prkg, Pvr14, Rab40b, Rb1, Rgs2, Rprm, Satb1, Sbk1, Sema3d, Sfrp2, Slc14a1, Sod3, Stmn4, Unc45b, Wnt9a, Zac1, and Zfp385.

17. The method of claim 15, wherein the agent inhibits the activity of the target.

18. The method of claim 17, wherein the target is a cooperation response gene selected from the group consisting of Ank, Cxcl1, Eno3, Fgf7, Gpr149, Hmga1, Id4, Igsf4a, Oaf, Pla2g7, Plac8, Pltp, Plxdc2, Rgs2, and Sod3.

19. The method of claim 15, wherein the agent enhances the activity of the target.

20. The method of claim 19, wherein the target is a cooperation response gene selected from the group consisting of Abca1, Arhgap24, Atp8a1, Bbs7, Daf1, Dapk1, Dffb, Dgka, Dixdc, Ephb2, Eva1, Fas, Hey2, Hmga1, Hoxc13, Id2, Jag2, Mcam, Notch3, Noxa, Pard6g, Perp, Pitx2, Pltp, Prkg, Pvr14, Rab40b, Rb1, Rprm, Satb1, Sbk1, Sema3d, Sfrp2, Slc14a1, Stmn4, Unc45b, Wnt9a, Zac1, and Zfp385.

21. A method of treating a cancer in a subject comprising administering to the subject one or more agents that modulate the activity of one or more cooperation response genes.

22. The method of claim 21, wherein the one or more cooperation response genes are selected from the group consisting of Abca1, Ank, Arhgap24, Atp8a1, Bbs7, Bnip3, Cox6b2, Cxcl1, Daf1, Dap, Dapk1, Dffb, Dgka, Dixdc, Eno3, Ephb2, Eva1, Fas, Fgf7, Gpr149, Hbegf, Hey2, Hmga1, Hoxc13, Id2, Id4, Igsf4a, Jag2, Mcam, Notch3, Noxa, Nrp2, Oaf, Pard6g, Perp, Pitx2, Plac8, Pla2g7, Pltp, Plxdc2, Prkg, Pvr14, Rab40b, Rb1, Rgs2, Rprm, Satb1, Sbk1, Sema3d, Sfrp2, Slc14a1, Sod3, Stmn4, Unc45b, Wnt9a, Zac1, and Zfp385.

23. The method of claim 21, wherein the activity of the cooperation response gene is modulated by modulating the expression of the gene.

24. The method of claim 21, wherein the expression of the cooperation response gene is inhibited.

25. The method of claim 24, wherein the cooperation response gene is selected from the group consisting of Ank, Cxcl1, Eno3, Fgf7, Gpr149, Hmga1, Id4, Igsf4a, Oaf, P1a2g7, Plac8, Pltp, Plxdc2, Rgs2, and Sod3.

26. The method of claim 21, wherein the expression of the cooperation response gene is enhanced.

27. The method of claim 26, wherein the cooperation response gene is selected from the group consisting of Abca1, Arhgap24, Atp8a1, Bbs7, Daf1, Dapk1, Dffb, Dgka, Dixdc, Ephb2, Eva1, Fas, Hey2, Hmga1, Hoxc13, Id2, Jag2, Mcam, Notch3, Noxa, Pard6g, Perp, Pitx2, Pltp, Prkg, Pyr14, Rab40b, Rb1, Rprm, Satb1, Sbk1, Sema3d, Sfrp2, Slc14a1, Stmn4, Unc45b, Wnt9a, Zac1, and Zfp385.

28. The method of claim 21, wherein the activity of the cooperation response gene is modulated by the administration of an antibody, siRNA, small molecule inhibitory drug, or peptide mimetic that is specific for the protein encoded by the cooperation response gene.

29. The method of claim 28, wherein the antibody is specific for the protein encoded by Ank, Cxcl1, Eno3, Fgf7, Gpr149, Hmga1, Id4, Igsf4a, Oaf, Pla2g7, Plac8, Pltp, Plxdc2, Rgs2, or Sod3.

30. The method of claim 21, wherein the cancer is selected form the group of cancers consisting of lymphoma, B cell lymphoma, T cell lymphoma, mycosis fungoides, Hodgkin's Disease, leukemias, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, gastric cancer, colon cancer, cervical cancer, cervical carcinoma, breast cancer, and epithelial cancer, bone cancers, renal cancer, bladder cancer, genitourinary cancer, esophageal carcinoma, large bowel cancer, metastatic cancers hematopoietic cancers, sarcomas, Ewing's sarcoma, synovial cancer, soft tissue cancers; and testicular cancer.

31. The method of claim 21, further comprising administering to the subject one or more anti-cancer agents.

32. The method of claim 31, wherein the anti-cancer agent is a chemotherapeutic or antioxidant compound.

33. The method of claim 31, wherein the anti-cancer agent is a histone deacetylase inhibitor.

34. The method of claim 31, wherein the agent that modulates the expression or activity of one or more cooperation response genes is selected from the group consisting of (+)-chelidonine, 0179445-0000, 0198306-0000, 1,4-chrysenequinone, 15-delta prostaglandin J2, 2,6-dimethylpiperidine, 4-hydroxyphenazone, 5186223, 6-azathymine, acenocoumarol, alpha-estradiol, altizide, alverine, alvespimycin, amikacin, aminohippuric acid, amoxicillin, amprolium, ampyrone, antimycin A, arachidonyltrifluoromethane, atractyloside, azathioprine, azlocillin, bacampicillin, baclofen, bambuterol, beclometasone, benzylpenicillin, betaxolol, betulinic acid, biperiden, boldine, bromocriptine, bufexamac, buspirone, butacaine, butirosin, calycanthine, canadine, canavanine, carbarsone, carbenoxolone, carbimazole, carcinine, carmustine, cefalotin, cefepime, ceftazidime, cephaeline, chenodeoxycholic acid, chlorhexidine, chlorogenic acid, chlorpromazine, chlortalidone, cinchonidine, cinchonine, clemizole, co-dergocrine mesilate, CP-320650-01, CP-690334-01, dacarbazine, demeclocycline, dexibuprofen, dextromethorphan, dicycloverine, diethylstilbestrol, diflorasone, diflunisal, dihydroergotamine, diloxanide, dinoprostone, diphemanil metilsulfate, diphenylpyraline, doxylamine, droperidol, epirizole, epitiostanol, esculetin, estradiol, estropipate, ethionamide, etofenamate, etomidate, eucatropine, famotidine, famprofazone, fendiline, fisetin, fludrocortisone, flufenamic acid, flupentixol, fluphenazine, fluticasone, fluvastatin, fosfosal, fulvestrant, gabexate, galantamine, gemfibrozil, genistein, glibenclamide, gliquidone, glycocholic acid, gossypol, gramine, guanadrel, halcinonide, haloperidol, harpagoside, hexamethonium bromide, homochlorcyclizine, hydroxyzine, idoxuridine, ifosfamide, indapamide, iobenguane, iopanoic acid, iopromide, isoetarine, isoxsuprine, isradipine, ketorolac, ketotifen, lanatoside
C, lansoprazole, laudanosine, letrozole, levodopa, levomepromazine, lidocaine, liothyronine, lisinopril, lisuride, LY-294002, lynestrenol, meclofenamic acid, meclofenoxate, medrysone, mefloquine, mepacrine, methapyrilene, methazolamide, methyldopa, methylergometrine, metoclopramide, mevalolactone, mometasone, monensin, monorden, naftopidil, nalbuphine, naltrexone, napelline, naphazoline, naringin, niclosamide, niflumic acid, nimesulide, nomifensine, noretynodrel, norfloxacin, orphenadrine, oxolinic acid, oxprenolol, papaverine, pentolonium, pepstatin, perphenazine, PF-00562151-00, phenelzine, phenindione, pheniramine, phthalylsulfathiazole, pinacidil, pioglitazone, piperine, piretanide, piribedil, pirlindole, PNU-0230031, pralidoxime, pramocaine, praziquantel, prednisone, Prestwick-1100, Prestwick-981, probenecid, prochlorperazine, proglumide, propofol, protriptyline, racecadotril, riboflavin, rifabutin, rimexolone, roxithromycin, santonin, SB-203580, SC-560, scopoletin, scriptaid, seneciphylline, sirolimus, sitosterol, sodium phenylbutyrate, solanine, spectinomycin, spiradoline, SR-95531, SR-95639A, sulfadimidine, sulfaguanidine, sulfanilamide, sulfathiazole, tanespimycin, terbutaline, terguride, thalidomide, thiamazole, thiamphenicol, thioridazine, ticarcillin, ticlopidine, timidazole, tiratricol, tolfenamic acid, tremorine, trichostatin A, trifluoperazine, troglitazone, tyloxapol, ursodeoxycholic acid, valproic acid, vanoxerine, vidarabine, vincamine, vorinostat, wortmannin, yohimbic acid, yohimbine, and zidovudine.

35. The method of claim 31, wherein the one or more agents that modulate the expression or activity of one or more cooperation response genes increases the expression or activity of a cooperation response gene.

36. The method of claim 35, wherein the agent is selected from the group consisting of 6-benzylaminopurine, 8-azaguanine, acetylsalicylic acid, allantoin, alpha-yohimbine, azlocillin, bemegride, benfluorex, benfotiamine, berberine, bromopride, cantharidin, carbachol, chloramphenicol, cinoxacin, citiolone, daunorubicin, desoxycortone, dicloxacillin, dosulepin, epitiostanol, ethaverine, ethotoin, etofylline, etynodiol, fenoprofen, fluorometholone, geldanamycin, ginkgolide A, hesperetin, iohexyl, ioversol, ioxaglic acid, ipratropium bromide, isoxsuprine, lisinopril, mebendazole, meclofenoxate, mephenesin, mestranol, meticrane, metoclopramide, metolazone, metoprolol, morantel, MS-275, napelline, neostigmine bromide, phenelzine, picrotoxinin, pimethixene, pipenzolate bromide, procainamide, pronetalol, propafenone, propantheline bromide, pyrimethamine, pyrvinium, quinidine, rifabutin, rolitetracycline, sanguinarine, skimmianine, S-propranolol, sulconazole, sulfametoxydiazine, sulfaphenazole, suloctidil, syrosingopine, tacrine, tanespimycin, thioguanosine, tolazamide, tracazolate, trichostatin A, trifluridine, triflusal, trimetazidine, trioxysalen, valproic acid, vidarabine, and vorinostat.

37. The method of claim 31, wherein the one or more agents that modulate the expression or activity of one or more cooperation response genes inhibits the expression of a cooperation response gene.

38. The method of claim 37, wherein the second agent is selected from the group consisting of (−)-MK-801, (+/−)-catechin, 0317956-0000, 15-delta prostaglandin J2, 2-aminobenzenesulfonamide, 3-acetamidocoumarin, 5155877, 5186324, 5194442, 7-aminocephalosporanic acid, abamectin, acebutolol, aceclofenac, acepromazine, adiphenine, AH-6809, alclometasone, alfuzosin, allantoin, alpha-ergocryptine, alprenolol, alprostadil, amantadine, ambroxol, amiloride, aminophylline, ampicillin, anabasine, arcaine, ascorbic acid, atovaquone, atracurium besilate, atropine, aztreonam, bambuterol, BCB000040, bemegride, benserazide, benzamil, benzbromarone, benzethonium chloride, benzocaine, benzonatate, benzydamine, bergenin, betamethasone, bethanechol, betonicine, brinzolamide, bucladesine, bumetanide, buspirone, butirosin, capsaicin, carbachol, carbarsone, carteolol, cefaclor, cefalonium, cefamandole, cefixime, ceforanide, cefotaxime, cefoxitin, cefuroxime, chlorcyclizine, chlorphenesin, chlortalidone, chlorzoxazone, ciclacillin, cimetidine, cinchonidine, cinchonine, clebopride, clemastine, clobetasol, clorsulon, clotrimazole, clozapine, clozapine, colchicines, colforsin, colistin, convolamine, coralyne, CP-690334-01, CP-863187, cyclopentolate, cytochalasin B, daunorubicin, decamethonium bromide, decitabine, demecarium bromide, dexamethasone, diazoxide, diclofenac, dicloxacillin, dicoumarol, dicycloverine, diethylcarbamazine, diflunisal, dihydroergocristine, dilazep, diloxanide, dinoprost, dinoprostone, diperodon, diphenhydramine, diphenylpyraline, disulfuram, dl-alpha tocopherol, dobutamine, dosulepin, doxepin, doxycycline, dropropizine, dyclonine, edrophonium chloride, enalapril, epivincamine, erythromycin, esculin, estradiol, estriol, estrone, ethotoin, etilefrine, F0447-0125, famprofazone, fasudil, felbinac, fenbendazole, fenofibrate, finasteride, florfenicol, flufenamic acid, fluocinonide, fluorocurarine, fluoxetine, fluphenazine, flurbiprofen, fluspirilene, flutamide, fluticasone, fluvastatin, fluvoxamine, foliosidine, fosfosal, fulvestrant, furosemide, fursultiamine, gabexate, geldanamycin, genistein, gentamicin, gibberellic acid, Gly-His-Lys, guanabenz, H-89, halcinonide, halofantrine, haloperidol, harmaline, harmalol, harmine, harpagoside, hecogenin, heliotrine, helveticoside, heptaminol, hydrocotamine, hydroquinine, ikarugamycin, iodixanol, iohexyl, iopamidol, ioversol, isoniazid, isopropamide iodide, isotretinoin, josamycin, kaempferol, kawain, ketanserin, ketoprofen, khellin, lactobionic acid, levobunolol, levodopa, lincomycin, lisuride, lisuride, lobelanidine, lomefloxacin, loperamide, loxapine, lumicolchicine, LY-294002, meclocycline, meclofenamic acid, mefloquine, mepyramine, merbromin, mesalazine, metamizole sodium, metampicillin, metanephrine, meteneprost, metergoline, methazolamide, methocarbamol, methoxamine, methoxsalen, methylbenzethonium chloride, methyldopate, methylergometrine, methylprednisolone, metitepine, metixene, metoclopramide, metolazone, metrizamide, metronidazole, mexiletine, mifepristone, mimosine, minaprine, minocycline, minoxidil, molindone, monastrol, monensin, moxonidine, myricetin, nabumetone, nadolol, nafcillin, naftidrofuryl, naftifine, naphazoline, naproxen, neomycin, neostigmine bromide, nimodipine, nitrofural, nizatidine, nomegestrol, norcyclobenzaprine, nordihydroguaiaretic acid, orlistat, orphenadrine, oxamniquine, oxaprozin, oxetacaine, oxolamine, oxprenolol, oxybutynin, oxymetazoline, palmatine, parbendazole, parthenolide, penbutolol, pentetrazol, pergolide, PF-00539745-00, PHA-00745360, PHA-00767505E, PHA-00851261E, phenazone, phenelzine, pheneticillin, phenoxybenzamine, phentolamine, pinacidil, pioglitazone, pirenperone, pivmecillinam, pizotifen, PNU-0230031, PNU-0251126, PNU-0293363, podophyllotoxin, practolol, prednicarbate, prenylamine, Prestwick-642, Prestwick-674, Prestwick-675, Prestwick-682, Prestwick-685, Prestwick-857, Prestwick-967, Prestwick-983, primidone, probenecid, probucol, prochlorperazine, propafenone, propranolol, pyrithyldione, quipazine, raloxifene, ramipril, R-atenolol, ribavirin, ribostamycin, rifampicin, riluzole, risperidone, rofecoxib, rolitetracycline, rosiglitazone, rotenone, rottlerin, santonin, SB-203580, scopolamine N-oxide, securinine, sertaconazole, simvastatin, sirolimus, sodium phenylbutyrate, sotalol, spiradoline, splitomicin, S-propranolol, SR-95639A, stachydrine, sulfachlorpyridazine, sulfadoxine, sulfamerazine, sulfamethoxypyridazine, sulfamonomethoxine, sulfathiazole, sulindac, syrosingopine, tacrine, tamoxifen, tanespimycin, terazosin, terguride, tetracycline, tetrandrine, tetryzoline, thapsigargin, thiamazole, thiamphenicol, thiostrepton, tiaprofenic acid, tiletamine, timidazole, tocamide, tolnaftate, topiramate, tracazolate, tranexamic acid, trapidil, tretinoin, tribenoside, trichostatin A, tridihexethyl, trifluoperazine, triflupromazine, trimethadione, trimethobenzamide, troglitazone, tubocurarine chloride, tyrphostin AG-1478, ursolic acid, valproic acid, vinblastine, vincamine, vinpocetine, vitexin, withaferin A, wortmannin, yohimbic acid, yohimbine, zalcitabine, zaprinast, zardaverine, zoxazolamine, and zuclopenthixol.

39. A method for determining whether a cancer is susceptible to treatment with an anti-cancer agent comprising measuring the expression of the cooperation response gene panel in the cancer relative to a control, wherein the responsiveness of one or more cooperation response genes indicates sensitivity to treatment.

40. The method of claim 39, wherein the anti-cancer agent is a histone deacetylase inhibitor (HDACi).

41. The method of claim 39, wherein the anti-cancer agent is selected from the group consisting of (+)-chelidonine, 0179445-0000, 0198306-0000, 1,4-chrysenequinone, 15-delta prostaglandin J2, 2,6-dimethylpiperidine, 4-hydroxyphenazone, 5186223, 6-azathymine, acenocoumarol, alpha-estradiol, altizide, alverine, alvespimycin, amikacin, aminohippuric acid, amoxicillin, amprolium, ampyrone, antimycin A, arachidonyltrifluoromethane, atractyloside, azathioprine, azlocillin, bacampicillin, baclofen, bambuterol, beclometasone, benzylpenicillin, betaxolol, betulinic acid, biperiden, boldine, bromocriptine, bufexamac, buspirone, butacaine, butirosin, calycanthine, canadine, canavanine, carbarsone, carbenoxolone, carbimazole, carcinine, carmustine, cefalotin, cefepime, ceftazidime, cephaeline, chenodeoxycholic acid, chlorhexidine, chlorogenic acid, chlorpromazine, chlortalidone, cinchonidine, cinchonine, clemizole, co-dergocrine mesilate, CP-320650-01, CP-690334-01, dacarbazine, demeclocycline, dexibuprofen, dextromethorphan, dicycloverine, diethylstilbestrol, diflorasone, diflunisal, dihydroergotamine, diloxanide, dinoprostone, diphemanil metilsulfate, diphenylpyraline, doxylamine, droperidol, epirizole, epitiostanol, esculetin, estradiol, estropipate, ethionamide, etofenamate, etomidate, eucatropine, famotidine, famprofazone, fendiline, fisetin, fludrocortisone, flufenamic acid, flupentixol, fluphenazine, fluticasone, fluvastatin, fosfosal, fulvestrant, gabexate, galantamine, gemfibrozil, genistein, glibenclamide, gliquidone, glycocholic acid, gossypol, gramine, guanadrel, halcinonide, haloperidol, harpagoside, hexamethonium bromide, homochlorcyclizine, hydroxyzine, idoxuridine, ifosfamide, indapamide, iobenguane, iopanoic acid, iopromide, isoetarine, isoxsuprine, isradipine, ketorolac, ketotifen, lanatoside C, lansoprazole, laudanosine, letrozole, levodopa, levomepromazine, lidocaine, liothyronine, lisinopril, lisuride, LY-294002, lynestrenol, meclofenamic acid, meclofenoxate, medrysone, mefloquine, mepacrine, methapyrilene, methazolamide, methyldopa, methylergometrine, metoclopramide, mevalolactone, mometasone, monensin, monorden, naftopidil, nalbuphine, naltrexone, napelline, naphazoline, naringin, niclosamide, niflumic acid, nimesulide, nomifensine, noretynodrel, norfloxacin, orphenadrine, oxolinic acid, oxprenolol, papaverine, pentolonium, pepstatin, perphenazine, PF-00562151-00, phenelzine, phenindione, pheniramine, phthalylsulfathiazole, pinacidil, pioglitazone, piperine, piretanide, piribedil, pirlindole, PNU-0230031, pralidoxime, pramocaine, praziquantel, prednisone, Prestwick-1100, Prestwick-981, probenecid, prochlorperazine, proglumide, propofol, protriptyline, racecadotril, riboflavin, rifabutin, rimexolone, roxithromycin, santonin, SB-203580, SC-560, scopoletin, scriptaid, seneciphylline, sirolimus, sitosterol, sodium phenylbutyrate, solanine, spectinomycin, spiradoline, SR-95531, SR-95639A, sulfadimidine, sulfaguanidine, sulfanilamide, sulfathiazole, tanespimycin, terbutaline, terguride, thalidomide, thiamazole, thiamphenicol, thioridazine, ticarcillin, ticlopidine, timidazole, tiratricol, tolfenamic acid, tremorine, trichostatin A, trifluoperazine, troglitazone, tyloxapol, ursodeoxycholic acid, valproic acid, vanoxerine, vidarabine, vincamine, vorinostat, wortmannin, yohimbic acid, yohimbine, and zidovudine.

42. The method of claim 39, wherein the cooperation response gene is selected from the group consisting of Abat, Abca1, Ank, Ankrd1, Arhgap24, Atp8a1, Bbs7, Bex1, Ccl9, Centd3, Chst1, Ckmt1, Col9a3, Cpz, Cxcl1, Cxcl15, Daf1, Dapk1, Dffb, Dgka, Dixdc, Dusp15, Elav12, Eno3, Ephb2, Espn, Eva1, Fas, F2r11, Fgf18, Fgf7, Fhod3, FHOS2, Garn13, Gca, Gpr149, Hbegf, Hey2, Hmga1, Hmga2, Hoxc13, Id2, Id4, Igfbp2, Igsf4a, Jag2, Kctd15, Lass4, Ldhb, Man2b1, Mcam, Mmp15, Mpp7, Mrpl15, Mrpplf4, Ms4a10, Mtus1, Nbea, Notch3, Noxa, Oaf, Parvb, Pard6g, Perp, Plac8, Pla2g7, Pitx2, Pltp, Plxdc2, Prkcm, Prkg, Prss22, Pvr14, Rab40b, Rai2, Rasl11a, Rb1, Rgs2, Rprm, Rspo3, Satb1, Sbk1, Sbsn, Scn3b, Sema3d, Sema7a, Serpinb2, Sfrp2, Slc14a1, Slc27a3, Sms, Sod3, Stmn4, Tex15, Tnfrsf18, Tnnt2, Unc45b, Wnt9a, Zac1, and Zfp385.

43. The method of claim 42, wherein the activated cooperation response gene has pro-apoptotic or anti-proliferation activity.

44. The method of claim 43, wherein the cooperation response gene is selected from the group consisting of Dapk1, Dffb, Fas, Noxa, Perp, Rprm, Sfrp2, and Zac1.

45. The method of claim 44, wherein expression of Dapk1, Dffb, Fas, Noxa, Perp, Rprm, Sfrp2, and Zac1 indicates susceptibility to histone deacetylase inhibitors.
